SEQUENCE LISTING (35 sequences)

SEQ ID NO: 1 - DNA, 1496 bp, Homo sapiens
  misc_feature 1..1496: note = C2CD6 (also known as ALS2CR11) (gene position chr2:201,485,421-201,621,182) which is positioned on the minus strand with the promoter region positioned at (chr2:201,619,183-201,620,677)
  source 1..1496: mol_type = genomic DNA
gttctatgaggacatttgcccagcaactccctgtccaatgtccaactggcaacatccttgttattgatccttgtagccaaggataattctctcaaaacaatcatttttgctttaaaaaccgttgtcttccttgacctccctgtatatgcacatagtttactgtggcacttgtattcttattgcaatgcctactcctgaataaacatcattttctttcagagagtctccctctctgttatttaggctgacaaggatatgccaagaagtagcttggatatagcagttaactctgcctttaggatatgtgtatggggatataagagttaactaaaagctgacctttgagttggtccttgaataaaagaagaatagattccaaaaataagaggaataaataatgatcctgagctaggagagtacattttgcagacctttggtttccatattaagaaattcagatttttatgtacaataaagaagttctggaattctctggttttgttctgtttgcatttatttatatttttaacttttaggttcaagggtacatgtgcaggtttgttatataggtaaattttgtgtcatgggggtgtacagattatttcatcacccaggtaataagcctagtgcccaaaaggtagtttttgatcctctcccgcctcccacccttcgccgtcaagtaggccccagtgtctgttgctcccctctttgtgtccatgtgttctcattgtttagctcgcatttataagtgagaacgtggtatttggttttctgttcctgtgttagtctgcttaggataatggccttcagctccattcgttactgcaaaggactaatacgggggcagtgaaagggatggatgacagcaggttggcagacccatgagtaaatcattgcaattgtctcgtgtgtgtgttgtcccaaattaggtgccataaagggaaaaggtaagtaagattcagatatttagggtcagaattttaaaatgtgtctgaatgtttaagattttgtggatatgacattgctgcaccaaatgagtttggaacctactagaggtttaagagggaagatgaatattaacgtgttatgtgtctgtggaacaattgggtaaagatgtcaaagtagactgttggatatttaacgtctggagctcacgggacagtgtagaggaagtaatgactgacactgtaaatcttctagaaagaaatggtgcagggagaaaaggactcgtgagggctacagggcggacaagacagtgaactaaagtggttgggccccatgaggggccttgctctccaccagcgtgctctgatttccttctaaaggagactgaaatggcctggcaagtgcttgcagcagaccctccaggctcgctaagcacgcatgcttccaaagctgcctccagccactcccagcatgggcccggcctgagtgggtcactaggcggcacgcacgcgctgggaaaatgtgagaaaggctgcgggagggccgcgcct

SEQ ID NO: 2 - DNA, 1751 bp, Homo sapiens
  misc_feature 1..1751: note = CAT (gene position chr11:34,438,934-34,472,060) which is positioned on the plus strand with the promoter region positioned (chr11:34,437,183-34,438,933)
  source 1..1751: mol_type = genomic DNA
tacaagatatgacaagatcagaaaccagtcttgtttctccatttctttctctagaaataattctttaggaattcaaatccatagtaacaaaaacaaaggaaaaaaaaacatgcttttttttatataatgaataaaggttaagaaatgactccaggtctcttatttattttgttgatatttgtgttggttagttaagaagatgatttgctcgattatttcaacatttatttattcaacaaactgtggactttggagatgaacagctgaagctagaatcctgactatcccttaatagctgtgtaactttgggcaagttatttaacctccctatacctctgtttcctaatctgtaaaatgaagataatattagtatctaactcatagggttgttgggagggttaactaagcaaatatacaaaaagctgagaatagtccctggtatatagtaagtgctcaataaattatggatgggaggatcctgacccacatggagtttacagtcttggcagtatggatcgggcaggtagaaaaagacaccaaattacacagccaacagcatctttataattgtttttaatataggaaaagtatagggtgctaaagatcaatttgtggctttcttttccaatgaactgctaaagttccacttttatgcccaagattcttatcatacttttgtaagatcaagaacaacatgtgaagtttccactattatatatccacccattatatgtagttgtactggaaaaaattaaaagctgataacttttaatttgaagatgatggtatatactatgtatatatattaaggtgcttaaagataaatcctagcacctgaggaggtgtagaaatcattcaaactctttgattacaatgacaaactagtcagtaacttactaaatattgtactatatattactaagtattttactcttcaacatagctttttaaagacacaaagcttttcaaaattcctgcttacctgggggtaaaatttggggaagcagatttctccagtgtttaaagaagccaatttggcagtgtaccagagttgaatacatttttcccatcacaagggaatacatttttcccatctaagttaagttgtttttctctggtaaaggaggaaatcaacacccatctgtacggagataaacgtttcagagtgtttttatattaaataattagtatactagtctagtaagtgataatccacgaataagttaagaagagctaagaaagaaaaagaaaagcatccatccatcctttggttgcaaataatacttacattagcgtatggcaaaatttaattttgtacagagtaatttaacccaggattgctgactttttaagagctgagaaagcatagctatggagcgcaaggccccacccagcagggtctaagtattccgtctgcaaaactggcaggccaccaacggccgcgtcccagggcggcctgaaggatgctgataaccgggagccccgccctgggttcggctatcccgggcaccccgggccggcggggcgaggctctccaattgctgggccagagcgggacccttcctttccgcaccctcctgggtatctccggtcttcaggcctccttcggagagccctgctccgagcccattgggcttccaatcttggcctgcctagcgccgagcagccaatcagaaggcagtcctcccgagggggcgggacgagggggtggtgctgattggctgagcctgaagtcgccacggactcggggc

SEQ ID NO: 3 - DNA, 1846 bp, Homo sapiens
  misc_feature 1..1846: note = COMT (gene position chr22:19,941,371-19,969,975) which is positioned on the plus strand with the promoter region positioned (chr22:19,939,926-19,941,771)
  source 1..1846: mol_type = genomic DNA
tgttaactcacatgcagtagtattaaaaaatgactcggctgggcgcagtggctcatgcctgtaatcccagcactttgggaggccaaggcaggtggatcatgaggtcaggagattgagaccatcctggctaatacagtgaaatcccgtctctactaaaaatacaaaaaaaattagccaggcgtggtggcagatgcctgtagtcccagctactcgggaggctgaggcaggagaatggcgtgaacccgggaggcggagcttgcagtgagctgagatcgcaccactgtactccagcttgggcgacagagcaagacactgtctcaaaaaaaaaaaaaaaaaaaaaaccccaaaaaaacaactcatatggtttagtaacagactgggcacgaaagggctatttaaaaaacagttgctcctctggcggaaaggaatgcctggagactacttattttatttcttactgggaggaccaggatcatcatcctattttgcatagaaggttcagtttgttctttcccttaaaataggaagataagggcattatccccctaagtctcgtatgatattccccattctgagtccagaatacctagaaatttggaatttggctattgccgtgtctggactgtgagtatgggaaggggaagcttttctgtctgttgtccccactaccgcccctcacatccgtgattctgaaccccatgataaatgccctttgaacctttttcctccttttgatgccgaatccccttttatgggactccgccagcacgggtgcatttaaccttcctgagcttgcccacctgacgcatgcagccaagaagctttgaaagatggagtgccctggctgagggttccaggtctcagcaaggccacgaggtggtgactgcctcccagaactctgttgggccaccctccgtcctggagacccccaaaacctccatagcaccaactctgcccattcacacacacagtcagccctccctcccgagtccaccgcgggcagagggccagccaccatgcctcccatagccgccatccctgggaaggagccctgaccccgtccttacacccctctacactcactgcctgtggggagagctgctcgctgtcacaagcatccagggggctgggtcgaggggccccctgcatggtgggggaccatgttgtcgtgcatggaatgaaactggccacacataacagatatggtggcgctctggtcggatctaggggagtctgtcctacaggatgaggcttgaactgtcctgagtgaccagacacccagcctctgcttcgagtgctcagttatgattattgtcctgatttagttaactgttcttcaggggctccaggaggacgagtgtgtatcctcccattgctctgtgcagcctctaacctctagagtctaggggtctggggagaagttgggaagtctggccagtggggccggtgcctggtgacctcgggaggtgggatatcatcatcttcagaactgtagttgttactgggataccagctctgggagaccacaggtgcagtcagcacagcaggaccttagacaaggcacccagccccagtttccccacctgggaagggggctacttgtggctagaagcagcccggactcctgagcaagactagaccaagaggccggtatgtggacacccccgcgtgggcacccccacggggacaccctggccaccgccgcgcggacaccctcacgaggacaccccggccgcgcggacacctaccgcggggacgccccgaccccatcctacctgctgcgccccgcgccgcgccccgcaccccgcccgccacggcctgcgtccgccaccggaagcgccctcct

SEQ ID NO: 4 - DNA, 1500 bp, Homo sapiens
  misc_feature 1..1500: note = COX7A1 (gene position chr19:36,150,922-36,152,449) which is positioned on the minus strand with the promoter region positioned (chr19:36,152,448-36,153,947)
  source 1..1500: mol_type = genomic DNA
ggaaaaacctaacattaaatttacaagcatagtttgaaagtgttaggggaatttaataatcataaaagccctctttaaaagagaccgtggctggaagcggtggctcaggcctgtaattccagcactttggaaggccaaagtgggcggatcacctgaggttgggagcttgagaccagcctggccaacatggtgtaaccccatctctactaaaaaatacaaaaatgagctgggtgtggtggcgggtgcctgtaatcccagctgctcaggaggctgaggtacaagaattgcctaaacccaggaggcagaaggtttcagtgagccgagatcacaccactgcactccaacgtgtacaacagagcaagactctgtctaaaaatataaattaaataaataaataaaagagattgttaaaaccttttacaagctgggtatagtggctcatgcctctaattctagcactttgggtgatagaggtaggaacattacttggaggagttggagaccaccctgggcaacatagcaagacactgcctcaaaaacaaacaaacaaacattgtctgctggctgggtgcgtggctcacgatgaggccggcaaatttcttgagctcaggagttcgacaccagcctgggcaacatggtgaaacccagcctctacaaaaaatacgaaaattagttcgagcatggtggtgggcgcctgtagtcccagccactcaggaggctgaggcaggaggatcgctgaacccaggaggtcgaggctgcagtgagctatgaccgcaccactgcactccagcctgtgcaacagagcaaaaccctgtctaaaaaaaaaagaaaaaaaagaaaagaaaaagaaaaccttgtagactaggccaggatctttcctggttgctaaaaccattaggacaactgtgctgcaccccgttgactgccagaattagagagctataaatagatatgatgtgtttcttagggtaacataaaataccgttttactcccaaagactttagcttgtgtttcctaaaaacaaggtcatttaatgtctcaacaaatctctgagaaagatcctgccttagccctcacgttacagatacggaaacaggctcggaggtgcaatctctggctgtgtagacagaacagggagttggaacacggccttatgcgtttcctcactctcacataccagcgggcagggagagatccggggaggagttgcacttgcccccggcggaaatctccaaaaatctgcaaaaatgtattccctggtaccgctttgggctccggtgggaccaagcgaaattccgagaccgaaacggatgcgcgctgcggcccagggtgcgggtctggaccgcctcctccgtggtggacgaatccgaggagcaggactccccgcaacccagccccagccccagccccagccccgccgcgtccccagctgtcacgctgcgcgcagcgggtggggcctggggttcctggacagaggaggactacgcgtgtccttgggc

SEQ ID NO: 5 - DNA, 1548 bp, Homo sapiens
  misc_feature 1..1548: note = GYPE (gene position chr4:143,870,864-143,912,131) which is positioned on the minus strand with the promoter region positioned (chr4:143,912,132-143,913,679)
  source 1..1548: mol_type = genomic DNA
tcttacttctcccgtgacttgtctactcatcctttaggtctcattgcttgctctggaaaacctttccagaacctcaaccctgagttaggtacccctcttatgtacccttataacaccctttgcttactactatcatccacttgacactccataatgcaattgccttcttaatttatttgtttccaaattaagctgtcagttcctgaagggaaacttgagttactgaaggttgtgtcttccacacttggcacagtgcccggtaggtgctcacaaatcattggttgaattattatttggatgtagaacaagctggacctatcaattctcagcagaaagattatgtatgatacctgattccagcagtatagcacgatggacttagaggcaactgggaacagttttccactgtctacatcaccattgtagtgatgagagtttctcacttctggggtttcttcaacgcacatcacatctcattataattttaaagaaaggaaactgaaaactgggttaaagaaatctcatcactagaaggtgttgaagaagactcctaaataaattagatttccaaacccaaatgccaaaagcttagaaagagtataaatgaatgcgatggattgtgtttttgctcccaattattagggcccttcctgtaagtgttttatatatcctcaccattgacctatgacttgcaggacttccatttggaagaattatatttacttgtcccattgaggtcagcttggccactgatttactttggctaatagaatgtgagaaaaaaaccataacacagtctgagaggaagcattaaaagccattccttatctggcctttttcccccagtttgcaacaagaacagcatattccaaatgggggtggctccttcagtctggatcctggaacttgtcaccgtcatagaccaaatgtaatgtgagcaggaaatgaaccagtgttgctgcaagacactgagattttgaggccttaccacagtagaacctaatctaagaaatggaagcaagcatcaagtggagcacttaccagactcaagaaagcctatttgtcccaagagacagccagtccttagctctcatgaattgtcacaatgtagaactgcacgctaactcttgccagatcttcctgtaagaggagccaaattttaaattaaatcaattcctgatttttaattaaaatataccttctttttaaatgttggcgctaaatccatacgtttaaaaaacacttagggcaaatcaaatcaagtaataactcataatctcttttccattccatagtttgctcatacataaaataagaatgcgctggcccggcgtcgctgggctttcctcaaggggtccccgagcagcgtcgcagagcgggccgacttccgggaaggaagtgaccagcgactgagcggcggccggcgcgtttagcgccctgaacatgcggcagtccctgcgggcgaccccaggctccggacaggcagcggcggaggcggcggctccggagggaaggaggcggcggcgccggcggaggtggcggcggagg

SEQ ID NO: 6 - DNA, 1473 bp, Homo sapiens
  misc_feature 1..1473: note = IHO1 (also known as CCDC36) (gene position chr3:49,198,428-49,258,106) which is positioned on the plus strand with the promoter region positioned (chr3:49,198,025-49,199,497)
  source 1..1473: mol_type = genomic DNA
attttctcgaattttatataaatgggatcatacagtatgtattcttttgtgtttggctttcactcagcgtaattattttgaaatttattcatgttgttgtatccattgttcattcctttttattgcttagtagttgtccattgtatggatatgccaaatctctcctcttgattttttgaatccatacacaaatcaggttttcatcctcactacatcactgtaactgctcctaccaaggtcactcaaagttaattctaactaatcttccctgaacacatccgcagcatttgtcatagctgatctctgcctcctctttgaagcagttccaagtgccttccagaatgcctcattctcctggtgtctttccccgtctcctgaccaggcttgctccaactccttggctgcttttccttcatctccaaagtcctctaaactggtgtgtcctggggctgaatccaatctcctttccatttatactccattggtgattgcatcctgtttttgtttgttttttgagacagggtctcattctgtcgcccaggctggagtgtagtgacgcgatctcggctcactgcaacctctacctcccaggttcaaacgatcctcccgcctcagcctccgagtagctgggaccacaggcatgtgttaccacgccggactaattttttgtagagctggggtttcgccatgctgcccaggctggtctcaagcatgttttttgctttctaagtcatctataagcaccagaggcagagttacatagatccttcctgcctacccgcactgtccctgccctcaggccactccagcatccagcaggccagaaacctcagaagatttcttgcctcctttctctttcctccaggggcccggttcgatgtccccatctgcccacggcgcctctcgtgccagctgctccgcctgtcctgcccttgctcagaccacccaagtatcatctgtgctctggcccctggccaccagttccaccccacatgctgtcctcacaggtggcgccttgccattcctggggaccagggcctccttgttctggtcatacacagtggagtgtaggtcgagcgacaagtgagtagatctgcatgccaggtgggccgagggtggccattccggcgtgagtgtgggggtgtgagggaacgttgcatggggtgcacgtgggctgggtgtggcgtgcagcggtgtggcgccaggcaggcagcttggcggggcccacaatcagcgcggttgagaccattagtacggttaaaaccgttagctgcctgtcgcggcgagggcggggccctggggactggacggaatcccagttggtcagaggagaccctgggggcggggccgcgcgagttgccgttaggacagttaaaaccgttagctgcctgtcgtggcggggcggggccctgggacaggacggaatccctattggtcagaagaggacctgggggcggggccgcgcgtgccgtt

SEQ ID NO: 7 - DNA, 1456 bp, Homo sapiens
  misc_feature 1..1456: note = KRBOX1 (gene position chr3:42,809,483-42,942,792) which is positioned on the plus strand with the promoter region positioned (chr3:42934886-42936341)
  source 1..1456: mol_type = genomic DNA
ataagattcttcttgccttaccttcaattgtatgtgtgtatgtgtgtgtgcacacatttgatgatgtccatagagaagaatatacaagtgggtggaaattctgcttgtgtctgtggttcacaggagttctgtagtctctatattctcaggctagccaacacttggacctcagcaactcattaaacattttagctgatttctccttaacagcttacatggcatccaatggcatctgtcctatgtaaacaagtgcttacaaccatctcttcttgaaagtgcctgtcttacatcagattttagattaagtcgtttgcccccagatcttagctttctgatgtgttcaagagaaaattttagattatctggctgttttttgttgttgtaagggtgaggccatgcacttttcagttttctacatcgtgagaaggtgaatttcttgatttttcaaaaagaatcacacttttaaaaaccctgaaaaataaagaaaagagactgagctaccagaacggcagtgactttgggtggctctcacttatgccaggcacagggtgaattcagggctctgggctatgaggggtcgactgcaaacccctgcagcagatgtgagctgcatgtgtgggcccctgtgtcagcctgtgctcatttgtcttcctacagtgatactgcatgtgatacacaacacccaaggacaatggaggtgggggcaaatgtcccttattcttcacacccttcgctgttggcatcaaagcagttactctaccttctccacaggctttggcacagcatgtcaggaagttctctggggtggccacacctatttcctctaagtgctttcaacaggtttttcatcatgataggtatcctttcttacctctgattgatgtagtcatgctgttttcctgggacagtgttaagctctgctggacaggggatgaatatttcttgggtaaactgactgctctcactttgcccatggtccattgctaatcctctctgctagtggtaatgtatggaagtgagaggcgtcagcaacattgaaagacatcacattctaggagtgcaatcattacagcaattcaagagaagtcctggttgtataatgtgtgattcgttcattatgctataacaatccaaatgctaaatttaacaagatcaataaaacgtataaccattcacattttcttgcatattctgggtaagacttgtagttgcagttattgtagcacagtaataaaggcactggaagccagtattcaaggttggtacgatgatgcagcgtcatgagcaaggtgcctcaactcagaactgggctctggaggaacgccaagagcctatctcgccagcgtgcccttttcctcacgggaattaggagggaagcaccaagatgtggggtcctccacgagtcctagagagaaatccagagagtgtatgggttcaccagtcagtgtgatgacgc

SEQ ID NO: 8 - DNA, 1512 bp, Homo sapiens
  misc_feature 1..1512: note = LINC00839 which is positioned on the plus strand with the promoter region positioned (chr10:42,473,979-42,475,490)
  source 1..1512: mol_type = genomic DNA
aaactttaagtataaaagcagtactgatagggtttttgcatatgttactttaattatgactttaattgcttaaagtatttcctggaaacacgaagttgttaacaagtgttagttatttttgtttgagagaatttcaagagattgaaattatcaacagaggaatgtgaaagtcgttgggcattaagctaagccataaaaactttaccctgaaaatcgtgggggtgtccttgaatgcccacgagtttaatttccattttctgcctgaggttttaaggagtatgcaggattaggagggtaaacactcatagctcattagtctaacagtgaagaaaccatgagggcctagagtaatggaaaggcaatgaagtgaaaagacagttaaaatgtgtaattttccaaatacagaactgagatatttaaaaggattgtatgtggtggttgggtgccattacaaaggaggtggatgaattttaggcgtctagatttgtggtgctgatggttgggcaggggacaagcacacagggaaacaaggaaaaaagatgtatttaggtttaggtaaaaatatcacatgctcagtatccaattcctgttaaacacctggagatgggagtctgtggtgcaaagggagttcgagtttaactatacacgattgggtgatgctggcatgcaaggagagatgaagccatggatttcattaaggaccccaatctgaatgtggaccgcacacaagaatcagaaccaaaacctggacacagccaagactgcaggtttctcttcccttgctccaaatccactttcagaagaattagagaagaattgactaaagaatgtaagtaaactccaggaaacagggttccacagaagctggggaaaggtcagtacgtggaagagagagatcagtagtacctgctcgctacaaggaggcgcctccaaatgtcaatgcctattttcaacaagtgggacccaaagtgattcatgccagtgctctaaaatacacctgacccttgaacaatgtgggggatatggatgctaaccccccacagtcaaaaatccaagtttaactctcaactcccccaaagcataactcctaacggcctgcggttgaccggaagtctttgtcactaataatacaaaaaattaagacctattttgcctgttattcacattacacactgaatatgctgctcccagcggaggccgagactcctgcccgggcaccccctcacagcccccactccagcctccccagactcaccctcggcggggccaccttcttctcatttcagtctggggccacattctccgggcctgcggggcatggcaggaggcaaagccaaaggcgaagtggccacaaattccggtgggggcgctggctcctgccccaccccagcatctataccctgagtctgataggacgagaaggaacatttcagccagccctgattgggtcatattatccaatcagagttgttttgcacagtggctctcatccaatccgaacatgccttacag

SEQ ID NO: 9 - DNA, 1166 bp, Homo sapiens
  misc_feature 1..1166: note = LINC00865 which is positioned on the plus strand with the promoter region positioned (chr10:89,836,052-89,837,217)
  source 1..1166: mol_type = genomic DNA
catgtcttggtgagcctgtgtttcatagaacctggctcttcatacatgctggcaatgtaatccacccaatatcattatcacaaacacctttcccattgaaattaacctgagttggtttctgttgcttgcagctgatgaatggatgaccttctcttcctctatgtcatctcctttagttatgtatttattaacttaaatctcatattactctctttggcctctgctacctgaatcttctggtatcagctgaagagggagtctgggaggcacagaactggaagtaaggccatactgggcctgggaggcagagagagtcctactggagggccctcagaaagtgaattcagcaggcaccagcctccagccacagggtcctatgcccacacttgcttccacaagcctggtccaggccaccatcatgtcgcctggaccatggccgtatttcctagggtctgcctactcctgtccttgtccctgtaaacactttatcaaacacagggatattgttaaaacataatcagatcatgtcaattctctgtgaaaacctcattctctcagagtaaaagtccttatagaagcctaaaggccatacatggtacccgcttcctccgaaaaatcgcattctctttattacgccttcgatccctaggagaacctaagttcaagaggagatgctgggcagggaggggccagccacttgctagggtgccccagcccctgtctgcttcaagagccaagtcattttctcctggacccctctgagctgaagacaggaagagccgggggagggggatctcttggtctgcgcctcagtttctcaagaacagcaacagcagaaaaataaattgagagatgaaggggctaccaccatccaagtggccacaaccatccactcccatgccggcctggagttagcagacgggcgccactttgctctgcttgtcccacgcccatggaggggggacaccgtccggaagtgaaggccaggaaaacagaggggagagacagcggggtggggtgggagcagtcgctgctgtgaactcgctggagcccagactcacgcaactaaggggcagcagtgggaagagaaacggaggggtggtgggggagctggcgctctgaacgcctgttccttctgttcagactagtctttgtttattttgcag

SEQ ID NO: 10 - DNA, 1393 bp, Homo sapiens
  misc_feature 1..1393: note = LRRK2 which is positioned on the plus strand with the promoter region positioned (chr12:40,223,497-40,224,889)
  source 1..1393: mol_type = genomic DNA
ataaatattttagcatgtctctgtaaaagttaaagactctataacaacaagacatagacccaatatccaggttgagtaggagtttgctatcttactaatctaatgtattaacaaagtctagagcattcttattaaagtggtagagcatgaagaaccttctacctggagttaggagatttgctgggttgaagtcctggctctcaacttcccacctgtgtgactccagcaaagctcttcacttctcaattccaaatctcaatttcctcactagtgaaagaaggatacatagtagtagttgtatccatttcagaattgtcatcaggattgaagtaacagatgtgaaaacaaaactaaaattgtgaagtagtaacttccagtccaatcctcttctttgtaagtgatcttgaggacagacacatcacctgaaggatagatcatctttgcctagccttgtctacaatgttcttgctaggtttcttttcctaaaaacacattacttaggtacttaacagaacaaacaaacaaacaatcaaaagacaaaaccaaaaaactttggggttatagacacccatactcataagtatttctgaataccaagagaagagtatttaggtttgcttctctcaacttttcacctttcatttcatgtaccctgtcctttgtctcagctctaatagctctgagagctgattacttttcgggtgtcccaagtatcaggatcctgcctagtgcaactcaaatttccaaaagttaatttagtggccttttggtgaccagagcttcagataactcacagggaaacaatgtttatttcctctcccactaacagtcacaaaaaatcataaaaaagagtagcgggggcagttttgatggctaacccctctttccatcctttgggggaaaattgctcatctccctataggtggaactctaaagacaatgtattcctaaaaggggccatctgggcggtgtcctcttttcccagcgccctgatttctattcttagatctggagataggcggctttcatttttcctgctcccagttcccagaccttccgtggggccgcaggatccccggctggcgggtcgcggagggtggccggccgggctgcgcactgcgcgcctccgctgcggggctccgggcctgtggactcagcggagtccgctgagtcagtttcttcccgcgcgactcccggccgcgccgccgctgcggtggaatctggtcccaggaggcggcgtccgcccggggtccggtctaggcgtgcgtgggggccacggtcacggtcatcccagccaggcccggctccagcagccccacggccgccgccagagttctgcgcggcccgtcgcctcggcggagcctctggcaggcccctgagctcgtttttgggg

SEQ ID NO: 11 - DNA, 2270 bp, Homo sapiens
  misc_feature 1..2270: note = MEG3 which is positioned on the plus strand with the promoter region positioned (chr14:100,823,838-100,826,107)
  source 1..2270: mol_type = genomic DNA
cagcgcggagggagcagagccgggattcaaacccgtggtgctttgtaggaaacccgtaatttccacggtctttttccttgggctgtcagtaaggccctgaaataggagagtggggtttactgtgaacctcgaaggagcaggctcaagagccagggggcccagaatagggcagaggctggcttctcactgggaggctcaacttcccgcaggggcgccaggccctgacaggagagactggacattaggtgtctggaagtttctccttctaggggctccaggagcatcccacctgaggcctggggctcccaccggaacgtcaactcatgcccttgtggggctgcagggctgacgcgggctggcactgtgtctacgacagcctcccgggcccccgggtgcgtggctgcggatgctctctggcggccaccacagttcccacgcgcggcgggtgaatgcgagccccttgtcaatgcgtgcccctcgcctctgcccccgagaggctagcaaccgggcctgccggcgtctgattggccgtgggggcctgacgacaggggctcctggttgctgattggccccggccagctctggctgtttcctagctattaatactgtggctaataaacgttctcctgttgacgcgggtgctcggagccatgattttttgaagcgttcttgacgtctgtggctggtgcgggcgtttctgctgtgcaaggcctgcttccggggctctagtccctggggctcctggcaagctccacaggctgtaaagggggtgttttctttctccttcgtgttacctgacatattaaagcagcgccccatgaggaccccaacgtcccacgtctctatctccccaacagtgcgcctgtttatgaaaaaacgagccccccacacgccgtcccaaggctcggcgcctctagtgacctgacggtcaatgttcacctccctagtcattagctgtggacgtagaaatagcctttctcccttcttttcagccctggaatctcccgtctgctttacagtcaccttgtgggatcgttgggcatggggggctccctctgacgccgcttaaaccccccaaagaagtgcgggggagcgattctgacttgatgccctcggcggggcctctctgctgtcctctttggggccccttgctcatcctcacctgctttctgtcaccgagcactccatccttgtcctcctggctgccacccacctgtcaccctaagatagatcctcggtccctttcaacagcattttgacctttgcgagaggacatttgattgacggccctgcacgctctgccactcagagccgggcagagctgccgagggctcccacctgttagggattaactcccatgtgccagctccggagccgaggccgcggcagggctcggcgcaacatgtgtcgctgcctgtttttcggggcgctggggctctttcaaagggcatgtgtgtggggatcaccccaactttgaggggtagctcaggcatctccacatgggacacagtccgccctctaaaagtactttccttagagggcacggctcctgtctggattcttattacaaccctggtgtgtgtggtggggggtacccctggttaaggtggaggaagcggacccagtggaagaggaaggggcatagcgggttccgcgaacatgagttgtaagcggcagagcccgggactccacagccagggttttctgactcggagacgcggagctctgtctcccatgtcaaagagaaccagtcagaaacgcacgggtgggctgggagggggtgacagcctctggcttacatctgggaaccagttatgtcgcccgggcattttattttcctgaacaataagagaaagcatgattttctttcgctaagcccgcatcctccgatggatgttccgaaaccgccaggtgtgggatctgcgccccgacagccccaccttggccatcggccgcctgaggacggccagccatcggggcgattccccccacacattgtgcctgaattcaccctgcctggcgagccgggctcacgcagggaaaaagcacccgcgaccacagggtgttggtcatggcggccaggggcactgcggcagaattttttcctcccttctttgctgcaatctgggtgcggctagagcaatttgtcatagaatctggggggctcatttttccggccaatcacttttagagaaatgagcgcattgcagcagaatgcgctgacgtcagagaccaccccttctgcgcctccatataaaccccacccagcc

SEQ ID NO: 12 - DNA, 7679 bp, Homo sapiens
  misc_feature 1..7679: note = MIRLET7BHG which is positioned on the plus strand with the promoter region positioned (chr22:46,078,318-46,085,996)
  source 1..7679: mol_type = genomic DNA
ggacctccccgcactgccaggagaagggcctgacggggattctggccccacacagtgcgatggttgcccagcctcaccgcctgcagctccatctgagaagggcctgatacgggctggggtccaggcccactgccctcatgtcctgagccccacggtggcctgcaggcctggtttggaggccattatctcctcggcggccagccgggcacagcagtggccacttctggtcgaggcagttctgtgggtgaggggcgagcatcgcctcctgcaaccgtaacccaggccctgcctcccctgccatgcccctgtgggtcccgtcgttgccatctcgtcctcagtggcttcccgatgttgcctgctggcagggcaggccccgtgccatggagcatgagggacccttcccctcccagggccaggcttttggcagccaggccaagccgctgctggaatggaattcccggcgttcctggtggggcgcatggcatcggaatcctacctgctttgtctctggcatcagctggtgagccagtcacgtggccgtgtgcgtgagaccagagcccaggactgagggctcctcccgggcgcttcctcctcgctcactcacccccaccccctttttttggctttcagcaggactggctctgagcaggcgtaaaacagtgttaaaactgaatccgggcagcagggagctcctgtccacggcggcaggctctcacagtccaccgggctctcgcggtccacccagaccaccctttacctcgagtccttatgcacagaaaggccctgatatgtcccatacactcaggagttaggcccagagctgggcagtggtcactccacgccattccctctggtgtagagctggccctgcctgcccccagacggccgtggggtgggtggcaccgcttcctggggaaccccttcccacacttctggccttgtttctcacccacacaaggacaccccagtggtcactgctgcagctcgcggtcacatagagggtgagaggggagagctggacaaacaggtgacccagcagacccagcctgatgcccgcaggagagagcaacggggtctgatattttgtctccaaatgaaagagccacagtgaaaccccaggcctgccaaccccagttgtagggccagagaacagggatgtttccctgaggcggtggcaaggtttggtttggtgaaaacgaaggatatgtgagggtctgagaggggagggtgactggcctagactccacccctggcgccctgtccaccgtggctggctggccactctcggacccctcggcgtcaagcgctgactgggtgcctgcctggggcttggggctctgtacgtgttaattctgccactccagcagccctgagtgggaggagccattatcccccttctttctgtagatggggaaactgaggcaggcttgcccatggtgaagtggccagtcggacacagggccagattgaaacctgcagcctgggctcccggctacaacagcgccagcctccacaggcattagaaggggactcactgcgagggccccagccagggcagctttcagggtggggtctctggcctcaccctggggaaacagccggggcgctggctgcctcctgctgagcctggcgtgggaacaatgtggcctctatccctggagcgagccaggccgcctggacgcccagcccttcagagcagcccggccaggcaggcgcccacagcatggcgcccgggccgcgctgtccgtccacggggtgcgggcgccttggccaggcccaggcaagccgctccccgtgtcctccctggctggccactgagtggccagacgccggcttcctcctccctctcccgcccggccagcctcctccttttttggtggtgggtttgggggcccagcccacccgccactgccacgtctgccatcctcccgcacccacgagcatctttcaaaaattcccggtgggcggggctgagctgcagcggccgcctctgccccccctccccgggcagccagtgccagatgagagcaggagacaaaagtagcattttcctcgtactccggggctggcagcggggccttcccccagcaccacatggctcctggcagccctgccccctgtgccggggtggccgaggcccagccgggcccactggaccgagggacggcggcggccagagactagggggcccgtgggcccatagctggggctggcaccgggcagcctgctggccggcgggacctgccctcctcccccacccgggtgaccggctgggcccagggacgtcattttcacttcctggctttggagaagacgcctccatcctccggcacctcgggtaagtcaccttctttccttctgtcgtgaagacgagccgagggtgctgccatcttgtgccccggtgcctgttacgtgtcctccctctaattggttctccaaactcagaattctatttcagagccctccatcttccgtgcaggaagccctgccaaaaactgcagggaaaatgcagaagccaagcccatttactttcaagatccaaagctacaggctttagaaatcacaataaaaatgaatgtttattatttcacagtagctgcgtcttccattttaatgggaacaaatgctttgggagtattaatagccatggcctactcctgccttcctggttgtggcagtttgtgtgggggtctcattattattattattattattattattattattattattattattgtcttaatttttggcggcgccaggagtggcaaaaggaaaaccaccaggaccatccacggctcctcccagcaaactcatcggcaggcagggccaggctggcacccgggagggaggactttgcatctagaaacagaggcccttttattttgtccggcatctttaatgctaccatgtccacagtttagggatctagttctttctccctttcgttttattttttggaaaaaaaagaaaaagaaaaaggaaaaagaaagaaagctccttttggccctcagctcagtgtggggaaaaatattttcggttgttttgggctttgtttttgagcacttatttcaacttgggggttccgcctgaaagtgctgggcctgtcttgtcaccgccctgaccgcagactgtgtgtggcgtgcgcgtgggcaccgttcactacacagacccttggggaagccctgagtgccctcatggcctgcccgtgtcctggatcccaccccagttcctcacctgcagagctgttgggggagacacccaggtcgtccagggcctgaaacgtggctggcagggccgggagtccctcaaagacctttgagtccccccgccttgccacctctgtccaggagctgggctcagtggctgcccgggtgtccctgttggtccctgcccctccaaaagtgctgatccttttgattcagaggtgggctttgcagagcacagccccatgtccacctccacgctgtggcaggctgacaaaagcgtcctcggatgcggcctggttccccacccttgaggggagccagggtcttgatatatagaaaatggaagaatcagacccggaaagttctaaacggcactgagaggaaaactcccatggaaatccatctttgttttcccagttcggaggcgcgtgggccctgcgtgcgcgggcctcggtctgagcggccggcctcggcagacagcaccaaatctgtaaacggagctaagctcacacgaggactctcggcggctgcttttgttcgtagatgagccagcagtcgatactggagggtctggggctgcagaaacaggcatatatagcagagataattaaaacatttcatgattatgacagccacattctgctgctgcaagctggaggattcatgccacccaaatatttagcaagcatcaaataaaacactggtggcaaaataaaagtaatagaaaaaaaaaacagagcgtaatacatttcaaaggcaaaagagtcaaggggatggatagggaatggatcgggggaaaagtggggagagttcagcattttaaaagaattcacatgtttaatagctggccactcatattttttttttggtataattaatgtaggcaaatagacccttaattctaagtcctggaatgagagatggtaaaatttcctgattattaatttatcatttttgttttttgtacagggtaggggcaagtagcagaaaaatcaattaattcacataaaggaacattttcattctaaattaatattgccactgcaattaaatttatctagtatttttaacatgtatggataacacactttcctgtgtacaccttggatacctgggaccgttatatgtgatcaactatcatcttagttttttagcttggttaacacttgacccctccccccacctcaaaatcagtaaaatgctgtggattctgctttttaaaaaaatcaggccaaaatctcagcattctccatggctcccagtgcttctcggcagtttttttttttttttttttttgaagaaagtaccacgcaaagcacttttcacatcatcgcttctatataaattccactatagactgaaattatacattctcgcagcacagatgggctggtctgtcattacggctgctgggtacttgttggaacacacagcatatttcaattgtggagtcgacaatttcgaagaactggggtcaaaataaacaagttgcatttgtgtatagaaacagacttattcaatgatcttctcatcccctggaaaattcggcagaggaacgatcatcaccttcattaaacttgaatttaatttttggtcttcatcttttaagaccccacatgccctgtgtcaaaggcccatcaatgttttaattaattaccaatttttctcttatgctgcagcattagcagtggaacacaagatttaaattcaaactcgtgggtgtggtttttaatatcttttgactttaattttaacatcttaataattttaatatcttactaatctctataggggaggtgtgaaacctctgttctgtgtcgagttgcctattgttttgaggcctttatgaaaaatcagatgattcatatttaggatggtctaaagatacatttaaatccaaacaccacccacaaaaaaactcagcaggtaaaatacatagagtagccataaacttaaaaaaaaattatctcaccaagataacaaagtgcagaatttattggtttgtatcagcctccttgagggcagagcagtttttacagccacaataatttgcaaatgatgataactgtttattttgcaaatatgctcatattatgaggccatcagtggaggcttttttcttttcccatacgtgtaagtgtttgcctctgtgtttctaaattctggattcaggctctgcagtttctcaccggctgagaccacgttcccacgttcctgcctcagtggcagtggccgggaggagggaagggcgcgccccctgtgttgcctcctgcgagctccagccgccacatccatttgttcgtagaagtgctttgttgtggcttgtttattttaagggatgtacctgttggtggagatgttagcaaatcataagaaaagtaaaaagttgatgaaatttgccagagccgcttgggagccagctctgctgtctggtggttttaacgacatcatcccttccagttctggtcttttagcctattgactcccgcctccttagcaagaggaatatggccctggtctgggcgctctgaaagatgaggcaaagtgaaaaggccccagcctctgggccgcagagcctctaagcagcggtgggcatggcgatggtggtaatagtagcaacagcactctctgctgctgttcacaggtgcccgctgtgtaccggggctcctgctttcagaaaagcaccggctctgggacccaaggtctcggttcagatggggtcgtctttgtttccaggccgtgttgatcaagggcaagtcatttcacctctccggcctcatttccttatctgtaaaatggaggcagtaagtgtcaactttctgaacacatggaaagctgggttcatggtgagcaccccataaatgctattgcttccttgccttatttctaaggctcagactatgcctgcaagggggcattttcattacctccattccaccaggggaagcagcgaacctctgagaggctaagtgggttccccaagattcaaccagctgataagtgttgggccagggcttgaacccagccctgccatccttcaaagcccatgtgctgccatagcattcaccgacgcgtgttgcggcacgcgtgtgcgtggtgccccgggtgtggacagcatgttggtacgcagaggtgcagaggtgtgtgcgtgtaatgtttgcacgtgtgcataggtgtcctcttatttctccattgcgtgaggttcctggtaggtcggcagtttccaaagaggtgagctggtgtttggaggtgcattggtcggagtgggccacacagtcctcagaccacacctggggatgactgtgccatgggctctccaccaccccagggatgcccgtgggaagaagggtggttttccccagtccagggctagccaaagctggcagcgggtggactgggcatccaggcgtgggcttctcgtaggtgctcagcactgctgagggaggctctggctgagtggcctgcatgctgggccttggccagacgcgctgctgcctccttatgtggacaggcggttctgggcgccggccatttgcagactgggaggagtggaagtgaggcctgcgggccccggagaggaaatggcggcgtgggcaggcccagaacaggaccgggaaggcgggccgagggagggagtggcccagaaggcctctgtcggccggtcagcgtgctagcggcctttctccaagaagcggccagctggccaggccttcccgggcaggtctgtttgtctgcccacatgcacacacgtgcacctgcgtgccagggcacggggaggtttagtggccttatttcctaatagggaacatagcagggcctggggtttgatgagacggcgtaacgcccagcacaccttcagacaaaagagaccgtgataaaaacggttcttactacaaggtcccagcttctgtctttaaaatggggcttccagcctccgcctcagtagatggttggaggcactgcatgagatcctggtctggggagcccctggttcgttggtgtttcccttgccttggcctagtaagaggggacgggaggcccaggctctgtggtgggcctgtctcagcccatttcaaaccctacccactgccttcccatgaccttaacaggatggcattacccagcactcacccagatgccagggtggcaccgggtgagtggcagtttcgggtggtcctggagaagggtcctggcatcggctggtatgagtctctgggtttccgagcttcaatgagggctggattcagggtgtgggctgggccagccattccggcaaaaccccagaaggaatccacaggaggtggggaccgggaaggcagccgggttgcgccagtgcctgcaagggaacctctggccccatttcctgggagcctgaccccgccctaggattcctggagtgggtgtgcccacgcttccggcccagacgtgggtggagggatcccaccctgccaggcctcccgcctgtgcggc

SEQ ID NO: 13 - DNA, 1501 bp, Homo sapiens
  misc_feature 1..1501: note = NKAPL which is positioned on the plus strand with the promoter region positioned (chr6:28,257,796-28,259,296)
  source 1..1501: mol_type = genomic DNA
gtgtcgctacctgaaagaagtatttcatcaaccagctttgtaattcaagtctacatcagatactaaatggcttaaatttacaattatccaagtaagacatatccatttcattggcctatcaagtagtttataactccaaaagaaaattttctttacttacacaaactctatagtactcaccctatccatatgatattttcactatacatgtgaccattcacatataatactccctgctgtttaccagttcctaaattcatataatttatgtatggaaccaattcttatccccttcaatcaaagtactaaaattctacttagagcaaatgatatgatttggtcttattcaatttaatttcccccattttgttgtttagagttctagcatcaggaaggcatcattcttaagttttgacctggctgtcaaacaactgagcagatcaacttaattagagagaaccaagatgatgttaatgcctatttacattttaagatgagagataaatgatcttgcataaaacgtttatcatttctgagagtgagatctaatgtgtatcgagaaagtggtttccaaactacaaagtttctgaagaagtctccctagatagaaagacatctgtctacaagatcgagtgggatgcttctgtgtgtttctagggtggaaaagtctgtgaatttaataattaaaatcatgactctgggccgggcatggtggctcacacctgtaattccagcactttgtaaggctgaggtgggaggatcgcttgaacccaggagtttgagaccagactcagaaacaaagtgacaacggtctctacaaataattattaaaaaaatcaacctggcgtggtggtgggcacctgcagtcccagctcctcgggaggatctcttgaacctaggaagtcaagtcggccctgagccatgatcgtgccactgcaccccaacctgggcgacagagtgagatcctgtctccaaaaaaaaaaaaaaaaagtacgactttggccttcacctagcttagagggtgagctcaaactgattccaaaagaagttttggttcttgcttccagtggtaacaaggggggttgggaagtgattaattaaacatatttcctccacgaagagggtggacaagttccttttttaggaaggaaaaaaggaggagcgttggtagtagggatcttccacagaggggtaaagttaaggaggtggaaatgaaagggaagaacaaagggtgagaagcaaaatggtaaaagcgaaatcgaaaaagagaagtgggtgttccaacagtagaggggatcattgacgcaaattcgtccttttatgttcccacacggctcaagttggggaatataagtcctaaaaacaggcgcaccctaatcctacctacttcccacaagaagccttcacgcagccaaggcccgccccacatagcacacgcgcggattcgttttccgtttggtaactgacaggaagcggaagccgtgcgtgggaggccgattct

SEQ ID NO: 14 - DNA, 1642 bp, Homo sapiens
  misc_feature 1..1642: note = PRR34-AS1 (gene position chr22:46,053,705-46,058,522) which is positioned on the plus strand with the promoter region positioned (chr22:46,052,204-46,053,845)
  source 1..1642: mol_type = genomic DNA
aaaggagggggtgataataatgctgcccagaccccagaggacccctaggaggctcttgctgggcagctggtcctgccccagagccctcccactccacacacccctgcatacgctcaagggcagatgggccccccacaaccaagccaggcctggtggccctaggacactcttggccacaagaattcagattattttctcagcaaatttctgaccagtgtcctggggcctcagcaggggcagggctgcatggtcctgggtctatctggacttgttctggctcagacctcagcccaccacaaggacaacgagtctgggaactcgggcaggctctctgccaaccaggagctgctcttgctcgccgggaccccacagggcaggaatagtgggcattcatatcattcccattttgcaaccaggcagaccgaggcccagagaggccagataacctgcctcaggccacacaaatggcaaacaataaggccaaaccttggtgatggccagactctaaatccggggccacttcttgccttaggaatatagtctttcctggcactcacttagccaaaaatctttctagaagggtggcaggccttcctgccccactcccggaacaggccggctgctttcagggccagaagagccagccctttggaggaggaggcttgtgagagtggggatgggccgcagagcctccccaggcagggcagcgttgcagcttgcatgtggcagggatgtagggtgaagctgtgcagggcaaaagacaggtcagttcccgctggaagtgccccactggggggcctcggtgtgtctctttccccagcactggcatggcacattttgacctgagcaatggtgagctgcccggaatacgaacaggaggaagagaaccttccccaggcagcatgtgaggctggggtggcactcgacacaggacagcaggcaaggacatgccgccctccaggctgagggtcttgggagcctcccctcccagcccctgctgtggcctccccctgggccctggcatgggaggagttaagctgccgcgggcaccttggcacattgcgctgcccagagtgtccggctccccacccccactgcttttcttcctttttgcctttttccttcttaattaatcatacgctgctgcccaatttaaatatggaaatgcccaggccgaggcgatgcaattattcatgagggccgggcggctgtccggggctctcgggctgtggggagtaccgtggcatcgcctccgttctgatgtgtgtgaaattatgtgcacaaaaggcgcctccttatctgggagtgtgaactaaataaataaataagggcttttgtttgtttgccggctcctgcacatggctgctgggactcaagcgctcgtgttgtctgcgcctctgtgggactctggggacgggaggcaggggaggcccccgcaggccggccaggggctgggggctgggggccgggggccggccgccgggcacctggggccctctccctccccaggcaccaccctcaccccgcagctcccccccatacacaccctcgctggaaagttcgccctgcccgggctgggctcccaggcagcctggcaccgtgccaggctcgccgccgagctcgggcagcagggtggggcaaa

SEQ ID NO: 15 - DNA, 1205 bp, Homo sapiens
  misc_feature 1..1205: note = ZNF300P1 (gene position chr5:150,930,436-150,946,584) which is positioned on the minus strand with the promoter region positioned (chr5:150,946,585-150,947,789)
  source 1..1205: mol_type = genomic DNA
tttatagccgtcattaaaatgtcttttcctggatgtctggatcatgctggtgttggtactataatcatacttatttttattgtctgaactcttagaaaacattgtcataatatttacaatgttaaaaattaaactagcacaataattggtagaaagcagcatagttaatccaacttaaacactcctgaatgcttatgatggcaatgcacttaagtcatacacttaaatcccaacattccaaagtgacccaacaatgggtaatccattattgtaaattgcaaattatggtcgccaacctcagggatgggggtaaaatagcaatagcgcctaggaaaaggcacagaatctatgccatattgacttgacaataataaccatccctttcaaccctactttttgggccacctagtgggttttgtatctttcgtcaaatttttcccactcatccagggaaactgggacttctgaagcctcagggagggacttcccgggacacagcggacttttgctgctaatcctataatagtttcaggcaaagagggaccgttggtcaccttacgcagtctccctctacattccatcagcgtcctccttttcccaggctccccacttccctctttctcccctctccttcgtcgtccccttgcctctgtccccagtctctcactcttggtcccttagtttcccattctctgttcttaggtcttgcgtctttttgtcccttagtctttctcatttccgcactttctcctttattttgtacgtccatctcctcttccctgtctcaattttcctcctttttttaaagtttgcccctgcaacttattaccgcgcagtattgtaatatctccctcggagtgtttcttatcattactcttaataaaagttcagtattactgttatcccctcccgaagtgaccgttcccaccatggtggccccagtctcccacagaggaaacgccccggagaagaacgtaccatttacaaacacgtttaattggaatcgccaacgaaaatattgtaccattctaaattcttcgcctttcaacataagcatccttaaatggaggacagtagcactccaacttcttctccaatcctgtgaaatctgtctgcaaaagtgtggaccttactgctcgtgtagtaggcctccttgccgtcgtcttcccctcccttcgcagtcactgtggtacagtccacattcc

SEQ ID NO: 16 - DNA, 82 bp, Homo sapiens
  misc_feature 1..82: note = Common motif
  source 1..82: mol_type = genomic DNA
amakarhawsayamtdtywmaaaaaawaaaraaaaaaaraaamakarhawsayamtdtywmaaaaaawaaaraaaaaaaraa

SEQ ID NO: 17 - DNA, 100 bp, Homo sapiens
  misc_feature 1..100: note = Common motif
  source 1..100: mol_type = genomic DNA
accyrggaggtvgagbytgcastgagchrwgatcgcdccactgcactccaaccyrggaggtvgagbytgcastgagchrwgatcgcdccactgcactcca

SEQ ID NO: 18 - DNA, 92 bp, Homo sapiens
  misc_feature 1..92: note = Common motif
  source 1..92: mol_type = genomic DNA
ccgagbagctgggactacaggcrhcygyyaccacgccngrctaattccgagbagctgggactacaggcrhcygyyaccacgccngrctaatt

SEQ ID NO: 19 - DNA, 30 bp, Homo sapiens
  misc_feature 1..30: note = Common motif
  source 1..30: mol_type = genomic DNA
aamawaataaatwaaaamawaataaatwaa

SEQ ID NO: 20 - DNA, 100 bp, Homo sapiens
  misc_feature 1..100: note = Common motif
  source 1..100: mol_type = genomic DNA
gtggctcabrcctgtaatyccagcactttrdaaggcbrarghdggnggrtgtggctcabrcctgtaatyccagcactttrdaaggcbrarghdggnggrt

SEQ ID NO: 21 - DNA, 548 bp, Homo sapiens
  misc_feature 1..548: note = A first promoter region associated with the gene CCDC144NL-AS1 (gene position chr17:20,868,432-21,002,276) which is positioned on the plus strand with the promoter region positioned (chr17:20,867,979-20,868,526)
  source 1..548: mol_type = genomic DNA
gtggcacaggttccagcctccagcatgtggcagtgcctcttccttctagtcctccagcctggcaggagaagctcctgctgctgaccgctctcctactgctctatcgccactaccaaccgcagcgaggtagtgacccaggctccaggctccatccatcctccaccctccagcaggtggaaggttgcggcttcttccagttctctaagctggacacagagttgctcctccgctggacacagaagagcctgaaatgacctgacgccacctcagcatgctttatatatgaggttatgcaaatgcagttcctggactacatgttctgattggatgagaaaaaaaacctctaggcctactctgattggactttattttcatgctgtgattggttgtgttaagacttgctctcatccaatcagaacatgatcataaagtccaatcagagtaagcctggaggtttttttctcatccaatcaagacatgcagtccaggaacctccgtgggcattaccgcagtatataaatgatgctgaagcggaaacacgttttttc

SEQ ID NO: 22 - DNA, 1082 bp, Homo sapiens
  misc_feature 1..1082: note = A second promoter region associated with the gene CCDC144NL-AS1 (gene position chr17:20,869,776-20,870,857) which is positioned on the plus strand with the promoter region positioned (chr17:20,867,979-20,868,526)
  source 1..1082: mol_type = genomic DNA
tgggcaaaaaatgatgaagtttgggggcagatggtgaaagaaaaagggtggtgagagggagggggccaaaggccgtttggaaaagaaggtagggaaataatggtgggggacaaaggtttggggtagatttttttaataagatcatttgtatgtttgcttttcagtagtttgagttctttatgcattttttgtatgaaccccttgcctgatgcatggtttgcaaatacttgcttccattatctgggttctttcatttttattaaattttaattcaatttaattttttttagacggagtctcggtctgtcgcccaggttggagtgcagtggcacgatctccgctcactgccaagctctgcctcctgggttcacgccattttcctgcctcggcctcccgagtagctgggactacaggcgcctgccaccacgccaggacactttttttgtatttttagcagagacggagtttcaccatgttgttagccagtatggtcttgatctcctggcctcatgatcggcgcccctcagcttcccaaagtgctgggtgtacaggggtgagccaccgctccctgcctgttttttcattctactgattgcttcctctgctttgctgaggcgttttttttgtttttgagacggagtcttgctctgtcgcccaggctggaatgcagtagcatggtctcagctcactgcaagctctgactccgggttcacgccattctcctgcctcagcctcgcgagtagctgggactgcaggcgcaggctaccaagcccggctaattttttgtatttttagtggagacggggtttcaccgtgttagccaggatggtctcaatctcgtgaactcatgatctgcctgcctcggcctcaaaaagtgctggggttacaggcgtgagccactgagcccggcctggaaacgtaactttattttttagtgttgtatttgtgcatatactttaatagccctgagttttaataaagttgcttttaaaaaatgtatcttatatttcagaaatacaccctaaggcatgtgattagttgggtggcatgttgtttagtttttacaattgaaggattgtcat

SEQ ID NO: 23 - DNA, 2901 bp, Homo sapiens
  misc_feature 1..2901: note = CASC9 (gene position chr8:75,117,286-75,352,327) which is positioned on the plus strand with the promoter region positioned (chr8:75,322,337-75,325,237)
  source 1..2901: mol_type = genomic DNA
aaaagagctaatctaagtaaacttggaaaacggtgttttgattacatatggcaagactacaaacaaataaacaaaaactatacacaaatattgttttatagtttgtaaatttgttttcatggaatacatcttagcaattctaaaaatacttcatgtatactctaggattaaacaattacataagtatattgtagataacaacagccaggcttctcactgttgaagaaagaagtttcaaataaggtaggagagaaggcgagaaggaacactataatgccagatggaattgaaaatattagtagaaactcatggttttttatatacatggacgtctgtgtgtgacccatatacatacatacatggataatatagaaattaatatacatatgtgtagatatatggttaaatatgcatatatatatgtataatcagctctgtccactgagtgcctataagcaatgatttcccagtagtaataagtacatccagccttcagatcttgacttttaaattttactctccaatataaggaactggggttccttggtaaaatggctgattctaggtctgggtcacggaaagtttcagaaagtaagaaattgctcagaaaaaaaaataataattgttgcagaccagaatcatcaatggatgctaaaattagtgggcaaagttgaataagaaataggatttttcatattttcaaggtcatcttcaactgatacttaataattacaaagggaatatagcaatgttaaagtggtaaaacctagaatcctccaatttatgcaagcaatcaatttaaaattaccagcaatgtgatatattgttatcatgtcttcgaatatgacatactgaggagctcacatcacttctgtaagattcttgcccaaatgcataaggtaaattatgttaagaagaaatacataaacacacactgaggaaaattctacaaaataagtagccaataatcttcaaaagtgttaaagacatgaaagacaaagtaaatttcagaactgtcccagaatgaaggagaccaaagagacatgccaactaaatacaatgtgtgattctggattagatatgaatcagtacaaagggcattagtgggacacttgggaacatgcgtataaagttccttgattggttgacagtgttacattgatgttaatttcctagttatgattattgtactatggttaagtaaggtgtaaaaatttaaggaagctttacgaatatagttaggtctgaaatgatgtcaaaatgaaaaattaaaaaccatataaaatagtgaaatcagattaaatgtgcatcaaaaaattatggcgcatatatcctgtggatatttcactgcatttaaaaagaaaacaaaagatccatatgtacagacatgaaaacgtgttgttgaaatgccaattaattaaaaaatacagttgcaatgtaatgtctatcatcccactttttttcaggcaaactatatacgagttcataggaaaaaattctggaataattctaaccaaactattcactactttgtcttgattagaggaagatgtgagaaaaatgaaggtttaaggaacacttgttttttattacctatacttgtatattcgtttaatttcataacaagaataaatttctttaaaaaaacagaaaacaatgaagagagaaaaaaatcttcacctgatccccagtggtttatagaattaaggtaaattttaaaatattatgtgctccacctaacttattcaaattcgaacctcatattccaccaatcagataatttttacttttgctacttatctttgtgaataatctattcacttgttctaaatcaatttactttaattctttttaatattatatttttaaagtataaaactttaccggaggagccaagatggccgaataggaacagctccggtctacagctcccagcgtgagcgacgcagaagacgggtgatttctgcatttccatctgaggtaccgggttcatctcactagggagtgccagacagtgggcgcgggtcagtgggtgcgtgcactgtgcgccagccgaagcagggcgaggcattgcctcacttgggaagcgcaaagggtcagggagttccctttcccagtcaaagaaaggggtgacagacggcacctggaaaatcgggtcactcccacccgaatattgcgcttttcggactggcttaaaaaaccacccaccacgagattatatcctgcacctggttcagagggtcctacgcccacggagtctcgctgattgctagcacagcagtctgagatcaaactgcaaggcggcagcgaggctgggggaggggcgcccgccattgcccaggcttgcttaggtaaacaaagcagcagggaagctcgaactgggtggagcccaccacagctcaaggaggcctgcctgcctctgtaggctccacctctgggggcagggcacagacaaacaaaaagacagcagtaacctttgcagacttaaatgtccctgtctgacagctttgaagagagcagtggttctcccagcccacagctggagatctgagaatgagcagactgcctcctcaagtgggtccctgacccctgacccgcgagcagcctaactgggaggcaccccccagcaggggcacactgacacctcacacggcagggtattccaacagacctgcagctgagggtcctgtctgttagaaggaaaactaacaaacagaaaggacatccacaccaaaaacccatctgtacatcaccatcatcaaagaccaaaagtagataaaaccacaaagatggggaaaaaacagaacagaaaaactggaaactctaaaaagcagagcgcctctcctcctccaaaggaacgcagttcctcaccagcaacggaacaaagctggatggagaatg

SEQ ID NO: 24 - DNA, 5874 bp, Homo sapiens
  misc_feature 1..5874: note = CPT1B (gene position chr22:50,568,861-50,578,612) which is positioned on the minus strand with the promoter region positioned (chr22:50,578,614-50,584,487)
  source 1..5874: mol_type = genomic DNA
ttgctatgttgcccaggctggtctcaaactcctgggctcaaacgatcctcctacttcggcttcccaaagtgcagggattaaaagcgtgacccactgcccccagccagaatcagttttcttttttttttttttttttttgagactgagtctgactctgtcgctcaggctggagtgcagtggctcgcggcaacaaggcctgggttcaagggattgtcacgcctcagcctcccgtgtagctgggattacaggcacaagccaccaaacccggctaatttttgtatttttagtagcgacggggtttcgctatgttggccagggtggtctcaaactcctgacctcaagtgatctgcccgcctcggcctcccaaaatgctgggagtacaggcgtgagccaccacgcccggaccagttactttttaaagtggctctaggtccctttagggaaggattggaggaatatttcttggtatatgctcagatgctggtattgtaagatccttcctgagaggaaccaagcctccttgtcaattaatgggctctaggtctcaaaactgactcagatttaaacagaatgatcatgattttccactgcagcaaaagtgacattagccttgtgatcaccaatttttgatttctttctggttatcaaacaatactctagtcagatgccccaaagaacaccatggttcgacagacattgccacagatctctgtgggtccagcgttcctggtcgccgctctagcctggggcagaagcagaaatgcaggaggcacgcgtttaaatcaggacgccctcaatgggaggtgcgcaggccgaggagggggctgcggcaagctgcgagtcttcactttcttcggctcttgggtttgactctgtaattggttctaggcctggaggagacgaggaggatagggggagaggggaatgaatgaagacgcgcatctcaagcggggcagcaaaagactcctcattcagaggtgcggctctaccggccagggagtccccgtgtagactgggcatttatgcaactggagtcatcggaactgtcccaagggtcacattccttcagcaaatgtttactgcgtacccaccgggtgcccgcatcccgcactctgcccgcgctgtgcggccctgcctagcgcgcgcgcctcctccaggcctcacccacgctcaggacggacgcgcgctggacggctcttccttgtcggagcgccccaggggtcggggaagagggcccggcaagggagccctcgcgccggagctgcagctgcagccgccgccccgccgccccgccggctcccacggggcagagacgcagctcctctccggtcttcccgtacgctaccgcgcccgggcagttcctcgcccgcgcacgcgccgctccgccaactgattggcctccggcgcctcggattggcccaggccgtccaacagcagccccgcccagagagagaccattggtccttgcccataggggcggggcccggcagagatctgcggaattcggccttcggaaagagcccccgggccggggcacggagagagccgagcgccgcagccgtgagccgaatagagccggagagacccgagtatgaccggagaagcccaggccggccggaagaggagccgagcgcggccggaaggaaccgagcccgtccgaagggagcggagcgcagcctggcctggggcccggtcgagcccgcgccatggcggccgaggcgacagctgtggccggaagcggggctgttggcggctgcctggccaaagacggcttgcagcagtctaagtgcccggacactaccccaaaacggcggcgcgcctcgtcgctgtcgcgtgacgccgagcgccgagcctaccaatggtgccgggagtacttgggcggggcctggcgccgagtgcagcccgaggagctgagggtttaccccgtgaggtgggaggtcaggggtcagcctctccggtgcgcggatcggggtcaggggtcagccgcggggccctcaggatgctccatgttttcgcccccctcttgcgcccgcgcctggggcggggcggggccggcctggccgggagggggccggggccgcggcaggtagggccggccgcgggctgagcgcgcctggtgtgggtctgcagcggaggcctcagcaacctgctcttccgctgctcgctcccggaccacctgcccagcgttggcgaggagccccgggaggtgcttctgcggctgtacggagccatcttgcaggtgaggggggtgtgagcgccgcagcaccagtggctttagggcctgtcgcttacgcgatgcgggtagtattgttcccgttgcgcagttgaggacaccgaggttcacggtctgagtaacacctcattacaccgaagcctgggcctgtattcccagagctttgggaggctgaggcgagaggatcacttgagcacaggagttcgagaccagcctggacaacatagtgagacccccatctctaaataaaaatagaccaacgctaaagcctgtgctccagagcctccaggcaattggatcagaagtcgcagctctggtgggaggaaggcgagccctcatgtgtgtccctgtgccactttgccttggcccctttgctgtccatcctttttcagggcgtggactccctggtgctagaaagcgtgatgttcgccatacttgcggagcggtcgctggggccccagctgtacggagtcttcccagagggccggctggaacagtacatcccagtacgggcccagtcctaccctctcctccccaaggcacctccaccccctaaccctaccccagtgcccaatgtctgcctccacatccctcaccccaagtagggaatttcccccaaaaccctgacttcccctcattgccccagcccttcccactcctgccccagccccatcaccaccctgatagcttcctgggtgcagagtcggccattgaaaactcaagagcttcgagagccagtgttgtcagcagccattgccacgaagatggcgcaatttcatggcatggagatgcctttcaccaaggagccccactggctgtttgggaccatggagcggtgagtcaggagcctcctcagggctcctgtactcctgagctgaacctccatgccagcggatgtgtcgggggctctatccagccctccacttgagtggatctgatgtcatgggctgtcacagaacaggcttttaaatgcttgccagaggccacatgcagtgcctcatgcctgtaatcccagaactttgggagccgaggtgggtgaggtgggaggatcacttgagcccaggaggcggaggttgcagtgagctgagctcatgccattgcacttcagcctgggtgagagcaagactctgtctcaaaacaccaaacaaaaaaagaaaaaagaaatgattgccaaggccaggcacggtggctcatgcctgtaatcctagcacttcgggaggctgaggtgagcagattgcctgagctcaggagttcaaaaccagcctggacaacacagtgaaaccctgtctctattaaaatacaaaaaaattagccaggcatggcagcatgcacctgtagtcccagctactcgggaggctgaggcaggagaattgcttgaacccgggaggcagaggttgcagtgagccaagatcgcgccactgcactccagcctgggtgacagagcaagactccatttcaaaagaaaaaaagaaatgattgccggacaagtcacacagtgataggagcaggggtgcagtagtctggcctgaggggtagataggcgaggggtgggaaagaatagcagagccagtgtatcctattctttgggcttcaggtacctaaaacagatccaggacctgcccccaactggcctccctgagatgaacctgctggagatgtacagcctgaaggatgagatgggcaacctcaggtgagggcaggcaggacaaggctaatggtaatggtgtccgcccttccagtagttgctgagggctggtgtcagggcctggcctgttagggtggctctgatcctcctctagtcactcctgctcaggacccatgctcccatacccctgtaggaagttactagagtctaccccatcgccagtcgtcttctgccacaatgacatccaggaaggtaggagaaggcatctgagtctcctaacccaagatggaagagccagagggctctggagtgagcagaacctcaccccattcccccagggaacatcttgctgctctcagagccagaaaatgctgacagcctcatgctggtggacttcgagtacagcagttataactataggtgaggctggaaagatggcttcccatagatctgttcccatagggctcttgaaaacaggccagctgcccagggcatttggggactgaatgtccaccttattctcccaggggctttgacattgggaaccatttttgtgagtgggtttatgattatactcacgaggaatggcctttctacaaagcaaggcccacagactaccccactcaagaacagcaggtatgtgggccagaggctggggagcaggacccatcctgtgaggaaggagggaggtggagtctggaaggaatggccggaaaggatgttacctgggaaatactccacagtctccccaattcctgactcttggccattgatcgtagttgcattttattcgtcattacctggcagaggcaaagaaaggtgagaccctctcccaagaggagcagagaaaactggaagaagatttgctggtagaagtcagtcggtgaggaaggaggggcagggtggggtagggcagagcagaggaaagagggattggggaagaggcagatttatcaagctgcagggaaggtggctgtggagagtggagttaggacagtgggggagaagttaaaactgtagggattggccaacctgggtgaggggatccaagcagtgctagacatgctcttgaatgcccctccttttcctgccctccccccaggtatgctctggcatcccatttcttctggggtctgtggtccatcctccaggcatccatgtccaccatagaatttggttacttggtaagtgaccctggggatgggaatgctagctggggggctggggagcagcagcagccacactcttccaggaggcctggggagtcccgggtggctgtgggcagcctgaggtggatgtagaatgctggtcccacgtcttctcaccactgtgtggggtgggtttccttccctaggactatgcccagtctcggttccagttctacttccagcagaaggggcagctgaccagtgtccactcctcatcctgactccaccctcccactccttggatttctcctggagcctccagggcaggaccttggagggaggaacaacgagcagaaggccctggcgactgggctgagcccccaagtgaaactgaggttcaggagaccggcctgttcctgagtttgagtaggtccccatggctggcaggccagagccccgtgctgtgtatgtaacacaataaacaagcttcttcttcccaccctgtcctggccctgctgagcagcagcagaaagtaccaaaccgagcagtacacacaaagggactcttcagtgctctgggattgaaagtggttagcgttcatgctgccagttggggtcccccatccctccccagtcccctggctgcagcttagaataataaatactaggacttggggaggaggagagtgatgggggtatgaagacgaccctgaggtggggatgccgcccggagcaccagcgatcccagaacaggcagcagctgacacatcggtgaccttttccctacatttggctatttttagctctaaagccaccatcctcacgagactctggggccccccaggctcc

SEQ ID NO: 25 - DNA, 1985 bp, Homo sapiens
  misc_feature 1..1985: note = FAR2P1 (gene position chr2:130,012,065-130,051,131) which is positioned on the minus strand with the promoter region positioned (chr2:130,036,639-130,039,003)
  source 1..1985: mol_type = genomic DNA
ctctatgtacagtaataacaatatctgtcccagttattatgtacaatattataaaaaatgtcgcagacagtacaaattaaggcccttatttctcaaaggacatcaagtcttatgccccgtgggagggaagatgccacttaattattgcaccattttgaaaaacagaactcgtcaaggcggatagtggtcttttcccacctggtcttcagtcacagatgggggctggggcggtggcgccgtcttcgtcgtcgaggtagaccctatggagccacaccgccctgccgagttctctgggtgcaataaataccagtcacagtttgggagggggccgcgtgcaggtggggaggcctctgggctgcatgtgccgcttcctgtgcagggcaaggtgggcagagcgcaagaaggcgtggtggcaccaatggccctgaaatggccggtggcccctgtgcttgtggtggtggagcgttagcttgtctgagtgagcacacttccagctgcttccatcccagtggcagtggtcgggcttgtcacttgtgtgctgcgcagatgtgcctgcaggggagaattcttgctgtaggtctggccgtagccacataggtatgcctggtgttgcagttcccaggccaggagcagctgccacgcttgggcgtggcctccagcagctccagcggggacgccggtgccaccacaaggaggccgcgggcgactgggggtgccaggcccagggctgtggcggcggcggccaccgcctaagagactaaagacaggggcgtggaggggcggagctgggcggggggcgcctcatgaaggccgggctgggtgtcccaaaccagggccacggaagggcagcgggaaggaggcgtgcgggtcgtaggggctgaggggcaggtgttgggggcggaatgggagggacccgggacctcgcatgcatgaagctgcaggcccggggcgccctagtgtttgagaggacgcgcgatgcatacctgaacctgggtgcgcagccgcagctgccgccatctgcttcagggtgctgggctttgaccaggtggctgggcagcgccagttgaaagcggttgagcaggcagggcgcagtggctcacgcctgtaatcccagcactttgggagaccaaggcgggccgatcacggggtcaggagatggagaccatcctggctaacagggtgaaaccgcgtctctactaaacatacaaaaaaaaaaaaagtagccgggcgtggtggtggacgcctgtagtcccagcactttggaaggctgaggcgggcggatcacgaggtcaggagatggagaacatcctggctaacaggtgaaaccccatctctactaaaagtacaaaaaccaattagccgggtgcggtggcgggcgcctgtagtcccagctactccggaggctgaggcaggagaatggcgtgaacccaggagatggagcttgcagtgagccgagatcgcgccaccgcactctagcctgggcaacccaaggagactccatctcaaaagaacaacaacaacaaaaaccagctgagcagcgcgggccatgtggcagtgggtccagctccagggcgccgtagggcagcggtgtgcagtgttcgggtaataggacgcagacggcggggtcgccgggggcttcggggtggcctcggccccaggccatccagccctgtggaccgaatggagtcccgcacgctgttgaggtagttgtgggttcccctggcctcgggctgggcgcggggtcagcgcacctgcaggcggcgcttgcggtacgggctggtgaaagtggagatggacggcaggatggattcacttggccacatggcgcgaagctgggaagacggacaccggtgagtggctgcccgggagggctggtcggggcgcggacaggcgggcatggttctgccaaggattttgctttatttatcgcaagatggggtatttcctcctttcttcagtttataattgcatgaattagtgcagtg

SEQ ID NO: 26 - DNA, 1040 bp, Homo sapiens
  misc_feature 1..1040: note = FAR2P2 (gene position chr2:130,416,750-130,431,363) which is positioned on the minus strand with the promoter region positioned (chr2:130,428,547-130,429,586)
  source 1..1040: mol_type = genomic DNA
ccactctatgtacagtaataacaatatctgtcccagttattatgtacaatattataaaaaatgtcgcagacagtacaaattaaggcacttatttctcaaaggacatcaagtcttatgccccgtgggagggaagatgccacttaattattgcaccattttgaaaaacagaactcgtcaaggcggatagtggtcttttcccacctggtcttcagtcacagatgggggctggggcagtggcgccgtcttcgtcgtcgaggtagaccctatggagccacaccgccctgccgagttctctgggtgcaataaataccagtcacagtttgggagggggccgtgtgcaggtggggaggcctctgggctgcatgtgccgcttcctgtgcagggcaaggtgggcagagcgcaagaaggcgtggtggcaccaatggccctgaaatggccagtggcccctgtgcttgtggtggtggagcgttagcttgtctgagtgagcacgcttccagctgcttccatcccagtggcagtggtcgggcttgtcacttgtgtgctgcgcagatgtgcctgcaagggagaattcttgctgtaggtctggccgtagccgcataggtatgcccggtgttgcggttcccaggccaggagcagctgtcatgcttgggcttggcctccagccgctccagcggggacgccggtgccaccacaaggagactgcgggcgactgggggtgccaggcccagggctgtggcggtggcggccaacgcctaagagactaaagacaggggcgtggaggggcggagctgggtggggggcgcctcatgaaggccgggctgggtgtcccaaaccagggccacggaaaggcagcgggaaggaggcgtgcgggccgtaggggctgaggggcgggtgttgggggcggaatgggagggacccgggacctcgcatgcatgaagccgcaggcccggggcgccctagtgtttgaggacgcgcggtccatgcctgaacctgggtgcgcagccgcagctgccgccatctgcctcagggggttgggcttt

SEQ ID NO: 27 - DNA, 2375 bp, Homo sapiens
  misc_feature 1..2375: note = FAR2P3 (gene position chr2:130,690,133-130,718,831) which is positioned on the plus strand with the promoter region positioned (chr2:130,691,713-130,694,087)
  source 1..2375: mol_type = genomic DNA
attctggttataatatggaggcaggatgaaattgtttttattcttttagaattttttttatcaggaaaacagaggtaaagtgctatcaattactatttaagagttctattttgaaaagtctttaataaggatttttcttttctttttaaaaaagactttcttaaaaattaaaaataaaagaagcaaaagtcttaggaaaatgaggcaagtagccctgccactctatgtacagtaataacaatatctgtcccagttattatgtacaatattataaaaaatgtcacagacagtacaaattaaggcacttatttctcaaaggacatcaagtcttatgccccgtgggagggaagatgccacttaattattgcaccattttgaaaaacagaactcgtcaaggcggatagtggtcttttcccacctggtcttcagtcacagatgggggctggggcggtggcgccgtcttcgtcatcgaggtagaccctatggagccacaccgccctgccgagttctctgggtgcaataaataccagtcacagtttgggagggggccgtgtgcaggtggggaggcctctgggctgcatgtgccgcttcctgtgcagggcaaggtgggcggagcgcaagaaggcgtggtggcaccaatggccctgaaatggccagtggcccctgtgcttgtggtggtggagcgttagcttgtctgagtgagcacgcttccagctgcttccatcccagtggcagtggtcgggcttgtcacttgtgtgctgcgcagatgtgcctgcaagggagaattcttgctgtaggtctggccgtagccgcataggtatgcccggtgttgcggttcccaggccaggagcagctgtcatgcttgggcttggcctccagcagctccagcggggacgccggtgccaccacaaggagactgcgggcgactgggggtgccaggcccagggctgtggcggtggcggccaacgcctaagagactaaagacaggggcgtggaggggcggagctgggtggggggcgcctcatgaaggccgggctgggtgtcccaaaccagggccacggaaaggcagcgggaaggaggcgtgcgggccgtaggggctgaggggcgggtgttgggggcggaatgggagggacccgggacctcgcatgcatgaagccgcaggcccggggcgccctagtgtttgaggacgcgcggtccatgcctgaacctgggtgcgcagccgcagctgccgccatctgcctcagggggctgggctttgaccaggcggctgggcagcgccagttgaaagcggttgagcaggccgggcgcagtggctcacgcctgtaatcccagcactttgggaggccaaggcgggccgatcacggggtcaggagatcgagaccatcctggctaacacggtgaaaccccatctctactaaacatacaaaaaaaaaaagtagccgggcgtggtggcgggcgcctgtagtctcagcactttggaaggctgaggcgggcggatcacgaggtcaggagatggagaacatcctggctaacaggtgaaaccccgtctctactaaaagtacaaaaaccaattagccgggtgcggtggcgggcgcctgtagtcccagctactccggaggctgaggcaggagaatggcgtgaacccaggagatggagcttgcagtgagccgagatcgcgccaatgcactctagcctgggcgacacaaggagactccatctcaaaacaacaacaacaacaacaacaacaacaacaacaaaaaccagctgagcagcgcgggccttctggcagcgggtccagctccagggcgccctagggcagcggcgtgcagtgttcgggtaataggacgcagacggcggggtcgccgggggcttcggggtggcctcggccccaggccatccagccctgtggaccgaatggagtcccgcacgctgttgaggtagtcgtgggttcccctggcctcgggctcggcgcggggtcagcgctcctgcaggtggcgcttgcggtacgggctggtgaaagtggagacggacggcaggatggattcacttggcgacatggcgaggagctgggaagacggacaccggtgagtggctgcccgggagggctggtcggggcgcggacaggcgggcatggttctgccaaggattttgctttatttatcgcaagatgggggtatttcctccttcctgcagtttataattgcatgaattagtgcagtgaattgaggatgcagtaaaaatatcttcaaagattattaaattcgttattataaaacacatagaagagtttatgtgtgtgtatggaaagcaggtatacatcaataattcttaatgaatacaagaaagaactaccaatattggggcaaatttttcaa

SEQ ID NO: 28 - DNA, 4442 bp, Homo sapiens
  misc_feature 1..4442: note = LIN28B (gene position chr6:104,936,616-105,083,332) which is positioned on the plus strand with the promoter region positioned (chr6:104,952,665-104,957,106)
  source 1..4442: mol_type = genomic DNA
ttttgaaatgtaaagggaaattttgaaatataaaggcaaattttgaaatataaaggcaatttgtgtggaaaacttaaaatttatgctaatccgtttctgtatttccataattttactttattattctggggaaagtgtgttaaggtatttaatgaaagtctcgagagatttttacctctgtataaaaattctaacgttggaccaattgtgtgaagccagagcattttgcctccagacaaggaaattaaatgagaatagaattttgctttttggaaacaaaagcaaagcgacccaactatattctgaagtgcctctgctgcaagacccaagcacccagtcgggcgcgccttgcatttgtctccatgtcgtaggaatgtcaagaaagttaccgccaagtggtttaccaaaacgcggcggaaggaatcaggacagtttgcccgacgggcgggtcggcggctttcaggcgggagagatcttgccaggggcagagagagatcccgaaaatcgcccccagccttgcgtccagccctttctcgccgacgttttttcttgatcctccgaaggcccagggcgacgtttgccatcgcggcttcccgaaaggacggcgccgggtttgctggaagacagctcagttttaggcatggcccggtgggggtgggggtgctcattttcttgggttcggattccctctctttgaccgcacttatcttagctttaggagattctggcatctcataggaaaagggatcaaattataagtgatatacgtctggaaattcagtaaaaatagtaggttgtgcgtagtgtttttgccatagtagtgaaggcagggatttcttttgtttggtgttcttggggacaagatgacaaatgtaaaagtccctaaattagtgagaaatcaccagcctttggatctaccccacttagggagaggagatggtaaagtgacgaataaagggaaaagtgactgaggaaagaaaggtgagaggaagctgcagaggcaggaggtggagggaaagcgcagatctgctggcgcggggtctggggtctcaggtctcggccgcctggggagcgacctgcctggagaggcgggttgcgatggcagcgctgggctacagaccccgccttatttgcggggaggagaggacgggtggagtcacttgccgcattgccacagcccctcctcctagttttaagaagaaccgaagcattgtttctgctttaacagaagtcactcattccttataattgaaaacaacgacggtgggctctggcacctctttcggctccttgtactgttggaaaaaaagtctgggaaaacgtgtaccttgttttttaaatggggatgattgtgatagccaacaggcatatggtaggttatttccttgctttttccccccagtacattgacgattctttgtagctacttgaactcattttaaagacattttgaaagaactataagtcaagctataaagtatttggcttttgcaggaataaaacaccccttttgagtcaaacacaggtctttcaggcactaaaaccaagtaggaaatgagaaaaaaattaaaaccagaagacttcaggaaagtgacaaattggaaagcattctagaagactcataggacgactttctgtgaaagaagcactctaggagctttaaaagcttatgggcaatttatattataaaaggattttaggctcaaaagttttgggggaaggcacagaaaaaacagtgccatattcttaaattggtgagtgattattttgtgtttctcaatgtgtctccttcctttacttctctccctttccaacttccttgctttcttcttccttttccctctacccttccctcgcctcttctcttcccctttcttttaccctcccccaaggtatgtaaatgcatttgtcttaaattcttcatgaacgaattttagaggatttttcagggttaccagttggaaaaagccttctacacaacttgaaaggctgatgtccctctcaggaaatgaatgagtcgataacctctgacaccagtgaccactccagtgaaccttaccttaattccactaatactggaggcccccaatcaggcaagactgtgaatctcataactatactgtacagtgtagttcagttatggtattccacttttcacccacatgttcataaatttgaatttcatcaaatttaacaataaacagattctgaatctacctacattattatgcttctggttttataatgattaaactgttttctaattaaaagcaaacttaaatgaatattttacaaagctaaactatactggcctgtgaattttgtgatcattacttttatgtaagtaatagcatttcacctcaatttacatgaatactttctgagatttttttcctcttataccagatgttgattttttaaagtgattattaaaatatgaaaaaatataaattactttctcgatttcatattatgtcacaagcttagcctgtaatgccatcattgagattactttcttgtcagtgtaatcagtataatagaagtaatttctaccatcaaaataatgcctagtaaatgttttgttagtaaataattttttcagagctttgagcttttgttaaactttcaaagtcattggatttttgcaccttttcttttctccctaacagctggcacaggattattcctcccaaaataaataaaatgagaaagtagccctttttcctgagaaataacaaaaggaataacagacaaaaaaaaaaatctttccaaattgattagttttcattaaaccttttatcaaaaacagctgaaaataacttctattttttatacatccattttccaatctgagccaattaaatttatatttagttgtttaatatggggagggagtataaataggtgtatacagctttttccatagctcttcgattcataatccctacattttagcttgtgtgaaacagtgctggagtgtggatgtgtttgaagagactgggagggggattgctggcatggtgggggagggtcaactcggtagctttgtcgaaggaagtggtatctgaacagttagtttcttggtaaactgcttgtttaagaaaatggggggtggggtaggcaagtctggtggtgcattttaaggttttaaaatagcgatattttatattttaaaggaaataaagtctttttagttaatctataatatttattatagtattcgataagggcagaaatactgttgccagcatcttgtaatcaatcgtttgaaccagtttgaaaggtaaagggggtaggcattttgaaactgggggtcagttgaggaaggacagtaggagcttcatccgttgttacattttcgtttattcaactcttctagccattggtagtggacttttaaaatgtcagggtacaagtcaatatgatatgaatttaccttttttctcccacttgtccttctttctccaccccaccccctgcccgcgccggctttttttctctcccacctctttctttgactctgctgccgcctatcctggcggctcctcttcacctcccctgcctggcgatcccctccccctcctgccctatgtcctccagctcttggggaacaatcgccactgattgaggttcactctatgttaggctggaaaactgggctgttatttaggaagtcattaatcacatctcctcccccggaaagagatggcagagaaacctgtgaagtacaattccttaccaaccccctctaaaattttccaaaatggtttacctaaacacaatttgactatatgaaggtgaaaacgatttttctagtaggattttattttaaatttgaaaaaatttcgggcataatcaggtaatttatgtgcctctgccttttcctaggtgtatgttttcattatctaaagtttcatggggatgtaattaggtttaattagatcttttataaattatatattttcctctaagaattgaattaaatatgaaattagcgttccaagcaggtttactgccatggaatagctgaattcttaaggggaaaaaaagcgttaacatcggttgttttatttaaacgaccccctcccatgattttagtcaggcttgtgtgtaaggcacgcgtgtgcacatatatgactgcctttttttctaaatctatgattcacaaggtccctccattgtgtttaagtagaagcacgaaatcagcattctgatttatcctaaagaagcgttccaaattgtctttaagataacatgtttgagtttttcgtgttcattcataaattattttgatgtcagcatagatgaaatggcgattggttatctcttcctcttgccagccccttaaggatacgaggtgaaattagtagcaagaaagcatgtaattgacaaagtcacgtgtgctcagggggccagaaactggagagaggagagaaaaaaat

SEQ ID NO: 29 - DNA, 562 bp, Homo sapiens
  misc_feature 1..562: note = LINC00649 (Gene position chr21:33,915,534-34,003,026) which is oriented on the plus strand with the promoter region positioned (chr21:33,930,560-33,931,121)
  source 1..562: mol_type = genomic DNA
tcgtctgaagccttcttctctcagctcgtcaaagtcattctccatccagctttgttccgttgctggtgaggaactgcgttcctttggaggaggagaggcgctctgcattttagagcttccagtttttctgttctgttttttccccatctttgtggttttatctacttttggtctttgatgatggtgatgtacagatgggattttggtgtggatgtcctttctgtttattagttttccttctaacagacaggaccctcagctgcaggtctgttggaataccctgccgtgtgaggtgtcagtgtgcccctgctggggggtgcctcccagttaggctgctcgggggtcaggggtcagggacccacttgaggaggcagtctgcccgttctcagatctgcagctgcgtgctgggagaaccactgctctcttcaaagctgtcagacagggacatttaagtctgcagaggttactgctgtctttttgtttgtctgtgccctgcccccagaggtggagcctacagaggcaggcaggcctccttgagctgtggtgggctccacccagttcc

SEQ ID NO: 30 - DNA, 850 bp, Homo sapiens
  misc_feature 1..850: note = POTEE (Gene position chr2:131,209,536-131,265,278) which is oriented on the plus strand with the promoter region positioned (chr2:131,208,731-131,209,580)
  source 1..850: mol_type = genomic DNA
atctacccaaaaccttttctccccactgtcttttcacaaaaccttctctctctactgctcaacgctgtttctcccccccaccaccctctctttcctcctcccttgccaccctcttttcctcctccatctacccataaacattttacccaccatctttctgcaaaaccttccctccctgccgctccccaccccgtttttctccctccatctacccaaaaactttttttcccactatcttttccccaccgcctttttgcaacgcgctctcctgctcactatcctctcttccctttggcactaaccaccctctttaccccctccatctatcccaaaactcttttcctcctcttaccgcttccgccgcactgccgtctcggtcgcggttaccaccagtcgcagcgaggcgagccacggtgtagcggctccagcctccagcgtacggctggtgattacccattcctggtcctctaagccgggcactgagcagctccacaggaaaatacgggaacgtggaagagcctgacttcccttcagcagcaggcgtataccgcggttatatacaggaggattcctgactgcatgttctgattggatgagaaaaaccctccagggttacttggattggactttattatcatgttctgattggatgagagccagtcttaagacaaccaatcacagcatgaaaataaagtccaatcagagtaggcctagaggtttttctctcatccaatcagaacatgtagtctgggaaccacatgtgcgtaacctcagtacgtaaagcatgcggaggtggcgtcaggtcatttcaggctcttaagtgtgggcgtttggtaaccggcatggctgc

SEQ ID NO: 31 - DNA, 400 bp, Homo sapiens
  misc_feature 1..400: note = POTEF (gene position chr2:130,073,535-130,129,222) oriented on the minus strand with the promoter region positioned (chr2:130,129,223-130,129,622)
  source 1..400: mol_type = genomic DNA
cttcagcagcagtcatataccgagattatatacaggaggattcctgactgcatgttctgattggatgagaaaaaccctccagggttacttggattggactttattatcatgttctgattggatgagagccagtcttaagacaaccaatcacagcatgaaaataaagtccaatcagagtaggcctagaggtttttctctcatccaatcagaacatgtagtctgggaaccacatgtgcgtaacctcagtaggtaaagcatgcggaggtggcgtcaggtcatttcaggctcttaagtgtgggcgtttggtatccggcatggctgctacctgtttctggctggagcctcggacactggctcactgcagttggtggtgtcgacagtggtaggagggcaaccagta

SEQ ID NO: 32 - DNA, 400 bp, Homo sapiens
  misc_feature 1..400: note = MED15P9 (gene position chr2:130,129,621-130,139,417) oriented on the plus strand with the promoter region positioned (chr2:130,129,223-130,129,622)
  source 1..400: mol_type = genomic DNA
tactggttgccctcctaccactgtcgacaccaccaactgcagtgagccagtgtccgaggctccagccagaaacaggtagcagccatgccggataccaaacgcccacacttaagagcctgaaatgacctgacgccacctccgcatgctttacctactgaggttacgcacatgtggttcccagactacatgttctgattggatgagagaaaaacctctaggcctactctgattggactttattttcatgctgtgattggttgtcttaagactggctctcatccaatcagaacatgataataaagtccaatccaagtaaccctggagggtttttctcatccaatcagaacatgcagtcaggaatcctcctgtatataatctcggtatatgactgctgctgaag

SEQ ID NO: 33 - DNA, 1952 bp, Homo sapiens
  misc_feature 1..1952: note = PCAT7 (gene position chr9:94,554,461-94,603,990) oriented on the plus strand with the promoter region positioned (chr9:94,553,093-94,555,044)
  source 1..1952: mol_type = genomic DNA
ccgctgctaccaacgctagcgccgatgccagtacaaccgctgttgccctcaatgcaccagcccaccctacaaggctcctatcacttggcccccacgggtgccctcctaccactctggtcaagctgcagtctctgttgctgccaccaacctcatgaggcaagctgcagagccacgccatctacaggctccagcataccacaggtgactcctcctcctcctccagcctggctgggagcagttgcgcaggcaaagccagagaaacctagaacaggatgcagggagtggaagtgttagagcttcaccttgtcacgctggccactgggcggcagggaccatttcattaaaggcactcacacccaccttccaaagtccagcctctctttctggcaaaagctggccaggaactggggcctgggctgggtgtgagtgccttcactgaaaccggctcctggccaagtccagctggccaggaattgctgggcccatcggcactgcgctcttcggggagcccgcgtaggagaaactcagacccagccagccctctccacccaagtgctggttcccattcctcacgcctccacccacagtgccctgtccccctccctcccccgtggtgccctctattccctgcccggtagtcccagatggtctccacaacacagagcgtgcgtgagggcgcgggccagggaaccacagtgggtgtgagagccctgcggtgctcaagattgcatgagcacgaagagatcaccctctagagtctggagtccgggaagagaagaactccatccttccatggaggccaccagaaagaggaaggaggccacagctgtcgctgccgctgctgcctctgcagcccaccaaccccactggcagtatagcccccgatagcacccctaacctgccccctgtaaccctgggatagcacacccaacagctgcaggcaatgtaaccaatacccccccaaaccatcccaactcctcggccgcaggcagtgttagcacccgatagtgcccacctgacgcagccacgggtattgctgcactagacagcgccccaaacccgccccaccctcaccccgccatgggcagtgcagctcctgatagcgcacccccagtcacaggcagtgcagcaccggaccacgcccttaaaccaccccccactgccagcattgtagccccagataactccacccaacccgcccctgccacggacagtgcagcagaagacaacgcccctaagccatccaccgccactggcagtacacgttagtgtacacaacctgcctctcccgaccacccctgccacctcgggcagtgtagccccccattccaccaccagcaatgcaaccccagagagtgcccccaaacagtccaatgccacaggcagtgtagcctttggcagtgagcccctatagtacacccaacccttacccccagaagagggcagtgcagccctggataactcacctaccctaccacatttctaccactgtggccgagctgcggtctctgatttcaccaccaaccgcagcgaggcgagccgcggaggcgcaagctccggctccagcctccagcacgcggcggtgcctcttctttctgtcttccagcccggcaggagaagctctcgctgctagcctccctcctaccgctccgtcaccaccaccaccaacagggagggagtgccccaggctccaggctccaagctccaggctccagggggcgaaaagtgacggagccttccagttctttaagccgggtacgtagtagctccccacacccacacataagtgccagaaatgacgcgccgctgcctcagcatgctttatatactgaggttaggagaccctggactacatgttctgattggatgagagaaaaacctctaggcctactctgattggactttattttcatgctgtgattggttgtcttaagacttgctctcatccaatc

SEQ ID NO: 34 - DNA, 1246 bp, Homo sapiens
  misc_feature 1..1246: note = PURPL (gene position chr5:27,217,629-27,498,637) oriented on the plus strand with the promoter region positioned (chr5:27,471,047-27,472,292)
  source 1..1246: mol_type = genomic DNA
tctttggcagtgaaagctccccatagtccatccatcctactaattgtaactataaactttgaatactttttttttttttgagacggtgtctcactctgtcgcccaggttggagtgcagtggcaagatctcggctcacttcaacctctgctgcctgggttcaagtgattctcctgcctcagcctcctgagtagctgggattacaggcgcttgccaccgcgcctggctaatttttgtagtttttctaagtagagacggggaactttggatacattttttagagtaaaaaaagtaaattaaaaataccctaattgaaaactctgaaaataaacaagttagttttttttaagagatgccaaaatttgaaaaagcaactgttattaaagtgatttgcattattttttttttccttgggatattttttcccattcacagagtggaaaagcataggtgactacaagtcttgctagacactctgctgtcaatttggctaggaaaaaagcaaaaacaaaaacaagtaatggacattcagtaaatattagcagagacaaaggaatgttacaaaggaagcaaatggaaattttttaaatgaaaaataaaatatctgaaattaaacatttattaagttcaacagcaaaaaaagaataatagtaggaaaatatgttgaacataaaatagtatgtccaaaaagccagatctaacttcttattgcctacaaggaatcaactttgaatataaagaaaatggtacaaattaatagatcaatagccattccccaatataaagaacacagaacaatgaatgagactaaagagatactttgaaacagaaaaataaggaaacctcaaaacgtgaaggacaatattaaaaggtctaacgtatttgtaatttcggtacccaaagttaaaattggggcatacttctaaatacccaaagactttcaaaatctgcagtacagtcaatgacatacatttacgaatgaaagaagctcagcaaattccaaatagtgtaagtgggagcaaaatgcctaggcagataaaaaggggtccctagagaatttctggccttcttcacaagtgtttacattagatgcttttgtgcagatgagggaacctgcccagggcttgtctgggcatgcccacagtggactggagcctgacatgctcactggggcaagtgggtggagccatgaggaattcatgccttgcaaaggggaggagcctgccctcttgagctcttgtggtgacc

SEQ ID NO: 35 - DNA, 3317 bp, Homo sapiens
  misc_feature 1..3317: note = WDR72 (gene position chr15:53,513,741 -54,762,878) oriented on the minus strand with the promoter region positioned (chr15:53,541,104-53,544,420)
  source 1..3317: mol_type = genomic DNA
taatcatgtggtttttgtctttggctctgtttatatgctggattacatttattgatttgcgtatattgaaccagccttgcatcccagggatgaagcccacttgatcatggtggataagctttttgatgtgctgctggattcgttttgccagtattttattgaggatttttgcatcaatgttcatcaaggatattggtctaaaattctcttttttggttgtgtctctgtccggctttggtatcagaatgatgctggcctcataaaatgagttagggaggattccctctttttctattgattggaatagtttcagaaggaatggtaccagttcctccttgtacctctggtagaattcggctgtgaatccatctggtcctggactctttttggttggtaaactattgattattgccacaatttcagctcctgttattggtctattcagagattcaacttcttcctggttcagtcttgggagagtgtatgtgtcaaggaatttatccatttcttctagattttctagtttatttgcgtagaggtgtttgtagtttgtagaggtgtttgtagtactctgatggtagtttgtatttctgtgggatcggtggtgacatcccctttatcattttttattgcgtctattagattcttctctctttttttctttattagtcttgctagcggtctatcaattttgttgatcttttcagaaaacagctcctggattcattaattttttgaacggttttttgtgtctctatttccttcagttctgctctgattttagttatttcttgccttctgctagcttttgaatgtgtttgctcttgcttttctagttcttttaattgtgatgttagggtgtcaattttggatctttcctgctttctcttgtgggcatttagtgctataaatttccctctacacactgctttgaatgcgtcccagagattctggtatgttgtgtctttgttctcgttggtttcaaagaacatcttcctttctaccttcattttcttatgtacccagaagtcattcaggagcaggttgttcagtttccatgtagttgagcggttttgagtgagattcttaatcctgagttctagtttcattgcactgtggtctgagagatagtttgttataatctctgttcttttacatttgctgaggagagctttacttccaagtatgtggtcaattttggaataggtgtggtgtggtgctgaaaaaaatgtatattctgttgatttggggtggagagttctgtagatgtctattaggtccgcttggtgcagagctgagttcaattcctgggtatccttgttgactttctgtctcgttgatctgtctaatgttgacagtggggtgttaaagtctcccattattaatgtgtgggagtctaagtctctttgtaggtcactcaggacttgctttacgaatctgggtgctcctgtgttgagtgcatatatatttaggatagttagctcttcttgttgaattgatccctttaccattatgtaatggccttctttgtctcttttgatctttgttggtttaaagtctgttttatcagagactaggattgcaacccctgcctttttttgttttccatttgcttggtagatcttcctccatccttttattttgagcctatgtgtgtctctgcacgtgagatgggtttcctgaatacagcacactgatgggtcttgactctttatccaatttgccagtctgtgtcttttaattggagcatttagtccatttacatttaaagttaatattgttatgtgtgaatttgatcctgtcattatgatgttagctggtgattttgctcgttagttgatgcagtttcttcctagtctcgatggtctttacattttggcatgattttgcagcgactggtaccggttgttcctttccatgtttagcgcttccttcaggagctcttttagggcaggcctggtggtgacaaaatctctcagcatttgcttgtctgtaaagtattttatttctccttcacttatgaagcttagtttggctggatatgaaattctgggttgaaaattcttttctttaagaatgttgaatattggcccccactctcttctggcttgtagggtttctgccgagagatccgctgttagtctgatgggcttccctttgagggtaacccgacctttctctctggctgcccttaacattttttccttcatttcaactttggtgaatctgacaattatgtgtcttggagttgctcttctcgaggagtatctttgtggcgttctctgtatttcctgattctgaacgttggcctgccttgctagattggggaagttctcctggataatatcctgcagagtgttttccaacttggttccattctccccatcactttcaggtacaccaatcagacgtagatttggtcttttcacatagtcccatatttcttggaggctttgctcatttctttttattcttttttctctaaacttcccttctcacttcatttcattcatttcatcttccatcgctgataccctttcttccagttgatcgcatcggctcctgaggcttctgcattcttcacgtagttctcgagccttggttttcagctccatcagctcctttaagcacttctctgtattggttattctagttatacattcttctaaatttttttcaaagttttcaacttctttgcctttggtttgaatgtcctcccatagctcagagtaatttgatcatctgaagccttcttctctcagctcgtcaaagtcattctccatccagctttgttccgttgctggtgaggaactgcgttcctttggaggaggagaggcgctctgcgttttagagtatccagtttttctgttctgttttttccccatctttgtggttttatctacttttgctctttgatgatggtgaggtacagatgagtttttgctgtggatgtcctttctgtttgttagttttccttctaacagacaggaccctcagctgcaggtctgttggaataccttgccttgtgaggtatcagtgtgcccctgctggggggtgcctcccagttaggctgctcgggggtcaggggtcagggacccacttgaggaggcagtctgcccgttctcagatctccagctgcgtactgggagaaccactgctctcttcaaagctgttagatagggacatttaagtctgcagaggttactgctgtctttttgtttgtctgtgccctgcccccagaggtggagcctacagaggcaggcaggccaccttgagctgtggtgggctccacccagttctagcttcccggctgcttt

(19) United States
(12) Patent Application Publication
(10) Pub. No.: US 2024/0398987 A1
(43) Pub. Date: Dec. 5, 2024

(54) METHODS FOR THE TEMPORAL REGULATION OF REPROGRAMMING FACTORS IN MAMMALIAN CELLS

(71) Applicant: REVERSE BIOENGINEERING, INC., Alameda, CA (US)

(72) Inventors: Michael D. West, Mill Valley, CA (US); Hal Sternberg, Berkeley, CA (US)

(21) Appl. No.: 18/701,215

(22) PCT Filed: Oct. 14, 2022
(86) PCT No.: PCT/US2022/046737

§ 371 Date: Apr. 12, 2024

Related U.S. Application Data
(60) Provisional application No. 63/256,284, filed on Oct. 15, 2021.
(60) Provisional application No. 63/274,736, filed on Nov. 2, 2021.

(51) Int. Cl. A61K 48/00; A61K 38/45; C12N 5/074; C12N 15/86

(52) U.S. Cl. CPC A61K 48/005; A61K 38/45; C12N 5/0696; C12N 15/86; C12Y 207/01021; C12N 2506/1307; C12N 2750/14143

(57) ABSTRACT

Methods are provided for the ex vivo reprogramming of adult mammalian cells, wherein the genes used in reprogramming the adult cells are expressed with heterologous promoters that increase expression of associated genes while the cell is in a fetal or adult non-regenerative state, but down-regulated the expression of genes once cells reach a regenerative state and before the cells are reprogrammed to pluripotency. In addition, heterologous promoters uniquely expressing genes when cells are in an embryonic (pre-fetal state) are used to increase expression of toxic gene products in cancer cells.

### RELATED APPLICATIONS

The instant application claims priority to U.S. Provisional Application No. 63/256,284, filed Oct. 15, 2021; and U.S. Provisional Application No. 63/274,736, filed Nov. 2, 2021; entire contents of each of which are expressly incorporated by reference herein in their entireties.

### BACKGROUND

Advances in stem cell technology, such as the isolation and propagation in vitro of human pluripotent stem (hPS) cells constitute an important new area of medical research. hPS cells have a demonstrated potential to be propagated in the undifferentiated state and then to be induced to subsequently differentiate into any and all of the cell types in the human body, including complex tissues. This has led, for example, to the prediction that many diseases resulting from the dysfunction of cells may be amenable to treatment by the administration of human embryonic stem cell-derived of various differentiated types (Thomson et al., Science 282:1145-1147 (1998)).

With regard to methods of differentiating hPS cells into desired cell types, the potential to clonally isolate lines of human embryonic progenitor cells provides a means to propagate novel highly purified cell lineages with a prenatal pattern of gene expression useful for regenerating tissues such as skin in a scarless manner. Such cell types have important applications in research, and for the manufacture of cell-based therapies (see PCT application Ser. No. PCT/US2006/013519 filed on Apr. 11, 2006 and titled “Novel Uses of Cells With Prenatal Patterns of Gene Expression”; U.S. patent application Ser. No. 11/604,047 filed on Nov. 21, 2006 and titled “Methods to Accelerate the Isolation of Novel Cell Strains from Pluripotent Stem Cells and Cells Obtained Thereby”; and U.S. patent application Ser. No. 12/504,630 filed on Jul. 16, 2009 and titled “Methods to Accelerate the Isolation of Novel Cell Strains from Pluripotent Stem Cells and Cells Obtained Thereby”, each incorporated herein by reference).

More recently, the potential of pluripotent stem cells and derived embryoid bodies for in vitro self-assembly into 3-dimensional organoids has generated interest as a potential pathway for both obtaining tissue for transplantation (Singh et al, Stem Cells Dev. 2015. 24(23): 2778-95) as well as modeling human embryonic development. In contrast to embryonic cells, fetal and adult derived cells often show reduced potential for organogenesis in vitro and epimorphic regeneration in vivo. Epimorphic regeneration, sometimes referred to as “epimorphosis,” refers to a type of tissue regeneration wherein a blastema of relatively undifferentiated mesenchyme proliferates at the site of injury and then the cells differentiate to restore the original tissue histology in a scarless manner. The developmental timing of the loss of epimorphic potential cannot be fixed precisely, and likely varies with tissue type, nevertheless, the embryonic-fetal transition (EFT), or eight weeks of human development (Carnegie Stage 23; O'Rahilly, R., F. Miiller (1987) Developmental Stages in Human Embryos, Including a Revision of Streeter's ‘Horizons’ and a Survey of the Carnegie Collection. Washington, Carnegie Institution of Washington) appears to temporally correspond to the loss of skin regeneration in placental mammals (Walmsley, G. G. et al 2015. Scarless Wound Healing: Chasing the Holy Grail Plast Reconstr Surg. 135(3):907-17). Correlations between species show increased regenerative potential in the embryonic or larval state (reviewed in Morgan, T. H. (1901). Regeneration (New York: The MacMillan Company); also Sanchez Alvarado, A., and Tsonis, P. A. (2006). Bridging the regeneration gap: genetic insights from diverse animal models (Nat. Rev. Genet. 7, 873-884) suggest that tissue regeneration, as opposed to scarring, reflects the presence of an embryonic as opposed to fetal or adult phenotype. In the case of some species, a change in developmental timing (heterochrony) correlates with profound regenerative potential such as is the case in the developmental arrest in larval development (heterochrony) and limb regeneration observed in the Mexican salamander axolotl A. mexicanum) (Voss, S. R. et al, Thyroid hormone responsive QTL and the evolution of paedomorphic salamanders. Heredity (2012) 109, 293-298. In contrast, some animals such as the African Spiny mouse (Acomys cahirinus) show a profound potential for skin regeneration in the absence of overt heterochrony, perhaps reflecting uncharacterized molecular alterations (Gawriluk, T. R., 2016. Comparative analysis of ear-hole closure identifies epimorphic regeneration as a discrete trait in mammals. Nature Commun. 7:1 1164). Despite these observations, there are limited markers of the EFT to test the role of specific molecules in epimorphic regeneration. We previously disclosed compositions and methods related to markers of the EFT in mammalian species and their use in modulating tissue regeneration (See, e.g. U.S. provisional patent application No. 61/831,421, filed Jun. 5, 2013, PCT patent application PCT/US2014/040601, filed Jun. 3, 2014 and U.S. patent application Ser. No. 14/896,664, filed on Dec. 7, 2015, the disclosures of which are hereby incorporated by reference in their entirety. Nevertheless, additional molecular regulators and methods for modulating the EFT are needed for research and therapy in regenerative medicine and cancer. Early candidates for regulators of heterochrony were identified in C. elegans. These included lin-28/let-7 (Ambros, V. and Horvitz, H. R. (1984). Heterochronic mutants of the nematode Caenorhabditis elegans. Science 226, 409-416). More recently, transgenic expression of the paralog Lin28a in mice has been reported to increase skin regeneration and amputated digit regrowth following wounding and to increase markers of oxidative phosphorylation (Shyh-Chang, N. et al 2013. Lin28 Enhances Tissue Repair by Reprogramming Cellular Metabolism. Cell 155, 778-792). However, the regenerative potential in said mice is not comparable to the profound epimorphosis observed in Acomys cahirinus. The lin-28/let-7 axis has also been observed to be activated in a number of cancer cell types (Jiang, S. and Baltimore, D. 2016. RNA-binding protein Lin28 in cancer and immunity, Cancer Lett. 375(1): 108-13). The abnormal expression of LIN28 in cancer suggests that perhaps the reason for the natural selection for repression of regenerative potential at the EFT is that in most vertebrates selection for this trait, while potentially limiting survival following injury, could function as a tumor suppression mechanism. Also consistent with this hypothesis is the well-known observation that many cancers show an embryonic reversion such as the Warburg effect. The full identification of such molecular mechanisms would facilitate the invention of novel methods for modulating said molecular mechanisms in cells and tissue in vivo, to cause an “induced tissue regeneration” (iTR) to facilitate the repair of said tissues afflicted with trauma or degenerative disease, including but not limited to age-related degenerative disease, as well as facilitate basic research in tissue regeneration.

The constitutive expression of combinations of reprogramming factors or the reprogramming of cells to pluripotency could lead to the generation of pluripotent stem cell (induced pluripotent stem (iPS) cell)-derived teratomas or even cancer. Current protocols described in the literature therefore use vectors with inducible promoters such as doxycycline-inducible promoters or the administration of mRNA or small molecules wherein the timing of expression of the mRNA can be controlled based on a dosage schedule. Such current strategies to control the extent of reprogramming, however, are not effective in controlling the extent of reprogramming in a diverse population of cells, such as in a mammalian tissue, where the cells are in differing states of reprogramming. Existing methods target all the cells in a population, and are not cell-specific. Therefore, there is a need for improved cell-specific regulation of reprogramming such that reprogramming on a cellular level is controlled by regulatory elements responsive to the target developmental destination of the cells. For example, if the goal is to reverse the aging of mammalian somatic cells to a state just prior to the embryonic-fetal transition (EFT), there is a need for methods of regulating the reprogramming genes in gene therapy constructs such that reprogramming factors are expressed in an individual cells only up to the point wherein genes normally activated after the EFT are inhibited. When the cells show the pre-EFT pattern of gene expression, said improved methods of regulation would then inhibit the expression of the reprogramming factors. Given the need to highly regulate the extent of reprogramming in mammalian cells and tissues to improve the extent of therapeutically-useful reprogramming and to inhibit over-reprogramming such as to pluripotency, there therefore remains a need for methods to improve the precise regulation of the extent of reprogramming in target cells.

### SUMMARY

The present invention provides methods for the ex vivo reprogramming of adult mammalian cells, wherein the genes used in reprogramming the adult cells are expressed with heterologous promoters that increase expression of associated genes while the cell is in a fetal or adult non-regenerative state, but down-regulated the expression of genes once cells reach a regenerative state and before the cells are reprogrammed to pluripotency. In addition, heterologous promoters uniquely expressing genes when cells are in an embryonic (pre-fetal state) are used to increase expression of toxic gene products in cancer cells.

More specifically, the present disclosure provides compounds, compositions, and methods relating to the use of a subset of genes differentially-regulated during the transition from embryonic phases of mammalian development to fetal stages, referred to herein as the embryonic-fetal transition (EFT). Furthermore, the methods of the present invention relate to those genes that are differentially-expressed in the majority of the hundreds of diverse somatic cell types in mammals during EFT, referred to herein as “global EFT genes.” Furthermore, the methods of the present invention describe the use of gene regulatory elements such as promoter and enhancer sequences associated with said global EFT genes to: 1) regulate the extent of the reprogramming of the developmental age of adult mammalian somatic cells or the modulation of tissue regeneration, referred to as “Developmentally-Regulated induced Tissue Regeneration” (DR-iTR), or 2) to utilize the unexpectedly common expression of global EFT genes in diverse types of cancer cells, wherein said global EFT genes are expressed primarily in the embryonic but not the fetal or adult state of the majority of somatic cell types. Furthermore, the present invention discloses methods and compositions to selectively express gene products in said cancer cells but not normal surrounding cells that result in the selective destruction of cancer cells.

The methods described herein for DR-iTR are useful for the in vivo and ex vivo reprogramming of mammalian somatic cells and tissues to reverse the aging and induce a regenerative phenotype in said cells and tissues, wherein the level of transcription, translation, or stability of one or more genes responsible for the reprogramming are differentially regulated at specific points on the developmental timeline in specific tissues. Said methods are herein collectively referred to as “Developmentally-Regulated iTR methods” (DR-iTR methods). Said regulated reprogramming is useful for research in the biology of aging and tissue regeneration as well as for therapeutic use in mammals wherein the risk of incomplete reprogramming or over-reprogramming such as the reprogramming to a pluripotent stem cell state is minimized or prevented entirely.

As summarized in FIG. 1A, one aspect of the present disclosure, describes methods of DR-iTR wherein the methods activate the transcription of segmental iTR genes in mammalian nonregenerative adult somatic cells wherein said segmental iTR genes induce tissue regeneration without globally reprogramming said adult somatic cells fully to an embryonic (pre-fetal) regenerative state. The nonlimiting example shown is the use of the promoter of the adult-onset gene COX7A1 to regulate the expression of the embryonic growth factor anti Mullerian hormone (AMH) using a gene therapy vector in adult nonregenerative cells.

Additionally, as summarized in FIG. 1B, another aspect of the present disclosure, describes methods to accomplish RNAi targeting iTR inhibitory genes in mammalian nonregenerative adult somatic cells wherein said decreased levels of inhibitory iTR gene transcripts induce tissue regeneration without globally reprogramming said adult somatic cells fully to an embryonic (pre-fetal) regenerative state. The nonlimiting example shown is the use of the promoter of the adult-onset gene COX7A1 to regulate the expression of an RNAi construct targeting the transcript of the gene PCDHGA12 in a gene therapy vector in adult nonregenerative cells.

Additionally, as summarized in FIG. 1C, another aspect of the present disclosure, describes methods to accomplish global iTR in mammalian nonregenerative adult somatic cells wherein said global iTR results in the full reprogramming of adult somatic cells to an embryonic (pre-fetal) regenerative state. Said global reprogramming genes utilize combinations of the genes OCT4, SOX2, KLF4, NANOG, ESRRB, NR5A2, CEBPA, MYC, SALL4, LIN28A or LIN28B regulated by the promoter or enhancer of post-embryonic onset genes (fetal or adult onset) such that the combination of said global reprogramming factors are expressed in the cells expressing a post-embryonic (fetal or adult) pattern of nonregenerative gene expression but said global reprogramming genes are downregulated when said cells with a post-embryonic (fetal or adult) pattern of nonregenerative gene expression are fully reprogrammed to an embryonic regenerative state in order to reduce the probability of reprogramming to a undesired undifferentiated state such as to pluripotency.

Also disclosed herein, is the transient expression of telomerase in aged nonregenerative adult somatic cells in combination with the three categories of DR-iTR described above.

Additionally, as summarized in FIG. 1D, methods are disclosed to utilize the promoters or enhancer elements of segmental iTR genes expressed primarily in the embryonic, but not fetal or adult stages of somatic cell development to promote the expression of suicide constructs in cancer cells wherein the normal adult cells surrounding said cancer cells do not express the iTR gene and therefore do not express the suicide construct gene. As an example, as shown in FIG. 1D, the promoter to the gene CPT1B which is primarily expressed in embryonic as opposed to fetal or adult somatic cell types is used to promote the expression in cancer cells of the herpes simplex virus thymidine kinase (HSV TK) gene in a viral vector which in the presence of ganciclovir results in the targeted death of cancer cells as opposed to surrounding normal cells.

In another aspect of the present disclosure, methods are described to regulate the level of expression of one or more segmental iTR factor genes, wherein promoter or enhancer sequences normally regulating genes differentially expressed in the majority of somatic cell types during defined temporal events in embryonic, fetal, or neonatal developmental transitions, are used to instead regulate the expression of said reprogramming factor genes in a gene therapy vector.

In another aspect of the present disclosure, methods are described to regulate the level of expression of the segmental iTR factor gene AMH, wherein the promoter or sequences of 10 or more nucleotides from the promoter of the gene COX7A1 are positioned in cis with the AMH gene in a gene therapy vector to specifically express AMH in cells with a nonregenerative adult pattern of gene expression.

In another aspect of the present disclosure, methods are described to regulate the level of expression of the segmental iTR factor gene CDK4, wherein the promoter or sequences of 10 or more nucleotides from the promoter of the gene COX7A1 are positioned in cis with the CDK4 gene in a gene therapy vector to specifically express CDK4 in cells with a nonregenerative adult pattern of gene expression.

In another aspect of the present disclosure, methods are described to regulate the level of expression of one or more inhibitory iTR factor genes, wherein promoter or enhancer sequences normally regulating genes differentially expressed in the majority of somatic cell types during defined temporal events in embryonic, fetal, or neonatal developmental transitions, are used to instead regulate the expression from a gene therapy vector of RNAi sequences targeting said inhibitory iTR factor gene transcripts in adult nonregenerative cells.

In another aspect of the present disclosure, methods are described to regulate the level of expression of one or more of the reprogramming factor genes: OCT4, SOX2, KLF4, NANOG, ESRRB, NR5A2, CEBPA, MYC, SALL4, LIN28A or LIN28B wherein promoter or enhancer sequences normally regulating genes differentially expressed in the majority of somatic cell types during defined temporal events in embryonic, fetal, or neonatal developmental transitions, are used to instead regulate the expression of said reprogramming factor genes in a gene therapy vector.

In one aspect of the present disclosure, methods are described to regulate the level of expression reprogramming factor genes wherein promoter or enhancer sequences normally regulating genes differentially expressed in the majority of somatic cell types during defined temporal events in embryonic, fetal, or neonatal developmental transitions, are used to instead regulate the expression of reprogramming factor genes to regulate the extent of partial reprogramming.

In another aspect of the present disclosure, methods are described to regulate the level of expression of one or more of the reprogramming factor genes: OCT4, SOX2, KLF4, NANOG, ESRRB, NR5A2, CEBPA, MYC, SALL4, LIN28A or LIN28B wherein promoter or enhancer sequences normally regulating genes differentially expressed in the majority of somatic cell types during defined temporal events in embryonic, fetal, or neonatal developmental transitions, are used to instead regulate the expression of said reprogramming factor genes transported into cells using gene therapy vectors.

In another aspect of the present disclosure, methods are described to regulate the level of expression of one or more of the reprogramming factor genes: OCT4, SOX2, KLF4, NANOG, ESRRB, NR5A2, CEBPA, MYC, SALL4, LIN28A or LIN28B wherein promoter or enhancer sequences of one or more of the genes: C2CD6, CAT, COMT, COX7A1, GYPE, IHO1, KRBOX1, LINC00839, LINC00865, LRRK2, MEG3, MIRLET7BHG, NKAPL, PRR34-AS1, or ZNF300P1 are placed in cis with said reprogramming factor genes in a gene therapy vector to promote expression of the reprogramming factor genes in adult somatic cell types until defined temporal events in embryonic, fetal, or neonatal developmental transitions are reached and then reduce expression of said reprogramming factor genes to prevent over-reprogramming, such as reprogramming to pluripotency.

In another aspect of the present disclosure, methods are described to regulate the level of expression of one or more of the reprogramming factor genes: OCT4, SOX2, KLF4, NANOG, ESRRB, NR5A2, CEBPA, MYC, SALL4, LIN28A or LIN28B wherein the promoter of the gene COX7A1 is combined in cis with said reprogramming factor genes in a gene therapy vector to promote expression of the reprogramming factor genes in adult somatic cell types until the cells are reprogrammed to a temporal point of development corresponding to the embryonic-fetal transition (EFT) and then reduce expression of said reprogramming factor genes to prevent over-reprogramming, such as reprogramming to pluripotency.

In another aspect of the present disclosure, methods are described to regulate the level of expression of the reprogramming factor genes: OCT4, SOX2, KLF4, and MYC, wherein the promoter of the gene COX7A1 is combined in cis with said reprogramming factor genes in a gene therapy vector to promote expression of the reprogramming factor genes in adult somatic cell types until the cells are reprogrammed to a temporal point of development corresponding to the embryonic-fetal transition (EFT) and then reduce expression of said reprogramming factor genes to prevent over-reprogramming, such as reprogramming to pluripotency.

In another aspect of the present disclosure, methods are described to regulate the level of expression of the reprogramming factor genes: OCT4, SOX2, and KLF4, wherein the promoter of the gene COX7A1 is combined in cis with said reprogramming factor genes in a gene therapy vector to promote expression of the reprogramming factor genes in adult somatic cell types until the cells are reprogrammed to a temporal point of development corresponding to the embryonic-fetal transition (EFT) and then reduce expression of said reprogramming factor genes to prevent over-reprogramming, such as reprogramming to pluripotency.

In another aspect of the present disclosure, methods are described to regulate the level of expression of the reprogramming factor genes: LIN28A, OCT4, and KLF4, wherein the promoter of the gene COX7A1 is combined in cis with said reprogramming factor genes in a gene therapy vector to promote expression of the reprogramming factor genes in adult somatic cell types until the cells are reprogrammed to a temporal point of development corresponding to the embryonic-fetal transition (EFT) and then reduce expression of said reprogramming factor genes to prevent over-reprogramming, such as reprogramming to pluripotency.

In another aspect of the present disclosure, methods are described to regulate the level of expression of the reprogramming factor genes: LIN28A, OCT4, SOX2, and NANOG, wherein the promoter of the gene COX7A1 is combined in cis with said reprogramming factor genes in a gene therapy vector to promote expression of the reprogramming factor genes in adult somatic cell types until the cells are reprogrammed to a temporal point of development corresponding to the embryonic-fetal transition (EFT) and then reduce expression of said reprogramming factor genes to prevent over-reprogramming, such as reprogramming to pluripotency.

In another aspect of the present disclosure, methods are described to regulate the level of expression of one or more of the reprogramming factor genes: OCT4, SOX2, KLF4, NANOG, ESRRB, NR5A2, CEBPA, MYC, SALL4, LIN28A or LIN28B wherein the promoter of the gene CAT is combined in cis with said reprogramming factor genes in a gene therapy vector to promote expression of the reprogramming factor genes in adult somatic cell types until the cells are reprogrammed to a temporal point of development corresponding to the embryonic-fetal transition (EFT) and then reduce expression of said reprogramming factor genes to prevent over-reprogramming, such as reprogramming to pluripotency.

In another aspect of the present disclosure, methods are described to regulate the level of expression of one or more of the reprogramming factor genes: OCT4, SOX2, KLF4, NANOG, ESRRB, NR5A2, CEBPA, MYC, SALL4, LIN28A or LIN28B wherein the promoter of the gene KRBOX1 is combined in cis with said reprogramming factor genes in a gene therapy vector to promote expression of the reprogramming factor genes in adult somatic cell types until the cells are reprogrammed to a temporal point of development corresponding to the embryonic-fetal transition (EFT) and then reduce expression of said reprogramming factor genes to prevent over-reprogramming, such as reprogramming to pluripotency.

In another aspect of the present disclosure, methods are described to regulate the level of expression of one or more of the reprogramming factor genes: OCT4, SOX2, KLF4, NANOG, ESRRB, NR5A2, CEBPA, MYC, SALL4, LIN28A or LIN28B wherein the promoter of the gene MEG3 is combined in cis with said reprogramming factor genes in a gene therapy vector to promote expression of the reprogramming factor genes in adult somatic cell types until the cells are reprogrammed to a temporal point of development corresponding to the embryonic-fetal transition (EFT) and then reduce expression of said reprogramming factor genes to prevent over-reprogramming, such as reprogramming to pluripotency.

In another aspect of the present disclosure, methods are described to regulate the level of expression of one or more of the reprogramming factor genes: OCT4, SOX2, KLF4, NANOG, ESRRB, NR5A2, CEBPA, MYC, SALL4, LIN28A or LIN28B wherein the promoter of the gene NKAPL is combined in cis with said reprogramming factor genes in a gene therapy vector to promote expression of the reprogramming factor genes in adult somatic cell types until the cells are reprogrammed to a temporal point of development corresponding to the embryonic-fetal transition (EFT) and then reduce expression of said reprogramming factor genes to prevent over-reprogramming, such as reprogramming to pluripotency.

In another aspect of the present disclosure, methods are described to regulate the level of expression of one or more of the reprogramming factor genes: OCT4, SOX2, KLF4, NANOG, ESRRB, NR5A2, CEBPA, MYC, SALL4, LIN28A or LIN28B wherein the promoter of the gene PRR34-AS1 is combined in cis with said reprogramming factor genes in a gene therapy vector to promote expression of the reprogramming factor genes in adult somatic cell types until the cells are reprogrammed to a temporal point of development corresponding to the embryonic-fetal transition (EFT) and then reduce expression of said reprogramming factor genes to prevent over-reprogramming, such as reprogramming to pluripotency.

In another aspect of the present disclosure, methods are described to regulate the level of expression of one or more of the reprogramming factor genes: OCT4, SOX2, KLF4, NANOG, ESRRB, NR5A2, CEBPA, MYC, SALL4, LIN28A or LIN28B wherein the promoter of the gene ZNF300P1 is combined in cis with said reprogramming factor genes in a gene therapy vector to promote expression of the reprogramming factor genes in adult somatic cell types until the cells are reprogrammed to a temporal point of development corresponding to the embryonic-fetal transition (EFT) and then reduce expression of said reprogramming factor genes to prevent over-reprogramming, such as reprogramming to pluripotency.

In another aspect of the present disclosure, methods are provided for regulating the extent of reprogramming the developmental age of mammalian cells and tissues being the introduction to said cells and tissues of gene therapy vector constructs expressing reprogramming factors regulated by promoter and/or enhancer sequences for genes normally differentially expressed at defined stages of prenatal development.

In another aspect of the present disclosure, methods are described to regulate the level of expression of the reprogramming factor genes: LIN28A, OCT4, and KLF4, wherein the promoter of the gene COX7A1 is combined in cis with said reprogramming factor genes in a gene therapy vector to promote expression of the reprogramming factor genes in adult dermal cells until the cells are reprogrammed to a temporal point of development corresponding to the embryonic-fetal transition (EFT) wherein the dermis is capable of scarless regeneration, and then reduce expression of said reprogramming factor genes to prevent over-reprogramming, such as reprogramming to pluripotency.

In another aspect of the present disclosure, methods are described to regulate the level of expression of the reprogramming factor genes: LIN28A, OCT4, and KLF4, wherein the promoter of the gene COX7A1 is combined in cis with said reprogramming factor genes in a gene therapy vector to promote expression of the reprogramming factor genes in adult dermal cells within microbiopsies cultured ex vivo until the cells are reprogrammed to a temporal point of development corresponding to the embryonic-fetal transition (EFT) wherein the dermis is capable of scarless regeneration, and then reduce expression of said reprogramming factor genes to prevent over-reprogramming, such as reprogramming to pluripotency.

In another aspect of the present disclosure, methods are provided for regulating the extent of reprogramming the developmental age of mammalian cells and tissues to induce a scarless regenerative phenotype being the introduction to said cells and tissues of gene therapy constructs expressing reprogramming factors regulated by promoter and/or enhancer sequences for the gene ADIRF, normally upregulated at the perinatal stage of development.

In another aspect of the present disclosure, methods are provided for regulating the extent of reprogramming the developmental age of mammalian cells and tissues to induce a scarless regenerative phenotype being the introduction to said cells and tissues of gene therapy constructs expressing reprogramming factors regulated by the promoter and/or enhancer sequences normally regulating the expression of the gene to inhibit the expression of reprogramming factors when reversion of the cells to a perinatal stage of development is achieved.

In another aspect of the present disclosure, methods are provided for regulating the extent of reprogramming the developmental age of mammalian cells and tissues to induce a scarless regenerative phenotype being the introduction to said cells and tissues of gene therapy constructs expressing reprogramming factors regulated by promoter and/or enhancer sequences for gene normally differentially expressed immediately prior to the embryonic-fetal transitional stage of development.

In another aspect of the present disclosure, methods are provided for regulating the extent of reprogramming the developmental age of mammalian cells and tissues to induce a scarless regenerative phenotype being the introduction to said cells and tissues of gene therapy constructs expressing reprogramming factors regulated by the promoter and/or enhancer sequences normally regulating the expression of the gene to inhibit the expression of reprogramming factors when reversion of the cells to a perinatal stage of development is achieved.

In another aspect of the present disclosure, methods are provided for regulating the extent of reprogramming the developmental age of mammalian cells and tissues to induce a scarless regenerative phenotype being the introduction to said cells and tissues of gene therapy constructs expressing reprogramming factors regulated by promoter and/or enhancer sequences for the gene COX7A1, normally differentially expressed immediately after the embryonic-fetal transitional stage of development in diverse somatic cell types.

In another aspect, the disclosure provides methods of temporally regulating the reprogramming mammalian somatic cells and tissues to a scarless regenerative state by regulating the administration of iTR factors utilizing regulatory elements selected from those normally temporally-regulating genes, wherein the factors include those capable in other conditions of inducing pluripotency in somatic cell types, that is, in generating iPS cells, said factors including vectors expressing combinations of the genes: OCT4, SOX2, KLF4, NANOG, ESRRB, NR5A2, CEBPA, MYC, LIN28A, TERT, and LIN28B, their encoded RNAs, or proteins.

In another aspect, the disclosure provides methods of eliminating cancer cells that have reverted to an embryonic state by the administration of toxic genes wherein said toxic genes are regulated in cis by promoters or enhancers that normally are expressed in diverse embryonic (pre-fetal) cell types but not their adult counterparts, with the result that said cancer cells expressing an embryonic phenotype are destroyed.

In another aspect, the disclosure provides methods of eliminating cancer cells that have reverted to an embryonic state by the administration of toxic genes in gene therapy vectors wherein said toxic genes are regulated in cis by promoters or enhancers that normally are expressed in diverse embryonic (pre-fetal) cell types but not their adult counterparts, with the result that said cancer cells expressing an embryonic phenotype are destroyed.

In another aspect, the disclosure provides methods of eliminating cancer cells that have reverted to an embryonic state by the administration of the herpes virus thymidine kinase gene (HSV TK) in a gene therapy vector wherein said HSV TK gene is regulated in cis by the promoter of the gene CPT1B, enhancers that normally are expressed in diverse embryonic (pre-fetal) cell types but not their adult counterparts, with the result that said cancer cells expressing an embryonic phenotype are destroyed in the presence of ganciclovir.

In another aspect, the disclosure provides methods of eliminating carcinoma cells that have reverted to an embryonic state by the administration of toxic genes in gene therapy vectors wherein said toxic genes are regulated in cis by promoters or enhancers that normally are expressed in diverse embryonic (pre-fetal) cell types but not their adult counterparts, with the result that said carcinoma cells expressing an embryonic phenotype are destroyed.

In another aspect, the disclosure provides methods of eliminating sarcoma cells that have reverted to an embryonic state by the administration of toxic genes in gene therapy vectors wherein said toxic genes are regulated in cis by promoters or enhancers that normally are expressed in diverse embryonic (pre-fetal) cell types but not their adult counterparts, with the result that said sarcoma cells expressing an embryonic phenotype are destroyed.

In one aspect, the present disclosure provides for a method of reprogramming adult mammalian somatic cells to a scarless regenerative state, the method comprising contacting the cells with one or more induced tissue regeneration (iTR) factors that comprise: one or more nucleic acids encoding OCT4, SOX2, KLF4, NANOG, ESRRB, NR5A2, CEBPA, MYC, SALL4, LIN28A or LIN28B, wherein the one or more iTR factors are operably linked to a heterologous promoter or enhancer sequence, wherein the heterologous promoter or enhancer sequence induces expression temporally during embryonic, fetal, or neonatal developmental transitions.

In one embodiment, the heterologous promoter or enhancer sequence comprises a sequence that has at least 90%, 91%, 92%, 93%, 94%, 95%, 96%, 97%, 98%, or 99% identity to, comprises, or consists of any one of SEQ ID NOs: 1-15. In one embodiment, the heterologous promoter or enhancer comprises a sequence that has at least 90%, 91%, 92%, 93%, 94%, 95%, 96%, 97%, 98%, or 99% identity to, comprises, or consists SEQ ID NO: 4.

In one embodiment, the one or more iTR factors comprise (a) a nucleic acid encoding OCT4, SOX2, KLF4, and MYC; (b) one or more nucleic acids encoding OCT4, SOX2, KLF4, and MYC; (c) one or more nucleic acids encoding LIN28A, OCT4, and KLF4; or (d) one or more nucleic acids encoding LIN28A, OCT4, SOX2, and NANOG.

In one embodiment, the mammal is human.

In one embodiment, the one or more iTR factor genes are delivered by viral vector. In one embodiment, the viral vector is an adeno-associated virus.

In one embodiment, the viral vector is present in a pharmaceutical composition. In one embodiment, the pharmaceutical composition comprises a lipid formulation. In one embodiment, the lipid formulation comprises one or more cationic lipids, non-cationic lipids, and/or PEG-lipids, or a combination thereof.

In one embodiment, the somatic cells reside in microbiopsied tissue cultured in vitro.

In another aspect, the present disclosure provides for a method of treating cancer in a mammal, a method comprising administering one or more toxic genes to cancer cells in the mammal, wherein the one or more toxic genes are operably linked to a heterologous promoter or enhancer sequence, wherein the heterologous promoter or enhancer sequence induces expression induces expression in embryonic cells but not adult cells, wherein the cancer cells have reverted to an embryonic state.

In one embodiment, the heterologous promoter or enhancer comprises a sequence that has at least 90%, 91%, 92%, 93%, 94%, 95%, 96%, 97%, 98%, or 99% identity to, comprises, or consists of any one of SEQ ID NOs: 21-35.

In one embodiment, the mammal is human.

In one embodiment, the toxic gene product is simplex virus thymidine kinase (HSV TK).

In one embodiment, the cancer is a carcinoma.

In one embodiment, the one or more toxic genes are delivered by viral vector. In one embodiment, the viral vector is an adeno-associated viral vector. In one embodiment, the viral vector is present in a pharmaceutical composition. In one embodiment, the pharmaceutical composition comprises a lipid formulation. In one embodiment, the lipid formulation comprises one or more cationic lipids, non-cationic lipids, and/or PEG-lipids, or a combination thereof.

### BRIEF DESCRIPTION OF THE DRAWINGS

FIG. 1A shows a fetal/adult-specific gene promoter, in this case the promoter of COX7A1 in cis with a segmental iTR factor (in this case anti-mullerian hormone (AMH)).

FIG. 1B shows a fetal/adult-specific gene promoter, in this case the promoter of COX7A1 in cis with an RNAi construct targeting an iTR inhibitor gene (in this case the clustered protocadherin gene PCDHGA12.

FIG. 1C shows a fetal/adult promoter in cis with a polycistronic construct with global reprogramming genes such as KLF4, OCT4, and LIN28A to limit the expression of the reprogramming factors to only fetal or adult non-regenerative cells.

FIG. 1D shows a DR-O construct wherein an embryonic promoter, in this case the promoter to the gene CPT1B in cis with a toxic gene product, in this case, HSV TK.

### DETAILED DESCRIPTION

### Abbreviations

AC—Adult-derived cells

DMEM—Dulbecco's modified Eagle's medium

DMSO—Dimethyl sulfoxide

DPBS—Dulbecco's Phosphate Buffered Saline

DR-iTR—Developmentally-Regulated induced Tissue Regeneration

DR-O—Developmentally-Regulated Oncolysis

EDTA—Ethylenediamine tetraacetic acid

EFT—Embryonic-Fetal Transition

EG Cells—Embryonic germ cells; hEG cells are human EG cells

EP—Embryonic progenitors

EP cells—Embryonic progenitor cells

ES Cells—Embryonic stem cells; hES cells are human ES cells

ESC—Embryonic Stem Cells

FACS—Fluorescence activated cell sorting

FBS—Fetal bovine serum

FPKM—Fragments Per Kilobase of transcript per Million mapped reads from RNA sequencing.

GFP—Green fluorescent protein

GMP—Good Manufacturing Practices

hEG Cells—Human embryonic germ cells are stem cells derived from the primordial germ cells of fetal tissue.

HES cells—Human Embryonic Stem Cells

HESC—Human Embryonic Stem Cells

hiPS Cells—Human induced pluripotent stem cells are cells with properties similar to hES cells obtained from somatic cells after exposure to hES-specific transcription factors such as SOX2, KLF4, OCT4, MYC, or NANOG, LIN28A, OCT4, and SOX2.

HSE—Human skin equivalents are mixtures of cells and biological or synthetic matrices manufactured for testing purposes or for therapeutic application in promoting wound repair.

iCM—Induced Cancer Maturation.

iPS Cells—Induced pluripotent stem cells are cells with properties similar to hES cells obtained from somatic cells after exposure to ES-specific transcription factors such as SOX2, KLF4, OCT4, MYC, or NANOG, LIN28, OCT4, and SOX2, SOX2, KLF4, OCT4, MYC, and (LIN28A or LIN28B), or other combinations of OCT4, SOX2, KLF4, NANOG, ESRRB, NR5A2, CEBPA, MYC, LIN28A and LIN28B.

IRES—Internal Ribosome Entry Site

iTM—Induced Tissue Maturation

iTR—Induced Tissue Regeneration

MEM—Minimal essential medium

MSCs—Mesenchymal stem cells

NT—Nuclear Transfer

PBS—Phosphate buffered saline

PS fibroblasts—Pre-scarring fibroblasts are fibroblasts derived from the skin of early gestational skin or derived from ED cells that display a prenatal pattern of gene expression in that they promote the rapid healing of dermal wounds without scar formation.

RFU—Relative Fluorescence Units

RNA-seq—RNA sequencing

SFM—Serum-Free Medium

TR—Tissue Regeneration

### Definitions

The term “adult phenotype” when used to describe mammalian somatic cells, refers to the state of somatic cell development wherein the cells are no longer in the embryonic/regenerative stages of development, but have instead progressed into fetal or adult/nonregenerative stages of development. The term “analytical reprogramming technology” refers to a variety of methods to

reprogram the pattern of gene expression of a somatic cell to that of a more pluripotent state, such as that of an iPS, ES, ED, EC or EG cell, wherein the reprogramming occurs in multiple and discrete steps and does not rely simply on the transfer of a somatic cell into an oocyte and the activation of that oocyte (see U.S. application Nos. 60/332,510, filed Nov. 26, 2001; Ser. No. 10/304,020, filed Nov. 26, 2002; PCT application no. PCT/US02/37899, filed Nov. 26, 2003; U.S. application No. 60/705,625, filed Aug. 3, 2005; U.S. application No. 60/729,173, filed Aug. 20, 2005; U.S. application No. 60/818,813, filed Jul. 5, 2006, PCT/US06/30632, filed Aug. 3, 2006, the disclosure of each of which is incorporated by reference herein). The term “blastomere/morula cells” refers to blastomere or morula cells in a mammalian embryo or blastomere or morula cells cultured in vitro with or without additional cells including differentiated derivatives of those cells.

The term “cell line” refers to a mortal or immortal population of cells that is capable of propagation and expansion in vitro.

The term “differentiated cells” when used in reference to cells made by methods of this disclosure from pluripotent stem cells refer to cells having reduced potential to differentiate when compared to the parent pluripotent stem cells. The differentiated cells of this disclosure comprise cells that could differentiate further (i.e., they may not be terminally differentiated).

The term “embryonic” or “embryonic stages of development” refers to prenatal stages of development of cells, tissues or animals, specifically, the embryonic phases of development of cells compared to fetal and adult cells. In the case of the human species, the transition from embryonic to fetal development occurs at about 8 weeks of prenatal development, in mouse it occurs on or about 16 days, and in the rat species, at approximately 17.5 days post coitum. (embryology.med.unsw.edu.au/embryology/index.php?title=Mouse_Timeline_Detailed).

The term “embryonic stem cells” (ES cells) refers to cells derived from the inner cell mass of blastocysts, blastomeres, or morulae that have been serially passaged as cell lines while maintaining an undifferentiated state (e.g. expressing TERT, OCT4, and SSEA and TRA antigens specific for ES cells of the species).

The term “global EFT genes” refers to genes differentially-regulated (either up- or down-regulated) in a majority of diverse somatic cell types at or around the EFT.

The term “global modulator of TR” or “global modulator of iTR” refers to agents including combinations of the expressed genes OCT4, SOX2, KLF4, NANOG, ESRRB, NR5A2, CEBPA, MYC, LIN28A, TERT, and LIN28B, including but not limited to OCT4, SOX2, KLF4, and MYC; or OCT4, SOX2, LIN28A, and NANOG; or OCT4, LIN28A, and KLF4; capable of modulating in cells in vivo or cultured in vitro, including cultured microbiopsies, a multiplicity of iTR marker genes from a pattern of expression of a non-regenerative adult state to that more closely matching that of an embryonic (pre-fetal) regenerative state. Said global modulators of iTR are capable of downregulating COX7A1 expression while simultaneously up-regulating expression of PCDHB2, or downregulating expression of NAALADL1 while simultaneously up-regulating expression of AMH in cells derived from fetal or adult sources and are capable of inducing a pattern of gene expression leading to increased scarless tissue regeneration in response to tissue damage or degenerative when transiently expressed, or alternatively, are capable of reprogramming cells to pluripotency if expressed in the somatic cells for a sufficient period of time.

The term “human embryonic stem cells” (hES cells) refers to human ES cells.

The term “human induced pluripotent stem cells” refers to cells with properties similar to hES cells, including the ability to form all three germ layers when transplanted into immunocompromised mice wherein said iPS cells are derived from cells of varied somatic cell lineages following exposure to de-differentiation factors, for example hES cell-specific transcription factor combinations: KLF4, SOX2, MYC; OCT4 or SOX2, OCT4, NANOG, and LIN28; or various combinations of OCT4, SOX2, KLF4, NANOG, ESRRB, NR5A2, CEBPA, MYC, LIN28A and LIN28B or other methods that induce somatic cells to attain a pluripotent stem cell state with properties similar to hES cells. However, the reprogramming of somatic cells by somatic cell nuclear transfer (SCNT) are typically referred to as NT-ES cells as opposed to iPS cells.

The term “induced tissue regeneration” refers to the use of the methods of the present disclosure as well as previous disclosures (see PCT/US14/40601, filed Jun. 3, 2014 and titled “Compositions and Methods for Induced Tissue Regeneration in Mammalian Species,” and PCT/US2017/036452, filed Jun. 7, 2017 and titled “Improved Methods for Detecting and Modulating the Embryonic-Fetal Transition in Mammalian Species,” contents of each of which are incorporated herein by reference) to alter the molecular composition of fetal or adult mammalian cells such that said cells are capable or regenerating functional tissue following damage to that tissue wherein said regeneration would not be the normal outcome in animals of that species.

The term “isolated” refers to a substance that is (i) separated from at least some other substances with which it is normally found in nature, usually by a process involving the hand of man, (ii) artificially produced (e.g., chemically synthesized), and/or (iii) present in an artificial environment or context (i.e., an environment or context in which it is not normally found in nature).

The term “iTR factors” refers to molecules that alter the levels of TR activators and TR inhibitors in a manner leading to TR in a tissue not naturally capable of TR.

The term “iTR genes” refers to genes that when altered in expression can cause induced tissue regeneration in tissues not normally capable of such regeneration.

The term “iTR microbiopsy” refers to a microbiopsy that has been exposed while remaining an intact three dimensional tissue in organ culture (as opposed to isolated cells in culture) to iTR factors to increase the capacity for tissue to expand in volume by means of cell division and/or to promote scarless tissue regeneration when the reprogrammed microbiopsy by DT-iTR is engrafted in vivo.

The term “nucleic acid” is used interchangeably with “polynucleotide” and encompasses in various embodiments naturally occurring polymers of nucleosides, such as DNA and RNA, and non-naturally occurring polymers of nucleosides or nucleoside analogs. In some embodiments, a nucleic acid comprises standard nucleosides (abbreviated A, G, C, T, U). In other embodiments, a nucleic acid comprises one or more non-standard nucleosides. In some embodiments, one or more nucleosides are non-naturally occurring nucleosides or nucleotide analogs. A nucleic acid can comprise modified bases (for example, methylated bases), modified sugars (2′-fluororibose, arabinose, or hexose), modified phosphate groups or other linkages between nucleosides or nucleoside analogs (for example, phosphorothioates or 5′-N-phosphoramidite linkages), locked nucleic acids, or morpholinos. In some embodiments, a nucleic acid comprises nucleosides that are linked by phosphodiester bonds, as in DNA and RNA. In some embodiments, at least some nucleosides are linked by non-phosphodiester bond(s). A nucleic acid can be single-stranded, double-stranded, or partially double-stranded. An at least partially double-stranded nucleic acid can have one or more overhangs, e.g., 5′ and/or 3′ overhang(s). Nucleic acid modifications (e.g., nucleoside and/or backbone modifications, including use of non-standard nucleosides) known in the art as being useful in the context of RNA interference (RNAi), aptamer, or antisense-based molecules for research or therapeutic purposes are contemplated for use in various embodiments of the instant disclosure. See, e.g., Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008. In some embodiments, a modification increases half-life and/or stability of a nucleic acid, e.g., in vivo, relative to RNA or DNA of the same length and strandedness. In some embodiments, a modification decreases immunogenicity of a nucleic acid relative to RNA or DNA of the same length and strandedness. In some embodiments, between 5% and 95% of the nucleosides in one or both strands of a nucleic acid are modified. Modifications may be located uniformly or nonuniformly, and the location of the modifications (e.g., near the middle, near or at the ends, alternating, etc.) can be selected to enhance desired propert(ies). A nucleic acid may comprise a detectable label, e.g., a fluorescent dye, radioactive atom, etc. “Oligonucleotide” refers to a relatively short nucleic acid, e.g., typically between about 4 and about 60 nucleotides long. Where reference is made herein to a polynucleotide, it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “microbiopsy” refers to a three dimensional sample of mammalian, including human tissue, with a greatest size on two of three dimension of no more than 2 mm, preferably 1 mm or less.

The term “pluripotent stem cells” refers to animal cells capable of differentiating into more than one differentiated cell type. Such cells include hES cells, blastomere/morula cells and their derived hED cells, hiPS cells, hEG cells, hEC cells, and adult-derived cells including mesenchymal stem cells, neuronal stem cells, and bone marrow-derived stem cells. Pluripotent stem cells may be genetically modified or not genetically modified. Genetically modified cells may include markers such as fluorescent proteins to facilitate their identification within the egg.

The term “polypeptide” refers to a polymer of amino acids. The terms “protein” and “polypeptide” are used interchangeably herein. A peptide is a relatively short polypeptide, typically between about 2 and 60 amino acids in length. Polypeptides used herein typically contain the standard amino acids (i.e., the 20 L-amino acids that are most commonly found in proteins). However, a polypeptide can contain one or more non-standard amino acids (which may be naturally occurring or non-naturally occurring) and/or amino acid analogs known in the art in certain embodiments. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated. A polypeptide may be cyclic or contain a cyclic portion. Where a naturally occurring polypeptide is discussed herein, it will be understood that the disclosure encompasses embodiments that relate to any isoform thereof (e.g., different proteins arising from the same gene as a result of alternative splicing or editing of mRNA or as a result of different alleles of a gene, e.g., alleles differing by one or more single nucleotide polymorphisms (typically such alleles will be at least 95%, 96%, 97%, 98%, 99%, or more identical to a reference or consensus sequence). A polypeptide may comprise a sequence that targets it for secretion or to a particular intracellular compartment (e.g., the nucleus) and/or a sequence targets the polypeptide for post-translational modification or degradation. Certain polypeptides may be synthesized as a precursor that undergoes post-translational cleavage or other processing to become a mature polypeptide. In some instances, such cleavage may only occur upon particular activating events. Where relevant, the disclosure provides embodiments relating to precursor polypeptides and embodiments relating to mature versions of a polypeptide.

The term “prenatal” refers to a stage of embryonic or fetal development of a placental mammal prior to birth.

The term “purified” refers to agents or entities (e.g., compounds) that have been separated from most of the components with which they are associated in nature or when originally generated. In general, such purification involves action of the hand of man. Purified agents or entities may be partially purified, substantially purified, or pure. Such agents or entities may be, for example, at least 50%, 60%, 70%, 75%, 80%, 85%, 90%, 95%, 96%, 97%, 98%, 99%, or more than 99% pure. In some embodiments, a nucleic acid or polypeptide is purified such that it constitutes at least 75%, 80%, 855%, 90%, 95%, 96%, 97%, 98%, 99%, or more, of the total nucleic acid or polypeptide material, respectively, present in a preparation. Purity can be based on, e.g., dry weight, size of peaks on a chromatography tracing, molecular abundance, intensity of bands on a gel, or intensity of any signal that correlates with molecular abundance, or any art-accepted quantification method. In some embodiments, water, buffers, ions, and/or small molecules (e.g., precursors such as nucleotides or amino acids), can optionally be present in a purified preparation. A purified molecule may be prepared by separating it from other substances (e.g., other cellular materials), or by producing it in such a manner to achieve a desired degree of purity. In some embodiments, a purified molecule or composition refers to a molecule or composition that is prepared using any art-accepted method of purification. In some embodiments “partially purified” means that a molecule produced by a cell is no longer present within the cell, e.g., the cell has been lysed and, optionally, at least some of the cellular material (e.g., cell wall, cell membrane(s), cell organelle(s)) has been removed.

The term “reprogramming factor genes” refers to genes or cDNA sequences corresponding to said genes, that when used in various combinations are capable of reversing the developmental aging of mammalian somatic cell types. Said reprogramming factor genes include those capable of reverting somatic cells to pluripotency (induced pluripotent stem cells (iPSCs)) and include: OCT4, SOX2, KLF4, NANOG, ESRRB, NR5A2, CEBPA, MYC, LIN28A and LIN28B.

The term “RNA interference” (RNAi) is used herein consistently with its meaning in the art to refer to a phenomenon whereby double-stranded RNA (dsRNA) triggers the sequence-specific degradation or translational repression of a corresponding mRNA having complementarity to a strand of the dsRNA. It will be appreciated that the complementarity between the strand of the dsRNA and the mRNA need not be 100% but need only be sufficient to mediate inhibition of gene expression (also referred to as “silencing” or “knockdown”). For example, the degree of complementarity is such that the strand can either (i) guide cleavage of the mRNA in the RNA-induced silencing complex (RISC); or (ii) cause translational repression of the mRNA. In certain embodiments the double-stranded portion of the RNA is less than about 30 nucleotides in length, e.g., between 17 and 29 nucleotides in length. In certain embodiments a first strand of the dsRNA is at least 80%, 85%, 90%, 95%, or 100% complementary to a target mRNA and the other strand of the dsRNA is at least 80%, 85%, 90%, 95%, or 100% complementary to the first strand. In mammalian cells, RNAi may be achieved by introducing an appropriate double-stranded nucleic acid into the cells or expressing a nucleic acid in cells that is then processed intracellularly to yield dsRNA therein. Nucleic acids capable of mediating RNAi are referred to herein as “RNAi agents”. Exemplary nucleic acids capable of mediating RNAi are a short hairpin RNA (shRNA), a short interfering RNA (siRNA), and a microRNA precursor. These terms are well known and are used herein consistently with their meaning in the art. siRNAs typically comprise two separate nucleic acid strands that are hybridized to each other to form a duplex. They can be synthesized in vitro, e.g., using standard nucleic acid synthesis techniques. siRNAs are typically double-stranded oligonucleotides having 16-30, e.g., 16, 17, 18, 19, 20, 21, 22, 23, 24, 25, 26, 27, 28, 29, or 30 nucleotides (nt) in each strand, wherein the double-stranded oligonucleotide comprises a double-stranded portion between 15 and 29 nucleotides long and either or both of the strands may comprise a 3′ overhang between, e.g., 1-5 nucleotides long, or either or both ends can be blunt. In some embodiments, an siRNA comprises strands between 19 and 25 nt, e.g., between 2 1 and 23 nucleotides long, wherein one or both strands comprises a 3′ overhang of 1-2 nucleotides. One strand of the double-stranded portion of the siRNA (termed the “guide strand” or “antisense strand”) is substantially complementary (e.g., at least 80% or more, e.g., 85%, 90%, 95%, or 100%) complementary to (e.g., having 3, 2, 1, or 0 mismatched nucleotide(s)) a target region in the mRNA, and the other double-stranded portion is substantially complementary to the first double-stranded portion. In many embodiments, the guide strand is 100% complementary to a target region in an mRNA and the other passenger strand is 100% complementary to the first double-stranded portion (it is understood that, in various embodiments, the 3′ overhang portion of the guide strand, if present, may or may not be complementary to the mRNA when the guide strand is hybridized to the mRNA). In some embodiments, a shRNA molecule is a nucleic acid molecule comprising a stem-loop, wherein the double-stranded stem is 16-30 nucleotides long and the loop is about 1-10 nucleotides long. siRNA can comprise a wide variety of modified nucleosides, nucleoside analogs and can comprise chemically or biologically modified bases, modified backbones, etc. Without limitation, any modification recognized in the art as being useful for RNAi can be used. Some modifications result in increased stability, cell uptake, potency, etc. Some modifications result in decreased immunogenicity or clearance. In certain embodiments the siRNA comprises a duplex about 19-23 (e.g., 19, 20, 21, 22, or 23) nucleotides in length and, optionally, one or two 3′ overhangs of 1-5 nucleotides in length, which may be composed of deoxyribonucleotides. shRNA comprise a single nucleic acid strand that contains two complementary portions separated by a predominantly non-self complementary region. The complementary portions hybridize to form a duplex structure and the non-self complementary region forms a loop connecting the 3′ end of one strand of the duplex and the 5′ end of the other strand. shRNAs undergo intracellular processing to generate siRNAs. Typically, the loop is between 1 and 8, e.g., 2-6 nucleotides long. MicroRNAs (miRNAs) are small, naturally occurring, non-coding, single-stranded RNAs of about 21-25 nucleotides (in mammalian systems) that inhibit gene expression in a sequence-specific manner. They are generated intracellularly from precursors (pre-miRNA) having a characteristic secondary structure comprised of a short hairpin (about 70 nucleotides in length) containing a duplex that often includes one or more regions of imperfect complementarity which is in turn generated from a larger precursor (pri-miRNA). Naturally occurring miRNAs are typically only partially complementary to their target mRNA and often act via translational repression. RNAi agents modelled on endogenous miRNA or miRNA precursors are of use in certain embodiments of the disclosure. For example, an siRNA can be designed so that one strand hybridizes to a target mRNA with one or more mismatches or bulges mimicking the duplex formed by a miRNA and its target mRNA. Such siRNA may be referred to as miRNA mimics or miRNA-like molecules. miRNA mimics may be encoded by precursor nucleic acids whose structure mimics that of naturally occurring miRNA precursors. In certain embodiments an RNAi agent is a vector (e.g., a plasmid or virus) that comprises a template for transcription of an siRNA (e.g., as two separate strands that can hybridize to each other), shRNA, or microRNA precursor. Typically the template encoding the siRNA, shRNA, or miRNA precursor is operably linked to expression control sequences (e.g., a promoter), as known in the art. Such vectors can be used to introduce the template into vertebrate cells, e.g., mammalian cells, and result in transient or stable expression of the siRNA, shRNA, or miRNA precursor. Precursors (shRNA or miRNA precursors) are processed intracellularly to generate siRNA or miRNA. In general, small RNAi agents such as siRNA can be chemically synthesized or can be transcribed in vitro or in vivo from a DNA template either as two separate strands that then hybridize, or as an shRNA which is then processed to generate an siRNA. Often RNAi agents, especially those comprising modifications, are chemically synthesized. Chemical synthesis methods for oligonucleotides are well known in the art.

The term “segmental induced tissue regeneration” or “segmental iTR” refers to the reprogramming of only a subset of the cellular processes that promote some aspect of tissue regeneration. Examples would include the reprogramming of metabolism by the down-regulation of COX7A1 or the induction of cell proliferation by the transient expression of CDK4.

The term “segmental iTR factor” refers to a small molecule, protein, RNA, or gene that when introduced or expressed in a nonregenerative adult somatic cell type reverts a subset of said adult cell's molecular pathways to that of an embryonic regenerative cell to promote some aspect of tissue regeneration. Examples of segmental iTR factors would be the down-regulation of COX7A1 using RNAi or the induction of cell proliferation by the transient expression of CDK4.

The term “small molecule” as used herein, is an organic molecule that is less than about 2 kilodaltons (KDa) in mass. In some embodiments, the small molecule is less than about 1.5 KDa, or less than about 1 KDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

In some embodiments, a small molecule is non-polymeric. In some embodiments, a small molecule is not an amino acid. In some embodiments, a small molecule is not a nucleotide. In some embodiments, a small molecule is not a saccharide.

The term “subject” can be any multicellular animal. Often a subject is a vertebrate, e.g., a mammal or avian. Exemplary mammals include, e.g., humans, non-human primates, rodents (e.g., mouse, rat, rabbit), ungulates (e.g., ovine, bovine, equine, caprine species), canines, and felines. Often, a subject is an individual to whom a compound is to be delivered, e.g., for experimental, diagnostic, and/or therapeutic purposes or from whom a sample is obtained or on whom a diagnostic procedure is performed (e.g., a sample or procedure that will be used to assess tissue damage and/or to assess the effect of a compound described in the disclosure). The term “tissue damage” is used herein to refer to any type of damage or injury to cells, tissues, organs, or other body structures. The term encompasses, in various embodiments, degeneration due to disease, damage due to physical trauma or surgery, damage caused by exposure to deleterious substance, and other disruptions in the structure and/or functionality of cells, tissues, organs, or other body structures.

The term “tissue regeneration” or “TR” refers to at least partial regeneration, replacement, restoration, or regrowth of a tissue, organ, or other body structure, or portion thereof, following loss, damage, or degeneration, where said tissue regeneration but for the methods described in the present disclosure would not take place. Examples of tissue regeneration include the regrowth of severed digits or limbs including the regrowth of cartilage, bone, muscle, tendons, and ligaments, the scarless regrowth of bone, cartilage, skin, or muscle that has been lost due to injury or disease, with an increase in size and cell number of an injured or diseased organ such that the tissue or organ approximates the normal size of the tissue or organ or its size prior to injury or disease. Depending on the tissue type, tissue regeneration can occur via a variety of different mechanisms such as, for example, the rearrangement of pre-existing cells and/or tissue (e.g., through cell migration), the division of adult somatic stem cells or other progenitor cells and differentiation of at least some of their descendants, and/or the dedifferentiation, transdifferentiation, and/or proliferation of cells.

The term “toxic gene” or “toxic gene product” refers to genes and their respective encoded proteins that when present in a cell at greater than normal levels, result in the death of the cells. Said toxic genes are also known as “suicide” genes. Nonlimiting examples of said “toxic genes” are: cytosine deaminase gene that converts 5-Fluorocytosine (5-FC) to 5-Fluorouracil (5-FU) and the herpes simplex virus thymidine kinase gene (HSV-tk), that modifies ganciclovir (GCV) to ganciclovir monophosphate, which is then further converted in cancer cells to ganciclovir triphosphate.

The term “TR activator genes” refers to genes whose lack of expression in fetal and adult cells but whose transient expression in embryonic phases of development facilitate TR. Examples of said TR genes include combinations of OCT4, SOX2, KLF4, NANOG, ESRRB, NR5A2, CEBPA, MYC, LIN28A, TERT, and LIN28B.

The term “treat”, “treating”, “therapy”, “therapeutic” and similar terms in regard to a subject refer to providing medical and/or surgical management of the subject. Treatment can include, but is not limited to, administering a compound or composition (e.g., a pharmaceutical composition) to a subject. Treatment of a subject according to the instant disclosure is typically undertaken in an effort to promote regeneration, e.g., in a subject who has suffered tissue damage or is expected to suffer tissue damage (e.g., a subject who will undergo surgery). The effect of treatment can generally include increased regeneration, reduced scarring, and/or improved structural or functional outcome following tissue damage (as compared with the outcome in the absence of treatment), and/or can include reversal or reduction in severity or progression of a degenerative disease.

The term “variant” as applied to a particular polypeptide refers to a polypeptide that differs from such polypeptide (sometimes referred to as the “original polypeptide”) by one or more amino acid alterations, e.g., addition(s), deletion(s), and/or substitution(s). Sometimes an original polypeptide is a naturally occurring polypeptide (e.g., from human or non-human animal) or a polypeptide identical thereto. Variants may be naturally occurring or created using, e.g., recombinant DNA techniques or chemical synthesis. An addition can be an insertion within the polypeptide or an addition at the N- or C-terminus. In some embodiments, the number of amino acids substituted, deleted, or added can be for example, about 1 to 30, e.g., about 1 to 20, e.g., about 1 to 10, e.g., about 1 to 5, e.g., 1, 2, 3, 4, or 5. In some embodiments, a variant comprises a polypeptide whose sequence is homologous to the sequence of the original polypeptide over at least 50 amino acids, at least 100 amino acids, at least 150 amino acids, or more, up to the full length of the original polypeptide (but is not identical in sequence to the original polypeptide), e.g., the sequence of the variant polypeptide is at least 50%, 60%, 70%, 75%, 80%, 85%, 90%, 95%, 96%, 97%, 98%, 99%, or more identical to the sequence of the original polypeptide over at least 50 amino acids, at least 100 amino acids, at least 150 amino acids, or more, up to the full length of the original polypeptide. In some embodiments, a variant comprises a polypeptide at least 50%, 60%, 70%, 75%, 80%, 85%, 90%, 91%, 92%, 93%, 94%, 95%, 96%, 97%, 98%, 99%, 99.5% or more identical to an original polypeptide over at least 50%, 60%, 70%, 80%, 85%, 90%, 91%, 92%, 93%, 94%, 95%, 96%, 97%, 98%, 99%, or 100% of the length of the original polypeptide. In some embodiments a variant comprises at least one functional or structural domain, e.g., a domain identified as such in the Conserved Domain Database (CDD) of the National Center for Biotechnology Information (www.ncbi.nih.gov), e.g., an NCBI-curated domain. In some embodiments one, more than one, or all biological functions or activities of a variant or fragment is substantially similar to that of the corresponding biological function or activity of the original molecule. In some embodiments, a functional variant retains at least 10%, 20%, 30%, 40%, 50%, 60%, 70%, 80%, 90%, 95%, 96%, 97%, 98%, 99%, or more of the activity of the original polypeptide, e.g., about equal activity. In some embodiments, the activity of a variant is up to approximately 100%, approximately 125%, or approximately 150% of the activity of the original molecule. In other nonlimiting embodiments an activity of a variant or fragment is considered substantially similar to the activity of the original molecule if the amount or concentration of the variant needed to produce a particular effect is within 0.5 to 5-fold of the amount or concentration of the original molecule needed to produce that effect.

In some embodiments, amino acid “substitutions” in a variant are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Within a particular group, certain substitutions may be of particular interest, e.g., replacements of leucine by isoleucine (or vice versa), serine by threonine (or vice versa), or alanine by glycine (or vice versa). Of course non-conservative substitutions are often compatible with retaining function as well. In some embodiments, a substitution or deletion does not alter or delete an amino acid important for activity. Insertions or deletions may range in size from about 1 to 20 amino acids, e.g., 1 to 10 amino acids. In some instances larger domains may be removed without substantially affecting function. In certain embodiments of the disclosure the sequence of a variant can be obtained by making no more than a total of 5, 10, 15, or 20 amino acid additions, deletions, or substitutions to the sequence of a naturally occurring enzyme. In some embodiments, no more than 1%, 5%, 10%, or 20% of the amino acids in a polypeptide are insertions, deletions, or substitutions relative to the original polypeptide. Guidance in determining which amino acid residues may be replaced, added, or deleted without eliminating or substantially reducing activities of interest, may be obtained by comparing the sequence of the particular polypeptide with that of homologous polypeptides (e.g., from other organisms) and minimizing the number of amino acid sequence changes made in regions of high homology (conserved regions) or by replacing amino acids with those found in homologous sequences since amino acid residues that are conserved among various species are more likely to be important for activity than amino acids that are not conserved.

In some embodiments, a variant of a polypeptide comprises a heterologous polypeptide portion. The heterologous portion often has a sequence that is not present in or homologous to the original polypeptide. A heterologous portion may be, e.g., between 5 and about 5,000 amino acids long, or longer. Often it is between 5 and about 1,000 amino acids long. In some embodiments, a heterologous portion comprises a sequence that is found in a different polypeptide, e.g., a functional domain. In some embodiments, a heterologous portion comprises a sequence useful for purifying, expressing, solubilizing, and/or detecting the polypeptide. In some embodiments, a heterologous portion comprises a polypeptide “tag”, e.g., an affinity tag or epitope tag. For example, the tag can be an affinity tag (e.g., HA, TAP, Myc, His, Flag, GST), fluorescent or luminescent protein (e.g., EGFP, ECFP, EYFP, Cerulean, DsRed, mCherry), solubility-enhancing tag (e.g., a SUMO tag, NUS A tag, SNUT tag, or a monomeric mutant of the Ocr protein of bacteriophage T7). See, e.g., Esposito D and Chatterjee D K. Curr Opin Biotechnol; 17(4):353-8 (2006). In some embodiments, a tag can serve multiple functions. A tag is often relatively small, e.g., ranging from a few amino acids up to about 100 amino acids long. In some embodiments a tag is more than 100 amino acids long, e.g., up to about 500 amino acids long, or more. In some embodiments, a polypeptide has a tag located at the N- or C-terminus, e.g., as an N- or C-terminal fusion. The polypeptide could comprise multiple tags. In some embodiments, a His tag and a NUS tag are present, e.g., at the N-terminus. In some embodiments, a tag is cleavable, so that it can be removed from the polypeptide, e.g., by a protease. In some embodiments, this is achieved by including a sequence encoding a protease cleavage site between the sequence encoding the portion homologous to the original polypeptide and the tag. Exemplary proteases include, e.g., thrombin, TEV protease, Factor Xa, PreScission protease, etc. In some embodiments, a “self-cleaving” tag is used. See, e.g., PCT/US05/05763. Sequences encoding a tag can be located 5′ or 3′ with respect to a polynucleotide encoding the polypeptide (or both). In some embodiments a tag or other heterologous sequence is separated from the rest of the polypeptide by a polypeptide linker. For example, a linker can be a short polypeptide (e.g., 15-25 amino acids). Often a linker is composed of small amino acid residues such as serine, glycine, and/or alanine. A heterologous domain could comprise a transmembrane domain, a secretion signal domain, etc.

In certain embodiments of the disclosure a fragment or variant, optionally excluding a heterologous portion, if present, possesses sufficient structural similarity to the original polypeptide so that when its 3-dimensional structure (either actual or predicted structure) is superimposed on the structure of the original polypeptide, the volume of overlap is at least 70%, preferably at least 80%, more preferably at least 90% of the total volume of the structure of the original polypeptide. A partial or complete 3-dimensional structure of the fragment or variant may be determined by crystallizing the protein, which can be done using standard methods. Alternately, an NMR solution structure can be generated, also using standard methods. A modeling program such as MODELER (Sali, A. and Blundell, T L, J. Mol. Biol, 234, 779-815, 1993), or any other modeling program, can be used to generate a predicted structure. If a structure or predicted structure of a related polypeptide is available, the model can be based on that structure. The PROSPECT-PSPP suite of programs can be used (Guo, J T, et al., Nucleic Acids Res. 32 (Web Server issue):W522-5, Jul. 1, 2004). Where embodiments of the disclosure relate to variants of a polypeptide, it will be understood that polynucleotides encoding the variant are provided.

The term “vector” is used herein to refer to a nucleic acid or a virus or portion thereof (-g-a viral capsid or genome) capable of mediating entry of, e.g., transferring, transporting, etc., a nucleic acid molecule into a cell. Where the vector is a nucleic acid, the nucleic acid molecule to be transferred is generally linked to, e.g., inserted into, the vector nucleic acid molecule. A nucleic acid vector may include sequences that direct autonomous replication (e.g., an origin of replication), or may include sequences sufficient to allow integration of part or all of the nucleic acid into host cell DNA. Useful nucleic acid vectors include, for example, DNA or RNA plasmids, cosmids, and naturally occurring or modified viral genomes or portions thereof or nucleic acids (DNA or RNA) that can be packaged into viral) capsids. Plasmid vectors typically include an origin of replication and one or more selectable markers. Plasmids may include part or all of a viral genome (e.g., a viral promoter, enhancer, processing or packaging signals, etc.).

Viruses or portions thereof that can be used to introduce nucleic acid molecules into cells are referred to as viral vectors. Useful viral vectors include adenoviruses, adeno-associated viruses such as AAV2 and AAV9 or other serotypes of AAV, retroviruses, lentiviruses, vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-defective, and such replication-defective viral vectors may be preferable for therapeutic use. Where sufficient information is lacking it may, but need not be, supplied by a host cell or by another vector introduced into the cell. The nucleic acid to be transferred may be incorporated into a naturally occurring or modified viral genome or a portion thereof or may be present within the virus or viral capsid as a separate nucleic acid molecule. It will be appreciated that certain plasmid vectors that include part or all of a viral genome, typically including viral genetic information sufficient to direct transcription of a nucleic acid that can be packaged into a viral capsid and/or sufficient to give rise to a nucleic acid that can be integrated into the host cell genome and/or to give rise to infectious virus, are also sometimes referred to in the art as viral vectors. Vectors may contain one or more nucleic acids encoding a marker suitable for use in the identifying and/or selecting cells that have or have not been transformed or transfected with the vector. Markers include, for example, proteins that increase or decrease either resistance or sensitivity to antibiotics (e.g., an antibiotic-resistance gene encoding a protein that confers resistance to an antibiotic such as puromycin, hygromycin or blasticidin) or other compounds, enzymes whose activities are detectable by assays known in the art (e.g., beta.-galactosidase or alkaline phosphatase), and proteins or RNAs that detectably affect the phenotype of transformed or transfected cells (e.g., fluorescent proteins). Expression vectors are vectors that include regulatory sequence(s), e.g., expression control sequences such as a promoter, sufficient to direct transcription of an operably linked nucleic acid. Regulatory sequences may also include enhancer sequences or upstream activator sequences. Vectors may optionally include 5′leader or signal sequences. Vectors may optionally include cleavage and/or polyadenylations signals and/or a 3′ untranslated regions. Vectors often include one or more appropriately positioned sites for restriction enzymes, to facilitate introduction into the vector of the nucleic acid to be expressed.

An expression vector comprises sufficient cis-acting elements for expression; other elements required or helpful for expression can be supplied by the host cell or in vitro expression system. Various techniques may be employed for introducing nucleic acid molecules into cells. Such techniques include chemical-facilitated transfection using compounds such as calcium phosphate, cationic lipids, cationic polymers, liposome-mediated transfection, non-chemical methods such as electroporation, particle bombardment, or microinjection, and infection with a virus that contains the nucleic acid molecule of interest (sometimes termed “transduction”). Markers can be used for the identification and/or selection of cells that have taken up the vector and, typically, express the nucleic acid. Cells can be cultured in appropriate media to select such cells and, optionally, establish a stable cell line.

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Tables

TABLE I lists genes expressed in the embryonic (pre-fetal) stages of development in diverse mammalian somatic cell types (embryonic segmental iTR factors) wherein the promoters of the genes are useful in regulating the expression of toxic gene products in cancer cells.

TABLE I
Embryonic Segmented iTR Factors

| | | | Disclosed | |
|---|---|---|---|---|
| | | | in U.S. | |
| | | | Provisional | |
| Disclosed in | | | Application | |
| PCT/US14/ | Disclosed in | | 63/155,628, | |
| 40601 filed | PCT/US2017/036452 | | filed | |
| Jun. 3, 2014 | filed Jun. 7, 2017 | | Mar. 2, 2021 | |
| AFF3 | ADGRV1 | PAQR6 | LMNB1 | AC108142.1 |
| CBCAQH03 5 | ALDH5A1 | PCDHA2 | TCF3 | AGA |
| DLX1 | ALX1 | PCDHA3 | POU2F1 | AQP7P1 |
| DRD1IP | AMH | PCDHA4 | | AQP7P3 |
| F2RL2 | B4GALNT4 | PCDHA5 | | BAHD1 |
| FOXD1 | C14orf39 | PCDHA10 | | BBOX1 |
| LOC728755 | CHKB- | PCDHA11 | | C11orf35 |
| | CPT1B | | | (LMNTD2) |
| LOC791120 | CPT1B | PCDHAC1 | | CASC9 |
| MN1 | DOC2GP | PCDHB2 | | CBX2 |
| Nbla10527 | | | | |
| OXTR | DPY19L2 | PCDHB5 | | CCDC144NL- |
| | | | | AS1 |
| PCDHB2 | DSG2 | PCDHB9 | | CHRM3 |
| PCDHB17 | | | | |
| RAB3IP | FAM157A | PCDHB10 | | CPAMD8 |
| SIX1 | FAM157B | PCDHB14 | | FAR2P3 |
| WSB1 | FAR2P1 | PCDHB16 | | FIRRE |
| | FOXD4L4 | PCDHGB4 | | IGF2BP1 |
| | FSIP2 | PCDHGB6 | | LINC00648 |
| | GDF1 | PLPPR3 | | LINC02315 |
| | GRIN3B | PRR5L | | LOC644919 |
| | H2BFXP | RGPD1 | | MED15P9 |
| | L3MBTL1 | SLCO1A2 | | PCAT7 |
| | LIN28B | TSPAN11 | | PKP3 |
| | LINC00649 | TUBB2B | | POTEE |
| | LINC01021 | ZCCHC18 | | POTEF |
| | MN1 | ZNF497 | | RGPD2 |
| | NAALAD2 | ZNF853 | | WDR72 |
| | | | | WRN |

TABLE II lists genes expressed in the fetal and adult stages of development in diverse mammalian somatic cell types (fetal and adult iTR inhibitory factors) wherein the promoters of the genes are useful in regulating the expression of iTR genes such that said iTR genes are expressed at a greater level in adult non-regenerative cells but are down-regulated when the adult cells are reprogrammed to a pre-fetal regenerative state.

TABLE II
(iTR Inhibitory Factors)
Fetal/Adult Markers (iTR Inhibitory Factors)

| | | Disclosed in | | |
|---|---|---|---|---|
| | | U.S. Provisional | | |
| Disclosed in | Disclosed in | Application | | |
| PCT/US14/40601 | PCT/US2017/036452 | 63/155,628, | | |
| filed Jun. 3, 2014 | filed Jun. 7, 2017 | filed Mar. 2, 2021 | | |
| ACAT2 | ADIRF | LMNA | ALS2CR11 | MAP10 |
| C18orf56 | C10orf11 | | ANKRD7 | MEG8 |
| CAT | CAT | | ANKRD65 | MEG9 |
| COMT | CCDC144B | | BACE2 | MIRLET7HG |
| COX7A1 | COMT | | BHMT2 | NKAPL |
| DYNLT3 | COX7A1 | | C2CD6 | PAX8-AS1 |
| ELOVL6 | KRBOX1 | | C22orf26 | PRPH2 |
| FDPS | LINC00654 | | CADPS2 | PRR34-AS1 |
| IAH1 | LINC00839 | | CALHM2 | RP5-1043L13.1 |
| INSIG1 | LINC01116 | | CCDC36 | RP11-134021.1 |
| LOC205251 | MEG3 | | CCDC89 | SVIL-AS1 |
| MAOA | MIR4458HG | | CCDC125 | TEKT4P2 |
| NAALADL1 | NAALADL1 | | CCDC144B | ZNF578 |
| PSMD5 | PCDHGA2 | | CLDN11 | ZNF585B |
| RPS7 | PCDHGA6 | | CTSF | ZNF736 |
| SHMT1 | PCDHGA7 | | DDX43 | ZNF790-AS1 |
| TRIM4 | PCDHGA9 | | DNAJC15 | |
| TSPYL5 | PCDHGA12 | | EGFLAM | |
| ZNF280D | PCDHGB3 | | ESPNL | |
| | PCDHGB5 | | FAM24B | |
| | PLPP7 (PAPPDC3) | | FGF7 | |
| | POMC | | FKBP9L | |
| | PRR34 | | FLG-AS1 | |
| | PRSS3 | | FRG1B | |
| | PTCHD3 | | GPAT2 | |
| | PTCHD3P1 | | GYPE | |
| | SPESP1 | | HENMT1 | |
| | TRIM4 | | HIST2H2BA | |
| | USP32P1 | | IRAK4 | |
| | ZNF300P1 | | LINC00865 | |
| | ZNF572 | | LOC283788 | |
| | | | LOC100233156 | |
| | | | LRRK2 | |

TABLE III lists preferred promoters of genes expressed in the fetal and adult stages of development in diverse mammalian somatic cell types wherein the promoters of the genes are useful in regulating the expression of iTR genes such that said iTR genes are expressed at a greater level in adult non-regenerative cells but are down-regulated when the adult cells are reprogrammed to a pre-fetal regenerative state.

TABLE III

| Gene Name | Position of Promoter Sequence (Hg38) | Strand |
|---|---|---|
| C2CD6 | chr2: 201,619,183-201,620,677 | Minus |
| CAT | chr11: 34,437,183-34,438,933 | Plus |
| COMT | chr22: 19,939,926-19,941,771 | Plus |
| COX7A1 | chr19: 36,152,448-36,153,947 | Minus |
| GYPE | chr4: 143,912,132-143,913,679 | Minus |
| IHO1 | chr3: 49,198,025-49,199,497 | Plus |
| KRBOX1 | chr3: 42,934,886-42,936,341 | Plus |
| LINC00839 | chr10: 42,473,979-42,475,490 | Plus |
| LINC00865 | chr10: 89,836,052-89,837,217 | Plus |
| LRRK2 | chr12: 40,223,497-40,224,889 | Plus |
| MEG3 | chr14: 100,823,838-100,826,107 | Plus |
| MIRLET7BHG | chr22: 46,078,318-46,085,996 | Plus |
| NKAPL | chr6: 28,257,796-28,259,296 | Plus |
| PRR34-AS1 | chr22: 46,052,204-46,053,845 | Plus |
| ZNF300P1 | chr5: 150,946,585-150,947,789 | Minus |

TABLE IV lists preferred promoters of genes expressed in the embryonic (pre-fetal) stages of development in diverse mammalian somatic cell types wherein the promoters of the genes are useful in regulating the expression of toxic gene products in cancer cells.

TABLE IV

| Gene Name | Position of Promoter Sequence (Hg38) | Strand |
|---|---|---|
| CCDC144NL-AS1 | chr17: 20,867,979-20,868,526 | Plus |
| CCDC144NL-AS1 | chr17: 20,869,776-20,870,857 | Plus |
| CASC9 | chr8: 75322337-75,325,237 | Plus |
| CPT1B | chr22: 50,578,614-50,584,487 | Minus |
| FAR2P1 | chr2: 130,036,639-130,039,003 | Minus |
| FAR2P2 | chr2: 130,428,547-130,429,586 | Minus |
| FAR2P3 | chr2: 130,691,713-130,694,087 | Plus |
| LIN28B | chr6: 104,952,665-104,957,106 | Plus |
| LINC00649 | chr21: 33,930,560-33,931,121 | Plus |
| POTEE | chr2: 131,208,731-131,209,580 | Plus |
| POTEF | chr2: 130,129,223-130,129,622 | Minus |
| MED15P9 | chr2: 130,129,223-130,129,622 | Plus |
| PCAT7 | chr9: 94,553,093-94,555,044 | Plus |
| PURPL | chr5: 27,471,047-27,472,292 | Plus |

### Methods

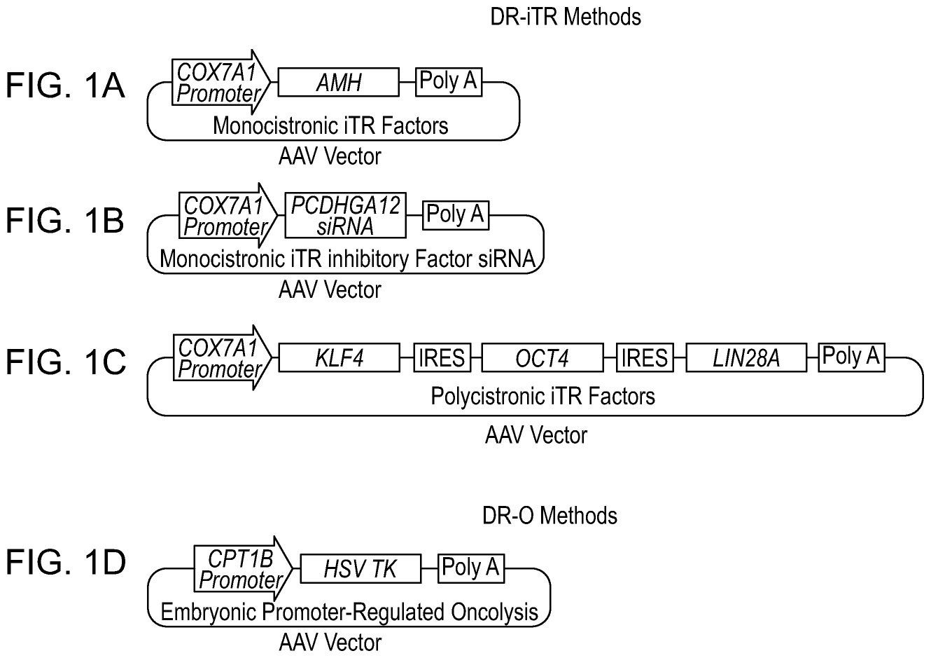

The methods of the present invention relate to the use of regulatory elements such as promoters and enhancers from developmentally-regulated genes to regulate the expression of other genes to either: 1) induce tissue regeneration, designated herein as Developmentally-Regulated induced Tissue Regeneration (DR-iTR herein) or, 2) to selectively induce the death of cancer cells while leaving the majority of normal adult somatic cell types alive, referred to herein as Developmentally-Regulated Oncolysis (DR-0). The strategies are summarized in FIGS. 1A-1D.

The methods of the present invention that relate to DR-iTR utilize the surprising discovery that there are families of genes that are widely expressed in diverse differentiated cell types that alternatively either are induced following the transition from the embryonic to the fetal stages of mammalian development, said transition referred to herein as the “embryonic-fetal transition” or “EFT” or are repressed following said transition. While not every such gene is precisely induced or repressed exactly at said EFT, and said alterations in gene expression may vary depending on the somatic cell type, it is generally the case that the alterations occur at or around said EFT, but in any event, generally occur in the prenatal stages of development. A list of said developmentally-regulated genes are disclosed in (see PCT/US14/40601, filed Jun. 3, 2014 and titled “Compositions and Methods for Induced Tissue Regeneration in Mammalian Species,” and PCT/US2017/036452, filed Jun. 7, 2017 and titled “Improved Methods for Detecting and Modulating the Embryonic-Fetal Transition in Mammalian Species,” the contents of each of which is incorporated herein by reference) and shown in Table I and a list of preferred promoters are shown in TABLE III.

The present invention also describes methods for the specific destruction of cancer cells that abnormally express genes normally expressed only in the embryonic (pre-fetal) stages of development in somatic cells. This method, designated herein as Developmentally-Regulated Oncolysis (DR-0), utilizes the promoters or enhancers from developmentally-regulated genes widely expressed in diverse cell types in the embryonic phases of development but repressed in the majority of adult somatic cell type, but re-expressed in diverse sarcoma, carcinoma, adenocarcinoma, and blood cell cancer types. Such useful genes are listed in TABLE I and preferred promoters are listed in TABLE IV.

### DR-iTR

As shown in FIGS. 1A-1C, DR-iTR can be performed using at least three different methods. FIG. 1A illustrates the use of a promoter or enhancer element naturally regulating a fetal or adult-onset gene widely expressed in diverse adult, as opposed to embryonic (pre-fetal) cell types, to regulate the expression of a factor that promotes tissue regeneration. As a nonlimiting example, FIG. 1A illustrates the use of the promoter region for the gene COX7A1 to regulate the expression of an iTR factor in a gene expression vector, in this case, the expression of the gene encoding the secreted growth factor Anti Mullerian Hormone (AMH) in an AAV vector. FIG. 1B illustrates the use of the promoter region for the gene COX7A1 to regulate the expression of an RNAi construct in a gene expression vector targeting an iTR inhibitor factor, in this case, by way of nonlimiting example, the expression of the gene encoding the clustered protocadherin gene (PCDHGA12) in an AAV vector. FIG. 1C illustrates the use of the promoter region for the gene COX7A1 to regulate the expression of multiple iTR factors in a gene expression vector, in this case, the expression of the genes encoding the reprogramming factors KLF4, OCT4, and LIN28A in an AAV vector.

DR-iTR using a monocistronic segmented iTR factor gene selected from Table I (FIG. 1A) is implemented by choosing a gene therapy vector described herein, including but not limited to adeno-associated virus (AAV), such as AAV2 or AAV9 that have been genetically modified to decrease their immunogenicity, reduce genomic integration, or increase gene expression, together with a promotor or gene enhancer sequence in cis chosen from the fetal/adult iTR inhibitory genes listed in Table II. Cells in vitro or in vivo, including microbiopsies of tissue cultured in vitro are then infected with said gene therapy constructs to increased scarless tissue regeneration by DR-iTR.

DR-iTR using a monocistronic RNAi sequence designed to decrease levels of a TR inhibitory gene transcript chosen from Table II (FIG. 1B) is implemented by choosing a gene therapy vector described herein, including but not limited to adeno-associated virus (AAV), such as AAV2 or AAV9 that have been genetically modified to decrease their immunogenicity, reduce genomic integration, or increase gene expression, together with a promotor or gene enhancer sequence in cis chosen from the fetal/adult iTR inhibitory genes listed in Table II. Cells in vitro or in vivo, including microbiopsies of tissue cultured in vitro are then infected with said gene therapy constructs to increased scarless tissue regeneration by DR-iTR.

DR-iTR (FIG. 1C) using a monocistronic or polycistronic gene therapy vector to cause global reprogramming but without reprogramming the cells to pluripotency is implemented by choosing a combination of the global reprogramming genes OCT4, SOX2, KLF4, NANOG, ESRRB, NR5A2, CEBPA, MYC, SALL4, LIN28A or LIN28B; by way of nonlimiting example, the genes OCT4, SOX2, KLF4, and MYC; or LIN28A, OCT4, SOX2, and NANOG; or KLF4, OCT4, and LIN28A; in a gene therapy vector described herein, including but not limited to adeno-associated virus (AAV), such as AAV2 or AAV9 that have been genetically modified to decrease their immunogenicity, reduce genomic integration, or increase gene expression, together with a promotor or gene enhancer sequence in cis chosen from the fetal/adult iTR inhibitory genes listed in Table II. Cells in vitro or in vivo, including microbiopsies of tissue cultured in vitro are then infected with said gene therapy constructs to increased scarless tissue regeneration with reduced expression of the global reprogramming genes when the developmental aging of the cells is reversed to approximate the developmental period of EFT.

Genes whose expression in embryonic phases of development facilitate TR are herein designated “TR activators.” Molecules that alter the levels of TR activators in a manner leading to TR are herein designated “iTR factors.” TR activators and iTR factors (collectively referred to as “iTR genes” and, the protein products of iTR genes, are often conserved in animals ranging from sea anemones to mammals. The gene-encoded protein sequences, and sequences of nucleic acids (e.g., mRNA) encoding genes referred to herein, including those from a number of different non-human animal species are known in the art and can be found, e.g., in publicly available databases such as those available at the National Center for Biotechnology Information (NCBI) (www.ncbi.nih.gov).

The disclosure provides a number of different methods of producing developmentally-regulated iTR (DR-iTR) in mammalian cells in vivo and ex vivo, including human microbiopsies cultured ex vivo. In general, an DR-iTR can be the sole source of reprogramming activity, or alternatively, one or more iTR factors can be developmentally-regulated while one or more other iTR factors can be applied to cells in vivo or in vitro without such regulation. An iTR factor can be, e.g., a small molecule, nucleic acid, oligonucleotide, polypeptide, peptide, lipid, carbohydrate, etc. In the case of the present invention, iTR genes are developmentally-regulated to control the extent of the modulation of TR. In some embodiments of the invention, iTR factors capable of reprogramming cells to pluripotency, such as combinations of OCT4, SOX2, KLF4, NANOG, ESRRB, NR5A2, CEBPA, MYC, SALL4, LIN28A or LIN28B, including without limitation the combinations: OCT4, SOX2, KLF4, MYC; and OCT4, SOX2, NANOG, LIN28A; and OCT4, KLF4, LIN28A; are introduced into adult non-regenerative somatic cells as genes in expression vectors wherein the aforementioned genes are regulated by promoter or enhancer elements from developmentally-regulated genes including, without limitation, the genes: C2CD6, CAT, COMT, COX7A1, GYPE, IHO1, KRBOX1, LINC00839, LINC00865, LRRK2, MEG3, MIRLET7BHG, NKAPL, PRR34-AS1, or ZNF300P1 such that the levels of expression of the iTR factors decreases upon reaching a defined stage of the reversal of developmental aging before pluripotency is achieved. Said developmental stage, by way of non-limiting example, be the stage wherein scarless regeneration is induced, such as is the case at the EFT.

In the case where the iTR genes being developmentally-regulated are iTR inhibitors, including, but not limited to COX7A1, PCDHGA12, or NAALADL1, RNAi constructs are introduced into adult non-regenerative somatic cells in expression vectors wherein the aforementioned sequences are regulated by promoter or enhancer elements from developmentally-regulated genes including, without limitation, the genes: C2CD6, CAT, COMT, COX7A1, GYPE, IHO1, KRBOX1, LINC00839, LINC00865, LRRK2, MEG3, MIRLET7BHG, NKAPL, PRR34-AS1, or ZNF300P1 such that the levels of expression of the RNAi constructs decreases upon reaching a defined stage of the reversal of developmental aging before pluripotency is achieved. Said developmental stage, by way of non-limiting example, be the stage wherein scarless regeneration is induced, such as is the case at the EFT. In the case of targeting TR inhibitors, factors are identified and used in research and therapy that reduce the levels of the product of the TR inhibitor gene. Said TR inhibitor gene can be any one or combination of TR inhibitor genes such as COX7A1 or NAALADL1 (see PCT application no. PCT/US2017/036452, filed Jun. 7, 2017 and titled “Improved Methods for Detecting and Modulating the Embryonic Fetal Transition in Mammalian Species,” the contents of which is incorporated herein by reference). In another example, said TR inhibitor gene may be LMNA (see U.S. Provisional Application 63/155,628, filed Mar. 2, 2021 and titled “Methods and Compositions Used to Modify Chromatin Architecture to Regulate Phenotype in Aging and Cancer,” the contents of which are incorporated herein by reference). The amount of TR inhibitor gene RNA can be decreased by inhibiting synthesis of TR inhibitor RNA synthesis by cells (also referred to as “inhibiting TR inhibitor gene expression”), e.g., by reducing the amount of mRNA encoding TR inhibitor genes or by reducing translation of mRNA encoding TR inhibitor genes. Said factor can be by way of nonlimiting example, RNAi targeting a sequence within the TR inhibitor genes such as COX7A1, NAALADL1, or LMNA (see PCT application no. PCT/US2017/036452, filed Jun. 7, 2017 and titled “Improved Methods for Detecting and Modulating the Embryonic Fetal Transition in Mammalian Species”; U.S. Provisional Application 63/155,628, filed Mar. 2, 2021 and titled “Methods and Compositions Used to Modify Chromatin Architecture to Regulate Phenotype in Aging and Cancer,” each of which is incorporated herein by reference).

In some embodiments, TR inhibitor gene expression is inhibited by developmentally-regulated RNA interference (RNAi). As known in the art, RNAi is a process in which the presence in a cell of double stranded RNA that has sequence correspondence to a gene leads to sequence-specific inhibition of the expression of the gene, typically as a result of cleavage or translational repression of the mRNA transcribed from the gene. Compounds useful for causing inhibition of expression by RNAi (“RNAi agents”) include short interfering RNAs (siRNAs), short hairpin RNAs (shRNAs), microRNAs (miRNAs), and miRNA-like molecules.

One of skill in the art can readily design sequences for developmentally-regulated RNAi agents, e.g., siRNAs, useful for inhibiting expression of mammalian TR inhibitor genes, e.g., human TR inhibitor genes once one has identified said TR inhibitor genes. In some embodiments, such sequences are selected to minimize “off-target” effects. For example, a sequence that is complementary to a sequence present in TR inhibitor gene mRNA and not present in other mRNAs expressed in a species of interest (or not present in the genome of the species of interest) may be used. Position-specific chemical modifications may be used to reduce potential off-target effects. In some embodiments, at least two different RNAi agents, e.g., siRNAs, targeted to TR inhibitor gene mRNA are used in combination. In some embodiments, a microRNA (which may be an artificially designed microRNA) is used to inhibit TR inhibitor gene expression.

In some embodiments of the invention, TR inhibitor gene expression is inhibited using a developmentally-regulated antisense molecule comprising a single-stranded oligonucleotide that is perfectly or substantially complementary to mRNA encoding TR inhibitor genes. The oligonucleotide hybridizes to TR inhibitor gene mRNA leading, e.g., to degradation of the mRNA by RNase H or blocking of translation by steric hindrance. In other embodiments of the invention, TR inhibitor gene expression is inhibited using a ribozyme or triplex nucleic acid.

In some embodiments, of the invention, a TR inhibitor inhibits at least one activity of an TR inhibitor protein. TR inhibitor activity can be decreased by contacting the TR inhibitor protein with a compound that physically interacts with the TR inhibitor protein. Such a compound may, for example, alter the structure of the TR inhibitor protein (e.g., by covalently modifying it) and/or block the interaction of the TR inhibitor protein with one or more other molecule(s) such as cofactors or substrates. In some embodiments, inhibition or reduction may be a decrease of at least about 5%, 10%, 15%, 20%, 25%, 30%, 35%, 40%, 45%, 50%, 55%, 60%, 65%, 70%, 75%, 80%, 85%, 90%, 95%, or 99% of a reference level (e.g., a control level). A control level may be the level of the TR inhibitor that occurs in the absence of the factor. For example, an TR factor may reduce the level of the TR inhibitor protein to no more than 95%, 90%, 85%, 80%, 75%, 70%, 65%, 60%, 55%, 50%, 40%, 30%, 25%, 20%, 10%, or 5% of the level that occurs in the absence of the factor under the conditions tested. In some embodiments, levels of the TR inhibitor are reduced to 75% or less of the level that occurs in the absence of the factor, under the conditions tested. In some embodiments, levels of the TR inhibitor are reduced to 50% or less of the level that occurs in the absence of the TR factor, under the conditions tested. In some embodiments, levels of the TR inhibitor are reduced to 25% or less of the level that occurs in the absence of the iTR factor, under the conditions tested. In some embodiments, levels of the TR inhibitor are reduced to 10% or less of the level that occurs in the absence of the iTR factor, under the conditions tested. In some cases the level of modulation (e.g., inhibition or reduction) as compared with a control level is statistically significant. As used herein, “statistically significant” refers to a p-value of less than 0.05, e.g., a p-value of less than 0.025 or a p-value of less than 0.01, using an appropriate statistical test (e.g, ANOVA, t-test, etc.).

In some embodiments of the invention, a developmentally-regulated compound directly inhibits TR inhibitor proteins, i.e., the compound inhibits TR inhibitor proteins by a mechanism that involves a physical interaction (binding) between the TR inhibitor and the iTR factor. For example, binding of a TR inhibitor to an iTR factor can interfere with the TR inhibitor's ability to catalyze a reaction and/or can occlude the TR inhibitors active site. A variety of compounds can be used to directly inhibit TR inhibitors. Exemplary compounds that directly inhibit TR inhibitors can be, e.g., small molecules, antibodies, or aptamers.

In some embodiments of the invention, an iTR factor binds covalently to the TR inhibitor. For example, the compound may modify amino acid residue(s) that are needed for enzymatic activity. In some embodiments, an iTR factor comprises one or more reactive functional groups such as an aldehyde, haloalkane, alkene, fluorophosphonate (e.g., alkyl fluorophosphonate), Michael acceptor, phenyl sulfonate, methylketone, e.g., a halogenated methylketone or diazomethylketone, fluorophosphonate, vinyl ester, vinyl sulfone, or vinyl sulfonamide, that reacts with an amino acid side chain of TR inhibitors. In some embodiments, an iTR factor inhibitor comprises a compound that physically interacts with a TR inhibitor, wherein the compound comprises a reactive functional group. In some embodiments, the structure of a compound that physically interacts with the TR inhibitor is modified to incorporate a reactive functional group. In some embodiments, the compound comprises a TR inhibitor substrate analog or transition state analog. In some embodiments, the compound interacts with the TR inhibitor in or near the TR inhibitor active site.

In other embodiments, an iTR factor binds non-covalently to a TR inhibitor and/or to a complex containing the TR inhibitor and a TR inhibitor substrate. In some embodiments, an iTR factor binds non-covalently to the active site of a TR inhibitor and/or competes with substrate(s) for access to the TR inhibitor active site. In some embodiments, an iTR factor binds to the TR inhibitor with an effective dose of approximately 10⁻³ M or less, e.g., 10⁻⁴M or less, e.g., 10⁻⁵ M or less, e.g., 10⁻⁶ M or less, 10⁻⁷ M or less, 10⁻⁸ M or less, or 10⁻⁹ M or less under the conditions tested, e.g., in a physiologically acceptable solution such as phosphate buffered saline. Binding affinity can be measured, e.g., using surface plasmon resonance (e.g., with a Biacore® system), isothermal titration calorimetry, or a competitive binding assay, as known in the art. In some embodiments, the inhibitor comprises a TR inhibitor substrate analog or transition state analog. In the case of increasing the activity of TR activators, any combination of the genes OCT4, SOX2, KLF4, NANOG, ESRRB, NR5A2, CEBPA, MYC, LIN28A, TERT, and LIN28 or their respective RNAs or proteins may be used. The levels of the products of these genes may be introduced using the vectors described herein.

In other embodiments, the iTR factors are constructs that introduce RNA into microbiopsies either directly or through gene expression constructs that are capable of inducing pluripotency if allowed to react with cells for a sufficient period of time, but for lesser times can cause iTR. Preferably, the RNAs do not include all of the RNAs needed for reprogramming to pluripotency and instead include only LIN28A or LIN28B optionally together with an agent to increase telomere length such as RNA for the catalytic component of telomerase (TERT). Most preferably, the agents to induce iTR are genes/factors induced by LIN28A or -encoded proteins such as GFER, optionally in combination with an agent that increases telomere length such as the RNA or gene encoding TERT, and/or in combination with the factors disclosed herein important for iTR such as 0.05-5 mM valproic acid, preferably 0.5 mM valproic acid, 1-100 ng/mL AMH, preferably 10 ng/mL AMH, and 2-200 ng/mL GFER, preferably 20 ng/mL. When administered in vivo, such factors are preferably administered in a slow-release hydrogel matrix such as one comprised of chemically modified and crosslinked hyaluronic acid and collagen such as HyStem matrices.

### DR-Oncolysis (DR-O)

As illustrated in FIGS. 1A-1D, the promoters of the present invention including those associated with the genes listed in Table I, more preferably, those listed in Table IV whose sequence is disclosed herein, are joined in cis with toxic gene products such as herpes simplex virus thymidine kinase (HSV TK) such that cancer cells expressing a pre-fetal pattern of gene expression preferentially express the toxic gene product while normal adjacent cells, since they no longer express the embryonic pattern of expression, are not destroyed. Alternatively, the embryonic promoters for the genes of Table I, and more preferably the gene promoters listed in Table IV are used to expressed immunoglobulin targeting antigens uniquely expressed in embryonic cells. Such embryonic antigens include the members of the clustered protocadherin locus (CPL) disclosed in (see U.S. Provisional Application No. 63/155,631 filed Mar. 2, 2021 and titled “Use of Photocadherins in Methods of Diagnosing and Treating Cancers,” incorporated by reference herein in its entirety). The representative embryonic CPL isoform transcript markers PCDHA4 and PCDHB2 are significantly down-regulated in adult cells compared to embryonic counterparts. Interestingly, diverse cancer lines such as those from sarcomas and carcinomas and adenocarcinomas when compared to normal adult cells show a highly significant shift toward an embryonic pattern of CPL gene expression. In the case of the a and P loci, PCDHA4 and PCDHB2 are significantly up-regulated in cancer cells as are other members of the alpha and beta clusters and are therefore useful antigens to target cancer in an adult using DR-O.

DR-O therapeutics can be used to treat cancer, particularly carcinomas. In some embodiments, the cancer is a basal cell carcinoma, a squamous cell carcinoma, a renal cell carcinoma, a ductal carcinoma in situ, an invasive ductal carcinoma, or a combination thereof. In some embodiments, the cancer is breast, colorectal, kidney, liver, lung, oral, pancreatic, prostate cancer, or a combination thereof.

### Reporter-Based Screening Assays for iTR Factors

The invention provides methods for identifying iTR factors using (a) a reporter molecule comprising a readily-detectable marker such as GFP or beta galactosidase whose expression is driven by the promoter of one of the TR activator genes described herein such as that for COX7A1. The invention provides screening assays that involve determining whether a test compound affects the expression of TR activator genes and/or inhibits the expression of TR inhibitory genes. The invention further provides reporter molecules and compositions useful for practicing the methods. In general, compounds identified using the inventive methods can act by any of mechanism that results in increased or decreased TR activator or inhibitor genes respectively. In the case of the COX7A1 promoter, a promoter sequence flanking the 5′ end of the human gene has been characterized to the position of −756 bases to the ATG translation start codon (Yu, M., et al. Biochimica and Biophysica Acta 1574 (2002) 345-353). Transcription start site of the most cDNAs were observed to be at −55 bases of the translation start codon. The promoter, as well as the rest of the gene sequence, lays in a CpG island, similarly to the promoters of many housekeeping genes, although the expression of COX7A1 is tissue specific. CpG islands are characterized by the abundance of CG dinucleotides that surpasses that of the average, expected content for the genome, over the span of at least 200 bases. The promoter comprises several regulatory binding site sequences: MEF2 at position −524, as well as three E boxes (characterized as E1, E2, and E3), at, respectively—positions −58, −279 and −585; E box is a DNA binding site (CAACTG) that binds members of the myogenic family of regulatory proteins. Additionally, in the region approximately −95 to −68 bases, there are multiple CG rich segments similar to the one recognized by the transcription factor Sp1. The gene itself, as characterized in GRCh38.p7 primary assembly, occupies 1948 bases between positions 36150922 and 36152869 on Human chromosome 18, and comprises 4 exons interspersed by three introns. Gene sequence, with the promoter sequence is curated at NCBI under locus identifier AF037372.

### Reporter Molecules, Cells, and Membranes

In general, detectable moieties useful in the reporter molecules of the invention include light-emitting or light-absorbing compounds that generate or quench a detectable fluorescent, chemiluminescent, or bioluminescent signal. In some embodiments, activation of TR activator genes or inhibition of TR inhibitory genes causes release of the detectable moiety into a liquid medium, and the signal generated or quenched by the released detectable moiety present in the medium (or a sample thereof) is detected. In some embodiments, the resulting signal causes an alteration in a property of the detectable moiety, and such alteration can be detected, e.g., as an optical signal. For example, the signal may alter the emission or absorption of electromagnetic radiation (e.g., radiation having a wavelength within the infrared, visible or UV portion of the spectrum) by the detectable moiety. In some embodiments, a reporter molecule comprises a fluorescent or luminescent moiety, and a second molecule serves as quencher that quenches the fluorescent or luminescent moiety. Such alteration can be detected using apparatus and methods known in the art.

In many embodiments of the invention, the reporter molecule is a genetically encodable molecule that can be expressed by a cell, and the detectable moiety comprises, e.g., a detectable polypeptide. Thus in some embodiments, the reporter molecule is a polypeptide comprising a fluorescent polypeptides such as green, blue, sapphire, yellow, red, orange, and cyan fluorescent proteins and derivatives thereof (e.g., enhanced GFP); monomeric red fluorescent protein and derivatives such as those known as “mFruits”, e.g., mCherry, mStrawberry, mTomato, etc., and luminescent proteins such as aequorin. (It will be understood that in some embodiments, the fluorescence or luminescence occurs in the presence of one or more additional molecules, e.g., an ion such as a calcium ion and/or a prosthetic group such as coelenterazine.) In some embodiments, the detectable moiety comprises an enzyme that acts on a substrate to produce a fluorescent, luminescent, colored, or otherwise detectable product. Examples of enzymes that may serve as detectable moieties include luciferase; beta-galactosidase; horseradish peroxidase; alkaline phosphatase; etc. (It will be appreciated that the enzyme is detected by detecting the product of the reaction.) In some embodiments, the detectable moiety comprises a polypeptide tag that can be readily detected using a second agent such as a labeled (e.g., fluorescently labeled) antibody. For example, fluorescently labeled antibodies that bind to the HA, Myc, or a variety of other peptide tags are available. Thus the invention encompasses embodiments in which a detectable moiety can be detected directly (i.e., it generates a detectable signal without requiring interaction with a second agent) and embodiments in which a detectable moiety interacts (e.g., binds and/or reacts) with a second agent and such interaction renders the detectable moiety detectable, e.g., by resulting in generation of a detectable signal or because the second agent is directly detectable. In embodiments in which a detectable moiety interacts with a second agent to produce a detectable signal, the detectable moiety may react with the second agent is acted on by a second agent to produce a detectable signal. In many embodiments, the intensity of the signal provides an indication of the amount of detectable moiety present e.g., in a sample being assessed or in area being imaged. In some embodiments, the amount of detectable moiety is optionally quantified, e.g., on a relative or absolute basis, based on the signal intensity.

The description provides nucleic acids comprising a sequence that encodes a reporter polypeptide of the invention. In some embodiments, a nucleic acid encodes a precursor polypeptide of a reporter polypeptide of the invention. In some embodiments, the sequence encoding the polypeptide is operably linked to expression control elements (e.g., a promoter or promoter/enhancer sequence) appropriate to direct transcription of mRNA encoding the polypeptide. The invention further provides expression vectors comprising the nucleic acids.

Selection of appropriate expression control elements may be based, e.g., on the cell type and species in which the nucleic acid is to be expressed. One of ordinary skill in the art can readily select appropriate expression control elements and/or expression vectors. In some embodiments, expression control element(s) are regulatable, e.g., inducible or repressible. Exemplary promoters suitable for use in bacterial cells include, e.g., Lac, Trp, Tac, araBAD (e.g., in a pBAD vectors), phage promoters such as T7 or T3. Exemplary expression control sequences useful for directing expression in mammalian cells include, e.g., the early and late promoters of SV40, adenovirus or cytomegalovirus immediate early promoter, or viral promoter/enhancer sequences, retroviral LTRs, promoters or promoter/enhancers from mammalian genes, e.g., actin, EF-1 alpha, phosphoglycerate kinase, etc. Regulatable (e.g., inducible or repressible) expression systems such as the Tet-On and Tet-Off systems (regulatable by tetracycline and analogs such as doxycycline) and others that can be regulated by small molecules such as hormones receptor ligands (e.g., steroid receptor ligands, which may or may not be steroids), metal-regulated systems (e.g., metallothionein promoter), etc. The description further provides cells and cell lines that comprise such nucleic acids and/or vectors. In some embodiments, the cells are eukaryotic cells, e.g., fungal, plant, or animal cells. In some embodiments, the cell is a vertebrate cell, e.g., a mammalian cell, e.g., a human cell, non-human primate cell, or rodent cell. Often a cell is a member of a cell line, e.g., an established or immortalized cell line that has acquired the ability to proliferate indefinitely in culture (e.g., as a result of mutation or genetic manipulation such as the constitutive expression of the catalytic component of telomerase). Numerous cell lines are known in the art and can be used in the instant invention. Mammalian cell lines include, e.g., HEK-293 (e.g., HEK-293T), CHO, NIH-3T3, COS, and HeLa cell lines. In some embodiments, a cell line is a tumor cell line. In other embodiments, a cell is non-tumorigenic and/or is not derived from a tumor. In some embodiments, the cells are adherent cells. In some embodiments, non-adherent cells are used. In some embodiments, a cell is of a cell type or cell line is used that has been shown to naturally have a subset of TR activator genes expressed or TR inhibitor genes not expressed. If a cell lacks one or more TR activator or inhibitor genes, the cell can be genetically engineered to express such protein(s). In some embodiments, a cell line of the invention is descended from a single cell. For example, a population of cells can be transfected with a nucleic acid encoding the reporter polypeptide and a colony derived from a single cell can be selected and expanded in culture. In some embodiments, cells are transiently transfected with an expression vector that encodes the reporter molecule. Cells can be co-transfected with a control plasmid, optionally expressing a different detectable polypeptide, to control for transfection efficiency (e.g., across multiple runs of an assay).

### TR Activator Polypeptides and Nucleic Acids

TR activators include combinations of. Under the headings “Embryonic Markers” and “Fetal/Adult Markers”, respectively. TR activator and TR inhibitor polypeptides useful in the inventive methods may be obtained by a variety of methods. In some embodiments, the polypeptides are produced using recombinant DNA techniques. Standard methods for recombinant protein expression can be used. A nucleic acid encoding a TR activator or TR inhibitor gene can readily be obtained, e.g., from cells that express the genes (e.g., by PCR or other amplification methods or by cloning) or by chemical synthesis or in vitro transcription based on the cDNA sequence polypeptide sequence. One of ordinary skill in the art would know that due to the degeneracy of the genetic code, the genes can be encoded by many different nucleic acid sequences. Optionally, a sequence is codon-optimized for expression in a host cell of choice. The genes could be expressed in bacterial, fungal, animal, or plant cells or organisms. The genes could be isolated from cells that naturally express it or from cells into which a nucleic acid encoding the protein has been transiently or stably introduced, e.g., cells that contain an expression vector encoding the genes. In some embodiments, the gene is secreted by cells in culture and isolated from the culture medium.

In some embodiments of the invention, the sequence of a TR activator or TR inhibitor polypeptide is used in the inventive screening methods. A naturally occurring TR activator or TR inhibitor polypeptide can be from any species whose genome encodes a TR activator or TR inhibitor polypeptide, e.g., human, non-human primate, rodent, etc. A polypeptide whose sequence is identical to naturally occurring TR activator or TR inhibitor is sometimes referred to herein as “native TR activator/inhibitor”. A TR activator or TR inhibitor polypeptide of use in the invention may or may not comprise a secretion signal sequence or a portion thereof. For example, mature TR activator or TR inhibitor comprising or consisting of amino acids 20-496 of human TR activator or TR inhibitor (or corresponding amino acids of TR activator or TR inhibitor of a different species) may be used.

In some embodiments, a polypeptide comprising or consisting of a variant or fragment of TR activator or TR inhibitor is used. TR activator or TR inhibitor variants include polypeptides that differ by one or more amino acid substitutions, additions, or deletions, relative to TR activator or TR inhibitor. In some embodiments, a TR activator or TR inhibitor variant comprises a polypeptide at least 70%, 75%, 80%, 85%, 90%, 95%, 96%, 97%, 98%, 99%, or more identical to at least amino acids 20-496 of TR activator or TR inhibitor (e.g., from human or mouse) over at least 50%, 60%, 70%, 80%, 85%, 90%, 95%, 96%, 97%, 98%, 99%, or 100% of at least amino acids 20-496 of human TR activator or TR inhibitor or amino acids 20-503 of mouse TR activator or TR inhibitor. In some embodiments, a TR activator or TR inhibitor variant comprises a polypeptide at least 80%, 85%, 90%, 95%, 96%, 97%, 98%, 99%, or more identical to at least amino acids 20-496 of human TR activator or TR inhibitor or amino acids 20-503 of mouse TR activator or TR inhibitor. In some embodiments, a TR activator or TR inhibitor polypeptide comprises a polypeptide at least 80%, 85%, 90%, 95%, 96%, 97%, 98%, 99%, or more identical to at least amino acids 20-496 of human TR activator or TR inhibitor or amino acids 20-503 of mouse TR activator or TR inhibitor. A nucleic acid that encodes a TR activator or TR inhibitor variant or fragment can readily be generated, e.g., by modifying the DNA that encodes native TR activator or TR inhibitor using, e.g., site-directed mutagenesis, or by other standard methods, and used to produce the TR activator or TR inhibitor variant or fragment. For example, a fusion protein can be produced by cloning sequences that encode TR activator or TR inhibitor into a vector that provides the sequence encoding the heterologous portion. In some embodiments a tagged TR activator or TR inhibitor is used. For example, in some embodiments a TR activator or TR inhibitor polypeptide comprising a His tag, e.g., at its C terminus, is used.

### Developmentally-Regulated iTR and iTR Inhibitor Genes

Genes useful in inducing global tissue regeneration when expressed in adult, non-regenerative mammalian cells through the introduction of expression vectors as described herein include those that when expressed for a sufficient period of time are capable of inducing pluripotency, but when transiently expressed can revert cells to a developmentally-younger and regenerative state, said factors including combinations of OCT4, SOX2, KLF4, NANOG, ESRRB, NR5A2, CEBPA, MYC, SALL4, LIN28A or LIN28B; by way of nonlimiting example, the genes OCT4, SOX2, KLF4, and MYC; or LIN28A, OCT4, SOX2, and NANOG; or KLF4, OCT4, and LIN28A.

Genes useful in inducing segmental TR when expressed in adult, nonregenerative mammalian cells by introducing expression vectors described herein are listed in Table I.

iTR inhibitory genes useful in designing RNAi constructs or for selecting promoter or enhancer sequences for the gene therapy vectors described herein are disclosed in Table II. Preferably said fetal/adult onset genes are widely expressed in diverse somatic cell types and therefore, preferably the genes from which promoter or enhancer sequences are used for DR-iTR are those genes or those promoters listed in Table III.

### Developmentally-Regulated Gene Regulatory Elements

The promoter sequences that may be used in DR-iTR may include promoters corresponding to any of the segmental fetal/adult genes listed in Table II, preferably, the promoter sequences for use in DR-iTR are those listed in Table III. The preferred promoter sequences for use in DR-iTR extracted from human genome Hg38 are described as follows. It is commonly-understood in the art that regions of the gene regulatory elements disclosed herein may be modified to maintain or even enhance the desired developmental regulation while retaining stretches of at least 10 nucleotide sequences from the disclosed regulatory sequences. Preferred examples of promoters useful in DR-iTR include the following.

| Description | Sequence |
|---|---|
| C2CD6 (also known as | gttctatgaggacatttgcccagcaactccctgtccaatgtccaactggcaacatccttgttattgatccttgtagccaag |
| ALS2CR11) (gene | gataattctctcaaaacaatcatttttgctttaaaaaccgttgtcttccttgacctccctgtatatgcacatagtttactgtgg |
| position | cacttgtattcttattgcaatgcctactcctgaataaacatcattttctttcagagagtctccctctctgttatttaggctgac |
| chr2:201,485,421- | aaggatatgccaagaagtagcttggatatagcagttaactctgcctttaggatatgtgtatggggatataagagttaact |
| 201,621,182) | aaaagctgacctttgagttggtccttgaataaaagaagaatagattccaaaaataagaggaataaataatgatcctgag |
| which is | ctaggagagtacattttgcagacctttggtttccatattaagaaattcagatttttatgtacaataaagaagttctggaattc |
| positioned on the | tctggttttgttctgtttgcatttatttatatttttaacttttaggttcaagggtacatgtgcaggtttgttatataggtaaattttg |
| minus strand with | tgtcatgggggtgtacagattatttcatcacccaggtaataagcctagtgcccaaaaggtagtttttgatcctctcccgc |
| the promoter | ctcccacccttcgccgtcaagtaggccccagtgtctgttgctcccctctttgtgtccatgtgttctcattgtttagctcgca |
| region positioned | tttataagtgagaacgtggtatttggttttctgttcctgtgttagtctgcttaggataatggccttcagctccattcgttactg |
| at | caaaggactaatacgggggcagtgaaagggatggatgacagcaggttggcagacccatgagtaaatcattgcaatt |
| (chr2:201,619,183- | gtctcgtgtgtgtgttgtcccaaattaggtgccataaagggaaaaggtaagtaagattcagatatttagggtcagaattt |
| 201,620,677) and a | taaaatgtgtctgaatgtttaagattttgtggatatgacattgctgcaccaaatgagtttggaacctactagaggtttaag |
| promoter sequence | agggaagatgaatattaacgtgttatgtgtctgtggaacaattgggtaaagatgtcaaagtagactgttggatatttaac |
| of: | gtctggagctcacgggacagtgtagaggaagtaatgactgacactgtaaatcttctagaaagaaatggtgcaggga |
| SEQ ID NO: 1 | gaaaaggactcgtgagggctacagggcggacaagacagtgaactaaagtggttgggccccatgaggggccttgc |
| | tctccaccagcgtgctctgatttccttctaaaggagactgaaatggcctggcaagtgcttgcagcagaccctccaggc |
| | tcgctaagcacgcatgcttccaaagctgcctccagccactcccagcatgggcccggcctgagtgggtcactaggcg |
| | gcacgcacgcgctgggaaaatgtgagaaaggctgcgggagggccgcgcct |
| CAT (gene position | tacaagatatgacaagatcagaaaccagtcttgtttctccatttctttctctagaaataattctttaggaattcaaatccata |
| chr11:34,438,934- | gtaacaaaaacaaaggaaaaaaaaacatgcttttttttatataatgaataaaggttaagaaatgactccaggtctcttatt |
| 34,472,060) which | tattttgttgatatttgtgttggttagttaagaagatgatttgctcgattatttcaacatttatttattcaacaaactgtggacttt |
| is positioned on | ggagatgaacagctgaagctagaatcctgactatcccttaatagctgtgtaactttgggcaagttatttaacctccctat |
| the plus strand | acctctgtttcctaatctgtaaaatgaagataatattagtatctaactcatagggttgttgggagggttaactaagcaaata |
| with the promoter | tacaaaaagctgagaatagtccctggtatatagtaagtgctcaataaattatggatgggaggatcctgacccacatgg |
| region positioned | agtttacagtcttggcagtatggatcgggcaggtagaaaaagacaccaaattacacagccaacagcatctttataatt |
| (chr11:34,437,183- | gtttttaatataggaaaagtatagggtgctaaagatcaatttgtggctttcttttccaatgaactgctaaagttccacttttat |
| 34,438,933) and a | gcccaagattcttatcatacttttgtaagatcaagaacaacatgtgaagtttccactattatatatccacccattatatgtag |
| promoter sequence | ttgtactggaaaaaattaaaagctgataacttttaatttgaagatgatggtatatactatgtatatatattaaggtgcttaaa |
| of: | gataaatcctagcacctgaggaggtgtagaaatcattcaaactctttgattacaatgacaaactagtcagtaacttacta |
| (SEQ ID NO: 2) | aatattgtactatatattactaagtattttactcttcaacatagctttttaaagacacaaagcttttcaaaattcctgcttacct |
| | gggggtaaaatttggggaagcagatttctccagtgtttaaagaagccaatttggcagtgtaccagagttgaatacatttt |
| | tcccatcacaagggaatacatttttcccatctaagttaagttgtttttctctggtaaaggaggaaatcaacacccatctgta |
| | cggagataaacgtttcagagtgtttttatattaaataattagtatactagtctagtaagtgataatccacgaataagttaag |
| | aagagctaagaaagaaaaagaaaagcatccatccatcctttggttgcaaataatacttacattagcgtatggcaaaatt |
| | taattttgtacagagtaatttaacccaggattgctgactttttaagagctgagaaagcatagctatggagcgcaaggcc |
| | ccacccagcagggtctaagtattccgtctgcaaaactggcaggccaccaacggccgcgtcccagggcggcctgaa |
| | ggatgctgataaccgggagccccgccctgggttcggctatcccgggcaccccgggccggcggggcgaggctctc |
| | caattgctgggccagagcgggacccttcctttccgcaccctcctgggtatctccggtcttcaggcctccttcggagag |
| | ccctgctccgagcccattgggcttccaatcttggcctgcctagcgccgagcagccaatcagaaggcagtcctcccg |
| | agggggcgggacgagggggtggtgctgattggctgagcctgaagtcgccacggactcggggc |
| COMT (gene position | tgttaactcacatgcagtagtattaaaaaatgactcggctgggcgcagtggctcatgcctgtaatcccagcactttggg |
| chr22:19,941,371- | aggccaaggcaggtggatcatgaggtcaggagattgagaccatcctggctaatacagtgaaatcccgtctctactaa |
| 19,969,975) which | aaatacaaaaaaaattagccaggcgtggtggcagatgcctgtagtcccagctactcgggaggctgaggcaggaga |
| is positioned on | atggcgtgaacccgggaggcggagcttgcagtgagctgagatcgcaccactgtactccagcttgggcgacagagc |
| the plus strand | aagacactgtctcaaaaaaaaaaaaaaaaaaaaaaccccaaaaaaacaactcatatggtttagtaacagactgggc |
| with the promoter | acgaaagggctatttaaaaaacagttgctcctctggcggaaaggaatgcctggagactacttattttatttcttactggg |
| region positioned | aggaccaggatcatcatcctattttgcatagaaggttcagtttgttctttcccttaaaataggaagataagggcattatcc |
| (chr22:19,939,926- | ccctaagtctcgtatgatattccccattctgagtccagaatacctagaaatttggaatttggctattgccgtgtctggact |
| 19,941,771) and a | gtgagtatgggaaggggaagcttttctgtctgttgtccccactaccgcccctcacatccgtgattctgaaccccatgat |
| promoter sequence | aaatgccctttgaacctttttcctccttttgatgccgaatccccttttatgggactccgccagcacgggtgcatttaacctt |
| of: | cctgagcttgcccacctgacgcatgcagccaagaagctttgaaagatggagtgccctggctgagggttccaggtctc |
| (SEQ ID NO: 3) | agcaaggccacgaggtggtgactgcctcccagaactctgttgggccaccctccgtcctggagacccccaaaacctc |
| | catagcaccaactctgcccattcacacacacagtcagccctccctcccgagtccaccgcgggcagagggccagcc |
| | accatgcctcccatagccgccatccctgggaaggagccctgaccccgtccttacacccctctacactcactgcctgt |
| | ggggagagctgctcgctgtcacaagcatccagggggctgggtcgaggggccccctgcatggtgggggaccatgt |
| | tgtcgtgcatggaatgaaactggccacacataacagatatggtggcgctctggtcggatctaggggagtctgtcctac |
| | aggatgaggcttgaactgtcctgagtgaccagacacccagcctctgcttcgagtgctcagttatgattattgtcctgatt |
| | tagttaactgttcttcaggggctccaggaggacgagtgtgtatcctcccattgctctgtgcagcctctaacctctagagt |
| | ctaggggtctggggagaagttgggaagtctggccagtggggccggtgcctggtgacctcgggaggtgggatatca |
| | tcatcttcagaactgtagttgttactgggataccagctctgggagaccacaggtgcagtcagcacagcaggaccttag |
| | acaaggcacccagccccagtttccccacctgggaagggggctacttgtggctagaagcagcccggactcctgagc |
| | aagactagaccaagaggccggtatgtggacacccccgcgtgggcacccccacggggacaccctggccaccgcc |
| | gcgcggacaccctcacgaggacaccccggccgcgcggacacctaccgcggggacgccccgaccccatcctac |
| | ctgctgcgccccgcgccggccccgcaccccgcccgccacggcctgcgtccgccaccggaagcgccctcct |
| COX7A1 (gene position | ggaaaaacctaacattaaatttacaagcatagtttgaaagtgttaggggaatttaataatcataaaagccctctttaaaa |
| chr19:36,150,922- | gagaccgtggctggaagcggtggctcaggcctgtaattccagcactttggaaggccaaagtgggcggatcacctg |
| 36,152,449) which | aggttgggagcttgagaccagcctggccaacatggtgtaaccccatctctactaaaaaatacaaaaatgagctgggt |
| is positioned on | gtggtggcgggtgcctgtaatcccagctgctcaggaggctgaggtacaagaattgcctaaacccaggaggcagaa |
| the minus strand | ggtttcagtgagccgagatcacaccactgcactccaacgtgtacaacagagcaagactctgtctaaaaatataaatta |
| with the promoter | aataaataaataaaagagattgttaaaaccttttacaagctgggtatagtggctcatgcctctaattctagcactttgggt |
| region positioned | gatagaggtaggaacattacttggaggagttggagaccaccctgggcaacatagcaagacactgcctcaaaaaca |
| (chr19:36,152,448- | aacaaacaaacattgtctgctggctgggtgcgtggctcacgatgaggccggcaaatttcttgagctcaggagttcga |
| 36,153,947) and a | caccagcctgggcaacatggtgaaacccagcctctacaaaaaatacgaaaattagttcgagcatggtggtgggcgc |
| promoter sequence | ctgtagtcccagccactcaggaggctgaggcaggaggatcgctgaacccaggaggtcgaggctgcagtgagctat |
| of: | gaccgcaccactgcactccagcctgtgcaacagagcaaaaccctgtctaaaaaaaaaagaaaaaaaagaaaagaa |
| (SEQ ID NO: 4) | aaagaaaaccttgtagactaggccaggatctttcctggttgctaaaaccattaggacaactgtgctgcaccccgttga |
| | ctgccagaattagagagctataaatagatatgatgtgtttcttagggtaacataaaataccgttttactcccaaagacttt |
| | agcttgtgtttcctaaaaacaaggtcatttaatgtctcaacaaatctctgagaaagatcctgccttagccctcacgttaca |
| | gatacggaaacaggctcggaggtgcaatctctggctgtgtagacagaacagggagttggaacacggccttatgcgt |
| | ttcctcactctcacataccagcgggcagggagagatccggggaggagttgcacttgcccccggcggaaatctccaa |
| | aaatctgcaaaaatgtattccctggtaccgctttgggctccggtgggaccaagcgaaattccgagaccgaaacggat |
| | gcgcgctgcggcccagggtgcgggtctggaccgcctcctccgtggtggacgaatccgaggagcaggactccccg |
| | caacccagccccagccccagccccagccccgccgcgtccccagctgtcacgctgcgcgcagcgggtggggcct |
| | ggggttcctggacagaggaggactacgcgtgtccttgggc |
| GYPE (gene position | tcttacttctcccgtgacttgtctactcatcctttaggtctcattgcttgctctggaaaacctttccagaacctcaaccctga |
| chr4:143,870,864- | gttaggtacccctcttatgtacccttataacaccctttgcttactactatcatccacttgacactccataatgcaattgcctt |
| 143,912,131) | cttaatttatttgtttccaaattaagctgtcagttcctgaagggaaacttgagttactgaaggttgtgtcttccacacttggc |
| which is | acagtgcccggtaggtgctcacaaatcattggttgaattattatttggatgtagaacaagctggacctatcaattctcag |
| positioned on the | cagaaagattatgtatgatacctgattccagcagtatagcacgatggacttagaggcaactgggaacagttttccactg |
| minus strand with | tctacatcaccattgtagtgatgagagtttctcacttctggggtttcttcaacgcacatcacatctcattataattttaaaga |
| the promoter | aaggaaactgaaaactgggttaaagaaatctcatcactagaaggtgttgaagaagactcctaaataaattagatttcca |
| region positioned | aacccaaatgccaaaagcttagaaagagtataaatgaatgcgatggattgtgtttttgctcccaattattagggcccttc |
| (chr4:143,912,132- | ctgtaagtgttttatatatcctcaccattgacctatgacttgcaggacttccatttggaagaattatatttacttgtcccattg |
| 143,913,679) and a | aggtcagcttggccactgatttactttggctaatagaatgtgagaaaaaaaccataacacagtctgagaggaagcatt |
| promoter sequence | aaaagccattccttatctggcctttttcccccagtttgcaacaagaacagcatattccaaatgggggtggctccttcagt |
| of: | ctggatcctggaacttgtcaccgtcatagaccaaatgtaatgtgagcaggaaatgaaccagtgttgctgcaagacact |
| (SEQ ID NO: 5) | gagattttgaggccttaccacagtagaacctaatctaagaaatggaagcaagcatcaagtggagcacttaccagact |
| | caagaaagcctatttgtcccaagagacagccagtccttagctctcatgaattgtcacaatgtagaactgcacgctaact |
| | cttgccagatcttcctgtaagaggagccaaattttaaattaaatcaattcctgatttttaattaaaatataccttctttttaaat |
| | gttggcgctaaatccatacgtttaaaaaacacttagggcaaatcaaatcaagtaataactcataatctcttttccattccat |
| | agtttgctcatacataaaataagaatgcgctggcccggcgtcgctgggctttcctcaaggggtccccgagcagcgtc |
| | gcagagcgggccgacttccgggaaggaagtgaccagcgactgagcggcggccggcgcgtttagcgccctgaac |
| | atgcggcagtccctgcgggcgaccccaggctccggacaggcagcggcggaggcggcggctccggagggaag |
| | gaggcggcggcgccggcggaggtggcggcggagg |
| IHO1 (also known | attttctcgaattttatataaatgggatcatacagtatgtattcttttgtgtttggctttcactcagcgtaattattttgaaattta |
| as CCDC36) (gene | ttcatgttgttgtatccattgttcattcctttttattgcttagtagttgtccattgtatggatatgccaaatctctcctcttgattttt |
| position | tgaatccatacacaaatcaggttttcatcctcactacatcactgtaactgctcctaccaaggtcactcaaagttaattcta |
| chr3:49,198,428- | actaatcttccctgaacacatccgcagcatttgtcatagctgatctctgcctcctctttgaagcagttccaagtgccttcc |
| 49,258,106) which | agaatgcctcattctcctggtgtctttccccgtctcctgaccaggcttgctccaactccttggctgcttttccttcatctcca |
| is positioned on | aagtcctctaaactggtgtgtcctggggctgaatccaatctcctttccatttatactccattggtgattgcatcctgtttttgt |
| the plus strand | ttgttttttgagacagggtctcattctgtcgcccaggctggagtgtagtgacgcgatctcggctcactgcaacctctacc |
| with the promoter | tcccaggttcaaacgatcctcccgcctcagcctccgagtagctgggaccacaggcatgtgttaccacgccggacta |
| region positioned | attttttgtagagctggggtttcgccatgctgcccaggctggtctcaagcatgttttttgctttctaagtcatctataagcac |
| (chr3:49,198,025- | cagaggcagagttacatagatccttcctgcctacccgcactgtccctgccctcaggccactccagcatccagcaggc |
| 49,199,497) and a | cagaaacctcagaagatttcttgcctcctttctctttcctccaggggcccggttcgatgtccccatctgcccacggcgc |
| promoter sequence | ctctcgtgccagctgctccgcctgtcctgcccttgctcagaccacccaagtatcatctgtgctctggcccctggccacc |
| of: | agttccaccccacatgctgtcctcacaggtggcgccttgccattcctggggaccagggcctccttgttctggtcataca |
| (SEQ ID NO: 6) | cagtggagtgtaggtcgagcgacaagtgagtagatctgcatgccaggtgggccgagggtggccattccggcgtga |
| | gtgtgggggtgtgagggaacgttgcatggggtgcacgtgggctgggtgtggcgtgcagcggtgtggcgccaggc |
| | aggcagcttggcggggcccacaatcagcgcggttgagaccattagtacggttaaaaccgttagctgcctgtcgcgg |
| | cgagggcggggccctggggactggacggaatcccagttggtcagaggagaccctgggggcggggccgcgcga |
| | gttgccgttaggacagttaaaaccgttagctgcctgtcgtggcggggggggccctgggacaggacggaatccct |
| | attggtcagaagaggacctgggggcggggccgcgcgtgccgtt |
| KRBOX1 (gene position | ataagattcttcttgccttaccttcaattgtatgtgtgtatgtgtgtgtgcacacatttgatgatgtccatagagaagaatat |
| chr3:42,809,483- | acaagtgggtggaaattctgcttgtgtctgtggttcacaggagttctgtagtctctatattctcaggctagccaacacttg |
| 42,942,792) which | gacctcagcaactcattaaacattttagctgatttctccttaacagcttacatggcatccaatggcatctgtcctatgtaaa |
| is positioned on | caagtgcttacaaccatctcttcttgaaagtgcctgtcttacatcagattttagattaagtcgtttgcccccagatcttagct |
| the plus strand | ttctgatgtgttcaagagaaaattttagattatctggctgttttttgttgttgtaagggtgaggccatgcacttttcagttttct |
| with the promoter | acatcgtgagaaggtgaatttcttgatttttcaaaaagaatcacacttttaaaaaccctgaaaaataaagaaaagagact |
| region positioned | gagctaccagaacggcagtgactttgggtggctctcacttatgccaggcacagggtgaattcagggctctgggctat |
| (chr3:42934886- | gaggggtcgactgcaaacccctgcagcagatgtgagctgcatgtgtgggcccctgtgtcagcctgtgctcatttgtct |
| 42936341) and a | tcctacagtgatactgcatgtgatacacaacacccaaggacaatggaggtgggggcaaatgtcccttattcttcacac |
| promoter sequence | ccttcgctgttggcatcaaagcagttactctaccttctccacaggctttggcacagcatgtcaggaagttctctggggtg |
| of: | gccacacctatttcctctaagtgctttcaacaggtttttcatcatgataggtatcctttcttacctctgattgatgtagtcatg |
| (SEQ ID NO: 7) | ctgttttcctgggacagtgttaagctctgctggacaggggatgaatatttcttgggtaaactgactgctctcactttgccc |
| | atggtccattgctaatcctctctgctagtggtaatgtatggaagtgagaggcgtcagcaacattgaaagacatcacatt |
| | ctaggagtgcaatcattacagcaattcaagagaagtcctggttgtataatgtgtgattcgttcattatgctataacaatcc |
| | aaatgctaaatttaacaagatcaataaaacgtataaccattcacattttcttgcatattctgggtaagacttgtagttgcag |
| | ttattgtagcacagtaataaaggcactggaagccagtattcaaggttggtacgatgatgcagcgtcatgagcaaggtg |
| | cctcaactcagaactgggctctggaggaacgccaagagcctatctcgccagcgtgcccttttcctcacgggaattag |
| | gagggaagcaccaagatgtggggtcctccacgagtcctagagagaaatccagagagtgtatgggttcaccagtca |
| | gtgtgatgacgc |
| LINC00839 which | aaactttaagtataaaagcagtactgatagggtttttgcatatgttactttaattatgactttaattgcttaaagtatttcctgg |
| is positioned on | aaacacgaagttgttaacaagtgttagttatttttgtttgagagaatttcaagagattgaaattatcaacagaggaatgtg |
| the plus strand | aaagtcgttgggcattaagctaagccataaaaactttaccctgaaaatcgtgggggtgtccttgaatgcccacgagttt |
| with the promoter | aatttccattttctgcctgaggttttaaggagtatgcaggattaggagggtaaacactcatagctcattagtctaacagtg |
| region positioned | aagaaaccatgagggcctagagtaatggaaaggcaatgaagtgaaaagacagttaaaatgtgtaattttccaaatac |
| (chr10:42,473,979- | agaactgagatatttaaaaggattgtatgtggtggttgggtgccattacaaaggaggtggatgaattttaggcgtctag |
| 42,475,490) and a | atttgtggtgctgatggttgggcaggggacaagcacacagggaaacaaggaaaaaagatgtatttaggtttaggtaa |
| promoter sequence | aaatatcacatgctcagtatccaattcctgttaaacacctggagatgggagtctgtggtgcaaagggagttcgagttta |
| of: | actatacacgattgggtgatgctggcatgcaaggagagatgaagccatggatttcattaaggaccccaatctgaatgt |
| (SEQ ID NO: 8) | ggaccgcacacaagaatcagaaccaaaacctggacacagccaagactgcaggtttctcttcccttgctccaaatcca |
| | ctttcagaagaattagagaagaattgactaaagaatgtaagtaaactccaggaaacagggttccacagaagctgggg |
| | aaaggtcagtacgtggaagagagagatcagtagtacctgctcgctacaaggaggcgcctccaaatgtcaatgccta |
| | ttttcaacaagtgggacccaaagtgattcatgccagtgctctaaaatacacctgacccttgaacaatgtgggggatatg |
| | gatgctaaccccccacagtcaaaaatccaagtttaactctcaactcccccaaagcataactcctaacggcctgcggtt |
| | gaccggaagtctttgtcactaataatacaaaaaattaagacctattttgcctgttattcacattacacactgaatatgctgc |
| | tcccagcggaggccgagactcctgcccgggcaccccctcacagcccccactccagcctccccagactcaccctcg |
| | gcggggccaccttcttctcatttcagtctggggccacattctccgggcctgcggggcatggcaggaggcaaagcca |
| | aaggcgaagtggccacaaattccggtgggggcgctggctcctgccccaccccagcatctataccctgagtctgata |
| | ggacgagaaggaacatttcagccagccctgattgggtcatattatccaatcagagttgttttgcacagtggctctcatc |
| | caatccgaacatgccttacag |
| LINC00865 which | Catgtcttggtgagcctgtgtttcatagaacctggctcttcatacatgctggcaatgtaatccacccaatatcattatcac |
| is positioned on | aaacacctttcccattgaaattaacctgagttggtttctgttgcttgcagctgatgaatggatgaccttctcttcctctatgt |
| the plus strand | catctcctttagttatgtatttattaacttaaatctcatattactctctttggcctctgctacctgaatcttctggtatcagctga |
| with the promoter | agagggagtctgggaggcacagaactggaagtaaggccatactgggcctgggaggcagagagagtcctactgg |
| region positioned | agggccctcagaaagtgaattcagcaggcaccagcctccagccacagggtcctatgcccacacttgcttccacaag |
| (chr10:89,836,052- | cctggtccaggccaccatcatgtcgcctggaccatggccgtatttcctagggtctgcctactcctgtccttgtccctgta |
| 89,837,217) and a | aacactttatcaaacacagggatattgttaaaacataatcagatcatgtcaattctctgtgaaaacctcattctctcagagt |
| promoter sequence | aaaagtccttatagaagcctaaaggccatacatggtacccgcttcctccgaaaaatcgcattctctttattacgccttcg |
| of: | atccctaggagaacctaagttcaagaggagatgctgggcagggaggggccagccacttgctagggtgccccagc |
| (SEQ ID NO: 9) | ccctgtctgcttcaagagccaagtcattttctcctggacccctctgagctgaagacaggaagagccgggggagggg |
| | gatctcttggtctgcgcctcagtttctcaagaacagcaacagcagaaaaataaattgagagatgaaggggctaccac |
| | catccaagtggccacaaccatccactcccatgccggcctggagttagcagacgggcgccactttgctctgcttgtcc |
| | cacgcccatggaggggggacaccgtccggaagtgaaggccaggaaaacagaggggagagacagcggggtgg |
| | ggtgggagcagtcgctgctgtgaactcgctggagcccagactcacgcaactaaggggcagcagtgggaagagaa |
| | acggaggggtggtgggggagctggcgctctgaacgcctgttccttctgttcagactagtctttgtttattttgcag |
| LRRK2 which is | ataaatattttagcatgtctctgtaaaagttaaagactctataacaacaagacatagacccaatatccaggtt |
| positioned on the | gagtaggagtttgctatcttactaatctaatgtattaacaaagtctagagcattcttattaaagtggtagagcatgaagaa |
| plus strand with | ccttctacctggagttaggagatttgctgggttgaagtcctggctctcaacttcccacctgtgtgactccagcaaagctc |
| the promoter | ttcacttctcaattccaaatctcaatttcctcactagtgaaagaaggatacatagtagtagttgtatccatttcagaattgtc |
| region positioned | atcaggattgaagtaacagatgtgaaaacaaaactaaaattgtgaagtagtaacttccagtccaatcctcttctttgtaa |
| (chr12:40,223,497- | gtgatcttgaggacagacacatcacctgaaggatagatcatctttgcctagccttgtctacaatgttcttgctaggtttctt |
| 40,224,889) and a | ttcctaaaaacacattacttaggtacttaacagaacaaacaaacaaacaatcaaaagacaaaaccaaaaaactttggg |
| promoter sequence | gttatagacacccatactcataagtatttctgaataccaagagaagagtatttaggtttgcttctctcaacttttcacctttc |
| of: | atttcatgtaccctgtcctttgtctcagctctaatagctctgagagctgattacttttcgggtgtcccaagtatcaggatcct |
| (SEQ ID NO: 10) | gcctagtgcaactcaaatttccaaaagttaatttagtggccttttggtgaccagagcttcagataactcacagggaaac |
| | aatgtttatttcctctcccactaacagtcacaaaaaatcataaaaaagagtagcgggggcagttttgatggctaacccct |
| | ctttccatcctttgggggaaaattgctcatctccctataggtggaactctaaagacaatgtattcctaaaaggggccatc |
| | tgggcggtgtcctcttttcccagcgccctgatttctattcttagatctggagataggcggctttcatttttcctgctcccagt |
| | tcccagaccttccgtggggccgcaggatccccggctggcgggtcgcggagggtggccggccgggctgcgcact |
| | gcgcgcctccgctgcggggctccgggcctgtggactcagcggagtccgctgagtcagtttcttcccgcgcgactcc |
| | cggccgcgccgccgctgcggtggaatctggtcccaggaggcggcgtccgcccggggtccggtctaggcgtgcg |
| | tgggggccacggtcacggtcatcccagccaggcccggctccagcagccccacggccgccgccagagttctgcg |
| | cggcccgtcgcctcggcggagcctctggcaggcccctgagctcgtttttgggg |
| MEG3 which is | cagcgcggagggagcagagccgggattcaaacccgtggtgctttgtaggaaacccgtaatttccacggtctttttcct |
| positioned on the | tgggctgtcagtaaggccctgaaataggagagtggggtttactgtgaacctcgaaggagcaggctcaagagccag |
| plus strand with | ggggcccagaatagggcagaggctggcttctcactgggaggctcaacttcccgcaggggcgccaggccctgaca |
| the promoter | ggagagactggacattaggtgtctggaagtttctccttctaggggctccaggagcatcccacctgaggcctggggct |
| region positioned | cccaccggaacgtcaactcatgcccttgtggggctgcagggctgacgcgggctggcactgtgtctacgacagcctc |
| (chr14:100,823,838- | ccgggcccccgggtgcgtggctgcggatgctctctggcggccaccacagttcccacgcgcggcgggtgaatgcg |
| 100,826,107) | agccccttgtcaatgcgtgcccctcgcctctgcccccgagaggctagcaaccgggcctgccggcgtctgattggcc |
| and a promoter | gtgggggcctgacgacaggggctcctggttgctgattggccccggccagctctggctgtttcctagctattaatactg |
| sequence of: | tggctaataaacgttctcctgttgacgcgggtgctcggagccatgattttttgaagcgttcttgacgtctgtggctggtg |
| (SEQ ID NO: 11) | cgggcgtttctgctgtgcaaggcctgcttccggggctctagtccctggggctcctggcaagctccacaggctgtaaa |
| | gggggtgttttctttctccttcgtgttacctgacatattaaagcagcgccccatgaggaccccaacgtcccacgtctcta |
| | tctccccaacagtgcgcctgtttatgaaaaaacgagccccccacacgccgtcccaaggctcggcgcctctagtgac |
| | ctgacggtcaatgttcacctccctagtcattagctgtggacgtagaaatagcctttctcccttcttttcagccctggaatct |
| | cccgtctgctttacagtcaccttgtgggatcgttgggcatggggggctccctctgacgccgcttaaaccccccaaaga |
| | agtgcgggggagcgattctgacttgatgccctcggggggcctctctgctgtcctctttggggccccttgctcatcct |
| | cacctgctttctgtcaccgagcactccatccttgtcctcctggctgccacccacctgtcaccctaagatagatcctcggt |
| | ccctttcaacagcattttgacctttgcgagaggacatttgattgacggccctgcacgctctgccactcagagccgggc |
| | agagctgccgagggctcccacctgttagggattaactcccatgtgccagctccggagccgaggccgcggcaggg |
| | ctcggcgcaacatgtgtcgctgcctgtttttcggggcgctggggctctttcaaagggcatgtgtgtggggatcacccc |
| | aactttgaggggtagctcaggcatctccacatgggacacagtccgccctctaaaagtactttccttagagggcacgg |
| | ctcctgtctggattcttattacaaccctggtgtgtgtggtggggggtacccctggttaaggtggaggaagcggaccca |
| | gtggaagaggaaggggcatagcgggttccgcgaacatgagttgtaagcggcagagcccgggactccacagcca |
| | gggttttctgactcggagacgcggagctctgtctcccatgtcaaagagaaccagtcagaaacgcacgggtgggctg |
| | ggagggggtgacagcctctggcttacatctgggaaccagttatgtcgcccgggcattttattttcctgaacaataaga |
| | gaaagcatgattttctttcgctaagcccgcatcctccgatggatgttccgaaaccgccaggtgtgggatctgcgcccc |
| | gacagccccaccttggccatcggccgcctgaggacggccagccatcggggcgattccccccacacattgtgcctg |
| | aattcaccctgcctggcgagccgggctcacgcagggaaaaagcacccgcgaccacagggtgttggtcatggcgg |
| | ccaggggcactgcggcagaattttttcctcccttctttgctgcaatctgggtgcggctagagcaatttgtcatagaatct |
| | ggggggctcatttttccggccaatcacttttagagaaatgagcgcattgcagcagaatgcgctgacgtcagagacca |
| | ccccttctgcgcctccatataaaccccacccagcc |
| MIRLET7BHG | ggacctccccgcactgccaggagaagggcctgacggggattctggccccacacagtgcgatggttgcccagcctc |
| which is | accgcctgcagctccatctgagaagggcctgatacgggctggggtccaggcccactgccctcatgtcctgagcccc |
| positioned on the | acggtggcctgcaggcctggtttggaggccattatctcctcggcggccagccgggcacagcagtggccacttctgg |
| plus strand with | tcgaggcagttctgtgggtgaggggcgagcatcgcctcctgcaaccgtaacccaggccctgcctcccctgccatgc |
| the promoter | ccctgtgggtcccgtcgttgccatctcgtcctcagtggcttcccgatgttgcctgctggcagggcaggccccgtgcca |
| region positioned | tggagcatgagggacccttcccctcccagggccaggcttttggcagccaggccaagccgctgctggaatggaattc |
| (chr22:46,078,318- | ccggcgttcctggtggggcgcatggcatcggaatcctacctgctttgtctctggcatcagctggtgagccagtcacgt |
| 46,085,996) and a | ggccgtgtgcgtgagaccagagcccaggactgagggctcctcccgggcgcttcctcctcgctcactcacccccac |
| promoter sequence | cccctttttttggctttcagcaggactggctctgagcaggcgtaaaacagtgttaaaactgaatccgggcagcaggga |
| of: | gctcctgtccacggcggcaggctctcacagtccaccgggctctcgcggtccacccagaccaccctttacctcgagt |
| (SEQ ID NO: 12) | ccttatgcacagaaaggccctgatatgtcccatacactcaggagttaggcccagagctgggcagtggtcactccacg |
| | ccattccctctggtgtagagctggccctgcctgcccccagacggccgtgggggggtggcaccgcttcctggggaa |
| | ccccttcccacacttctggccttgtttctcacccacacaaggacaccccagtggtcactgctgcagctcgcggtcaca |
| | tagagggtgagaggggagagctggacaaacaggtgacccagcagacccagcctgatgcccgcaggagagagc |
| | aacggggtctgatattttgtctccaaatgaaagagccacagtgaaaccccaggcctgccaaccccagttgtagggcc |
| | agagaacagggatgtttccctgaggcggtggcaaggtttggtttggtgaaaacgaaggatatgtgagggtctgaga |
| | ggggagggtgactggcctagactccacccctggcgccctgtccaccgtggctggctggccactctcggacccctc |
| | ggcgtcaagcgctgactgggtgcctgcctggggcttggggctctgtacgtgttaattctgccactccagcagccctg |
| | agtgggaggagccattatcccccttctttctgtagatggggaaactgaggcaggcttgcccatggtgaagtggccag |
| | tcggacacagggccagattgaaacctgcagcctgggctcccggctacaacagcgccagcctccacaggcattaga |
| | aggggactcactgcgagggccccagccagggcagctttcagggtggggtctctggcctcaccctggggaaacag |
| | ccggggcgctggctgcctcctgctgagcctggcgtgggaacaatgtggcctctatccctggagcgagccaggccg |
| | cctggacgcccagcccttcagagcagcccggccaggcaggcgcccacagcatggcgcccgggccgcgctgtcc |
| | gtccacggggtgcgggcgccttggccaggcccaggcaagccgctccccgtgtcctccctggctggccactgagtg |
| | gccagacgccggcttcctcctccctctcccgcccggccagcctcctccttttttggtggtgggtttgggggcccagcc |
| | cacccgccactgccacgtctgccatcctcccgcacccacgagcatctttcaaaaattcccggtgggcggggctgag |
| | ctgcagcggccgcctctgccccccctccccgggcagccagtgccagatgagagcaggagacaaaagtagcatttt |
| | cctcgtactccggggctggcagcggggccttcccccagcaccacatggctcctggcagccctgccccctgtgccg |
| | gggtggccgaggcccagccgggcccactggaccgagggacggcggcggccagagactagggggcccgtgg |
| | gcccatagctggggctggcaccgggcagcctgctggccgggggacctgccctcctcccccacccgggtgaccg |
| | gctgggcccagggacgtcattttcacttcctggctttggagaagacgcctccatcctccggcacctegggtaagtcac |
| | cttctttccttctgtcgtgaagacgagccgagggtgctgccatcttgtgccccggtgcctgttacgtgtcctccctctaat |
| | tggttctccaaactcagaattctatttcagagccctccatcttccgtgcaggaagccctgccaaaaactgcagggaaa |
| | atgcagaagccaagcccatttactttcaagatccaaagctacaggctttagaaatcacaataaaaatgaatgtttattatt |
| | tcacagtagctgcgtcttccattttaatgggaacaaatgctttgggagtattaatagccatggcctactcctgccttcctg |
| | gttgtggcagtttgtgtgggggtctcattattattattattattattattattattattattattattattgtcttaatttttggcggc |
| | gccaggagtggcaaaaggaaaaccaccaggaccatccacggctcctcccagcaaactcatcggcaggcagggc |
| | caggctggcacccgggagggaggactttgcatctagaaacagaggcccttttattttgtccggcatctttaatgctacc |
| | atgtccacagtttagggatctagttctttctccctttcgttttattttttggaaaaaaaagaaaaagaaaaaggaaaaaga |
| | aagaaagctccttttggccctcagctcagtgtggggaaaaatattttcggttgttttgggctttgtttttgagcacttatttc |
| | aacttgggggttccgcctgaaagtgctgggcctgtcttgtcaccgccctgaccgcagactgtgtgtggcgtgcgcgt |
| | gggcaccgttcactacacagacccttggggaagccctgagtgccctcatggcctgcccgtgtcctggatcccaccc |
| | cagttcctcacctgcagagctgttgggggagacacccaggtcgtccagggcctgaaacgtggctggcagggccg |
| | ggagtccctcaaagacctttgagtccccccgccttgccacctctgtccaggagctgggctcagtggctgcccgggtg |
| | tccctgttggtccctgcccctccaaaagtgctgatccttttgattcagagggggctttgcagagcacagccccatgtc |
| | cacctccacgctgtggcaggctgacaaaagcgtcctcggatgcggcctggttccccacccttgaggggagccagg |
| | gtcttgatatatagaaaatggaagaatcagacccggaaagttctaaacggcactgagaggaaaactcccatggaaat |
| | ccatctttgttttcccagttcggaggcgcgtgggccctgcgtgcgcgggcctcggtctgagcggccggcctcggca |
| | gacagcaccaaatctgtaaacggagctaagctcacacgaggactctcggcggctgcttttgttcgtagatgagccag |
| | cagtcgatactggagggtctggggctgcagaaacaggcatatatagcagagataattaaaacatttcatgattatgac |
| | agccacattctgctgctgcaagctggaggattcatgccacccaaatatttagcaagcatcaaataaaacactggtggc |
| | aaaataaaagtaatagaaaaaaaaaacagagcgtaatacatttcaaaggcaaaagagtcaaggggatggataggg |
| | aatggatcgggggaaaagtggggagagttcagcattttaaaagaattcacatgtttaatagctggccactcatattttttt |
| | tttggtataattaatgtaggcaaatagacccttaattctaagtcctggaatgagagatggtaaaatttcctgattattaattt |
| | atcatttttgttttttgtacagggtaggggcaagtagcagaaaaatcaattaattcacataaaggaacattttcattctaaat |
| | taatattgccactgcaattaaatttatctagtatttttaacatgtatggataacacactttcctgtgtacaccttggatacctg |
| | ggaccgttatatgtgatcaactatcatcttagttttttagcttggttaacacttgacccctccccccacctcaaaatcagta |
| | aaatgctgtggattctgctttttaaaaaaatcaggccaaaatctcagcattctccatggctcccagtgcttctcggcagtt |
| | tttttttttttttttttttgaagaaagtaccacgcaaagcacttttcacatcatcgcttctatataaattccactatagactgaaa |
| | ttatacattctcgcagcacagatgggctggtctgtcattacggctgctgggtacttgttggaacacacagcatatttcaa |
| | ttgtggagtcgacaatttcgaagaactggggtcaaaataaacaagttgcatttgtgtatagaaacagacttattcaatga |
| | tcttctcatcccctggaaaattcggcagaggaacgatcatcaccttcattaaacttgaatttaatttttggtcttcatctttta |
| | agaccccacatgccctgtgtcaaaggcccatcaatgttttaattaattaccaatttttctcttatgctgcagcattagcagt |
| | ggaacacaagatttaaattcaaactcgtgggtgtggtttttaatatcttttgactttaattttaacatcttaataattttaatatc |
| | ttactaatctctataggggaggtgtgaaacctctgttctgtgtcgagttgcctattgttttgaggcctttatgaaaaatcag |
| | atgattcatatttaggatggtctaaagatacatttaaatccaaacaccacccacaaaaaaactcagcaggtaaaataca |
| | tagagtagccataaacttaaaaaaaaattatctcaccaagataacaaagtgcagaatttattggtttgtatcagcctcctt |
| | gagggcagagcagtttttacagccacaataatttgcaaatgatgataactgtttattttgcaaatatgctcatattatgag |
| | gccatcagtggaggcttttttcttttcccatacgtgtaagtgtttgcctctgtgtttctaaattctggattcaggctctgcagt |
| | ttctcaccggctgagaccacgttcccacgttcctgcctcagtggcagtggccgggaggagggaagggegcgcccc |
| | ctgtgttgcctcctgcgagctccagccgccacatccatttgttcgtagaagtgctttgttgtggcttgtttattttaaggga |
| | tgtacctgttggtggagatgttagcaaatcataagaaaagtaaaaagttgatgaaatttgccagagccgcttgggagc |
| | cagctctgctgtctggtggttttaacgacatcatcccttccagttctggtcttttagcctattgactcccgcctccttagca |
| | agaggaatatggccctggtctgggcgctctgaaagatgaggcaaagtgaaaaggccccagcctctgggccgcag |
| | agcctctaagcagcggtgggcatggcgatggtggtaatagtagcaacagcactctctgctgctgttcacaggtgccc |
| | gctgtgtaccggggctcctgctttcagaaaagcaccggctctgggacccaaggtctcggttcagatggggtcgtcttt |
| | gtttccaggccgtgttgatcaagggcaagtcatttcacctctccggcctcatttccttatctgtaaaatggaggcagtaa |
| | gtgtcaactttctgaacacatggaaagctgggttcatggtgagcaccccataaatgctattgcttccttgccttatttctaa |
| | ggctcagactatgcctgcaagggggcattttcattacctccattccaccaggggaagcagcgaacctctgagaggct |
| | aagtgggttccccaagattcaaccagctgataagtgttgggccagggcttgaacccagccctgccatccttcaaagc |
| | ccatgtgctgccatagcattcaccgacgcgtgttgcggcacgcgtgtgcgtggtgccccgggtgtggacagcatgtt |
| | ggtacgcagaggtgcagaggtgtgtgcgtgtaatgtttgcacgtgtgcataggtgtcctcttatttctccattgcgtgag |
| | gttcctggtaggtcggcagtttccaaagaggtgagctggtgtttggaggtgcattggtcggagtgggccacacagtc |
| | ctcagaccacacctggggatgactgtgccatgggctctccaccaccccagggatgcccgtgggaagaagggtggt |
| | tttccccagtccagggctagccaaagctggcagcgggtggactgggcatccaggcgtgggcttctcgtaggtgctc |
| | agcactgctgagggaggctctggctgagtggcctgcatgctgggccttggccagacgcgctgctgcctccttatgtg |
| | gacaggcggttctgggcgccggccatttgcagactgggaggagtggaagtgaggcctgcgggccccggagagg |
| | aaatggcggcgtgggcaggcccagaacaggaccgggaaggcgggccgagggagggagtggcccagaaggc |
| | ctctgtcggccggtcagcgtgctagcggcctttctccaagaagcggccagctggccaggccttcccgggcaggtct |
| | gtttgtctgcccacatgcacacacgtgcacctgcgtgccagggcacggggaggtttagtggccttatttcctaatagg |
| | gaacatagcagggcctggggtttgatgagacggcgtaacgcccagcacaccttcagacaaaagagaccgtgataa |
| | aaacggttcttactacaaggtcccagcttctgtctttaaaatggggcttccagcctccgcctcagtagatggttggagg |
| | cactgcatgagatcctggtctggggagcccctggttcgttggtgtttcccttgccttggcctagtaagaggggacggg |
| | aggcccaggctctgtggtgggcctgtctcagcccatttcaaaccctacccactgccttcccatgaccttaacaggatg |
| | gcattacccagcactcacccagatgccagggtggcaccgggtgagtggcagtttcgggtggtcctggagaagggt |
| | cctggcatcggctggtatgagtctctgggtttccgagcttcaatgagggctggattcagggtgtgggctgggccagc |
| | cattccggcaaaaccccagaaggaatccacaggaggtggggaccgggaaggcagccgggttgcgccagtgcct |
| | gcaagggaacctctggccccatttcctgggagcctgaccccgccctaggattcctggagtgggtgtgcccacgcttc |
| | cggcccagacgtgggtggagggatcccaccctgccaggcctcccgcctgtgcggc |
| NKAPL which is | gtgtcgctacctgaaagaagtatttcatcaaccagctttgtaattcaagtctacatcagatactaaatggcttaaatttaca |
| positioned on the | attatccaagtaagacatatccatttcattggcctatcaagtagtttataactccaaaagaaaattttctttacttacacaaa |
| plus strand with | ctctatagtactcaccctatccatatgatattttcactatacatgtgaccattcacatataatactccctgctgtttaccagtt |
| the promoter | cctaaattcatataatttatgtatggaaccaattcttatccccttcaatcaaagtactaaaattctacttagagcaaatgata |
| region positioned | tgatttggtcttattcaatttaatttcccccattttgttgtttagagttctagcatcaggaaggcatcattcttaagttttgacct |
| (chr6:28,257,796- | ggctgtcaaacaactgagcagatcaacttaattagagagaaccaagatgatgttaatgcctatttacattttaagatgag |
| 28,259,296) and a | agataaatgatcttgcataaaacgtttatcatttctgagagtgagatctaatgtgtatcgagaaagtggtttccaaactac |
| promoter sequence | aaagtttctgaagaagtctccctagatagaaagacatctgtctacaagatcgagtgggatgcttctgtgtgtttctaggg |
| of: | tggaaaagtctgtgaatttaataattaaaatcatgactctgggccgggcatggtggctcacacctgtaattccagcactt |
| (SEQ ID NO: 13) | tgtaaggctgaggtgggaggatcgcttgaacccaggagtttgagaccagactcagaaacaaagtgacaacggtctc |
| | tacaaataattattaaaaaaatcaacctggcgtggtggtgggcacctgcagtcccagctcctcgggaggatctcttga |
| | acctaggaagtcaagtcggccctgagccatgatcgtgccactgcaccccaacctgggcgacagagtgagatcctgt |
| | ctccaaaaaaaaaaaaaaaaagtacgactttggccttcacctagcttagagggtgagctcaaactgattccaaaaga |
| | agttttggttcttgcttccagtggtaacaaggggggttgggaagtgattaattaaacatatttcctccacgaagagggtg |
| | gacaagttccttttttaggaaggaaaaaaggaggagcgttggtagtagggatcttccacagaggggtaaagttaagg |
| | aggtggaaatgaaagggaagaacaaagggtgagaagcaaaatggtaaaagcgaaatcgaaaaagagaagtggg |
| | tgttccaacagtagaggggatcattgacgcaaattcgtccttttatgttcccacacggctcaagttggggaatataagtc |
| | ctaaaaacaggcgcaccctaatcctacctacttcccacaagaagccttcacgcagccaaggcccgccccacatagc |
| | acacgcgcggattcgttttccgtttggtaactgacaggaagcggaagccgtgcgtgggaggccgattct |
| PRR34-AS1 (gene position | aaaggagggggtgataataatgctgcccagaccccagaggacccctaggaggctcttgctgggcagc |
| chr22:46,053,705- | tggtcctgccccagagccctcccactccacacacccctgcatacgctcaagggcagatgggccccccacaaccaa |
| 46,058,522) which | gccaggcctggtggccctaggacactcttggccacaagaattcagattattttctcagcaaatttctgaccagtgtcct |
| is positioned on | ggggcctcagcaggggcagggctgcatggtcctgggtctatctggacttgttctggctcagacctcagcccaccac |
| the plus strand | aaggacaacgagtctgggaactcgggcaggctctctgccaaccaggagctgctcttgctcgccgggaccccacag |
| with the promoter | ggcaggaatagtgggcattcatatcattcccattttgcaaccaggcagaccgaggcccagagaggccagataacct |
| region positioned | gcctcaggccacacaaatggcaaacaataaggccaaaccttggtgatggccagactctaaatccggggccacttctt |
| (chr22:46,052,204- | gccttaggaatatagtctttcctggcactcacttagccaaaaatctttctagaagggtggcaggccttcctgccccactc |
| 46,053,845) and a | ccggaacaggccggctgctttcagggccagaagagccagccctttggaggaggaggcttgtgagagtggggatg |
| promoter sequence | ggccgcagagcctccccaggcagggcagcgttgcagcttgcatgtggcagggatgtagggtgaagctgtgcagg |
| of: | gcaaaagacaggtcagttcccgctggaagtgccccactggggggcctcggtgtgtctctttccccagcactggcat |
| (SEQ ID NO: 14) | ggcacattttgacctgagcaatggtgagctgcccggaatacgaacaggaggaagagaaccttccccaggcagcat |
| | gtgaggctggggtggcactcgacacaggacagcaggcaaggacatgccgccctccaggctgagggtcttggga |
| | gcctcccctcccagcccctgctgtggcctccccctgggccctggcatgggaggagttaagctgccgcgggcacctt |
| | ggcacattgcgctgcccagagtgtccggctccccacccccactgcttttcttcctttttgcctttttccttcttaattaatcat |
| | acgctgctgcccaatttaaatatggaaatgcccaggccgaggcgatgcaattattcatgagggccgggcggctgtcc |
| | ggggctctcgggctgtggggagtaccgtggcatcgcctccgttctgatgtgtgtgaaattatgtgcacaaaaggcgc |
| | ctccttatctgggagtgtgaactaaataaataaataagggcttttgtttgtttgccggctcctgcacatggctgctggga |
| | ctcaagcgctcgtgttgtctgcgcctctgtgggactctggggacgggaggcaggggaggcccccgcaggccggc |
| | caggggctgggggctgggggccgggggccggccgccgggcacctggggccctctccctccccaggcaccacc |
| | ctcaccccgcagctcccccccatacacaccctcgctggaaagttcgccctgcccgggctgggctcccaggcagcc |
| | tggcaccgtgccaggctcgccgccgagctcgggcagcagggtggggcaaa |
| ZNF300P1 (gene position | Tttatagccgtcattaaaatgtcttttcctggatgtctggatcatgctggtgttggtactataatcatacttatttttattgtct |
| chr5:150,930,436- | gaactcttagaaaacattgtcataatatttacaatgttaaaaattaaactagcacaataattggtagaaagcagcatagtt |
| 150,946,584) | aatccaacttaaacactcctgaatgcttatgatggcaatgcacttaagtcatacacttaaatcccaacattccaaagtga |
| which is | cccaacaatgggtaatccattattgtaaattgcaaattatggtcgccaacctcagggatgggggtaaaatagcaatag |
| positioned on the | cgcctaggaaaaggcacagaatctatgccatattgacttgacaataataaccatccctttcaaccctactttttgggcca |
| minus strand with | cctagtgggttttgtatctttcgtcaaatttttcccactcatccagggaaactgggacttctgaagcctcagggagggac |
| the promoter | ttcccgggacacagcggacttttgctgctaatcctataatagtttcaggcaaagagggaccgttggtcaccttacgca |
| region positioned | gtctccctctacattccatcagegtcctccttttcccaggetccccacttccctetttctcccctctcettegtegtcccctt |
| (chr5:150,946,585- | gcctctgtccccagtctctcactcttggtcccttagtttcccattctctgttcttaggtcttgcgtctttttgtcccttagtcttt |
| 150,947,789) and a | ctcatttccgcactttctcctttattttgtacgtccatctcctcttccctgtctcaattttcctcctttttttaaagtttgcccctgc |
| promoter sequence | aacttattaccgcgcagtattgtaatatctccctcggagtgtttcttatcattactcttaataaaagttcagtattactgttatc |
| of: | ccctcccgaagtgaccgttcccaccatggtggccccagtctcccacagaggaaacgccccggagaagaacgtacc |
| (SEQ ID NO: 15) | atttacaaacacgtttaattggaatcgccaacgaaaatattgtaccattctaaattcttcgcctttcaacataagcatcctta |
| | aatggaggacagtagcactccaacttcttctccaatcctgtgaaatctgtctgcaaaagtgtggaccttactgctcgtgt |
| | agtaggcctccttgccgtcgtcttcccctcccttcgcagtcactgtggtacagtccacattcc |

Common motifs from the above preferred promoter sequences may also be used in the practice of the present invention. Such common motifs include:

| SEQ ID | Sequence |
|---|---|
| SEQ ID NO: 16 | AMAKARHAWSAYAMTDTYWMAAAAAAWAAARAAAAAAARAAAMA |
| | KARHAWSAYAMTDTYWMAAAAAAWAAARAAAAAAARAA |
| SEQ ID NO: 17 | ACCYRGGAGGTVGAGBYTGCASTGAGCHRWGATCGCDCCACTGCACT |
| | CCAACCYRGGAGGTVGAGBYTGCASTGAGCHRWGATCGCDCCACTGC |
| | ACTCCA |
| SEQ ID NO: 18 | CCGAGBAGCTGGGACTACAGGCRHCYGYYACCACGCCNGRCTAATTC |
| | CGAGBAGCTGGGACTACAGGCRHCYGYYACCACGCCNGRCTAATT |
| SEQ ID NO: 19 | AAMAWAATAAATWAAAAMAWAATAAATWAA |
| SEQ ID NO: 20 | GTGGCTCABRCCTGTAATYCCAGCACTTTRDAAGGCBRARGHDGGNGG |
| | RTGTGGCTCABRCCTGTAATYCCAGCACTTTRDAAGGCBRARGHDGGN |
| | GGRT |

Substitutions are as follows: “M” refers to A or C; “D” refers to A, G, or T; “W” refers to A or T; “Y” refers to C or T; “H” refers to A, C, or T; “B” refers to G, C, or T; “K” refers to G or T; “R” refers to A or G; “5” refers to G or C; and “N” refers to G, C, A or T.

The promoter sequences that may be used in DR-O may include promoters corresponding to any of the segmental embryonic (pre-fetal) genes listed in Table I, preferably, the promoter sequences for use in DR-O are those listed in Table IV and the sequences extracted from human genome Hg38 are described as follows. It is commonly-understood in the art that regions of the gene regulatory elements disclosed herein may be modified to maintain or even enhance the desired developmental regulation while retaining stretches of at least 10 nucleotide sequences from the disclosed regulatory sequences. Preferred examples of promoters useful in DR-O include the following.

| Description | Sequence |
|---|---|
| A first promoter | gtggcacaggttccagcctccagcatgtggcagtgcctcttccttctagtcctccagcctggcaggagaagctcctgc |
| region associated | tgctgaccgctctcctactgctctatcgccactaccaaccgcagcgaggtagtgacccaggctccaggctccatccat |
| with the gene | cctccaccctccagcaggtggaaggttgcggcttcttccagttctctaagctggacacagagttgctcctccgctgga |
| CCDC144NL-AS1 | cacagaagagcctgaaatgacctgacgccacctcagcatgctttatatatgaggttatgcaaatgcagttcctggacta |
| (gene position | catgttctgattggatgagaaaaaaaacctctaggcctactctgattggactttattttcatgctgtgattggttgtgttaag |
| chr17:20,868,432- | acttgctctcatccaatcagaacatgatcataaagtccaatcagagtaagcctggaggtttttttctcatccaatcaagac |
| 21,002,276) which | atgcagtccaggaacctccgtgggcattaccgcagtatataaatgatgctgaagcggaaacacgttttttc |
| is positioned on | |
| the plus strand | |
| with the promoter | |
| region positioned | |
| (chr17:20,867,979- | |
| 20,868,526) and a | |
| promoter sequence | |
| of: | |
| (SEQ ID NO: 21) | |
| A second | tgggcaaaaaatgatgaagtttgggggcagatggtgaaagaaaaagggtggtgagagggagggggccaaaggc |
| promoter region | cgtttggaaaagaaggtagggaaataatggtgggggacaaaggtttggggtagatttttttaataagatcatttgtatgtt |
| associated with | tgcttttcagtagtttgagttctttatgcattttttgtatgaaccccttgcctgatgcatggtttgcaaatacttgcttccattat |
| the gene | ctgggttctttcatttttattaaattttaattcaatttaattttttttagacggagtctcggtctgtcgcccaggttggagtgcag |
| CCDC144NL-AS1 | tggcacgatctccgctcactgccaagctctgcctcctgggttcacgccattttcctgcctcggcctcccgagtagctgg |
| (gene position | gactacaggcgcctgccaccacgccaggacactttttttgtatttttagcagagacggagtttcaccatgttgttagcca |
| chr17:20,869,776- | gtatggtcttgatctcctggcctcatgatcggcgcccctcagcttcccaaagtgctgggtgtacaggggtgagccacc |
| 20,870,857) which | gctccctgcctgttttttcattctactgattgcttcctctgctttgctgaggcgttttttttgtttttgagacggagtcttgctctg |
| is positioned on | tcgcccaggctggaatgcagtagcatggtctcagctcactgcaagctctgactccgggttcacgccattctcctgcct |
| the plus strand | cagcctcgcgagtagctgggactgcaggcgcaggctaccaagcccggctaattttttgtatttttagtggagacgggg |
| with the promoter | tttcaccgtgttagccaggatggtctcaatctcgtgaactcatgatctgcctgcctcggcctcaaaaagtgctggggtta |
| region positioned | caggcgtgagccactgagcccggcctggaaacgtaactttattttttagtgttgtatttgtgcatatactttaatagccctg |
| (chr17:20,867,979- | agttttaataaagttgcttttaaaaaatgtatcttatatttcagaaatacaccctaaggcatgtgattagttgggtggcatgtt |
| 20,868,526) and a | gtttagtttttacaattgaaggattgtcat |
| promoter sequence | |
| of: | |
| (SEQ ID NO: 22) | |
| CASC9 (gene position | aaaagagctaatctaagtaaacttggaaaacggtgttttgattacatatggcaagactacaaacaaataaacaaaaact |
| chr8:75,117,286- | atacacaaatattgttttatagtttgtaaatttgttttcatggaatacatcttagcaattctaaaaatacttcatgtatactctag |
| 75,352,327) which | gattaaacaattacataagtatattgtagataacaacagccaggcttctcactgttgaagaaagaagtttcaaataaggt |
| is positioned on | aggagagaaggcgagaaggaacactataatgccagatggaattgaaaatattagtagaaactcatggttttttatatac |
| the plus strand | atggacgtctgtgtgtgacccatatacatacatacatggataatatagaaattaatatacatatgtgtagatatatggttaa |
| with the promoter | atatgcatatatatatgtataatcagctctgtccactgagtgcctataagcaatgatttcccagtagtaataagtacatcca |
| region positioned | gccttcagatcttgacttttaaattttactctccaatataaggaactggggttccttggtaaaatggctgattctaggtctgg |
| (chr8:75,322,337- | gtcacggaaagtttcagaaagtaagaaattgctcagaaaaaaaaataataattgttgcagaccagaatcatcaatggat |
| 75,325,237) and a | gctaaaattagtgggcaaagttgaataagaaataggatttttcatattttcaaggtcatcttcaactgatacttaataattac |
| promoter sequence | aaagggaatatagcaatgttaaagtggtaaaacctagaatcctccaatttatgcaagcaatcaatttaaaattaccagca |
| of: | atgtgatatattgttatcatgtcttcgaatatgacatactgaggagctcacatcacttctgtaagattcttgcccaaatgcat |
| (SEQ ID NO: 23) | aaggtaaattatgttaagaagaaatacataaacacacactgaggaaaattctacaaaataagtagccaataatcttcaa |
| | aagtgttaaagacatgaaagacaaagtaaatttcagaactgtcccagaatgaaggagaccaaagagacatgccaact |
| | aaatacaatgtgtgattctggattagatatgaatcagtacaaagggcattagtgggacacttgggaacatgcgtataaa |
| | gttccttgattggttgacagtgttacattgatgttaatttcctagttatgattattgtactatggttaagtaaggtgtaaaaattt |
| | aaggaagctttacgaatatagttaggtctgaaatgatgtcaaaatgaaaaattaaaaaccatataaaatagtgaaatca |
| | gattaaatgtgcatcaaaaaattatggcgcatatatcctgtggatatttcactgcatttaaaaagaaaacaaaagatccat |
| | atgtacagacatgaaaacgtgttgttgaaatgccaattaattaaaaaatacagttgcaatgtaatgtctatcatcccacttt |
| | ttttcaggcaaactatatacgagttcataggaaaaaattctggaataattctaaccaaactattcactactttgtcttgatta |
| | gaggaagatgtgagaaaaatgaaggtttaaggaacacttgttttttattacctatacttgtatattcgtttaatttcataaca |
| | agaataaatttctttaaaaaaacagaaaacaatgaagagagaaaaaaatcttcacctgatccccagtggtttatagaatt |
| | aaggtaaattttaaaatattatgtgctccacctaacttattcaaattcgaacctcatattccaccaatcagataatttttacttt |
| | tgctacttatctttgtgaataatctattcacttgttctaaatcaatttactttaattctttttaatattatatttttaaagtataaaact |
| | ttaccggaggagccaagatggccgaataggaacagctccggtctacagctcccagcgtgagcgacgcagaagac |
| | gggtgatttctgcatttccatctgaggtaccgggttcatctcactagggagtgccagacagtgggcgcgggtcagtgg |
| | gtgcgtgcactgtgcgccagccgaagcagggcgaggcattgcctcacttgggaagcgcaaagggtcagggagttc |
| | cctttcccagtcaaagaaaggggtgacagacggcacctggaaaatcgggtcactcccacccgaatattgcgcttttc |
| | ggactggcttaaaaaaccacccaccacgagattatatcctgcacctggttcagagggtcctacgcccacggagtctc |
| | gctgattgctagcacagcagtctgagatcaaactgcaaggcggcagcgaggctgggggaggggcgcccgccatt |
| | gcccaggcttgcttaggtaaacaaagcagcagggaagctcgaactgggtggagcccaccacagctcaaggaggc |
| | ctgcctgcctctgtaggctccacctctgggggcagggcacagacaaacaaaaagacagcagtaacctttgcagactt |
| | aaatgtccctgtctgacagctttgaagagagcagtggttctcccagcccacagctggagatctgagaatgagcagac |
| | tgcctcctcaagtgggtccctgacccctgacccgcgagcagcctaactgggaggcaccccccagcaggggcaca |
| | ctgacacctcacacggcagggtattccaacagacctgcagctgagggtcctgtctgttagaaggaaaactaacaaac |
| | agaaaggacatccacaccaaaaacccatctgtacatcaccatcatcaaagaccaaaagtagataaaaccacaaagat |
| | ggggaaaaaacagaacagaaaaactggaaactctaaaaagcagagcgcctctcctcctccaaaggaacgcagttc |
| | ctcaccagcaacggaacaaagctggatggagaatg |
| CPT1B (gene position | ttgctatgttgcccaggctggtctcaaactcctgggctcaaacgatcctcctacttcggcttcccaaagtgcagggatta |
| chr22:50,568,861- | aaagcgtgacccactgcccccagccagaatcagttttcttttttttttttttttttttgagactgagtctgactctgtcgctcag |
| 50,578,612) which | gctggagtgcagtggctcgcggcaacaaggcctgggttcaagggattgtcacgcctcagcctcccgtgtagctggg |
| is positioned on | attacaggcacaagccaccaaacccggctaatttttgtatttttagtagcgacggggtttcgctatgttggccagggtgg |
| the minus strand | tctcaaactcctgacctcaagtgatctgcccgcctcggcctcccaaaatgctgggagtacaggcgtgagccaccacg |
| with the promoter | cccggaccagttactttttaaagtggctctaggtccctttagggaaggattggaggaatatttcttggtatatgctcagat |
| region positioned | gctggtattgtaagatccttcctgagaggaaccaagcctccttgtcaattaatgggctctaggtctcaaaactgactcag |
| (chr22:50,578,614- | atttaaacagaatgatcatgattttccactgcagcaaaagtgacattagccttgtgatcaccaatttttgatttctttctggtt |
| 50,584,487) and a | atcaaacaatactctagtcagatgccccaaagaacaccatggttcgacagacattgccacagatctctgtgggtccag |
| promoter sequence | cgttcctggtcgccgctctagcctggggcagaagcagaaatgcaggaggcacgcgtttaaatcaggacgccctcaa |
| of: | tgggaggtgcgcaggccgaggagggggctgcggcaagctgcgagtcttcactttcttcggctcttgggtttgactct |
| (SEQ ID NO: 24) | gtaattggttctaggcctggaggagacgaggaggatagggggagaggggaatgaatgaagacgcgcatctcaag |
| | cggggcagcaaaagactcctcattcagaggtgcggctctaccggccagggagtccccgtgtagactgggcatttat |
| | gcaactggagtcatcggaactgtcccaagggtcacattccttcagcaaatgtttactgcgtacccaccgggtgcccgc |
| | atcccgcactctgcccgcgctgtgcggccctgcctagcgcgcgcgcctcctccaggcctcacccacgctcaggac |
| | ggacgcgcgctggacggctcttccttgtcggagcgccccaggggtcggggaagagggcccggcaagggagccc |
| | tcgcgccggagctgcagctgcagccgccgccccgccgccccgccggctcccacggggcagagacgcagctcct |
| | ctccggtcttcccgtacgctaccgcgcccgggcagttcctcgcccgcgcacgcgccgctccgccaactgattggcc |
| | tccggcgcctcggattggcccaggccgtccaacagcagccccgcccagagagagaccattggtccttgcccatag |
| | gggcggggcccggcagagatctgcggaattcggccttcggaaagagcccccgggccggggcacggagagagc |
| | cgagcgccgcagccgtgagccgaatagagccggagagacccgagtatgaccggagaagcccaggccggccgg |
| | aagaggagccgagcgcggccggaaggaaccgagcccgtccgaagggagcggagcgcagcctggcctggggc |
| | ccggtcgagcccgcgccatggcggccgaggcgacagctgtggccggaagcggggctgttggcggctgcctggc |
| | caaagacggcttgcagcagtctaagtgcccggacactaccccaaaacggcggcgcgcctcgtcgctgtcgcgtga |
| | cgccgagcgccgagcctaccaatggtgccgggagtacttgggcggggcctggcgccgagtgcagcccgaggag |
| | ctgagggtttaccccgtgaggtgggaggtcaggggtcagcctctccggtgcgcggatcggggtcaggggtcagcc |
| | gcggggccctcaggatgctccatgttttcgcccccctcttgcgcccgcgcctggggggggggggccggcctgg |
| | ccggggggggccggggccgcggcaggtagggccggccgcgggctgagcgcgcctggtgtgggtctgcagc |
| | ggaggcctcagcaacctgctcttccgctgctcgctcccggaccacctgcccagcgttggcgaggagccccgggag |
| | gtgcttctgcggctgtacggagccatcttgcaggtgaggggggtgtgagcgccgcagcaccagtggctttagggcc |
| | tgtcgcttacgcgatgcgggtagtattgttcccgttgcgcagttgaggacaccgaggttcacggtctgagtaacacctc |
| | attacaccgaagcctgggcctgtattcccagagctttgggaggctgaggcgagaggatcacttgagcacaggagttc |
| | gagaccagcctggacaacatagtgagacccccatctctaaataaaaatagaccaacgctaaagcctgtgctccaga |
| | gcctccaggcaattggatcagaagtcgcagctctggtgggaggaaggcgagccctcatgtgtgtccctgtgccactt |
| | tgccttggcccctttgctgtccatcctttttcagggcgtggactccctggtgctagaaagcgtgatgttcgccatacttgc |
| | ggagcggtcgctggggccccagctgtacggagtcttcccagagggccggctggaacagtacatcccagtacggg |
| | cccagtcctaccctctcctccccaaggcacctccaccccctaaccctaccccagtgcccaatgtctgcctccacatcc |
| | ctcaccccaagtagggaatttcccccaaaaccctgacttcccctcattgccccagcccttcccactcctgccccagcc |
| | ccatcaccaccctgatagcttcctgggtgcagagtcggccattgaaaactcaagagcttcgagagccagtgttgtcag |
| | cagccattgccacgaagatggcgcaatttcatggcatggagatgcctttcaccaaggagccccactggctgtttggg |
| | accatggagcggtgagtcaggagcctcctcagggctcctgtactcctgagctgaacctccatgccagcggatgtgtc |
| | gggggctctatccagccctccacttgagtggatctgatgtcatgggctgtcacagaacaggcttttaaatgcttgccag |
| | aggccacatgcagtgcctcatgcctgtaatcccagaactttgggagccgaggtgggtgaggtgggaggatcacttg |
| | agcccaggaggcggaggttgcagtgagctgagctcatgccattgcacttcagcctgggtgagagcaagactctgtc |
| | tcaaaacaccaaacaaaaaaagaaaaaagaaatgattgccaaggccaggcacggtggctcatgcctgtaatcctag |
| | cacttcgggaggctgaggtgagcagattgcctgagctcaggagttcaaaaccagcctggacaacacagtgaaacc |
| | ctgtctctattaaaatacaaaaaaattagccaggcatggcagcatgcacctgtagtcccagctactcgggaggctgag |
| | gcaggagaattgcttgaacccgggaggcagaggttgcagtgagccaagatcgcgccactgcactccagcctgggt |
| | gacagagcaagactccatttcaaaagaaaaaaagaaatgattgccggacaagtcacacagtgataggagcagggg |
| | tgcagtagtctggcctgaggggtagataggcgaggggtgggaaagaatagcagagccagtgtatcctattctttggg |
| | cttcaggtacctaaaacagatccaggacctgcccccaactggcctccctgagatgaacctgctggagatgtacagcc |
| | tgaaggatgagatgggcaacctcaggtgagggcaggcaggacaaggctaatggtaatggtgtccgcccttccagta |
| | gttgctgagggctggtgtcagggcctggcctgttagggtggctctgatcctcctctagtcactcctgctcaggacccat |
| | gctcccatacccctgtaggaagttactagagtctaccccatcgccagtcgtcttctgccacaatgacatccaggaagg |
| | taggagaaggcatctgagtctcctaacccaagatggaagagccagagggctctggagtgagcagaacctcacccc |
| | attcccccagggaacatcttgctgctctcagagccagaaaatgctgacagcctcatgctggtggacttcgagtacagc |
| | agttataactataggtgaggctggaaagatggcttcccatagatctgttcccatagggctcttgaaaacaggccagctg |
| | cccagggcatttggggactgaatgtccaccttattctcccaggggctttgacattgggaaccatttttgtgagtgggttta |
| | tgattatactcacgaggaatggcctttctacaaagcaaggcccacagactaccccactcaagaacagcaggtatgtg |
| | ggccagaggctggggagcaggacccatcctgtgaggaaggagggaggtggagtctggaaggaatggccggaa |
| | aggatgttacctgggaaatactccacagtctccccaattcctgactcttggccattgatcgtagttgcattttattcgtcatt |
| | acctggcagaggcaaagaaaggtgagaccctctcccaagaggagcagagaaaactggaagaagatttgctggta |
| | gaagtcagtcggtgaggaaggaggggcagggtggggtagggcagagcagaggaaagagggattggggaaga |
| | ggcagatttatcaagctgcagggaaggtggctgtggagagtggagttaggacagtgggggagaagttaaaactgta |
| | gggattggccaacctgggtgaggggatccaagcagtgctagacatgctcttgaatgcccctccttttcctgccctccc |
| | cccaggtatgctctggcatcccatttcttctggggtctgtggtccatcctccaggcatccatgtccaccatagaatttggt |
| | tacttggtaagtgaccctggggatgggaatgctagctggggggctggggagcagcagcagccacactcttccagg |
| | aggcctggggagtcccgggtggctgtgggcagcctgaggtggatgtagaatgctggtcccacgtcttctcaccact |
| | gtgtggggtgggtttccttccctaggactatgcccagtctcggttccagttctacttccagcagaaggggcagctgacc |
| | agtgtccactcctcatcctgactccaccctcccactccttggatttctcctggagcctccagggcaggaccttggaggg |
| | aggaacaacgagcagaaggccctggcgactgggctgagcccccaagtgaaactgaggttcaggagaccggcct |
| | gttcctgagtttgagtaggtccccatggctggcaggccagagccccgtgctgtgtatgtaacacaataaacaagcttct |
| | tcttcccaccctgtcctggccctgctgagcagcagcagaaagtaccaaaccgagcagtacacacaaagggactcttc |
| | agtgctctgggattgaaagtggttagcgttcatgctgccagttggggtcccccatccctccccagtcccctggctgca |
| | gcttagaataataaatactaggacttggggaggaggagagtgatgggggtatgaagacgaccctgaggtggggat |
| | gccgcccggagcaccagcgatcccagaacaggcagcagctgacacatcggtgaccttttccctacatttggctatttt |
| | tagctctaaagccaccatcctcacgagactctggggccccccaggctcc; |

The region of the human genome spanning chr2:130,000,719-131,557,000 contains two duplicated gene families (FAR2P1, FAR2P2, and FAR2P3) as well as (POTEE and POTEF), as well as the gene MED15P9, all of which are markedly up-regulated in embryonic as opposed to fetal and adult somatic cells. While the POTE gene family members are known in the art as “cancer testis” antigens due to their expression in the testis and cancer, it is not known in the art that the genes are normally only expressed in the embryonic phases of development of diverse somatic cell types, and that the DR-O therapeutic methods described herein are useful in specifically destroying numerous cancer cell types while leaving diverse adult somatic cell types alive.

| Description | Sequence |
|---|---|
| FAR2P1 (gene position | ctctatgtacagtaataacaatatctgtcccagttattatgtacaatattataaaaaatgtcgcagacagtacaaattaagg |
| chr2:130,012,065- | cccttatttctcaaaggacatcaagtcttatgccccgtgggagggaagatgccacttaattattgcaccattttgaaaaa |
| 130,051,131) | cagaactcgtcaaggcggatagtggtcttttcccacctggtcttcagtcacagatgggggctggggcggtggcgccg |
| which is | tcttcgtcgtcgaggtagaccctatggagccacaccgccctgccgagttctctgggtgcaataaataccagtcacagt |
| positioned on the | ttgggagggggccgcgtgcaggtggggaggcctctgggctgcatgtgccgcttcctgtgcagggcaaggtgggc |
| minus strand with | agagcgcaagaaggcgtggtggcaccaatggccctgaaatggccggtggcccctgtgcttgtggtggtggagcgt |
| the promoter | tagcttgtctgagtgagcacacttccagctgcttccatcccagtggcagtggtcgggcttgtcacttgtgtgctgcgca |
| region positioned | gatgtgcctgcaggggagaattcttgctgtaggtctggccgtagccacataggtatgcctggtgttgcagttcccagg |
| (chr2:130,036,639- | ccaggagcagctgccacgcttgggcgtggcctccagcagctccagcggggacgccggtgccaccacaaggagg |
| 130,039,003) and | ccgcgggcgactgggggtgccaggcccagggctgtggcggcggcggccaccgcctaagagactaaagacagg |
| a promoter | ggcgtggaggggcggagctgggcggggggcgcctcatgaaggccgggctgggtgtcccaaaccagggccacg |
| sequence of: | gaagggcagcgggaaggaggcgtgcgggtcgtaggggctgaggggcaggtgttgggggcggaatgggaggg |
| (SEQ ID NO: 25) | acccgggacctcgcatgcatgaagctgcaggcccggggcgccctagtgtttgagaggacgcgcgatgcatacctg |
| | aacctgggtgcgcagccgcagctgccgccatctgcttcagggtgctgggctttgaccaggtggctgggcagcgcc |
| | agttgaaagcggttgagcaggcagggcgcagtggctcacgcctgtaatcccagcactttgggagaccaaggcggg |
| | ccgatcacggggtcaggagatggagaccatcctggctaacagggtgaaaccgcgtctctactaaacatacaaaaaa |
| | aaaaaaagtagccgggcgtggtggtggacgcctgtagtcccagcactttggaaggctgaggcgggcggatcacga |
| | ggtcaggagatggagaacatcctggctaacaggtgaaaccccatctctactaaaagtacaaaaaccaattagccgg |
| | gtgcggtggcgggcgcctgtagtcccagctactccggaggctgaggcaggagaatggcgtgaacccaggagatg |
| | gagcttgcagtgagccgagatcgcgccaccgcactctagcctgggcaacccaaggagactccatctcaaaagaac |
| | aacaacaacaaaaaccagctgagcagcgcgggccatgtggcagtgggtccagctccagggcgccgtagggcag |
| | cggtgtgcagtgttcgggtaataggacgcagacggcggggtcgccgggggcttcggggtggcctcggccccagg |
| | ccatccagccctgtggaccgaatggagtcccgcacgctgttgaggtagttgtgggttcccctggcctcgggctgggc |
| | gcggggtcagcgcacctgcaggcggcgcttgcggtacgggctggtgaaagtggagatggacggcaggatggatt |
| | cacttggccacatggcgcgaagctgggaagacggacaccggtgagtggctgcccgggagggctggtcggggcg |
| | cggacaggcgggcatggttctgccaaggattttgctttatttatcgcaagatggggtatttcctcctttcttcagtttataat |
| | tgcatgaattagtgcagtg |
| FAR2P2 (gene position | Ccactctatgtacagtaataacaatatctgtcccagttattatgtacaatattataaaaaatgtcgcagacagtacaaatt |
| chr2:130,416,750- | aaggcacttatttctcaaaggacatcaagtcttatgccccgtgggagggaagatgccacttaattattgcaccattttga |
| 130,431,363) | aaaacagaactcgtcaaggcggatagtggtcttttcccacctggtcttcagtcacagatgggggctggggcagtggc |
| which is | gccgtcttcgtcgtcgaggtagaccctatggagccacaccgccctgccgagttctctgggtgcaataaataccagtca |
| positioned on the | cagtttgggagggggccgtgtgcaggtggggaggcctctgggctgcatgtgccgcttcctgtgcagggcaaggtg |
| minus strand with | ggcagagcgcaagaaggcgtggtggcaccaatggccctgaaatggccagtggcccctgtgcttgtggtggtggag |
| the promoter | cgttagcttgtctgagtgagcacgcttccagctgcttccatcccagtggcagtggtcgggcttgtcacttgtgtgctgcg |
| region positioned | cagatgtgcctgcaagggagaattcttgctgtaggtctggccgtagccgcataggtatgcccggtgttgcggttccca |
| (chr2:130,428,547- | ggccaggagcagctgtcatgcttgggcttggcctccagccgctccagcggggacgccggtgccaccacaaggag |
| 130,429,586) and | actgcgggcgactgggggtgccaggcccagggctgtggcggtggcggccaacgcctaagagactaaagacagg |
| a promoter | ggcgtggaggggcggagctgggtggggggcgcctcatgaaggccgggctgggtgtcccaaaccagggccacg |
| sequence of: | gaaaggcagcgggaaggaggcgtgcgggccgtaggggctgaggggcgggtgttgggggcggaatgggaggg |
| (SEQ ID NO: 26) | acccgggacctcgcatgcatgaagccgcaggcccggggcgccctagtgtttgaggacgcgcggtccatgcctgaa |
| | cctgggtgcgcagccgcagctgccgccatctgcctcagggggttgggcttt |
| FAR2P3 (gene position | attctggttataatatggaggcaggatgaaattgtttttattcttttagaattttttttatcaggaaaacagaggtaaagtgct |
| chr2:130,690,133- | atcaattactatttaagagttctattttgaaaagtctttaataaggatttttcttttctttttaaaaaagactttcttaaaaattaaa |
| 130,718,831) | aataaaagaagcaaaagtcttaggaaaatgaggcaagtagccctgccactctatgtacagtaataacaatatctgtcc |
| which is | cagttattatgtacaatattataaaaaatgtcacagacagtacaaattaaggcacttatttctcaaaggacatcaagtctta |
| positioned on the | tgccccgtgggagggaagatgccacttaattattgcaccattttgaaaaacagaactcgtcaaggcggatagtggtctt |
| plus strand with | ttcccacctggtcttcagtcacagatgggggctggggcggtggcgccgtcttcgtcatcgaggtagaccctatggag |
| the promoter | ccacaccgccctgccgagttctctgggtgcaataaataccagtcacagtttgggagggggccgtgtgcaggtgggg |
| region positioned | aggcctctgggctgcatgtgccgcttcctgtgcagggcaaggtgggcggagcgcaagaaggcgtggtggcacca |
| (chr2:130,691,713- | atggccctgaaatggccagtggcccctgtgcttgtggtggtggagcgttagcttgtctgagtgagcacgcttccagct |
| 130,694,087) and | gcttccatcccagtggcagtggtcgggcttgtcacttgtgtgctgcgcagatgtgcctgcaagggagaattcttgctgt |
| a promoter | aggtctggccgtagccgcataggtatgcccggtgttgcggttcccaggccaggagcagctgtcatgcttgggcttgg |
| sequence of: | cctccagcagctccagcggggacgccggtgccaccacaaggagactgcgggcgactgggggtgccaggccca |
| (SEQ ID NO: 27) | gggctgtggcggtggcggccaacgcctaagagactaaagacaggggcgtggaggggcggagctgggtggggg |
| | gcgcctcatgaaggccgggctgggtgtcccaaaccagggccacggaaaggcagcgggaaggaggcgtgcggg |
| | ccgtaggggctgaggggcgggtgttgggggcggaatgggagggacccgggacctcgcatgcatgaagccgcag |
| | gcccggggcgccctagtgtttgaggacgcgcggtccatgcctgaacctgggtgcgcagccgcagctgccgccatc |
| | tgcctcagggggctgggctttgaccaggcggctgggcagcgccagttgaaagcggttgagcaggccgggcgcag |
| | tggctcacgcctgtaatcccagcactttgggaggccaaggcgggccgatcacggggtcaggagatcgagaccatc |
| | ctggctaacacggtgaaaccccatctctactaaacatacaaaaaaaaaaagtagccgggcgtggtggcgggcgcct |
| | gtagtctcagcactttggaaggctgaggcgggcggatcacgaggtcaggagatggagaacatcctggctaacagg |
| | tgaaaccccgtctctactaaaagtacaaaaaccaattagccgggtgcggtgggggcgcctgtagtcccagctactc |
| | cggaggctgaggcaggagaatggcgtgaacccaggagatggagcttgcagtgagccgagatcgcgccaatgca |
| | ctctagcctgggcgacacaaggagactccatctcaaaacaacaacaacaacaacaacaacaacaacaacaaaaac |
| | cagctgagcagcgcgggccttctggcagcgggtccagctccagggcgccctagggcagcggcgtgcagtgttcg |
| | ggtaataggacgcagacggcggggtcgccgggggcttcggggtggcctcggccccaggccatccagccctgtg |
| | gaccgaatggagtcccgcacgctgttgaggtagtcgtgggttcccctggcctcgggctcggcgcggggtcagcgct |
| | cctgcaggtggcgcttgcggtacgggctggtgaaagtggagacggacggcaggatggattcacttggcgacatgg |
| | cgaggagctgggaagacggacaccggtgagtggctgcccgggagggctggtcggggcgcggacagggggc |
| | atggttctgccaaggattttgctttatttatcgcaagatgggggtatttcctccttcctgcagtttataattgcatgaattagt |
| | gcagtgaattgaggatgcagtaaaaatatcttcaaagattattaaattcgttattataaaacacatagaagagtttatgtgt |
| | gtgtatggaaagcaggtatacatcaataattcttaatgaatacaagaaagaactaccaatattggggcaaatttttcaa |
| LIN28B (gene position | ttttgaaatgtaaagggaaattttgaaatataaaggcaaattttgaaatataaaggcaatttgtgtggaaaacttaaaattt |
| chr6:104,936,616- | atgctaatccgtttctgtatttccataattttactttattattctggggaaagtgtgttaaggtatttaatgaaagtctcgagag |
| 105,083,332) | atttttacctctgtataaaaattctaacgttggaccaattgtgtgaagccagagcattttgcctccagacaaggaaattaa |
| which is | atgagaatagaattttgctttttggaaacaaaagcaaagcgacccaactatattctgaagtgcctctgctgcaagaccc |
| positioned on the | aagcacccagtcgggcgcgccttgcatttgtctccatgtcgtaggaatgtcaagaaagttaccgccaagtggtttacc |
| plus strand with | aaaacgcggcggaaggaatcaggacagtttgcccgacgggcgggtcggcggctttcaggcgggagagatcttgc |
| the promoter | caggggcagagagagatcccgaaaatcgcccccagccttgcgtccagccctttctcgccgacgttttttcttgatcctc |
| region positioned | cgaaggcccagggcgacgtttgccatcgcggcttcccgaaaggacggcgccgggtttgctggaagacagctcagt |
| (chr6:104,952,665- | tttaggcatggcccggtgggggtgggggtgctcattttcttgggttcggattccctctctttgaccgcacttatcttagctt |
| 104,957,106) and | taggagattctggcatctcataggaaaagggatcaaattataagtgatatacgtctggaaattcagtaaaaatagtaggt |
| a promoter | tgtgcgtagtgtttttgccatagtagtgaaggcagggatttcttttgtttggtgttcttggggacaagatgacaaatgtaaa |
| sequence of: | agtccctaaattagtgagaaatcaccagcctttggatctaccccacttagggagaggagatggtaaagtgacgaataa |
| (SEQ ID NO: 28) | agggaaaagtgactgaggaaagaaaggtgagaggaagctgcagaggcaggaggtggagggaaagcgcagatc |
| | tgctggcgcggggtctggggtctcaggtctcggccgcctggggagcgacctgcctggagaggcgggttgcgatg |
| | gcagcgctgggctacagaccccgccttatttgcggggaggagaggacgggtggagtcacttgccgcattgccaca |
| | gcccctcctcctagttttaagaagaaccgaagcattgtttctgctttaacagaagtcactcattccttataattgaaaacaa |
| | cgacggtgggctctggcacctctttcggctccttgtactgttggaaaaaaagtctgggaaaacgtgtaccttgttttttaa |
| | atggggatgattgtgatagccaacaggcatatggtaggttatttccttgctttttccccccagtacattgacgattctttgta |
| | gctacttgaactcattttaaagacattttgaaagaactataagtcaagctataaagtatttggcttttgcaggaataaaaca |
| | ccccttttgagtcaaacacaggtctttcaggcactaaaaccaagtaggaaatgagaaaaaaattaaaaccagaagact |
| | tcaggaaagtgacaaattggaaagcattctagaagactcataggacgactttctgtgaaagaagcactctaggagctt |
| | taaaagcttatgggcaatttatattataaaaggattttaggctcaaaagttttgggggaaggcacagaaaaaacagtgc |
| | catattcttaaattggtgagtgattattttgtgtttctcaatgtgtctccttcctttacttctctccctttccaacttccttgctttct |
| | tcttccttttccctctacccttccctcgcctcttctcttcccctttcttttaccctcccccaaggtatgtaaatgcatttgtctta |
| | aattcttcatgaacgaattttagaggatttttcagggttaccagttggaaaaagccttctacacaacttgaaaggctgatg |
| | tccctctcaggaaatgaatgagtcgataacctctgacaccagtgaccactccagtgaaccttaccttaattccactaata |
| | ctggaggcccccaatcaggcaagactgtgaatctcataactatactgtacagtgtagttcagttatggtattccacttttc |
| | acccacatgttcataaatttgaatttcatcaaatttaacaataaacagattctgaatctacctacattattatgcttctggtttt |
| | ataatgattaaactgttttctaattaaaagcaaacttaaatgaatattttacaaagctaaactatactggcctgtgaattttgt |
| | gatcattacttttatgtaagtaatagcatttcacctcaatttacatgaatactttctgagatttttttcctcttataccagatgttg |
| | attttttaaagtgattattaaaatatgaaaaaatataaattactttctcgatttcatattatgtcacaagcttagcctgtaatgc |
| | catcattgagattactttcttgtcagtgtaatcagtataatagaagtaatttctaccatcaaaataatgcctagtaaatgtttt |
| LINC00649 (Gene position | gttagtaaataattttttcagagctttgagcttttgttaaactttcaaagtcattggatttttgcaccttttcttttctccctaaca |
| chr21:33,915,534- | gctggcacaggattattcctcccaaaataaataaaatgagaaagtagccctttttcctgagaaataacaaaaggaataa |
| 34,003,026) which | cagacaaaaaaaaaaatctttccaaattgattagttttcattaaaccttttatcaaaaacagctgaaaataacttctatttttt |
| is oriented on the | atacatccattttccaatctgagccaattaaatttatatttagttgtttaatatggggagggagtataaataggtgtatacag |
| plus strand with | ctttttccatagctcttcgattcataatccctacattttagcttgtgtgaaacagtgctggagtgtggatgtgtttgaagaga |
| the promoter | ctgggagggggattgctggcatggtgggggagggtcaactcggtagctttgtcgaaggaagtggtatctgaacagtt |
| region positioned | agtttcttggtaaactgcttgtttaagaaaatggggggtggggtaggcaagtctggtggtgcattttaaggttttaaaata |
| (chr21:33,930,560- | gcgatattttatattttaaaggaaataaagtctttttagttaatctataatatttattatagtattcgataagggcagaaatact |
| 33,931,121) and a | gttgccagcatcttgtaatcaatcgtttgaaccagtttgaaaggtaaagggggtaggcattttgaaactgggggtcagtt |
| promoter sequence | gaggaaggacagtaggagcttcatccgttgttacattttcgtttattcaactcttctagccattggtagtggacttttaaaat |
| of: | gtcagggtacaagtcaatatgatatgaatttaccttttttctcccacttgtccttctttctccaccccaccccctgcccgcg |
| (SEQ ID NO: 29) | ccggctttttttctctcccacctctttctttgactctgctgccgcctatcctggcggctcctcttcacctcccctgcctggcg |
| | atcccctccccctcctgccctatgtcctccagctcttggggaacaatcgccactgattgaggttcactctatgttaggct |
| | ggaaaactgggctgttatttaggaagtcattaatcacatctcctcccccggaaagagatggcagagaaacctgtgaag |
| | tacaattccttaccaaccccctctaaaattttccaaaatggtttacctaaacacaatttgactatatgaaggtgaaaacgat |
| | ttttctagtaggattttattttaaatttgaaaaaatttcgggcataatcaggtaatttatgtgcctctgccttttcctaggtgtat |
| | gttttcattatctaaagtttcatggggatgtaattaggtttaattagatcttttataaattatatattttcctctaagaattgaatta |
| | aatatgaaattagcgttccaagcaggtttactgccatggaatagctgaattcttaaggggaaaaaaagcgttaacatcg |
| | gttgttttatttaaacgaccccctcccatgattttagtcaggcttgtgtgtaaggcacgcgtgtgcacatatatgactgcct |
| | ttttttctaaatctatgattcacaaggtccctccattgtgtttaagtagaagcacgaaatcagcattctgatttatcctaaag |
| | aagcgttccaaattgtctttaagataacatgtttgagtttttcgtgttcattcataaattattttgatgtcagcatagatgaaat |
| | ggcgattggttatctcttcctcttgccagccccttaaggatacgaggtgaaattagtagcaagaaagcatgtaattgaca |
| | aagtcacgtgtgctcagggggccagaaactggagagaggagagaaaaaaat |
| POTEE (Gene position | Tcgtctgaagccttcttctctcagctcgtcaaagtcattctccatccagctttgttccgttgctggtgaggaactgcgttc |
| chr2:131,209,536- | ctttggaggaggagaggcgctctgcattttagagcttccagtttttctgttctgttttttccccatctttgtggttttatctactt |
| 131,265,278) | ttggtctttgatgatggtgatgtacagatgggattttggtgtggatgtcctttctgtttattagttttccttctaacagacagg |
| which is oriented | accctcagctgcaggtctgttggaataccctgccgtgtgaggtgtcagtgtgcccctgctggggggtgcctcccagtt |
| on the plus strand | aggctgctcgggggtcaggggtcagggacccacttgaggaggcagtctgcccgttctcagatctgcagctgcgtgc |
| with the promoter | tgggagaaccactgctctcttcaaagctgtcagacagggacatttaagtctgcagaggttactgctgtctttttgtttgtct |
| region positioned | gtgccctgcccccagaggtggagcctacagaggcaggcaggcctccttgagctgtggtgggctccacccagttcc |
| (chr2:131,208,731- | atctacccaaaaccttttctccccactgtcttttcacaaaaccttctctctctactgctcaacgctgtttctcccccccacca |
| 131,209,580) and | ccctctctttcctcctcccttgccaccctcttttcctcctccatctacccataaacattttacccaccatctttctgcaaaacc |
| a promoter | ttccctccctgccgctccccaccccgtttttctccctccatctacccaaaaactttttttcccactatcttttccccaccgcct |
| sequence of: | ttttgcaacgcgctctcctgctcactatcctctcttccctttggcactaaccaccctctttaccccctccatctatcccaaaa |
| (SEQ ID NO: 30) | ctcttttcctcctcttaccgcttccgccgcactgccgtctcggtcgcggttaccaccagtcgcagcgaggcgagccac |
| | ggtgtagcggctccagcctccagcgtacggctggtgattacccattcctggtcctctaagccgggcactgagcagct |
| | ccacaggaaaatacgggaacgtggaagagcctgacttcccttcagcagcaggcgtataccgcggttatatacagga |
| | ggattcctgactgcatgttctgattggatgagaaaaaccctccagggttacttggattggactttattatcatgttctgatt |
| | ggatgagagccagtcttaagacaaccaatcacagcatgaaaataaagtccaatcagagtaggcctagaggtttttctc |
| | tcatccaatcagaacatgtagtctgggaaccacatgtgcgtaacctcagtacgtaaagcatgcggaggtggcgtcag |
| | gtcatttcaggctcttaagtgtgggcgtttggtaaccggcatggctgc |
| POTEF (gene position | Cttcagcagcagtcatataccgagattatatacaggaggattcctgactgcatgttctgattggatgagaaaaaccctc |
| chr2:130,073,535- | cagggttacttggattggactttattatcatgttctgattggatgagagccagtcttaagacaaccaatcacagcatgaa |
| 130,129,222) | aataaagtccaatcagagtaggcctagaggtttttctctcatccaatcagaacatgtagtctgggaaccacatgtgcgt |
| oriented on the | aacctcagtaggtaaagcatgcggaggtggcgtcaggtcatttcaggctcttaagtgtgggcgtttggtatccggcat |
| minus strand with | ggctgctacctgtttctggctggagcctcggacactggctcactgcagttggtggtgtcgacagtggtaggagggca |
| the promoter | accagta |
| region positioned | |
| (chr2:130,129,223- | |
| 130,129,622) and | |
| a promoter | |
| sequence of: | |
| (SEQ ID NO: 31) | |
| MED15P9 (gene position | tactggttgccctcctaccactgtcgacaccaccaactgcagtgagccagtgtccgaggctccagccagaaacaggt |
| chr2:130,129,621- | agcagccatgccggataccaaacgcccacacttaagagcctgaaatgacctgacgccacctccgcatgctttaccta |
| 130,139,417) | ctgaggttacgcacatgtggttcccagactacatgttctgattggatgagagaaaaacctctaggcctactctgattgg |
| oriented on the | actttattttcatgctgtgattggttgtcttaagactggctctcatccaatcagaacatgataataaagtccaatccaagtaa |
| plus strand with | ccctggagggtttttctcatccaatcagaacatgcagtcaggaatcctcctgtatataatctcggtatatgactgctgctg |
| the promoter | aag |
| region positioned | |
| (chr2:130,129,223- | |
| 130,129,622) and | |
| a promoter | |
| sequence of: | |
| (SEQ ID NO: 32) | |
| PCAT7 (gene position | ccgctgctaccaacgctagcgccgatgccagtacaaccgctgttgccctcaatgcaccagcccaccctacaaggct |
| chr9:94,554,461- | cctatcacttggcccccacgggtgccctcctaccactctggtcaagctgcagtctctgttgctgccaccaacctcatga |
| 94,603,990) | ggcaagctgcagagccacgccatctacaggctccagcataccacaggtgactcctcctcctcctccagcctggctg |
| oriented on the | ggagcagttgcgcaggcaaagccagagaaacctagaacaggatgcagggagtggaagtgttagagcttcaccttg |
| plus strand with | tcacgctggccactgggcggcagggaccatttcattaaaggcactcacacccaccttccaaagtccagcctctctttct |
| the promoter | ggcaaaagctggccaggaactggggcctgggctgggtgtgagtgccttcactgaaaccggctcctggccaagtcc |
| region positioned | agctggccaggaattgctgggcccatcggcactgcgctcttcggggagcccgcgtaggagaaactcagacccagc |
| (chr9:94,553,093- | cagccctctccacccaagtgctggttcccattcctcacgcctccacccacagtgccctgtccccctccctcccccgtg |
| 94,555,044) and a | gtgccctctattccctgcccggtagtcccagatggtctccacaacacagagcgtgcgtgagggcgcgggccaggg |
| promoter sequence | aaccacagtgggtgtgagagccctgcggtgctcaagattgcatgagcacgaagagatcaccctctagagtctggag |
| of: | tccgggaagagaagaactccatccttccatggaggccaccagaaagaggaaggaggccacagctgtcgctgccg |
| (SEQ ID NO: 33) | ctgctgcctctgcagcccaccaaccccactggcagtatagcccccgatagcacccctaacctgccccctgtaaccct |
| | gggatagcacacccaacagctgcaggcaatgtaaccaatacccccccaaaccatcccaactcctcggccgcaggc |
| | agtgttagcacccgatagtgcccacctgacgcagccacgggtattgctgcactagacagcgccccaaacccgcccc |
| | accctcaccccgccatgggcagtgcagctcctgatagcgcacccccagtcacaggcagtgcagcaccggaccac |
| | gcccttaaaccaccccccactgccagcattgtagccccagataactccacccaacccgcccctgccacggacagtg |
| | cagcagaagacaacgcccctaagccatccaccgccactggcagtacacgttagtgtacacaacctgcctctcccga |
| | ccacccctgccacctcgggcagtgtagccccccattccaccaccagcaatgcaaccccagagagtgcccccaaac |
| | agtccaatgccacaggcagtgtagcctttggcagtgagcccctatagtacacccaacccttacccccagaagaggg |
| | cagtgcagccctggataactcacctaccctaccacatttctaccactgtggccgagctgcggtctctgatttcaccacc |
| | aaccgcagcgaggcgagccgcggaggcgcaagctccggctccagcctccagcacgcggcggtgcctcttctttct |
| | gtcttccagcccggcaggagaagctctcgctgctagcctccctcctaccgctccgtcaccaccaccaccaacaggg |
| | agggagtgccccaggctccaggctccaagctccaggctccagggggcgaaaagtgacggagccttccagttcttta |
| | agccgggtacgtagtagctccccacacccacacataagtgccagaaatgacgcgccgctgcctcagcatgctttata |
| | tactgaggttaggagaccctggactacatgttctgattggatgagagaaaaacctctaggcctactctgattggacttta |
| | ttttcatgctgtgattggttgtcttaagacttgctctcatccaatc |
| PURPL (gene position | tctttggcagtgaaagctccccatagtccatccatcctactaattgtaactataaactttgaatactttttttttttttgagacg |
| chr5:27,217,629- | gtgtctcactctgtcgcccaggttggagtgcagtggcaagatctcggctcacttcaacctctgctgcctgggttcaagt |
| 27,498,637) | gattctcctgcctcagcctcctgagtagctgggattacaggcgcttgccaccgcgcctggctaatttttgtagtttttcta |
| oriented on the | agtagagacggggaactttggatacattttttagagtaaaaaaagtaaattaaaaataccctaattgaaaactctgaaaa |
| plus strand with | taaacaagttagttttttttaagagatgccaaaatttgaaaaagcaactgttattaaagtgatttgcattattttttttttccttg |
| the promoter | ggatattttttcccattcacagagtggaaaagcataggtgactacaagtcttgctagacactctgctgtcaatttggctag |
| region positioned | gaaaaaagcaaaaacaaaaacaagtaatggacattcagtaaatattagcagagacaaaggaatgttacaaaggaag |
| (chr5:27,471,047- | caaatggaaattttttaaatgaaaaataaaatatctgaaattaaacatttattaagttcaacagcaaaaaaagaataatagt |
| 27,472,292) and a | aggaaaatatgttgaacataaaatagtatgtccaaaaagccagatctaacttcttattgcctacaaggaatcaactttga |
| promoter sequence | atataaagaaaatggtacaaattaatagatcaatagccattccccaatataaagaacacagaacaatgaatgagactaa |
| of: | agagatactttgaaacagaaaaataaggaaacctcaaaacgtgaaggacaatattaaaaggtctaacgtatttgtaatt |
| (SEQ ID NO: 34) | tcggtacccaaagttaaaattggggcatacttctaaatacccaaagactttcaaaatctgcagtacagtcaatgacatac |
| | atttacgaatgaaagaagctcagcaaattccaaatagtgtaagtgggagcaaaatgcctaggcagataaaaaggggt |
| | ccctagagaatttctggccttcttcacaagtgtttacattagatgcttttgtgcagatgagggaacctgcccagggcttgt |
| | ctgggcatgcccacagtggactggagcctgacatgctcactggggcaagtgggtggagccatgaggaattcatgcc |
| | ttgcaaaggggaggagcctgccctcttgagctcttgtggtgacc |
| WDR72 (gene | taatcatgtggtttttgtctttggctctgtttatatgctggattacatttattgatttgcgtatattgaaccagccttgcatccca |
| position | gggatgaagcccacttgatcatggtggataagctttttgatgtgctgctggattcgttttgccagtattttattgaggattttt |
| chr15:53,513,741- | gcatcaatgttcatcaaggatattggtctaaaattctcttttttggttgtgtctctgtccggctttggtatcagaatgatgctg |
| 54,762,878) | gcctcataaaatgagttagggaggattccctctttttctattgattggaatagtttcagaaggaatggtaccagttcctcct |
| oriented on the | tgtacctctggtagaattcggctgtgaatccatctggtcctggactctttttggttggtaaactattgattattgccacaattt |
| minus strand with | cagctcctgttattggtctattcagagattcaacttcttcctggttcagtcttgggagagtgtatgtgtcaaggaatttatcc |
| the promoter | atttcttctagattttctagtttatttgcgtagaggtgtttgtagtttgtagaggtgtttgtagtactctgatggtagtttgtattt |
| region positioned | clgtgggatcggtggtgacatcccctttatcattttttattgcgtctattagattcttctctctttttttctttattagtcttgctag |
| (chr15:53,541,104- | cggtctatcaattttgttgatcttttcagaaaacagctcctggattcattaattttttgaacggttttttgtgtctctatttccttc |
| 53,544,420) and a | agttctgctctgattttagttatttcttgccttctgctagcttttgaatgtgtttgctcttgcttttctagttcttttaattgtgatgtt |
| promoter sequence | agggtgtcaattttggatctttcctgctttctcttgtgggcatttagtgctataaatttccctctacacactgcttgaatgcgt |
| of: | cccagagattctggtatgttgtgtctttgttctcgttggtttcaaagaacatcttcctttctaccttcattttcttatgtacccag |
| (SEQ ID NO: 35) | aagtcattcaggagcaggttgttcagtttccatgtagttgagcggttttgagtgagattcttaatcctgagttctagtttcat |
| | tgcactgtggtctgagagatagtttgttataatctctgttcttttacatttgctgaggagagctttacttccaagtatgtggtc |
| | aattttggaataggtgtggtgtggtgctgaaaaaaatgtatattctgttgatttggggtggagagttctgtagatgtctatt |
| | aggtccgcttggtgcagagctgagttcaattcctgggtatccttgttgactttctgtctcgttgatctgtctaatgttgacag |
| | tggggtgttaaagtctcccattattaatgtgtgggagtctaagtctctttgtaggtcactcaggacttgctttacgaatctg |
| | ggtgctcctgtgttgagtgcatatatatttaggatagttagctcttcttgttgaattgatccctttaccattatgtaatggcctt |
| | ctttgtctcttttgatctttgttggtttaaagtctgttttatcagagactaggattgcaacccctgcctttttttgttttccatttgc |
| | ttggtagatcttcctccatccttttattttgagcctatgtgtgtctctgcacgtgagatgggtttcctgaatacagcacactg |
| | atgggtcttgactctttatccaatttgccagtctgtgtcttttaattggagcatttagtccatttacatttaaagttaatattgtta |
| | tgtgtgaatttgatcctgtcattatgatgttagctggtgattttgctcgttagttgatgcagtttcttcctagtctcgatggtctt |
| | tacattttggcatgattttgcagcgactggtaccggttgttcctttccatgtttagcgcttccttcaggagctcttttagggc |
| | aggcctggtggtgacaaaatctctcagcatttgcttgtctgtaaagtattttatttctccttcacttatgaagcttagtttggc |
| | tggatatgaaattctgggttgaaaattcttttctttaagaatgttgaatattggcccccactctcttctggcttgtagggtttct |
| | gccgagagatccgctgttagtctgatgggcttccctttgagggtaacccgacctttctctctggctgcccttaacatttttt |
| | ccttcatttcaactttggtgaatctgacaattatgtgtcttggagttgctcttctcgaggagtatcuttgtggcgttctctgtat |
| | ttcctgattctgaacgttggcctgccttgctagattggggaagttctcctggataatatcctgcagagtgttttccaacttg |
| | gttccattctccccatcactttcaggtacaccaatcagacgtagatttggtcttttcacatagtcccatatttcttggaggct |
| | ttgctcatttctttttattcttttttctctaaacttcccttctcacttcatttcattcatttcatcttccatcgctgataccctttcttcc |
| | agttgatcgcatcggctcctgaggcttctgcattcttcacgtagttctcgagccttggttttcagctccatcagctccttta |
| | agcacttctctgtattggttattctagttatacattcttctaaatttttttcaaagttttcaacttctttgcctttggtttgaatgtcc |
| | tcccatagctcagagtaatttgatcatctgaagccttcttctctcagctcgtcaaagtcattctccatccagctttgttccgt |
| | tgctggtgaggaactgcgttcctttggaggaggagaggcgctctgcgttttagagtatccagtttttctgttctgttttttcc |
| | ccatctttgtggttttatctacttttgctctttgatgatggtgaggtacagatgagtttttgctgtggatgtcctttctgtttgtta |
| | gttttccttctaacagacaggaccctcagctgcaggtctgttggaataccttgccttgtgaggtatcagtgtgcccctgct |
| | ggggggtgcctcccagttaggctgctcgggggtcaggggtcagggacccacttgaggaggcagtctgcccgttct |
| | cagatctccagctgcgtactgggagaaccactgctctcttcaaagctgttagatagggacatttaagtctgcagaggtt |
| | actgctgtctttttgtttgtctgtgccctgcccccagaggtggagcctacagaggcaggcaggccaccttgagctgtg |
| | gtgggctccacccagttctagcttcccggctgcttt |

### Test Compounds

A wide variety of test compounds can be used in the inventive methods for identifying iTR factors and global modulators of iTR using the DR-iTR method. For example, a test compound can be a small molecule, polypeptide, peptide, nucleic acid, oligonucleotide, lipid, carbohydrate, antibody, or hybrid molecule including but not limited to those described herein, including mRNA for the genes OCT4, SOX2, KLF4, NANOG, ESRRB, NR5A2, CEBPA, MYC, SALL4, LIN28A and LIN28B alone and in diverse combinations, and in diverse combinations with small molecule compounds such as combinations of the following compounds: inhibitors of glycogen synthase 3 (GSK3) including but not limited to CHIR99021; inhibitors of TGF-beta signaling including but not limited to SB431542, A-83-01, and E616452; HDAC inhibitors including but not limited to aliphatic acid compounds including but not limited to: valproic acid, phenylbutyrate, and n-butyrate; cyclic tetrapeptides including trapoxin B and the depsipeptides; hydroxamic acids such as trichostatin A, vorinostat (SAHA), belinostat (PXD101), LAQ824, panobinostat (LBH589), and the benzamides entinostat (MS-275), C1994, mocetinostat (MGCD0103); those specifically targeting Class I (HDACl, HDAC2, HDAC3, and HDAC8), IIA (HDAC4, HDAC5, HDAC7, and HDAC9), IIB (HDAC6 and HDACIO), PI (SIRT1, SIRT2, SIRT3, SIRT4, SIRTS, SIRT6, or SIRT7) including the sirtuin inhibitors nicotinomide, vitamin B-12, diverse derivatives of NAD, dihydrocoumarin, naphthopyranone, and 2-hydroxynaphthaldehydes, or IV (HDACll) deacetylases; inhibitors of H3K4/9 histone demethylase LSD1 including but not limited to parnate; inhibitors of Dot1L including but not limited to EPZ004777; inhibitors of G9a including but not limited to Bix01294; inhibitors of EZH2 including but not limited to DZNep, inhibitors of DNA methyltransferase including but not limited to RG108; 5-aza-2′deoxycytidine (trade name Vidaza and Azadine); vitamin C which can inhibit DNA methylation, increase Tetl which increases 5hmC which is a first step of demethylation; activators of 3′ phosphoinositide-dependent kinase 1 including but not limited to PS48; promoters of glycolysis including but not limited to Quercetin and fructose 2, 6-bisphosphate (an activator of phosphofructokinase 1); agents that promote the activity of the HIFI transcription complex including but not limited to Quercetin; RAR agonists including but not limited to AM580, CD437, and TTNPB; agents that mimic hypoxia including but not limited to Resveratrol; agents that increase telomerase activity including but not limited to the exogenous expression of the catalytic component of telomerase (TERT), agents that promote epigenetic modifications via downregulation of LSD1, a H3K4-specific histone demethylase including but not limited to lithium; or inhibitors of the MAPK/ERK pathway including but not limited to PD032590. Such compounds may be administered in diverse combinations, concentrations, and for differing periods of time, to optimize the effect of iTR on cells cultured in vitro using markers of global iTR such as by assaying for decreased expression of COX7A1 or NAALADL1, or other inhibitors of iTR as described herein, and/or assaying for increased expression of PCDHB2 or AMH or other activators or iTR as described herein, or in injured or diseased tissues in vivo, or in modulating the lifespan of animals in vivo.

In vitro assays for iTR patterns of expression of the genes COX7A1, PLPP7, and NAALADL1 as well as gene expression or protein markers of pluripotency including DNMT3B, and HELLS or Tra-1-60, Tra-1-81, and SSEA4 respectively are performed to optimize global patterns of iTR gene expression without reverting the target cells to pluripotency. Examples of individual agents and combinations of agents screened are: OCT4, SOX2, KLF4, MYC and LIN28A; OCT4; KLF4; OCT4, KLF4; OCT4, KLF4, LIN28A; OCT4, KLF4, LIN28B; SOX2; MYC; NANOG; ESRRB; NT5A2; OCT4, SOX2, KLF4, and LIN28A; OCT4, SOX2, KLF4, and LIN28B; OCT4, KLF4, MYC and LIN28A; and each of the preceding combinations of agents together with 0.25 mM NaB, 5 mM PS48 and 0.5 mM A-83-01 during the first four weeks, followed by treatment with 0.25 mM sodium butyrate, 5 mM PS48, 0.5 mM A-83-01 and 0.5 mM PD0325901 each of which is assayed at 0, 1, 2, 4, 7, 10, and 14 days for markers of global modulation of iTR gene expression.

Compounds can be obtained from natural sources or produced synthetically. Compounds can be at least partially pure or may be present in extracts or other types of mixtures whose components are at least in part unknown or uncharacterized. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. The library may comprise, e.g., between 100 and 500,000 compounds, or more. Compounds are often arrayed in multiwell plates (e.g., 384 well plates, 1596 well plates, etc.). They can be dissolved in a solvent (e.g., DMSO) or provided in dry form, e.g., as a powder or solid. Collections of synthetic, semi-synthetic, and/or naturally occurring compounds can be tested. Compound libraries can comprise structurally related, structurally diverse, or structurally unrelated compounds. Compounds may be artificial (having a structure invented by man and not found in nature) or naturally occurring. In some embodiments, a library comprises at least some compounds that have been identified as “hits” or “leads” in other drug discovery programs and/or derivatives thereof. A compound library can comprise natural products and/or compounds generated using non-directed or directed synthetic organic chemistry. Often a compound library is a small molecule library. Other libraries of interest include peptide or peptoid libraries, cDNA libraries, antibody libraries, and oligonucleotide libraries. A library can be focused (e.g., composed primarily of compounds having the same core structure, derived from the same precursor, or having at least one biochemical activity in common).

Compound libraries are available from a number of commercial vendors such as Tocris Bioscience, Nanosyn, BioFocus, and from government entities. For example, the Molecular Libraries Small Molecule Repository (MLSMR), a component of the U.S. National Institutes of Health (NIH) Molecular Libraries Program is designed to identify, acquire, maintain, and distribute a collection of >300,000 chemically diverse compounds with known and unknown biological activities for use, e.g., in high-throughput screening (HTS) assays (see commonfund.nih.gov/molecularlibraries/index). The NIH Clinical Collection (NCC) is a plated array of approximately 450 small molecules that have a history of use in human clinical trials. These compounds are highly drug-like with known safety profiles. In some embodiments, a collection of compounds comprising “approved human drugs” is tested. An “approved human drug” is a compound that has been approved for use in treating humans by a government regulatory agency such as the US Food and Drug Administration, European Medicines Evaluation Agency, or a similar agency responsible for evaluating at least the safety of therapeutic agents prior to allowing them to be marketed. The test compound may be, e.g., an antineoplastic, antibacterial, antiviral, antifungal, antiprotozoal, antiparasitic, antidepressant, antipsychotic, anesthetic, antianginal, antihypertensive, antiarrhythmic, anti-inflammatory, analgesic, antithrombotic, antiemetic, immunomodulator, antidiabetic, lipid- or cholesterol-lowering (e.g., statin), anticonvulsant, anticoagulant, antianxiety, hypnotic (sleep-inducing), hormonal, or anti-hormonal drug, etc. In some embodiments, a compound is one that has undergone at least some preclinical or clinical development or has been determined or predicted to have “drug-like” properties. For example, the test compound may have completed a Phase I trial or at least a preclinical study in non-human animals and shown evidence of safety and tolerability.

In some embodiments, a test compound is substantially non-toxic to cells of an organism to which the compound may be administered and/or to cells with which the compound may be tested, at the concentration to be used or, in some embodiments, at concentrations up to 10-fold, 100-fold, or 1,000-fold higher than the concentration to be used. For example, there may be no statistically significant effect on cell viability and/or proliferation, or the reduction in viability or proliferation can be no more than 1%, 5%, or 10% in various embodiments. Cytotoxicity and/or effect on cell proliferation can be assessed using any of a variety of assays. For example, a cellular metabolism assay such as AlamarBlue, MTT, MTS, XTT, and CellTitre Glo assays, a cell membrane integrity assay, a cellular ATP-based viability assay, a mitochondrial reductase activity assay, a BrdU, EdU, or H3-Thymidine incorporation assay could be used. In some embodiments, a test compound is not a compound that is found in a cell culture medium known or used in the art, e.g., culture medium suitable for culturing vertebrate, e.g., mammalian cells or, if the test compound is a compound that is found in a cell culture medium known or used in the art, the test compound is used at a different, e.g., higher, concentration when used in a method of the present invention.

### Assays for Global Modulators of DR-iTR in Microbiopsies: Aspects of Assay Implementation and Controls

Various inventive screening assays described above involve determining whether a test iTR factor or combination of factors generate DR-iTR microbiopsies. Suitable cells for expression of a reporter molecule are described above. In performing an inventive assay, assay components (e.g., cells, TR activator or TR inhibitor polypeptide, and test compounds) are typically dispensed into multiple vessels or other containers. Any type of vessel or article capable of containing cells can be used. In many embodiments of the invention, the vessels are wells of a multi-well plate (also called a “microwell plate”, “microtiter plate”, etc. For purposes of description, the term “well” will be used to refer to any type of vessel or article that can be used to perform an inventive screen, e.g., any vessel or article that can contain the assay components. It should be understood that the invention is not limited to use of wells or to use of multi-well plates. In some embodiments, any article of manufacture in which multiple physically separated cavities (or other confining features) are present in or on a substrate can be used. For example, assay components can be confined in fluid droplets, which may optionally be arrayed on a surface and, optionally, separated by a water resistant substance that confines the droplets to discrete locations, in channels of a microfluidic device, etc.

In general, assay components can be added to wells in any order. For example, DT-iTR microbiopsies can be added first and maintained in culture for a selected time period (e.g., between 6 and 48 hours) prior to addition of a test compound and target TR activator. In some embodiments, compounds are added to wells prior to addition of polypeptides of cells. In some embodiments, expression of a reporter polypeptide is induced after plating the cells, optionally after addition of a test compound to a well. In some embodiments, expression of the reporter molecule is achieved by transfecting the cells with an expression vector that encodes the reporter polypeptide. In some embodiments, the cells have previously been genetically engineered to express the reporter polypeptide. In some embodiments, expression of the reporter molecule is under control of regulatable expression control elements, and induction of expression of the reporter molecule is achieved by contacting the cells with an agent that induces (or derepresses) expression. The assay composition comprising cells, test compound, or polypeptide is maintained for a suitable time period during which test compound may (in the absence of a test compound that inhibits its activity) cause an increase or decrease of the level or activity of the target TR activator or TR inhibitor. The number of cells, amount of TR activator or TR inhibitor polypeptide, and amount of test compound to be added will depend, e.g., on factors such as the size of the vessel, cell type, and can be determined by one of ordinary skill in the art. In some embodiments, the ratio of the molar concentration of TR activator or TR inhibitor polypeptide to test compound is between 1:10 and 10:1. In some embodiments, the number of cells, amount of test compound, and length of time for which the composition is maintained can be selected so that a readily detectable level signal after a selected time period in the absence of a test compound. In some embodiments, cells are at a confluence of about 25%-75%, e.g., about 50%, at the time of addition of compounds. In some embodiments, between 1,000 and 10,000 cells/well (e.g., about 5,000 cells/well) are plated in about 100 mĩ medium per well in 96-well plates. In other exemplary embodiments, cells are seeded in about 301-50 mĩ of medium at between 500 and 2,000 (e.g., about 1000) cells per well into 384-well plates. In some embodiments, compounds are tested at multiple concentrations (e.g., 2-10 different concentrations) and/or in multiple replicates (e.g., 2-10 replicates). Multiple replicates of some or all different concentrations can be performed. In some embodiments, candidate TR factors are used at a concentration between 0.1 mg/ml and 100 mg/ml, e.g., 1 mg/ml and 10 mg/ml. In some embodiments, candidate TR factors are used at multiple concentrations. In some embodiments, compounds are added to cells between 6 hours and one day (24 hr) after seeding.

In some aspects of any of the inventive compound screening and/or characterization methods, a test compound is added to an assay composition in an amount sufficient to achieve a predetermined concentration. In some embodiments the concentration is up to about 1 nM. In some embodiments the concentration is between about 1 nM and about 100 nM. In some embodiments the concentration is between about 100 nM and about 10 mM. In some embodiments the concentration is at least 10 mM, e.g., between 10 mM and 100 mM. The assay composition can be maintained for various periods of time following addition of the last component thereof. In certain embodiments the assay composition is maintained for between about 10 minutes and about 4 days, e.g., between 1 hour and 3 days, e.g., between 2 hours and 2 days, or any intervening range or particular value, e.g., about 4-8 hours, after addition of all components. Multiple different time points can be tested. Additional aliquots of test compound can be added to the assay composition within such time period. In some embodiments, cells are maintained in cell culture medium appropriate for culturing cells of that type. In some embodiments, a serum-free medium is used. In some embodiments, the assay composition comprises a physiologically acceptable liquid that is compatible with maintaining integrity of the cell membrane and, optionally, cell viability, instead of cell culture medium. Any suitable liquid could be used provided it has the proper osmolarity and is otherwise compatible with maintaining reasonable integrity of the cell membrane and, optionally, cell viability, for at least a sufficient period of time to perform an assay. One or more measurements indicative of an increase in the level of active TR activator or decrease in TR inhibitor can be made during or following the incubation period.

In some embodiments, the compounds screened for potential to be global modulators of iTR are chosen from agents capable in other conditions of inducing pluripotency in somatic cell types. Such agents include the following compounds individually or in combination: the genes OCT4, SOX2, KLF4, NANOG, ESRRB, NR5A2, CEBPA, TERT, MYC, LIN28A and LIN28B alone and in combination with small molecule compounds such as combinations of the following compounds: inhibitors of glycogen synthase 3 (GSK3) including but not limited to CHIR99021; inhibitors of TGF-beta signaling including but not limited to SB431542, A-83-01, and E616452; HDAC inhibitors including but not limited to aliphatic acid compounds including but not limited to: valproic acid, phenylbutyrate, and n-butyrate; cyclic tetrapeptides including trapoxin B and the depsipeptides; hydroxamic acids such as trichostatin A, vorinostat (SAHA), belinostat (PXDIOI), LAQ824, panobinostat (LBH589), and the benzamides entinostat (MS-275), C1994, mocetinostat (MGCD0103); those specifically targeting Class I (HDACl, HDAC2, HDAC3, and HDAC8), PA (HDAC4, HDAC5, HDAC7, and HDAC9), IIB (HDAC6 and HDACIO), III (SIRT1, SIRT2, SIRT3, SIRT4, SIRT5, SIRT6, or SIRT7) including the sirtuin inhibitors nicotinomide, diverse derivatives of NAD, dihydrocoumarin, naphthopyranone, and 2-hydroxynaphthaldehydes, or IV (HDACl 1) deacetylases; inhibitors of H3K4/9 histone demethylase LSD1 including but not limited to parnate; inhibitors of Dot1L including but not limited to EPZ004777; inhibitors of G9a including but not limited to Bix01294; inhibitors of EZH2 including but not limited to DZNep, inhibitors of DNA methyltransferase including but not limited to RG108; 5-aza-2′deoxycytidine (trade name Vidaza and Azadine); vitamin C which can inhibit DNA methylation, increase Tetl which increases 5hmC which is a first step of demethylation; activators of 3′ phosphoinositide-dependent kinase 1 including but not limited to PS48; promoters of glycolysis including but not limited to Quercetin and fructose 2, 6-bisphosphate (an activator of phosphofructokinase 1); agents that promote the activity of the HIF1 transcription complex including but not limited to Quercetin; RAR agonists including but not limited to AM580, CD437, and TTNPB; agents that mimic hypoxia including but not limited to Resveratrol; agents that increase telomerase activity including but not limited to the exogenous expression of the catalytic component of telomerase (TERT), agents that promote epigenetic modifications via downregulation of LSD1, a H3K4-specific histone demethylase including but not limited to lithium; or inhibitors of the MAPK/ERK pathway including but not limited to PD032590. Such compounds may be administered in diverse combinations, concentrations, and for differing periods of time, to optimize the effect of iTR on cells cultured in vitro using markers of global iTR such as by assaying for decreased expression of COX7A1 or NAALADL1, or other inhibitors of iTR as described herein, and/or assaying for increased expression of PCDHB2 or AMH or other activators or iTR as described herein, or in injured or diseased tissues in vivo, or in modulating the lifespan of animals in vivo.

In some embodiments, individual compounds, each typically of known identity (e.g., structure and/or sequence), are added to each of a multiplicity of wells. In some embodiments, two or more compounds may be added to one or more wells. In some embodiments, one or more compounds of unknown identity may be tested. The identity may be determined subsequently using methods known in the art.

In various embodiments, foregoing assay methods of the invention are amenable to high throughput screening (HTS) implementations. In some embodiments, the screening assays of the invention are high throughput or ultra-high throughput (see, e.g., Fernandes, P. B., Curr Opin Chem. Biol. 1998, 2:597; Sundberg, S A, Curr Opin Biotechnol. 2000, 11:47). High throughput screens (HTS) often involve testing large numbers of compounds with high efficiency, e.g., in parallel. For example, tens or hundreds of thousands of compounds can be routinely screened in short periods of time, e.g, hours to days. In some embodiments, HTS refers to testing of between 1,000 and 100,000 compounds per day. In some embodiments, ultra-high throughput refers to screening in excess of 100,000 compounds per day, e.g., up to 1 million or more compounds per day. The screening assays of the invention may be carried out in a multi-well format, for example, a 96-well, 384-well format, 1,536-well format, or 3,456-well format and are suitable for automation. In some embodiments, each well of a microwell plate can be used to run a separate assay against a different test compound or, if concentration or incubation time effects are to be observed, a plurality of wells can contain test samples of a single compound, with at least some wells optionally being left empty or used as controls or replicates. Typically, HTS implementations of the assays disclosed herein involve the use of automation. In some embodiments, an integrated robot system including one or more robots transports assay microwell plates between multiple assay stations for compound, cell and/or reagent addition, mixing, incubation, and readout or detection. In some aspects, an HTS system of the invention may prepare, incubate, and analyze many plates simultaneously. Suitable data processing and control software may be employed. High throughput screening implementations are well known in the art. Without limiting the invention in any way, certain general principles and techniques that may be applied in embodiments of a HTS of the present invention are described in Macarron R & Hertzberg R P. Design and implementation of high-throughput screening assays. Methods Mol Biol., 565: 1-32, 2009 and/or An W F & Tolliday N J., Introduction: cell-based assays for high-throughput screening. Methods Mol Biol. 486: 1-12, 2009, and/or references in either of these. Exemplary methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006).

An additional compound may, for example, have one or more improved pharmacokinetic and/or pharmacodynamic properties as compared with an initial hit or may simply have a different structure. An “improved property” may, for example, render a compound more effective or more suitable for one or more purposes described herein. In some embodiments, for example, a compound may have higher affinity for the molecular target of interest (e.g., TR activator or TR inhibitor gene products), lower affinity for a non-target molecule, greater solubility (e.g., increased aqueous solubility), increased stability (e.g., in blood, plasma, and/or in the gastrointestinal tract), increased half-life in the body, increased bioavailability, and/or reduced side effect(s), etc. Optimization can be accomplished through empirical modification of the hit structure (e.g., synthesizing compounds with related structures and testing them in cell-free or cell-based assays or in non-human animals) and/or using computational approaches. Such modification can in some embodiments make use of established principles of medicinal chemistry to predictably alter one or more properties. In some embodiments, one or more compounds that are “hit” are identified and subjected to systematic structural alteration to create a second library of compounds (e.g., refined lead compounds) structurally related to the hit. The second library can then be screened using any of the methods described herein. In some embodiments, an iTR factor is modified or incorporates a moiety that enhances stability (e.g., in serum), increases half-life, reduces toxicity or immunogenicity, or otherwise confers a desirable property on the compound.

### Uses of DR-iTR Microbiopsies

### Pharmaceutical Compositions

DR-iTR microbiopsies have a variety of different uses. Non-limiting examples of such uses are discussed herein. In some embodiments, a DR-iTR microbiopsy is used to enhance regeneration of an organ or tissue. In some embodiments, a DR-iTR microbiopsy is used to enhance regeneration of a limb, digit, cartilage, heart, blood vessel, bone, esophagus, stomach, liver, gallbladder, pancreas, intestines, rectum, anus, endocrine gland (e.g., thyroid, parathyroid, adrenal, endocrine portion of pancreas), skin, hair follicle, thymus, spleen, skeletal muscle, focal damaged cardiac muscle, smooth muscle, brain, spinal cord, peripheral nerve, ovary, fallopian tube, uterus, vagina, mammary gland, testes, vas deferens, seminal vesicle, prostate, penis, pharynx, larynx, trachea, bronchi, lungs, kidney, ureter, bladder, urethra, eye (e.g., retina, cornea), or ear (e.g., organ of Corti). In some embodiments, a DR-iTR microbiopsy is used to enhance regeneration of a stromal layer, e.g., a connective tissue supporting the parenchyma of a tissue. In some embodiments, a DR-iTR microbiopsy is used to enhance regeneration following surgery, e.g., surgery that entails removal of at least a portion of a diseased or damaged tissue, organ, or other structure such as a limb, digit, etc. For example, such surgery might remove at least a portion of a liver, lung, kidney, stomach, pancreas, intestine, mammary gland, ovary, testis, bone, limb, digit, muscle, skin, etc. In some embodiments, the surgery is to remove a tumor. In some embodiments, a DR-iTR microbiopsy is used to promote scarless regeneration of skin following trauma, surgery, disease, and burns.

Enhancing regeneration can include any one or more of the following, in various embodiments: (a) increasing the rate of regeneration; (b) increasing the extent of regeneration; (c) promoting establishment of appropriate structure (e.g., shape, pattern, tissue architecture, tissue polarity) in a regenerating tissue or organ or other body structure; (d) promoting growth of new tissue in a manner that retains and/or restores function; e) expansion of the DT-iTR microbiopsy to obtain more tissue for transplantation. While use of a DR-iTR microbiopsy to enhance regeneration is of particular interest, the invention encompasses use of a DR-iTR microbiopsy to enhance repair, closure of a wound, or wound healing in general, without necessarily producing a detectable enhancement of epimorphic regeneration. Thus, the invention provides methods of enhancing repair or wound healing, wherein a DR-iTR microbiopsy is administered to a subject in need thereof according to any of the methods described herein.

In some embodiments, the invention provides a method of enhancing regeneration in a subject in need thereof, the method comprising administering an effective amount of a DR-iTR microbiopsy to the subject. In some embodiments, an effective amount of a compound (e.g., a DR-iTR microbiopsy) is an amount that results in an increased rate or extent of regeneration of damaged tissue as compared with a reference value (e.g., a suitable control value). In some embodiments, the reference value is the expected (e.g., average or typical) rate or extent of regeneration in the absence of the DT-iTR microbiopsy (optionally with administration of a placebo). In some embodiments, an effective amount of DR-iTR microbiopsies transplanted is an amount that results in an improved structural and/or functional outcome as compared with the expected (e.g., average or typical) structural or functional outcome in the absence of the compound. In some embodiments, an effective amount of microbiopsies engrafted, e.g., a DR-iTR microbiopsy, results in enhanced blastema formation and/or reduced scarring. Extent or rate of regeneration can be assessed based on dimension(s) or volume of regenerated tissue, for example. Structural and/or functional outcome can be assessed based on, e.g., visual examination (optionally including use of microscopy or imaging techniques such as X-rays, CT scans, MRI scans, PET scans) and/or by evaluating the ability of the tissue, organ, or other body part to perform one or more physiological processes or task(s) normally performed by such tissue, organ, or body part. Typically, an improved structural outcome is one that more closely resembles normal structure (e.g., structure that existed prior to tissue damage or structure as it exists in a normal, healthy individual) as compared with the structural outcome that would be expected (e.g., average or typical outcome) in the absence of treatment with a DR-iTR microbiopsy engraftment.

One of ordinary skill in the art can select an appropriate assay or test for function. In some embodiments, an increase in the rate or extent of regeneration as compared with a control value is statistically significant (e.g., with a p value of <0.05, or with a p value of <0.01) and/or clinically significant. In some embodiments, an improvement in structural and/or functional outcome as compared with a control value is statistically significant and/or clinically significant. “Clinically significant improvement” refers to an improvement that, within the sound judgement of a medical or surgical practitioner, confers a meaningful benefit on a subject (e.g., a benefit sufficient to make the treatment worthwhile).

In some embodiments, the DR-iTR microbiopsy is used to enhance skin regeneration, e.g., after a burn (thermal or chemical), scrape injury, or other situations involving skin loss, e.g., infections such as necrotizing fasciitis or purpura fulminans. In some embodiments, a burn is a second or third degree burn. In some embodiments a region of skin loss has an area of at least 10 cm². In one aspect, DR-iTR microbiopsies enhance regeneration of grafted skin. In one aspect, a DR-iTR factor reduces excessive and/or pathological wound contraction or scarring.

In some embodiments, a DR-iTR microbiopsy is used to enhance bone regeneration, e.g., in a situation such as non-union fracture, implant fixation, periodontal or alveolar ridge augmentation, craniofacial surgery, or other conditions in which generation of new bone is considered appropriate. In some embodiments, a DR-iTR factor is applied to a site where bone regeneration is desired. In some embodiments, a DR-iTR factor is incorporated into or used in combination with a bone graft material. Bone graft materials include a variety of ceramic and proteinaceous materials. Bone graft materials include autologous bone (e.g., bone harvested from the iliac crest, fibula, ribs, etc.), allogeneic bone from cadavers, and xenogeneic bone. Synthetic bone graft materials include a variety of ceramics such as calcium phosphates (e.g. hydroxyapatite and tricalcium phosphate), bioglass, and calcium sulphate, and proteinaceous materials such as demineralized bone matrix (DBM). DBM can be prepared by grinding cortical bone tissues (generally to 100-500 mih sieved particle size), then treating the ground tissues with hydrochloric acid (generally 0.5 to 1 N). In some embodiments, a DR-iTR factor is administered to a subject together with one or more bone graft materials. The DR-iTR factor may be combined with the bone graft material (in a composition comprising an DR-iTR factor and a bone graft material) or administered separately, e.g., after placement of the graft. In some embodiments, the invention provides a bone paste comprising a DR-iTR factor. Bone pastes are products that have a suitable consistency and composition such that they can be introduced into bone defects, such as voids, gaps, cavities, cracks etc., and used to patch or fill such defects, or applied to existing bony structures. Bone pastes typically have sufficient malleability to permit them to be manipulated and molded by the user into various shapes. The desired outcome of such treatments is that bone formation will occur to replace the paste, e.g., retaining the shape in which the paste was applied. The bone paste provides a supporting structure for new bone formation and may contain substance(s) that promote bone formation. Bone pastes often contain one or more components that impart a paste or putty-like consistency to the material, e.g., hyaluronic acid, chitosan, starch components such as amylopectin, in addition to one or more of the ceramic or proteinaceous bone graft materials (e.g., DBM, hydroxyapatite) mentioned above.

In some embodiments, a DR-iTR factor enhances the formation and/or recruitment of osteoprogenitor cells from undifferentiated mesenchymal cells and/or enhances the differentiation of osteoprogenitor cells into cells that form new bone (osteoblasts). In some embodiments, a DR-iTR factor is administered to a subject with osteopenia or osteoporosis, e.g., to enhance bone regeneration in the subject.

In some embodiments, a DR-iTR factor is used to enhance regeneration of a joint (e.g., a fibrous, cartilaginous, or synovial joint). In some embodiments, the joint is an intervertebral disc. In some embodiments, a joint is a hip, knee, elbow, or shoulder joint. In some embodiments, a DR-iTR microbiopsy is used to enhance regeneration of dental and/or periodontal tissues or structures (e.g., pulp, periodontal ligament, teeth, periodontal bone). In some embodiments, a DR-iTR microbiopsy is used to reduce glial scarring in CNS and PNS injuries. In some embodiments, a DR-iTR microbiopsy is used to reduce adhesions and stricture formation in internal surgery. In some embodiments, a DR-iTR microbiopsy is used to decrease scarring in tendon and ligament repair improving mobility. In some embodiments, a DR-iTR factor is used to reduce vision loss following eye injury. In some embodiments, a DR-iTR factor is administered to a subject in combination with other cells. The iTR factor and the cells may be administered separately or in the same composition. If administered separately, they may be administered at the same or different locations. The cells can be autologous, allogeneic, or xenogeneic in various embodiments. The cells can comprise progenitor cells or stem cells, e.g., adult stem cells. As used herein, a stem cell is a cell that possesses at least the following properties: (i) self-renewal, i.e., the ability to go through numerous cycles of cell division while still maintaining an undifferentiated state; and (ii) multipotency or multidifferentiative potential, i.e., the ability to generate progeny of several distinct cell types (e.g., many, most, or all of the distinct cell types of a particular tissue or organ). An adult stem cell is a stem cell originating from non-embryonic tissues (e.g., fetal, post-natal, or adult tissues). As used herein, the term “progenitor cell” encompasses multipotent cells that are more differentiated than pluripotent stem cells but not fully differentiated. Such more differentiated cells (which may arise from embryonic progenitor cells) have reduced capacity for self-renewal as compared with embryonic progenitor cells. In some embodiments, a DR-iTR microbiopsy is administered in combination with mesenchymal progenitor cells, neural progenitor cells, endothelial progenitor cells, hair follicle progenitor cells, neural crest progenitor cells, mammary stem cells, lung progenitor cells (e.g., bronchioalveolar stem cells), muscle progenitor cells (e.g., satellite cells), adipose-derived progenitor cells, epithelial progenitor cells (e.g., keratinocyte stem cells), and/or hematopoietic progenitor cells (e.g., hematopoietic stem cells). In some embodiments, the cells comprise induced pluripotent stem cells (iPS cells), or cells that have been at least partly differentiated from iPS cells. In some embodiments, the progenitor cells comprise adult stem cells. In some embodiments, at least some of the cells are differentiated cells, e.g., chondrocytes, osteoblasts, keratinocytes, hepatocytes. In some embodiments, the cells comprise myoblasts.

In some embodiments the iTR microbiopsy is genetically modified to evade immune rejection and therefore be utilized as an allogeneic graft. Said genetic modifications may include the modification or elimination of one or more HLA antigens or beta 2 microglobulin, the introduction of immune suppressive modulators such as PD1. PDL1, or the exogenous expression of HLA-G.

In some embodiments, a DR-iTR microbiopsy is administered in a composition (e.g., a solution) comprising one or more compounds that polymerizes or becomes cross-linked or undergoes a phase transition in situ following administration to a subject, typically forming a hydrogel. The composition may comprise monomers, polymers, initiating agents, cross-linking agents, etc. The composition may be applied (e.g., using a syringe) to an area where regeneration is needed, where it forms a gel in situ, from which a DR-iTR factor is released over time. Gelation may be triggered, e.g., by contact with ions in body fluids or by change in temperature or pH, or by light, or by combining reactive precursors (e.g., using a multi-barreled syringe). (See, e.g., U.S. Pat. No. 6,129,761; Yu L, Ding J. Injectable hydrogels as unique biomedical materials. Chem Soc Rev. 37(8): 1473-81 (2008)). In some embodiments the hydrogel is a hyaluronic acid or hyaluronic acid and collagen I-containing hydrogel such as HyStem-C described herein. In some embodiments, the composition further comprises cells.

In some embodiments, a DR-iTR microbiopsy is administered to a subject in combination with vectors expressing the catalytic component of telomerase (TERT). The expression of TERT is especially useful during the extensive expansion of DR-iTR microbiopsies, or when said microbiopsy is obtained from an aged human patient. The vector expressing TERT may be administered separately or at the same time the DT-iTR microbiopsy is reprogrammed. Other inventive methods comprise the cryopreservation of DR-iTR microbiopsy tissue for subsequent allo- or autologous transplantation. Said cryopreservation may include the use of vitrification. Other inventive methods comprise use of a DR-iTR microbiopsy in the ex vivo production of living, functional tissues, organs, or cell-containing compositions to repair or replace a tissue or organ lost due to damage. For example, cells or tissues removed from an individual (either the future recipient, an individual of the same species, or an individual of a different species) may be cultured in vitro, optionally with an matrix, scaffold (e.g., a three dimensional scaffold) or mold (e.g., comprising a biocompatible, optionally biodegradable, material, e.g., a polymer such as HyStem-C), and their development into a regenerative and expandable tissue or organ can be promoted by contacting an iTR factor. The scaffold, matrix, or mold may be composed at least in part of naturally occurring proteins such as collagen, hyaluronic acid, or alginate (or chemically modified derivatives of any of these), or synthetic polymers or copolymers of lactic acid, caprolactone, glycolic acid, etc., or self-assembling peptides, or decellularized matrices derived from tissues such as heart valves, intestinal mucosa, blood vessels, and trachea. In some embodiments, the scaffold comprises a hydrogel. The scaffold may, in certain embodiments, be coated or impregnated with an iTR factor, which may diffuse out from the scaffold over time. After production ex vivo, the tissue or organ is grafted into or onto a subject. For example, the tissue or organ can be implanted or, in the case of certain tissues such as skin, placed on a body surface. The tissue or organ may continue to develop in vivo. In some embodiments, the tissue or organ to be produced at least in part ex vivo is a bladder, blood vessel, bone, fascia, liver, muscle, skin patch, etc. Suitable scaffolds may, for example, mimic the extracellular matrix (ECM). Optionally, an DR-iTR factor is administered to the subject prior to, during, and/or following grafting of the ex vivo-generated DR-iTR microbiopsy. In some aspects, a biocompatible material is a material that is substantially non-toxic to cells in vitro at the concentration used or, in the case of a material that is administered to a living subject, is substantially nontoxic to the subject's cells in the quantities and at the location used and does not elicit or cause a significant deleterious or untoward effect on the subject, e.g., an immunological or inflammatory reaction, unacceptable scar tissue formation, etc. It will be understood that certain biocompatible materials may elicit such adverse reactions in a small percentage of subjects, typically less than about 5%, 1%, 0.5%, or 0.1%.

In some embodiments, a matrix or scaffold coated or impregnated with a DR-iTR factor or combinations of factors including those capable of causing a global pattern of DR-iTR gene expression is implanted, optionally in combination with cells, into a subject in need of regeneration. The matrix or scaffold may be in the shape of a tissue or organ whose regeneration is desired. The cells may be stem cells of one or more type(s) that gives rise to such tissue or organ and/or of type(s) found in such tissue or organ.

In some embodiments, a DR-iTR formulation or combination with other iTR factors is administered directly to or near a site of tissue damage. “Directly to a site of tissue damage” encompasses injecting a compound or composition into a site of tissue damage or spreading, pouring, or otherwise directly contacting the site of tissue damage with the compound or composition. In some embodiments, administration is considered “near a site of tissue damage” if administration occurs within up to about 10 cm away from a visible or otherwise evident edge of a site of tissue damage or to a blood vessel (e.g., an artery) that is located at least in part within the damaged tissue or organ. Administration “near a site of tissue damage” is sometimes administration within a damaged organ, but at a location where damage is not evident. In some embodiments, following damage or loss of a tissue, organ, or other structure, a DR-iTR factor is applied to the remaining portion of the tissue, organ, or other structure. In some embodiments, a DR-iTR factor is applied to the end of a severed digit or limb) that remains attached to the body, to enhance regeneration of the portion that has been lost. In some embodiments, the severed portion is reattached surgically, and a DR-iTR factor is applied to either or both faces of the wound. In some embodiments, a DR-iTR factor is administered to enhance engraftment or healing or regeneration of a transplanted organ or portion thereof. In some embodiments, a DR-iTR factor is used to enhance nerve regeneration. For example, a DR-iTR factor may be infused into a severed nerve, e.g., near the proximal and/or distal stump. In some embodiments, a DR-iTR factor is placed within an artificial nerve conduit, a tube composed of biological or synthetic materials within which the nerve ends and intervening gap are enclosed. The factor or factors may be formulated in a matrix to facilitate their controlled release over time. Said matrix may comprise a biocompatible, optionally biodegradable, material, e.g., a polymer such as that comprised of hyaluronic acid, including crosslinked hyaluronic acid or carboxymethyl hyaluronate crosslinked with PEGDA, or a mixture of carboxymethyl hyaluronate crosslinked by PEGDA with carboxymethyl-modified gelatin (HyStem-C).

In some embodiments the DR-iTR factor is anti-Mullerian hormone (AMH) which may or may not be formulated for localization and slow release in carboxymethyl hyaluronate crosslinked by PEGDA with carboxymethyl-modified gelatin (HyStem-C) to induce iTR, typically at a concentration sufficient to expose cells in vitro or in vivo at a concentrations ranging from 0.05-5 mM valproic acid, preferably 1-100 ng/mL, preferably 10 ng/mL.

In some embodiments the DR-iTR factor is GFER (Augmenter of Liver Regeneration (ALR)) in either the shorter secreted form or the longer form that localizes to the mitochondrial intermembrane space which is expressed in relatively higher levels in embryonic tissue and may or may not be formulated for localization and slow release in carboxymethyl hyaluronate crosslinked by PEGDA with carboxymethyl-modified gelatin (HyStem-C) to induce iTR, typically at a concentration sufficient to expose cells in vitro or cells in tissues in vivo at a concentration ranging from 2-200 ng/mL, preferably 20 ng/mL.

In some embodiments the iTR factor is valproic acid and may or may not be formulated for localization and slow release in carboxymethyl hyaluronate crosslinked by PEGDA with carboxymethyl-modified gelatin (HyStem-C) to induce iTR, typically at a concentration sufficient to expose cells in vitro or cells in tissue in vivo at a concentration ranging from 0.05-5 mM, preferably 0.5 mM.

In some embodiments the iTR factors are administered together with formulations described herein for DR-iTR wherein said iTR factors are any combination of valproic acid at a concentration of 0.05-5 mM, preferably 0.5 mM, GFER protein (either the long or short form) at a concentration of 2-200 ng/mL, preferably 20 ng/mL and AMH protein at a concentration of 1-100 ng/mL, preferably 10 ng/mL. Said combination of the factors valproic acid, GFER, and AMH and may or may not be formulated for localization and slow release in carboxymethyl hyaluronate crosslinked by PEGDA with carboxymethyl-modified gelatin (HyStem-C) to induce iTR.

In some embodiments, tissue regeneration is augmented through the administration of prolotherapeutic agents including but not limited to hyperosmolar dextrose, glycerine, lidocaine, phenol, local anesthetic phenol, and sodium morrhuate; sclerotherapeutic agents including but not limited to those used to treat blood vessel and lymphatic malformations (vascular malformations) including Klippel Trenaunay syndrome, spider veins, smaller varicose veins, hemorrhoids and hydroceles wherein the agents used include such agents as sodium tetradecyl sulfate or polidocanol wherein the sclerosant is injected into the vessels; and platelet rich plasma-derived factors; wherein the prolotherapeutic, sclerotherapeutic or platelet rich plasma-derived factors are formulated in a matrix to localize their effects or facilitate their controlled release over time. Said matrix may comprise a biocompatible, optionally biodegradable, material, e.g., a polymer such as that comprised of hyaluronic acid, including crosslinked hyaluronic acid or carboxymethyl hyaluronate crosslinked with PEGDA, or a mixture of carboxymethyl hyaluronate crosslinked by PEGDA with carboxymethyl-modified gelatin (HyStem-C).

In some embodiments, a DR-iTR factor or combinations of factors is used to promote production of hair follicles and or growth of hair. In some embodiments, a DR-iTR factor triggers regeneration of hair follicles from epithelial cells that do not normally form hair. In some embodiments, a DR-iTR factor is used to treat hair loss, hair sparseness, partial or complete baldness in a male or female. In some embodiments, baldness is the state of having no or essentially no hair or lacking hair where it often grows, such as on the top, back, and/or sides of the head. In some embodiments, hair sparseness is the state of having less hair than normal or average or, in some embodiments, less hair than an individual had in the past or, in some embodiments, less hair than an individual considers desirable. In some embodiments, an iTR factor is used to promote growth of eyebrows or eyelashes. In some embodiments, a DR-iTR factor is used to treat androgenic alopecia or “male pattern baldness” (which can affect males and females). In some embodiments, a DR-iTR factor is used to treat alopecia areata, which involves patchy hair loss on the scalp, alopecia totalis, which involves the loss of all head hair, or alopecia universalis, which involves the loss of all hair from the head and the body. In some embodiments, a DR-iTR formulation is applied to a site where hair growth is desired, e.g., the scalp or eyebrow region. In some embodiments, a DR-iTR factor is applied to or near the edge of the eyelid, to promote eyelash growth. In some embodiments, a DR-iTR factor is applied in a liquid formulation. In some embodiments a DR-iTR factor is applied in a cream, ointment, paste, or gel. In some embodiments, a DR-iTR factor is used to enhance hair growth after a burn, surgery, chemotherapy, or other event causing loss of hair or hear-bearing skin.

In some embodiments, a DR-iTR factor or combination of factors are administered to tissues afflicted with age-related degenerative changes to regenerate youthful function. Said age-related degenerative changes includes by way of nonlimiting example, age-related macular degeneration, coronary disease, osteoporosis, osteonecrosis, heart failure, emphysema, peripheral artery disease, vocal cord atrophy, hearing loss, Alzheimer's disease, Parkinson's disease, skin ulcers, and other age-related degenerative diseases. In some embodiments, said DR-iTR factors are co administered with a vector expressing the catalytic component of telomerase to extend cell lifespan.

In some embodiments, a formulation for delivering DR-iTR to cells or tissues in vitro or in vivo including microbiopsies cultured in vitro are administered to enhance replacement of cells that have been lost or damaged due to insults such as chemotherapy, radiation, or toxins. In some embodiments such cells are stromal cells of solid organs and tissues. Inventive methods of treatment can include a step of identifying or providing a subject suffering from or at risk of a disease or condition in which in which enhancing regeneration would be of benefit to the subject. In some embodiments, the subject has experienced injury (e.g., physical trauma) or damage to a tissue or organ. In some embodiments the damage is to a limb or digit. In some embodiments, a subject suffers from a disease affecting the cardiovascular, digestive, endocrine, musculoskeletal, gastrointestinal, hepatic, integumentary, nervous, respiratory, or urinary system. In some embodiments, tissue damage is to a tissue, organ, or structure such as cartilage, bone, heart, blood vessel, esophagus, stomach, liver, gallbladder, pancreas, intestines, rectum, anus, endocrine gland, skin, hair follicle, tooth, gum, lip, nose, mouth, thymus, spleen, skeletal muscle, smooth muscle, joint, brain, spinal cord, peripheral nerve, ovary, fallopian tube, uterus, vagina, mammary gland, testes, vas deferens, seminal vesicle, prostate, penis, pharynx, larynx, trachea, bronchi, lungs, kidney, ureter, bladder, urethra, eye (e.g., retina, cornea), or ear (e.g., organ of Corti). In some embodiments, a DR-iTR microbiopsy is administered to a subject at least once within approximately 2, 4, 8, 12, 24, 48, 72, or 96 hours after a subject has suffered tissue damage (e.g., an injury or an acute disease-related event such as a myocardial infarction or stroke) and, optionally, at least once thereafter. In some embodiments a DR-iTR microbiopsy is administered to a subject at least once within approximately 1-2 weeks, 2-6 weeks, or 6-12 weeks, after a subject has suffered tissue damage and, optionally, at least once thereafter.

In some embodiments of the invention, it may useful to stimulate or facilitate regeneration or de novo development of a missing or hypoplastic tissue, organ, or structure by, for example, removing the skin, removing at least some tissue at a site where regeneration or de novo development is desired, abrading a joint or bone surface where regeneration or de novo development is desired, and/or inflicting another type of wound on a subject. In the case of regeneration after tissue damage, it may be desirable to remove (e.g., by surgical excision or debridement) at least some of the damaged tissue.

In some embodiments, a DR-iTR factor is administered at or near the site of such removal or abrasion.

In some embodiments, a formulation to generate DR-iTR in cells in vitro or in vivo or microbiopsies culture in vitro are used to enhance generation of a tissue or organ in a subject in whom such tissue or organ is at least partially absent as a result of a congenital disorder, e.g., a genetic disease. Many congenital malformations result in hypoplasia or absence of a variety of tissues, organs, or body structures such as limbs or digits. In other instances a developmental disorder resulting in hypoplasia of a tissue, organ, or other body structure becomes evident after birth. In some embodiments, a DR-iTR microbiopsy is administered to a subject suffering from hypoplasia or absence of a tissue, organ, or other body structure, in order to stimulate growth or development of such tissue, organ, or other body structure. In some aspects, the invention provides a method of enhancing generation of a tissue, organ, or other body structure in a subject suffering from hypoplasia or congenital absence of such tissue, organ, or other body structure, the method comprising administering a DR-iTR microbiopsy to the subject. In some embodiments, a DR-iTR microbiopsy is administered to the subject prior to birth, i.e., in utero. The various aspects and embodiments of the invention described herein with respect to regeneration are applicable to such de novo generation of a tissue, organ, or other body structure and are encompassed within the invention.

In some aspects, a DR-iTR microbiopsy is used to enhance generation of tissue in any of a variety of situations in which new tissue growth is useful at locations where such tissue did not previously exist. For example, generating bone tissue between joints is frequently useful in the context of fusion of spinal or other joints. iTR microbiopsies may be tested in a variety of animal models of regeneration. In one aspect, a iTR microbiopsies are tested in murine species. For example, mice can be wounded (e.g., by incision, amputation, transection, or removal of a tissue fragment). A DR-iTR microbiopsy is applied to the site of the wound and/or to a removed tissue fragment and its effect on regeneration is assessed.

The effect of a modulator of vertebrate TR can be tested in a variety of vertebrate models for tissue or organ regeneration. For example, fin regeneration can be assessed in zebrafish, e.g., as described in (Mathew L K, Unraveling tissue regeneration pathways using chemical genetics. J Biol Chem. 282(48):35202-10 (2007)), and can serve as a model for limb regeneration. Rodent, canine, equine, caprine, fish, amphibian, and other animal models useful for testing the effects of treatment on regeneration of tissues and organs such as heart, lung, limbs, skeletal muscle, bone, etc., are widely available. For example, various animal models for musculoskeletal regeneration are discussed in Tissue Eng Part B Rev. 16(1) (2010). A commonly used animal model for the study of liver regeneration involves surgical removal of a larger portion of the rodent liver. Other models for liver regeneration include acute or chronic liver injury or liver failure caused by toxins such as carbon tetrachloride. In some embodiments, a model for hair regeneration or healing of skin wounds involves excising a patch of skin, e.g., from a mouse. Regeneration of hair follicles, hair growth, re-epithelialization, gland formation, etc., can be assessed.

The compounds and compositions disclosed herein and/or identified using a method and/or assay system described herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or by inhalation, e.g., as an aerosol. The particular mode selected will depend, of course, upon the particular compound selected, the particular condition being treated and the dosage required for therapeutic efficacy. The methods of this invention, generally speaking, may be practiced using any mode of administration that is medically or veterinarily acceptable, meaning any mode that produces acceptable levels of efficacy without causing clinically unacceptable (e.g., medically or veterinarily unacceptable) adverse effects. Suitable preparations, e.g., substantially pure preparations, of one or more compound(s) may be combined with one or more pharmaceutically acceptable carriers or excipients, etc., to produce an appropriate pharmaceutical composition suitable for administration to a subject. Such pharmaceutically acceptable compositions are an aspect of the invention. The term “pharmaceutically acceptable carrier or excipient” refers to a carrier (which term encompasses carriers, media, diluents, solvents, vehicles, etc.) or excipient which does not significantly interfere with the biological activity or effectiveness of the active ingredient(s) of a composition and which is not excessively toxic to the host at the concentrations at which it is used or administered. Other pharmaceutically acceptable ingredients can be present in the composition as well. Suitable substances and their use for the formulation of pharmaceutically active compounds are well-known in the art (see, for example, “Remington's Pharmaceutical Sciences”, E. W. Martin, 19th Ed., 1995, Mack Publishing Co.: Easton, Pa., and more recent editions or versions thereof, such as Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types). Furthermore, compounds and compositions of the invention may be used in combination with any compound or composition used in the art for treatment of a particular disease or condition of interest.

In some embodiments, LIN28B is exogenously expressed in blood cell types including CD34+ hematopoietic cells to promote their proliferation and engraftment into bone marrow in vivo comparable to the proliferative and engraftment capacity of their fetal liver-derived counterparts.

A pharmaceutical composition is typically formulated to be compatible with its intended route of administration. For example, preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's. Examples of non-aqueous solvents are propylene glycol, polyethylene glycol, vegetable oils such as olive oil, and injectable organic esters such as ethyl oleate. fixed oils, polyethylene glycols, glycerine, propylene glycol or other synthetic solvents; preservatives, e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. pH can be adjusted with acids or bases, such as hydrochloric acid or sodium hydroxide. Such parenteral preparations can be enclosed in ampoules, disposable syringes or multiple dose vials made of glass or plastic.

For oral administration, compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like. Suitable excipients for oral dosage forms are, e.g., fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP).

For administration by inhalation, inventive compositions may be delivered in the form of an aerosol spray from a pressured container or dispenser which contains a suitable propellant, e.g., a gas such as carbon dioxide, a fluorocarbon, or a nebulizer. Liquid or dry aerosol (e.g., dry powders, large porous particles, etc.) can be used. The present invention also contemplates delivery of compositions using a nasal spray or other forms of nasal administration. For topical applications, pharmaceutical compositions may be formulated in a suitable ointment, lotion, gel, or cream containing the active components suspended or dissolved in one or more pharmaceutically acceptable carriers suitable for use in such composition. For local delivery to the eye, the pharmaceutically acceptable compositions may be formulated as solutions or micronized suspensions in isotonic, pH adjusted sterile saline, e.g., for use in eye drops, or in an ointment, or for intra-ocularly administration, e.g., by injection. Pharmaceutical compositions may be formulated for transmucosal or transdermal delivery. For transmucosal or transdermal administration, penetrants appropriate to the barrier to be permeated may be used in the formulation. Such penetrants are generally known in the art. Inventive pharmaceutical compositions may be formulated as suppositories (e.g., with conventional suppository bases such as cocoa butter and other glycerides) or as retention enemas for rectal delivery.

In some embodiments, a composition includes one or more agents intended to protect the active agent(s) against rapid elimination from the body, such as a controlled release formulation, implants, microencapsulated delivery system, etc. Compositions may incorporate agents to improve stability (e.g., in the gastrointestinal tract or bloodstream) and/or to enhance absorption. Compounds may be encapsulated or incorporated into particles, e.g., microparticles or nanoparticles. Biodegradable, biocompatible polymers can be used, such as ethylene vinyl acetate, polyanhydrides, polyglycolic acid, PLGA, collagen, polyorthoesters, polyethers, and polylactic acid. Methods for preparation of such formulations will be apparent to those skilled in the art. For example, and without limitation, a number of particle, lipid, and/or polymer-based delivery systems are known in the art for delivery of siRNA. The invention contemplates use of such compositions. Liposomes or other lipid-based particles can also be used as pharmaceutically acceptable carriers.

Pharmaceutical compositions and compounds for use in such compositions may be manufactured under conditions that meet standards, criteria, or guidelines prescribed by a regulatory agency. For example, such compositions and compounds may be manufactured according to Good Manufacturing Practices (GMP) and/or subjected to quality control procedures appropriate for pharmaceutical agents to be administered to humans and can be provided with a label approved by a government regulatory agency responsible for regulating pharmaceutical, surgical, or other therapeutically useful products.

Pharmaceutical compositions of the invention, when administered to a subject for treatment purposes, are preferably administered for a time and in an amount sufficient to treat the disease or condition for which they are administered. Therapeutic efficacy and toxicity of active agents can be assessed by standard pharmaceutical procedures in cell cultures or experimental animals. The data obtained from cell culture assays and animal studies can be used in formulating a range of dosages suitable for use in humans or other subjects. Different doses for human administration can be further tested in clinical trials in humans as known in the art. The dose used may be the maximum tolerated dose or a lower dose. Those of ordinary skill in the art will appreciate that appropriate doses in any particular circumstance depend upon the potency of the agent(s) utilized, and may optionally be tailored to the particular recipient. The specific dose level for a subject may depend upon a variety of factors including the activity of the specific agent(s) employed, the particular disease or condition and its severity, the age, body weight, general health of the subject, etc. It may be desirable to formulate pharmaceutical compositions, particularly those for oral or parenteral compositions, in unit dosage form for ease of administration and uniformity of dosage. Unit dosage form, as that term is used herein, refers to physically discrete units suited as unitary dosages for the subject to be treated; each unit containing a predetermined quantity of active agent(s) calculated to produce the desired therapeutic effect in association with an appropriate pharmaceutically acceptable carrier. It will be understood that a therapeutic regimen may include administration of multiple doses, e.g., unit dosage forms, over a period of time, which can extend over days, weeks, months, or years. A subject may receive one or more doses a day, or may receive doses every other day or less frequently, within a treatment period. For example, administration may be biweekly, weekly, etc. Administration may continue, for example, until appropriate structure and/or function of a tissue or organ has been at least partially restored and/or until continued administration of the compound does not appear to promote further regeneration or improvement. In some embodiments, a subject administers one or more doses of a composition of the invention to him or herself.

In some embodiments, two or more DR-iTR microbiopsies or compositions are administered in combination, e.g., for purposes of enhancing regeneration. Compounds or compositions administered in combination may be administered together in the same composition, or separately. In some embodiments, administration “in combination” means, with respect to administration of first and second compounds or compositions, administration performed such that (i) a dose of the second compound is administered before more than 90% of the most recently administered dose of the first agent has been metabolized to an inactive form or excreted from the body; or (ii) doses of the first and second compound are administered within 48, 72, 96, 120, or 168 hours of each other, or (iii) the agents are administered during overlapping time periods (e.g., by continuous or intermittent infusion); or (iv) any combination of the foregoing. In some embodiments, two or more iTR factors, or vectors expressing the catalytic component of telomerase and an iTR factor, are administered. In some embodiments a DR-iTR microbiopsy is administered in combination with a combination with one or more growth factors, growth factor receptor ligands (e.g., agonists), hormones (e.g., steroid or peptide hormones), or signaling molecules, useful to promote regeneration and polarity. Of particular utility are organizing center molecules useful in organizing regeneration competent cells such as those produced using the methods of the present invention. In some embodiments, a growth factor is an epidermal growth factor family member (e.g., EGF, a neuregulin), a fibroblast growth factor (e.g., any of FGF1-FGF23), a hepatocyte growth factor (HGF), a nerve growth factor, a bone morphogenetic protein (e.g., any of BMP1-BMP7), a vascular endothelial growth factor (VEGF), a wnt ligand, a wnt antagonist, retinoic acid, NOTUM, follistatin, sonic hedgehog, or other organizing center factors. Those skilled in the art will recognize, or be able to ascertain using no more than routine experimentation, many equivalents to the specific embodiments of the invention described herein.

The scope of the present invention is not intended to be limited to the Description or the details set forth therein. Articles such as “a”, “an” and “the” may mean one or more than one unless indicated to the contrary or otherwise evident from the context. Certain of the inventive methods are often practiced using populations of cells, e.g., in vitro or in vivo. Thus references to “a cell” should be understood as including embodiments in which the cell is a member of a population of cells, e.g., a population comprising or consisting of cells that are substantially genetically identical. However, the invention encompasses embodiments in which inventive methods is/are applied to an individual cell. Thus, references to “cells” should be understood as including embodiments applicable to individual cells within a population of cells and embodiments applicable to individual isolated cells.

Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. It is contemplated that all embodiments described herein are applicable to all different aspects of the invention. It is also contemplated that any of the embodiments can be freely combined with one or more other such embodiments whenever appropriate. Furthermore, it is to be understood that the invention encompasses all variations, combinations, and permutations in which one or more limitations, elements, clauses, descriptive terms, etc., from one or more of the claims (whether original or subsequently added claims) is introduced into another claim (whether original or subsequently added). For example, any claim that is dependent on another claim can be modified to include one or more elements or limitations found in any other claim that is dependent on the same base claim, and any claim that refers to an element present in a different claim can be modified to include one or more elements or limitations found in any other claim that is dependent on the same base claim as such claim. Furthermore, where the claims recite a composition, the invention provides methods of making the composition, e.g., according to methods disclosed herein, and methods of using the composition, e.g., for purposes disclosed herein. Where the claims recite a method, the invention provides compositions suitable for performing the method, and methods of making the composition. Also, where the claims recite a method of making a composition, the invention provides compositions made according to the inventive methods and methods of using the composition, unless otherwise indicated or unless one of ordinary skill in the art would recognize that a contradiction or inconsistency would arise. Where elements are presented as lists, e.g., in Markush group format, each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where phrases such as “less than X”, “greater than X”, or “at least X” is used (where X is a number or percentage), it should be understood that any reasonable value can be selected as the lower or upper limit of the range. It is also understood that where a list of numerical values is stated herein (whether or not prefaced by “at least”), the invention includes embodiments that relate to any intervening value or range defined by any two values in the list, and that the lowest value may be taken as a minimum and the greatest value may be taken as a maximum. Furthermore, where a list of numbers, e.g., percentages, is prefaced by “at least”, the term applies to each number in the list. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% or in some embodiments 10% of a number in either direction (greater than or less than the number) unless otherwise stated or otherwise evident from the context (e.g., where such number would impermissibly exceed 100% of a possible value). A “composition” as used herein, can include one or more than one component unless otherwise indicated. For example, a “composition comprising an activator or a TR activator” can consist or consist essentially of an activator of a TR activator or can contain one or more additional components. It should be understood that, unless otherwise indicated, an inhibitor or a TR inhibitor (or other compound referred to herein) in any embodiment of the invention may be used or administered in a composition that comprises one or more additional components including the presence of an activator of a TR activator.

### Novel Cancer Therapeutic Strategies

The methods and compositions of the present invention also provide for novel cancer therapeutics and companion diagnostics. The present invention teaches that certain molecular pathways associated with the EFT evolved in part as a method to restrain the replication of endogenous transposable elements and viruses including Class I transposable elements (retrotransposons), Class II transposable elements (DNA transposons), LINES, SINES, as well as other viruses such as retroviruses. Prior to the EFT and in mammalian pre-implantation embryos, some cells, such as cells of the inner cell mass or cells isolated from the inner cell mass such as cultured hES cells, are permissive for viral replication. The relative permissivity of some embryonic (pre-fetal) cells to endogenous transposable element replication is known in the art. For example, it is documented that human endogenous retroviruses such as HERVK replicate in some pluripotent stem cell lines (Grow, E. J. et al, (2015) Nature 522:221-225). However, the association of Lamin-A with the EFT and the suppression of viral replication has not been described.

The present invention teaches that lamin-A, in particular, its processing into mature filaments and association with LRRK2 and PLPP7 evolved as a means of guarding the integrity of the genome, in particular, regions of repetitive sequences such as those associated with telomeric repeats and tandemly-repeated paralogs such as those of the clustered protocadherin locus or regions of tandemly-repeated paralogs of zinc finger proteins that evolved to inactivate diverse viral sequences. In addition, Lamin A evolved as a means of limiting the plasticity of diverse differentiated somatic types, that is, stabilizing them in their differentiated state. In limiting their plasticity, it limited the potential of diverse somatic cell types and tissues to regenerate after injury or disease by utilizing diverse pathways. These pathways included the downregulation of the embryonic cell-cell recognition system of the clustered protocadherin locus (See, e.g. U.S. provisional patent application No. 63/155,631, filed Mar. 2, 2021, the disclosure of which is incorporated by reference in its entirety) and increased signaling associated with the epithelial-mesenchymal transformation (EMT) such as increased expression of extracellular matrix proteins such as those encoded by the genes: FN1, COL1A1, SPARC, and VIM that result in a fibrotic scarring of adult tissue in lieu of regeneration as seen in embryonic tissue following injury. As a result, Lamin A plays an important regulatory role as an inhibitor of tissue regeneration (See, e.g. U.S. provisional patent application No. 63/155,628, filed Mar. 2, 2021, the disclosure of which is incorporated by reference in its entirety), but also the formation of cancer stem cells (CSC) which have been disclosed to be not a more undifferentiated cell type as is the current consensus belief, but rather a more mature cell type corresponding to fetal/adult cells, as opposed to the embryonic (pre-fetal) state of many malignant cell types from diverse somatic cell origins.

### Oncolytic Viral Therapy

The permissive state of pre-EFT somatic cells therefore is consistent with the permissive replication of diverse viruses in cancer cells. While there are currently no efficient means of determining in advance which tumors or cancer cells types will be efficiently destroyed by said vectors, the embryonic and adult gene expression markers in previously disclosures (See, e.g. U.S. provisional patent application No. 61/831,421, filed Jun. 5, 2013, PCT patent application PCT/US2014/040601, filed Jun. 3, 2014 and U.S. Pat. No. 10,961,531, filed on Dec. 7, 2015, PCT patent application PCT/US2017/036452, filed Jun. 7, 2017 and U.S. patent application Ser. No. 16/211,690, filed on Dec. 6, 2018, and U.S. provisional patent application No. 63/256,286, filed Oct. 15, 2021, the disclosures of which are incorporated by reference in their entirety), as well as the differentially-methylated DNA sequences associated with embryonic vs fetal/adult cells (see, e.g. PCT patent application PCT/US2020/047707, filed Aug. 25, 2020, the disclosure of which is incorporated by reference in its entirety), provide useful means of determining which cancer cells or tumors will respond to oncolytic viral therapy. Cancer cells or tumors that express embryonic (pre-fetal) markers such as a lack of COX7A1 expression, relatively low expression of LMNA, or alternatively express embryonic (pre-fetal) markers such as the expression of PCAT7, are permissive for the replication of viruses and are therefore sensitive to oncolytic viral therapy. In addition, methods of inducing tissue regeneration such as those disclosed in (See, e.g. U.S. provisional patent application No. 61/831,421, filed Jun. 5, 2013, PCT patent application PCT/US2014/040601, filed Jun. 3, 2014 and U.S. Pat. No. 10,961,531, filed on Dec. 7, 2015, e.g. PCT patent application PCT/US2017/036452, filed Jun. 7, 2017 and U.S. patent application Ser. No. 16/211,690, filed on Dec. 6, 2018, U.S. provisional patent application No. 63/155,628, filed Mar. 2, 2021, and U.S. provisional patent application No. 63/256,286, filed Oct. 15, 2021, the disclosures of which are incorporated by reference in their entirety) are useful in transforming CSCs into their embryonic counterparts wherein the cancer cells will be responsive to oncolytic viral therapy.

The novel oncolytic viral therapies of the present invention include the use of viruses currently-disclosed as selectively destroying malignant cancer cells including: Herpes Simplex Virus Type I (HSV-1) such as Talimogene laherparepvec (T-VEC) modified to express GM-CSF with a promoter of an embryonic (pre-fetal) gene promoter such as the PCAT7, CPT1B, or PURPL promoters or other embryonic promoters previously disclosed herein.

In addition, viruses useful in targeting cancer cells such as HSV-1, reovirus, picornaviruses (coxsackeievirus, rigavirus) rhabdoviruses such as vesicular stomatitis virus and Maraba virus, and paramyxoviruses such as Newcastle disease virus and Measles virus, and vaccinia virus may be modified to express toxic gene products or genes useful to express specifically in cancer cells such as GM-CSF that are useful in promoting dendritic cell activation wherein said introduced genes are expressed from a gene promoter such as the PCAT7, CPT1B, or PURPL promoters or other embryonic promoters previously disclosed herein.

In addition, viruses useful in targeting cancer cells such as HSV-1, reovirus, picornaviruses (coxsackeievirus, rigavirus) rhabdoviruses such as vesicular stomatitis virus and Maraba virus, and paramyxoviruses such as Newcastle disease virus and Measles virus, and vaccinia virus may be modified to express RNAi to zinc finger protein genes that are activated in fetal/adult cells wherein said zinc finger proteins inhibit viral replication. As a result, infected cells, such as cancer cells with an fetal/adult-like phenotype are rendered more susceptible to lysis. Said fetal/adult-onset zinc finger genes activated by Lamin A include: ZNF280D (See, e.g. U.S. provisional patent application No. 61/831,421, filed Jun. 5, 2013, PCT patent application PCT/US2014/040601, filed Jun. 3, 2014 and U.S. Pat. No. 10,961,531, filed on Dec. 7, 2015, the disclosures of which are incorporated by reference in their entirety), ZNF300P1, ZNF-572 (See, e.g. PCT patent application PCT/US2017/036452, filed Jun. 7, 2017 and U.S. patent application Ser. No. 16/211,690, filed on Dec. 6, 2018, the disclosures of which are incorporated by reference in their entirety), and ZNF578, ZNF585B, ZNF736, and ZNF790-AS1 (See, e.g. U.S. provisional patent application No. 63/256,286, filed Oct. 15, 2021, the disclosure of which is incorporated by reference in its entirety).

In addition, the present invention provides for novel oncolytic viral therapy which when used alone or in combination with immune checkpoint inhibition, or adoptive immunotherapy, are useful in selectively destroying cancer cells with an embryonic phenotype. Numerous immune checkpoint inhibitors useful in treating cancer are known in the art and may be utilized as a combination therapy with the cancer therapeutics described herein. Nonlimiting examples of immune checkpoint inhibitors antibodies targeting PD-1 such as Nivolumab, Cemiplimab, Spartalizumab, and Pembrolizumab and antibodies targeting PD-L1 such as Atezolizumab, Avelumab, and Durvalumab, and antibodies targeting CTLA4 such as Ipilimumab. Additional immune checkpoint inhibition can be achieved by T-Cell Adoptive Cancer Immunotherapy. Said T-Cells are used wherein they express decreased levels of or have a knock-out of CISH (cytokine-inducible SH2-containing protein) or CBLB (Cbl Proto-oncogene, E3 Ubiquitin Protein Ligase B).

Additional combinations that are useful in achieving greater levels of reduction in tumor burden can be achieved by combining the oncolytic viruses of the present invention with the above mentioned immune checkpoint inhibitors, together with dendritic cell therapy and/or CAR-T cells targeting embryonic (pre-fetal) antigens such as those described in (See, e.g. U.S. provisional patent application No. 63/155,631, filed Mar. 2, 2021, the disclosure of which is incorporated by reference in its entirety).

The phenotypic alterations of the EFT are shared in common with the majority of all somatic cell types. Similarly, the abnormal embryonic phenotype (embryo-onco phenotype) of many cancer cells and the fetal/adult phenotype of CSCs are shared by many cancer types (i.e. are pan-cancer phenotypic alterations). They are useful in the diagnosis of primary and metastatic cancers including: Acanthoma, Acinar adenocarcinoma, Acinic cell carcinoma, Acrospiroma, Acute eosinophilic leukemia, Acute erythroid leukemia, Acute Lymphoblastic Leukemia (ALL), Acute megakaryoblastic leukemia, Acute monocytic leukemia, Acute Myeloid Leukemia (AML), Acute promyelocytic leukemia, Adamantinoma, Adenoid cystic carcinoma, Adenomatoid odontogenic tumor, Adenosquamous carcinoma, Adenosquamous lung carcinoma, Adipose tissue neoplasm, Adrenocortical carcinoma, Adrenocortical carcinoma childhood, Aggressive NK-cell leukemia, AIDS-related cancers, Alveolar rhabdomyosarcoma, Alveolar soft part sarcoma, Ameloblastic fibroma, Anal cancer, Anaplastic carcinoma, Anaplastic large-cell lymphoma, Anaplastic thyroid cancer, Angioimmunoblastic T-cell lymphoma, Angiosarcoma, Appendix cancer, Attenuated familial adenomatous polyposis, Atypical teratoid/rhabdoid tumor central nervous system childhood, B-cell chronic lymphocytic leukemia, B-cell lymphoma, Bellini duct carcinoma, Bile duct cancer, Bile duct cancer—Cholangiocarcinoma, Bladder cancer, Bladder cancer—Small cell carcinoma, Bladder cancer—Transitional cell carcinoma, Bladder cancer childhood, Blastoma, Bone cancer, Bone cancer—Osteosarcoma, Brain stem glioma, Brain tumors—other, Brain tumor—Glioblastoma multiforme, Brain tumor—Oligodendroglioma anaplastic, Brain tumor—cerebellar astrocytoma (childhood & adult), Brain tumor—cerebral astrocytoma/malignant glioma (childhood & adult), Brain tumor—ependymoma, Brain tumor—medulloblastoma, Brain tumor—supratentorial primitive neuroectodermal tumors, Brain tumor—visual pathway and hypothalamic glioma, Brain and spinal cord tumors childhood, Breast cancer, Breast cancer ductal adenocarcinoma, Breast cancer childhood, Brenner tumour, Bronchial adenomas/carcinoids, Bronchial tumors, Bronchial tumors childhood, Bronchioloalveolar carcinoma, Brown tumor, Burkitt lymphoma, Carcinoid tumor, Carcinoid tumor childhood, Carcinoid tumor gastrointestinal, Carcinoma of the penis, Carcinosarcoma, Cementoma, Central nervous system cancer, Cervical cancer—adenocarcinoma, Cervical cancer—squamous cell, Cervical Cancer—Neuroendocrine, Carcinoma of the cervix, Cervical cancer childhood, Childhood cancers, Childhood leukemia, Cholangiocarcinoma, Cholangiosarcoma, Chondromyxoid fibroma, Chondrosarcoma, Chordoma, Chorioadenoma destruens, Chorioblastoma, Choriocarcinoma, Choroid plexus tumor, Chorioepithelioma, Clear cell adenocarcinoma, Clear cell adenocarcinoma of the vagina, Clear-cell ovarian carcinoma, Clear-cell sarcoma of the kidney, Colon cancer, Colon cancer—adenocarcinoma, Colorectal cancer, Colorectal cancer childhood, Comedocarcinoma, Craniopharyngioma, Craniopharyngioma childhood, Cutaneous lymphoma, Cystadenocarcinoma, Degos disease, Dermatofibrosarcoma protuberans, Desmoplastic small round cell tumor, Diffuse large B-cell lymphoma, Digestive system neoplasm, Diktyoma, Ductal carcinoma In situ (DCIS), “Ductal, lobular, and medullary neoplasms”, Duodenal cancer, Dysembryoplastic neuroepithelial tumour, Dysgerminoma, ELM4-ALK positive lung cancer, Embryoma, Embryonal carcinoma, Embryonal rhabdomyosarcoma, Embryonal tumors central nervous system childhood, Endocrine gland neoplasm, Endodermal sinus tumor, Endometrial cancer, Endometrial—Stromal sarcoma, Endometrial—Adenocarcinoma, Endometrioid tumor, Enteropathy-associated T-cell lymphoma, Ependymoblastoma childhood, Ependymoma childhood, Epithelial-myoepithelial carcinoma of the lung, Epithelioid sarcoma, Epithelioma, Esophageal cancer, Esophageal cancer childhood, Esthesioneuroblastoma childhood, Ewing family of tumors, Ewing's sarcoma in the Ewing family of tumors, Exocrine cancer, Extracranial germ cell tumor childhood, Extragonadal germ cell tumor, Extrahepatic bile duct cancer, Extramammary Paget's disease, Eye cancer, “Eye cancer, intraocular melanoma”, “Eye cancer, retinoblastoma”, Fallopian tube cancer, Familial adenomatous polyposis, Fetal adenocarcinoma, Fibroepithelial neoplasms, Fibrolamellar hepatocellular carcinoma, Fibrosarcoma, Fibrous tissue neoplasm, Follicular lymphoma, Follicular thyroid cancer, GCB Diffuse Large B-Cell Lymphoma (DLBCL), Gallbladder cancer, Ganglioglioma, Ganglioneuroma, Gardner's syndrome, Gastric carcinoid, Gastric (stomach) cancer, Gastric (stomach) cancer—Adenocarcinoma, Gastric (stomach) cancer—Adenocarcinoma of gastroesophageal junction, Gastric (stomach) cancer childhood, Gastric lymphoma, Gastrinoma, Gastrointestinal carcinoid tumor, Gastrointestinal stromal tumors (GIST), Germ cell tumor, Extragonadal germ cell tumor, Ovarian germ cell tumor, Germinoma, Gestational choriocarcinoma, Gestational trophoblastic tumor, Giant-cell fibroblastoma, Giant-cell glioblastoma, Giant-cell tumor of bone, Gigantiform cementoma, Glial tumor, Gliomatosis cerebri, Glioblastoma Multiforme, Glioma, Glioma childhood visual pathway and hypothalamic, Gliosarcoma, Glucagonoma, Goblet cell carcinoid, Gonadoblastoma, Granulosa cell tumour, Gynandroblastoma, Head and neck cancer, Head and neck cancer childhood, Heart cancer, Hemangioblastoma, Hemangiopericytoma, Hemangiosarcoma, Hematological malignancy, Hepatic cancer—Cholangiocarcinoma, Hepatoblastoma, Hepatocellular (liver) cancer, Hepatosplenic T-cell lymphoma, Hereditary breast-ovarian cancer syndrome, Hereditary nonpolyposis colorectal cancer, Histiocytic sarcoma, Histiocytoma, Hypopharyngeal cancer, Inflammatory breast cancer, Inflammatory myeloblastic tumor, Intraductal carcinoma, Intraductal papillary mucinous neoplasm, Intraocular melanoma, Intratubular germ cell neoplasia, Invasive lobular carcinoma, Islet cell carcinoma, Islet cell tumors (endocrine pancreas), Juvenile granulosa cell tumor, Juvenile myelomonocytic leukemia, Juxtaglomerular cell tumor, Kaposi sarcoma, Kidney cancer childhood, Klatskin tumor, Krukenberg tumor, Langerhans cell histiocytosis, Large-cell lung carcinoma with rhabdoid phenotype, Laryngeal cancer, Laryngeal cancer—squamous cell carcinoma, Laryngeal cancer childhood, Leiomyosarcoma, Lentigo malignant melanoma, Leptomeningeal cancer, Leukemias, Leydig cell tumour, Chronic lymphocytic leukemia (CLL), Chronic myelogenous leukemia (CML), Hairy cell leukemia, Linitis plastica, Lip and oral cavity cancer, Liposarcoma, Liver cancer (primary), Lobular carcinoma, Lobular carcinoma In situ (LCIS), Giant-cell carcinoma of the lung, Large-cell lung carcinoma, Large-cell lung carcinoma with rhabdoid phenotype, Non-small cell lung cancer, Lung—Adenocarcinoma, Lung—Large cell_carcinoma, Lung—Small cell_carcinoma, Lung—Squamous cell_carcinoma, Luteoma, Lymphangioma, Lymphangiosarcoma, Lymphoepithelioma, Lymphomas, Lymphoma—Extranodal marginal zone B-cell of lymphoid tissue, Lymphoma—Follicular cancer of lymphoid tissue, AIDS-related_lymphoma, Cutaneous T-cell lymphoma, Hodgkin_lymphoma, Non-hodgkin lymphoma, Primary central nervous system lymphoma (CNS), Macroglobulinemia Waldenstrim, Male breast cancer, Malignant fibrous histiocytoma of bone and osteosarcoma, Malignant peripheral nerve sheath tumor, Malignant triton tumor, MALT lymphoma, Mammary ductal carcinoma, Mantle cell lymphoma, Marginal zone B-cell lymphoma, “Marcus Whittle, deadly disease”, Mast cell leukemia, Mediastinal germ cell tumor, Mediastinal tumor, Medullary carcinoma, Medullary carcinoma of the breast, Medullary thyroid cancer, Medulloblastoma, Medulloblastoma childhood, Medulloepithelioma, Medulloepithelioma childhood, Melanoma, Melanoma childhood, Meningioma, Merkel cell carcinoma, Mesenchymal chondrosarcoma, Mesothelioma adult malignant, Mesothelioma adult malignant—pleural mixed, Mesothelioma childhood, Metastatic breast cancer, Metastatic squamous neck cancer with occult primary, Metastatic tumor of jaws, Metastatic urothelial carcinoma, Mixed Mullerian tumor, Mouth cancer, Mucinous cystadenocarcinoma of the lung, Mucinous tumor, Multiple endocrine neoplasia syndromes childhood, Multiple endocrine neoplasia type 2b, Multiple myeloma/plasma cell neoplasm, Muscle tissue neoplasm, Mycosis fungoides, Myelodysplastic/myeloproliferative neoplasms, Myelodysplastic syndromes, Myeloid leukemia adult acute, Myeloid leukemia childhood acute, Myeloid sarcoma, Chronic myeloproliferative disorders, Myosarcoma, Myxoid chondrosarcoma, Myxoid liposarcoma, Myxoma, Myxosarcoma, Nasal cavity and paranasal sinus cancer, Nasopharyngeal angiofibroma, Nasopharyngeal cancer, Nasopharyngeal cancer childhood, Nerve sheath tumor, Nervous system neoplasm, Neuroblastoma, Neurocytoma, Neurofibroma, Neuroma, Nipple adenoma, Nodular lymphocyte predominant Hodgkin's lymphoma, Nodular melanoma, Odontogenic tumor, Oncocytoma, Optic nerve sheath meningioma, Optic nerve tumor, Oral cancer, Oral cancer childhood, Oropharyngeal cancer, Oropharyngeal squamous cell carcinomas, Osteolipochondroma, Osteoma, Osteosarcoma, Ovarian cancer, Ovarian cancer—Adenocarcinoma of ovary serous, Ovarian cancer childhood, Ovarian cancer epithelial, Ovarian cancer germ cell tumor, Paget's disease of the breast, Pancoast tumor, Pancreatic cancer, Pancreatic cancer childhood, Pancreatic cancer—Neuroendocrine, Pancreatic cancer islet cell tumors, Pancreatic—Adenocarcinoma of pancreas ductal, Pancreatic serous cystadenoma, Papillary adenocarcinoma, Papillary serous cystadenocarcinoma, Papillary thyroid cancer, Papillomatosis childhood, Paraganglioma, Parathyroid adenoma, Parathyroid cancer, Parathyroid neoplasm, PEComa, Periampullary cancer, Peritoneal mesothelioma, Pharyngeal Cancer, Pheochromocytoma, Pineal astrocytoma, Pineal germinoma, Pineal parenchymal tumors of intermediate differentiation childhood, Pinealoblastoma, Pineoblastoma and supratentorial primitive neuroectodermal tumors childhood, Pineocytoma, Pituicytoma, Pituitary adenoma, Pituitary tumor, Plasma cell dyscrasia, Plasma cell leukemia, Plasma cell neoplasm/multiple myeloma, Plasmacytoma, Pleomorphic undifferentiated sarcoma, Pleomorphic xanthoastrocytoma, Pleuropulmonary blastoma, Pleuropulmonary blastoma childhood, Polyembryoma, Posterior urethral cancer, Precursor T-lymphoblastic lymphoma, Primary peritoneal carcinoma, Primitive neuroectodermal tumor, Prostate cancer, Prostate cancer—adenocarcinoma, Pseudomyxoma peritonei, Rectal cancer, Rectal cancer—adenocarcinoma, Renal cell carcinoma (kidney cancer), Renal medullary carcinoma, Renal pelvis and ureter transitional cell cancer, Reninoma, Respiratory tract neoplasm, Retinoblastoma, Rhabdomycin, Rhabdomyosarcoma childhood, Richter's transformation, Salivary gland cancer, Salivary gland cancer childhood, Salivary gland-like carcinoma of the lung, Salivary gland neoplasm, Sacrococcygeal teratoma, Sarcoma, Sarcoma botryoides, Sarcoma soft tissue, Sarcomatoid carcinoma, Schwannomatosis, Sclerosing rhabdomyosarcoma, Secondary neoplasm, Seminoma, Serous carcinoma, Serous cystadenocarcinoma, Serous tumour, Sertoli cell tumour, Sertoli-Leydig cell tumour, Sex cord-gonadal stromal tumour, SCzary syndrome, Signet ring cell carcinoma, Skin cancer, Skin cancer childhood, Skin cancer—basal cell carcinoma, Skin cancer—basal-like carcinoma, Skin cancer—melanoma, Small-cell carcinoma, Small intestine cancer, “Small-, round-, blue-cell tumour”, Somatostatinoma, Soot wart, Spermatocytic seminoma, Spinal tumor, Spindle cell cancer, Spindle cell rhabdomyosarcoma, Splenic lymphoma with villous lymphocytes, Splenic marginal zone lymphoma, Squamous cell carcinoma, Squamous neck cancer with occult primary metastatic, Stewart_Treves syndrome, Stromal tumor, Supratentorial primitive neuroectodermal tumors childhood, Surface epithelial-stromal tumor, Synovial sarcoma, T-cell lymphoma, T-lymphoblastic lymphoma, Teratocarcinoma, Testicular cancer, Testicular cancer—Seminoma, Testicular cancer childhood, Thecoma, Throat cancer, “Thymoma, childhood”, Thymoma and thymic carcinoma, Thymoma and thymic carcinoma childhood, Thyroid cancer, Thyroid cancer—follicular, Thyroid cancer—papillary, Thyroid cancer childhood, Tonsil—Carcinoma of tonsil squamous cell, Trabecular cancer, Tracheal tumor, Transitional cell carcinoma, Trophoblastic tumor gestational, Tubulovillous adenoma, Urachal cancer, Ureteral cancer, Ureteral neoplasm, Urethral cancer, Urogenital neoplasm, Urothelial carcinoma, Urothelial cell carcinoma, Uterine cancer, Uterine cancer endometrial, Uterine clear cell carcinoma, Uterine sarcoma, Uterine serous carcinoma, Uveal melanoma, Vaginal cancer, Vaginal cancer childhood, Verrucous carcinoma, Vestibular schwannoma, VIPoma, Visual pathway glioma, Von Hippel_Lindau disease, Vulvar Cancer, “Wilms tumor (kidney cancer), childhood”

### EXAMPLES

### Example 1. Ex Vivo Induction of Developmentally-Regulated Tissue Regeneration in Microbiopsies

Microbiopsies are obtained from the medial aspect of the upper arm of a human utilizing a hollow tube with a diameter of 0.5 mm and cultured in non-adherent culture plates in DMEM medium supplemented with 5% human serum at 37 deg C. at ambient oxygen tension. Prior to administration of the iTR factors, accessibility of the cells within the microbiopsy is increased by transient digestion of hyaluronic acid and/or interstitial collagen by treatment with hyaluronidase and/or collagenase respectively. Briefly, DMEM media supplemented with 10 ml/gram of tissue collagenase and 0.5 mg/ml hyaluronidase (50 units/ml) is prepared supplemented with 2% human serum. Tissue is cultured on a rocking platform in non-adherent culture vessels at 37 deg C. at ambient oxygen tension for approximately 2-14 hours, preferably 4 hours. The site of the biopsy and the variable nature of skin will determine the actual digestion time. Following digestion, microbiopsies and media are transferred to 50m conical tubes and centrifuged at 80 g for 30 seconds and enzyme digestion is terminated by adding surplus 5% serum-containing media for enzyme dilution. The DR-iTR factors LIN28A, OCT4, and KLF4 in an AAV9 gene therapy vector using the promoter sequence from COX7A1 disclosed herein together with another AAV9 vector expressing TERT are introduced with daily feeding for 14 days. Microbiopsies are then immersed in HyStem hydrogel prior to completion of crosslinking and transplanted on the backs of immunocompromised nude mice. Tissue is harvested at 1, 2, 4, and 8 weeks for histological analysis of regenerative effects.

### Example 2: The DR-iTR Factors KLF4, OCT4, LIN28A are Introduced into Nonregenerative Adult Cells by a Viral Gene Therapy Vector to Increase Regeneration

An In vitro assay of human cell regeneration through the modulation of DR-iTR genes is utilized. The regenerative potential of up-regulating i TR inducing genes or down-regulating iTR inhibitory genes is assayed using the in vitro wound repair assay described herein. In brief, a scratch test is utilized as described (Nature Protocols 2, 329-333 (2007) Liang C C et al “In vitro scratch assay: a convenient and inexpensive method for analysis of cell migration in vitro”). The assay utilizes neonatal human foreskin fibroblasts (Xgene Corp, Sausalito CA) that express the fetal/adult markers (iTR inhibitors) but not the embryonic markers (iTR factors) described herein (Table I The fibroblasts are grown to confluence using DMEM medium supplemented with 10% FBS cultured in 6 well plates previously coated with 0.1% gelatin then cultured in a humidified incubator with 5% O2 and 10% CO2.

On Day 0 the reagents below were used to alter the expression of the DR-iTR factor genes KLF4, OCT4, and LIN28A:
Lentiviral vector construction and transduction are carried out as described below with the cDNAs for the genes KL_F4, OCT4, and LIN28A with IRES sequences positioned as shown in FIG. 1 and cDNAs for the three genes in cis with the COX7A1 promoter described herein.
Lentiviral vector with the control gene Luciferase in cis with the COX7A1 promoter described herein

In this example, we describe the procedure for DR-iTR, containing the pFB-Luc retroviral vector (Stratagene, La Jolla, CA) stably integrated into the cellular genomic DNA. Luciferase levels and cell transduction efficiencies are determined by measuring luciferase activity in lysates of virus infected cells, by immunocytochemically staining cells for Luciferase expression, and by direct detection of luminescent cells in culture.

Transduction of Target Cells with a Viral Supernatant. This transduction is performed to demonstrate that cell lines are able to be transduced, that the viral supernatants are able to be transduced. and to assess the quality of the viral supernatants.

Day 1: Preparing for Transduction 1. X-gene dermal fibroblasts are seeded in 6 wells using 6-well tissue culture plates with 1×105 cells per well ˜20% confluency at the time of infection is desirable. 2. Return the plates to the 37° C. incubator overnight.

Day 2: Transducing the Target Cells Prior to thawing the viral supernatant the area around the cap should be carefully inspected for any sign of leakage, and thoroughly wiped with 70% ethanol. Media should be prepared and aliquoted into prelabeled Falcon® 2054 polystyrene tubes prior to thawing the virus. Quickly thaw the pFB-Luc supernatant (nominal titer approximately 2×107/ml) by rapid agitation in a 37° C. H2O bath. Screw caps should be removed in the hood only, and any fluid around the outside lip of the tube or the inside surface of the cap should be carefully wiped with a tissue wetted with 70% ethanol, and the tissue should be disposed of in the hood. Thawed virus should be temporarily stored on ice if not used immediately. Prepare a dilution series from 1:10 to 1:104 in growth medium (2.0 ml dilution per tube in 2054 tubes) supplemented with DEAE-dextran at a final concentration of 10 np/ml (1:1000 dilution of the 10 mg/ml DEAE-dextran stock). Add 0.8-10 ml undiluted supernatant to an additional tube. and supplement with DEAE-dextran to 10 μg/ml. 3. Remove the plates containing the target X-gene fibroblast cells from the incubator. 4. Remove and discard the medium from the wells. For tubes containing undiluted supernatant and for each dilution, add 1.0 ml per well of the KLF4, OCT4, LIN28A vector as well as the control vector to the X-gene fibroblasts. Add 1.0 nil media (no virus) to the sixth well for an uninfected control. The remaining supernatant should be aliquoted and refrozen at −80° C. It should be noted that the titer will drop, resulting in a loss of <50% of the remaining infectious particles with each subsequent freeze-thaw cycle. 5. Return the plates to the 37° C. incubator and incubate for 3 hours. 6. After the 3 hour incubation, add an additional 1.0 m growth medium to each well. 7. Return the plates to the 37° C. incubator and allow 24-72 hours for analysis of expression of the luciferase protein by luciferase assay, immunocytochemistry, or direct visualization of luminescent cells.

After 6 hours the plate is removed from the incubator and a “scratch” is made using a 200 ul pipet tip in the center of each well, Photos are taken at 4×. The plate is placed back into the incubator after 2× wash with PBS and fresh growth medium fed (3 ml/well. Photographs are taken to observed mobility. proliferation, and morphology of the cells were taken on D0, D1 and D2. RNA is extracted at days 2 and 4 for subsequent qPCR analysis.

### Example 3. In Vivo Induction of Developmentally-Regulated Toxic Genes in Cancerous Microbiopsies

Immunocompromised nude mice are implanted with a carcinoma tumor directed under their skin. Tumor are allowed to grow to about 50 mm³-100 mm³ in size. DR-O therapeutics, such as HSV TK, in an AAV9 gene therapy vector using the promoter sequence from CPT1B disclosed herein are introduced into the tumor via injection. Tumor size is monitored over 1, 2, 4, 8, 12, 16, and 18 weeks. Mortality is monitored over the course of the study. After 2-6 months, mice are euthanized and any remaining tumors are analyzed for presence of DR-O therapeutics and expression of HSV TK.

What is claimed is:

1. A method of reprogramming adult mammalian somatic cells to a scarless regenerative state, the method comprising contacting the cells with one or more induced tissue regeneration (iTR) factors that comprise: one or more nucleic acids encoding OCT4, SOX2, KLF4, NANOG, ESRRB, NR5A2, CEBPA, MYC, SALL4, LIN28A or LIN28B, wherein the one or more iTR factors are operably linked to a heterologous promoter or enhancer sequence, wherein the heterologous promoter or enhancer sequence induces expression temporally during embryonic, fetal, or neonatal developmental transitions.

2. The method of claim 1, wherein the heterologous promoter or enhancer sequence comprises a sequence that has at least 90%, 91%, 92%, 93%, 94%, 95%, 96%, 97%, 98%, or 99% identity to, comprises, or consists of any one of SEQ ID NOs: 1-15.

3. The method of claim 2, wherein the heterologous promoter or enhancer comprises a sequence that has at least 90%, 91%, 92%, 93%, 94%, 95%, 96%, 97%, 98%, or 99% identity to, comprises, or consists SEQ ID NO: 4.

4. The method of any one of claims 1-3, wherein the one or more iTR factors comprise (a) a nucleic acid encoding OCT4, SOX2, KLF4, and MYC; (b) one or more nucleic acids encoding OCT4, SOX2, KLF4, and MYC; (c) one or more nucleic acids encoding LIN28A, OCT4, and KLF4; or (d) one or more nucleic acids encoding LIN28A, OCT4, SOX2, and NANOG.

5. The method of any one of claims 1-4, wherein the mammal is human.

6. The method of any one of claims 1-5, wherein the one or more iTR factor genes are delivered by viral vector.

7. The method of claim 6, wherein the viral vector is an adeno-associated virus.

8. The method of claim 6 or claim 7, wherein the viral vector is present in a pharmaceutical composition.

9. The method of claim 8, wherein the pharmaceutical composition comprises a lipid formulation.

10. The method of claim 9, wherein the lipid formulation comprises one or more cationic lipids, non-cationic lipids, and/or PEG-lipids, or a combination thereof.

11. The method of any one of claims 1-10, wherein the somatic cells reside in microbiopsied tissue cultured in vitro.

12. A method of treating cancer in a mammal, a method comprising administering one or more toxic genes to cancer cells in the mammal, wherein the one or more toxic genes are operably linked to a heterologous promoter or enhancer sequence, wherein the heterologous promoter or enhancer sequence induces expression induces expression in embryonic cells but not adult cells, wherein the cancer cells have reverted to an embryonic state.

13. The method of claim 12, wherein the heterologous promoter or enhancer comprises a sequence that has at least 90%, 91%, 92%, 93%, 94%, 95%, 96%, 97%, 98%, or 99% identity to, comprises, or consists of any one of SEQ ID NOs: 21-35.

14. The method of claim 12 or 13, wherein the mammal is human.

15. The method of any one of claims 12-14, wherein the toxic gene product is simplex virus thymidine kinase (HSV TK).

16. The method of any one of claims 12-15, wherein the cancer is a carcinoma.

17. The method of any one of claims 12-16, wherein the one or more toxic genes are delivered by viral vector.

18. The method of any one of claims 12-17, wherein the viral vector is an adeno-associated viral vector.

19. The method of claim 17 or claim 18, wherein the viral vector is present in a pharmaceutical composition.

20. The method of claim 19, wherein the pharmaceutical composition comprises a lipid formulation.

21. The method of claim 20, wherein the lipid formulation comprises one or more cationic lipids, non-cationic lipids, and/or PEG-lipids, or a combination thereof.
